(19) United States
(12) Patent Application Publication
(10) Pub. No.: US 2012/0115918 A1
(43) Pub. Date: May 10, 2012

(54) Anti-Viral Compounds

(75) Inventors: David A. DeGoey, Salem, WI (US); Allan C. Krueger, Gurnee, IL (US); Charles W. Hutchins, Green Oaks, IL (US); Warren M. Kati, Gurnee, IL (US); William A. Carroll, Evanston, IL (US)

(73) Assignee: ABBOTT LABORATORIES, Abbott Park, IL (US)

(21) Appl. No.: 13/328,767

(22) Filed: Dec. 16, 2011

Related U.S. Application Data
(63) Continuation-in-part of application No. 12/759,986, filed on Apr. 14, 2010.
(60) Provisional application No. 61/423,900, filed on Dec. 16, 2010.
(60) Provisional application No. 61/169,449, filed on Apr. 15, 2009.
(60) Provisional application No. 61/222,591, filed on Jul. 2, 2009.

(51) Int. Cl. A61K 31/4184; A61K 31/4025; A61P 31/12; A61K 31/4178; C07D 235/04; C07D 403/12; C07D 403/14

(52) U.S. Cl. 514/394; 548/518; 514/422; 548/314.7; 514/397; 548/313.1; 548/305.4

(57) ABSTRACT

Compounds effective in inhibiting replication of Hepatitis C virus (“HCV”) are described. This invention also relates to processes of making such compounds, compositions comprising such compounds, and methods of using such compounds to treat HCV infection.

This application claims priority from U.S. Provisional Application No. 61/423,900, filed Dec. 16, 2010; this application is also a continuation-in-part of U.S. patent application Ser. No. 12/759,986, filed Apr. 14, 2010, which claims the benefit from U.S. Provisional Application No. 61/169,449, filed Apr. 15, 2009, and U.S. Provisional Application No. 61/222,591, filed Jul. 2, 2009. All of these applications are incorporated herein by reference in their entireties.

### FIELD

The present invention relates to compounds effective in inhibiting replication of Hepatitis C virus (“HCV”). The present invention also relates to compositions comprising these compounds and methods of using these compounds to treat HCV infection.

### BACKGROUND

HCV is an RNA virus belonging to the Hepacivirus genus in the Flaviviridae family. HCV has enveloped virions that contain a positive stranded RNA genome encoding all known virus-specific proteins in one single, uninterrupted, open reading frame. The open reading frame comprises approximately 9500 nucleotides encoding a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects and viral elimination from the body is often inadequate. Therefore, there is a need for new drugs to treat HCV infection.

### SUMMARY

The present invention features compounds of Formulae I, I_{A}, I_{B}, I_{C}, I_{D}, I_{E}, I_{F}, I_{G}, I_{H} and I_{I}, and pharmaceutically acceptable salts thereof. These compounds and salts are capable of inhibiting the replication of HCV and therefore can be used to treat HCV infection.

The present invention also features compositions comprising the compounds or salts of the present invention. The compositions can also include other therapeutic agents, such as HCV helicase inhibitors, HCV polymerase inhibitors, HCV protease inhibitors, HCV NS5A inhibitors, CD81 inhibitors, cyclophilin inhibitors, or internal ribosome entry site (IRES) inhibitors.

The present invention further features methods of using the compounds or salts of the present invention to inhibit HCV replication. The methods comprise contacting cells infected with HCV virus with a compound or salt of the present invention, thereby inhibiting the replication of HCV virus in the cells.

In addition, the present invention features methods of using the compounds or salts of the present invention, or compositions comprising the same, to treat HCV infection. The methods comprise administering a compound or salt of the present invention, or a pharmaceutical composition comprising the same, to a patient in need thereof, thereby reducing the blood or tissue level of HCV virus in the patient.

The present invention also features use of the compounds or salts of the present invention for the manufacture of medicaments for the treatment of HCV infection.

Furthermore, the present invention features processes of making the compounds or salts of the invention.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

The present invention features compounds having Formula I, and pharmaceutically acceptable salts thereof,

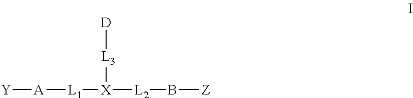

wherein:
X is C(H) and is optionally substituted with R_{A} or R_{F};
L₁ and L₂ are each independently selected from bond; or C₁-C₆alkylene, C₂-C₆alkenylene or C₂-C₆alkynylene, each of which is independently optionally substituted at each occurrence with one or more R_{L};
L₃ is bond or -L_{S}-K-L_{S}′-, wherein K is selected from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—;
A and B are each independently C₃-C₁₂carbocycle or 3- to 12-membered heterocycle, and are each independently optionally substituted with one or more R_{A};
D is C₃-C₁₂carbocycle or 3- to 12-membered heterocycle, and is optionally substituted with one or more R_{A}; or D is C₃-C₁₂carbocycle or 3- to 12-membered heterocycle which is substituted with J and optionally substituted with one or more R_{A}, where J is C₃-C₁₂carbocycle or 3- to 12-membered heterocycle and is optionally substituted with one or more R_{A}, or J is —SF₅; or D is hydrogen or R_{A};
Y is selected from -T′-C(R₁R₂)N(R₅)-T-R_{D}, -T′-C(R₃R₄)C(R₆R₇)-T-R_{D}, -L_{K}-T-R_{D}, or -L_{K}-E;
R₁ and R₂ are each independently R_{C}, and R₅ is R_{B}; or R₁ is R_{C}, and R₂ and R₅, taken together with the atoms to which they are attached, form a 3- to 12-membered heterocycle which is optionally substituted with one or more R_{A};
R₃, R₄, R₆, and R₇ are each independently R_{C}; or R₃ and R₆ are each independently R_{C}, and R₄ and R₇, taken together with the atoms to which they are attached, form a 3- to 12-membered carbocycle or heterocycle which is optionally substituted with one or more R_{A};
Z is selected from -T′-C(R₈R₉)N(R₁₂)-T-R_{D}, -T′-C(R₁₀R₁₁)C(R₁₃R₁₄)-T-R_{D}, -L_{K}-T—R_{D}, or -L_{K}-E;
R₈ and R₉ are each independently R_{C}, and R₁₂ is R_{B}; or R₈ is R_{C}, and R₉ and R₁₂, taken together with the atoms to which they are attached, form a 3- to 12-membered heterocycle which is optionally substituted with one or more R_{A};
R₁₀, R₁₁, R₁₃, and R₁₄ are each independently R_{C}; or R₁₀ and R₁₃ are each independently R_{C}, and R₁₁ and R₁₄, taken together with the atoms to which they are attached, form a 3- to 12-membered carbocycle or heterocycle which is optionally substituted with one or more R_{A};
T and T′ are each independently selected at each occurrence from bond, -L_{S}-, -L_{S}-M-L_{S}′-, or -L_{S}-M-L_{S}′-M′-L_{S}″-, wherein M and M′ are each independently selected at each occurrence from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₂carbocycle or 3- to 12-membered heterocycle, and wherein said C₃-C₁₂carbocycle and 3- to 12-membered heterocycle are each independently optionally substituted at each occurrence with one or more R_{A};
L_{K} is independently selected at each occurrence from bond, -L_{S}-N(R_{B})C(O)-L_{S}′- or -L_{S}-C(O)N(R_{B})-L_{S}′-; or C₁-C₆alkylene, C₂-C₆alkenylene or C₂-C₆alkynylene, each of which is independently optionally substituted at each occurrence with one or more R_{L}; or C₃-C₁₂carbocycle or 3- to 12-membered heterocycle, each of which is independently optionally substituted at each occurrence with one or more R_{A};
E is independently selected at each occurrence from C₃-C₁₂carbocycle or 3- to 12-membered heterocycle, and is independently optionally substituted at each occurrence with one or more R_{A};
R_{D} is each independently selected at each occurrence from hydrogen or R_{A};
R_{A} is independently selected at each occurrence from halogen, nitro, oxo, phosphonoxy, phosphono, thioxo, cyano, or -L_{S}-R_{E}, wherein two adjacent R_{A}, taken together with the atoms to which they are attached and any atoms between the atoms to which they are attached, can optionally form carbocycle or heterocycle;
R_{B} and R_{B}′ are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{C} is independently selected at each occurrence from hydrogen, halogen, hydroxy, mercapto, amino, carboxy, nitro, oxo, phosphonoxy, phosphono, thioxo, formyl or cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{C} is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S}, or —N(R_{S}R_{S}′);
R_{F} is independently selected at each occurrence from C₁-C₁₀alkyl, C₂-C₁₀alkenyl or C₂-C₁₀alkynyl, each of which contains 0, 1, 2, 3, 4 or 5 heteroatoms selected from O, S or N and is independently optionally substituted with one or more R_{L}; or —(R_{X}—R_{Y})_{Q}—(R_{X}—R_{Y}′), wherein Q is 0, 1, 2, 3 or 4, and each R_{X} is independently O, S or N(R_{B}), wherein each R_{Y} is independently C₁-C₆alkylene, C₂-C₆alkenylene or C₂-C₆alkynylene each of which is independently optionally substituted with one or more substituents selected from halogen, hydroxy, mercapto, amino, carboxy, nitro, oxo, phosphonoxy, phosphono, thioxo, formyl or cyano, and wherein each R_{Y}′ is independently C₁-C₆alkyl, C₂-C₆alkenyl or C₂-C₆alkynyl each of which is independently optionally substituted with one or more substituents selected from halogen, hydroxy, mercapto, amino, carboxy, nitro, oxo, phosphonoxy, phosphono, thioxo, formyl or cyano;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; wherein two adjacent R_{L}, taken together with the atoms to which they are attached and any atoms between the atoms to which they are attached, can optionally form carbocycle or heterocycle;
L_{S}, L_{S}′ and L_{S}″ are each independently selected at each occurrence from bond; or C₁-C₆alkylene, C₂-C₆alkenylene or C₂-C₆alkynylene, each of which is independently optionally substituted at each occurrence with one or more R_{L}; and
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene —O—C₁-C₆alkyl, or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Preferably, Formula I encompasses compounds, wherein:
A and B are each independently C₃-C₁₀carbocycle or 3- to 10-membered heterocycle, and are each independently optionally substituted with one or more R_{A};
D is C₃-C₁₀carbocycle or 3- to 10-membered heterocycle, and is optionally substituted with one or more R_{A}; or D is R_{D}; or D is C₃-C₁₀carbocycle or 3- to 10-membered heterocycle which is substituted with J and optionally substituted with one or more R_{A}, where J is C₃-C₁₀carbocycle or 3- to 10-membered heterocycle and is optionally substituted with one or more R_{A}, or J is —SF₅; or preferably, D is C₅-C₆carbocycle, 5- to 6-membered heterocycle or 6- to 10-membered bicycle, and is substituted with J and optionally substituted with one or more R_{A}, and J is C₃-C₆carbocycle or 3- to 6-membered heterocycle and is optionally substituted with one or more R_{A}; or more preferably, D is C₅-C₆carbocycle or 5- to 6-membered heterocycle, and is substituted with J and optionally substituted with one or more R_{A}, and J is C₃-C₆carbocycle or 3- to 6-membered heterocycle and is optionally substituted with one or more R_{A}; or highly preferably, D is phenyl substituted with J and optionally substituted with one or more R_{A}, where J is C₃-C₆carbocycle or 3- to 6-membered heterocycle and is
   optionally substituted with one or more R_{A}; or D is

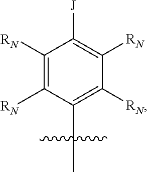

wherein each R_{N} is independently selected from R_{D} and preferably is hydrogen, and J is as defined above and preferably is C₃-C₆carbocycle or 3- to 6-membered heterocycle optionally substituted with one or more R_{A}; or D is

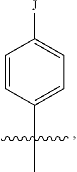

and J is C₃-C₆carbocycle or 3- to 6-membered heterocycle and is optionally substituted with one or more R_{A}
X is C(R_{C});
L₁ and L₂ are each independently selected from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
L₃ is bond or -L_{S}-K-L_{S}′-, wherein K is selected from a bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—;
Y is selected from -T′-C(R₁R₂)N(R₅)-T-R_{D}, -T′-C(R₃R₄)C(R₆R₇)-T-R_{D}, -L_{K}-T-R_{D}, or -L_{K}-E;
R₁ and R₂ are each independently R_{C}, and R₅ is R_{B}; or R₁ is R_{C}, and R₂ and R₅, taken together with the atoms to which they are attached, form a 3- to 10-membered heterocyclic ring (e.g., a 3- to 8-membered heterocyclic) which is optionally substituted with one or more R_{A};
R₃, R₄, R₆, and R₇ are each independently R_{C}; or R₃ and R₆ are each independently R_{C}, and R₄ and R₇, taken together with the atoms to which they are attached, form a 3- to 10-membered carbocyclic or heterocyclic ring (e.g., a 3- to 8-membered carbocyclic or heterocyclic ring) which is optionally substituted with one or more R_{A};
Z is selected from -T′-C(R₈R₉)N(R₁₂)-T-R_{D}, -T′-C(R₁₀R₁₁)C(R₁₃R₁₄)-T-R_{D}, -L_{K}-T-R_{D}, or -L_{K}-E;
R₈ and R₉ are each independently R_{C}, and R₁₂ is R_{B}; or R₈ is R_{C}, and R₉ and R₁₂, taken together with the atoms to which they are attached, form a 3- to 8-membered heterocyclic ring which is optionally substituted with one or more R_{A};
R₁₀, R₁₁, R₁₃, and R₁₄ are each independently R_{C}; or R₁₀ and R₁₃ are each independently R_{C}, and R₁₁ and R₁₄, taken together with the atoms to which they are attached, form a 3- to 8-membered carbocyclic or heterocyclic ring which is optionally substituted with one or more R_{A};
L_{K} is independently selected at each occurrence from a bond; —N(R_{B})C(O)-L_{S}-; —C(O)N(R_{B})-L_{S}-; or C₁-C₆alkylene, C₂-C₆alkenylene, C₂-C₆alkynylene, C₃-C₁₀carbocycle or 3- to 10-membered heterocycle, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
E is independently selected at each occurrence from C₃-C₁₀carbocycle or 3- to 10-membered heterocycle, and is independently optionally substituted at each occurrence with one or more R_{A};
T and T′ are each independently selected at each occurrence from a bond, -L_{S}-, -L_{S}-M-L_{S}′-, -L_{S}-M-L_{S}′-M′-L_{S}″-, wherein M and M′ are each independently selected at each occurrence from a bond, —O—, —S—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₀carbocycle, or 3- to 10-membered heterocycle, and wherein said C₃-C₁₀carbocycle and 3- to 10-membered heterocycle are each independently optionally substituted at each occurrence with one or more R_{A};
R_{A} is independently selected at each occurrence from halogen, hydroxy, mercapto, amino, carboxy, nitro, oxo, phosphonoxy, phosphono, thioxo, formyl, cyano, -L_{A}, or -L_{S}-R_{E}, wherein two adjacent R_{A}, taken together with the atoms to which they are attached and any atoms between the atoms to which they are attached, optionally form a C₃-C₁₀carbocycle or 3- to 10-membered heterocycle;
R_{B} and R_{B}′ are each independently selected at each occurrence from hydrogen or R_{F};
R_{C} is independently selected at each occurrence from hydrogen, halogen, hydroxy, mercapto, amino, carboxy, nitro, oxo, phosphonoxy, phosphono, thioxo, formyl, cyano, or R_{F};
R_{D} is each independently selected at each occurrence from hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, C₃-C₁₀carbocyclyl, or 3- to 10-membered heterocyclyl, wherein said C₃-C₁₀carbocyclyl and 3- to 10-membered heterocyclyl are each independently optionally substituted at each occurrence with one or more substituents selected from halogen, R_{T}, —O—R_{B}, —S—R_{B}, —N(R_{B}R_{B}), —OC(O)R_{B}, —C(O)OR_{B}, nitro, phosphonoxy, phosphono, oxo, thioxo, formyl or cyano;
R_{F} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl or (3- or 6-membered heterocyclyl)C₁-C₆alkyl, each of which is independently optionally substituted at each occurrence with one or more substituents selected from halogen, hydroxy, mercapto, amino, carboxy, nitro, oxo, phosphonoxy, phosphono, thioxo, formyl or cyano;
L_{A} is independently selected at each occurrence from C₁-C₆alkyl, C₂-C₆alkenyl, or C₂-C₆alkynyl, each of which is independently optionally substituted at each occurrence with one or more substituents selected from halogen, R_{T}, —O—R_{S}, —N(R_{S}R_{S}′), —OC(O)R_{S}, —C(O)OR_{S}, nitro, phosphonoxy, phosphono, oxo, thioxo, formyl or cyano;
L_{S}, L_{S}′ and L_{S}″ are each independently selected at each occurrence from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
R_{S}, R_{S}′ and R_{S}″ are each independently selected at each occurrence from hydrogen or R_{T};
R_{T} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl, or (3- or 6-membered heterocyclyl)C₁-C₆alkyl, each of which is independently optionally substituted at each occurrence with one or more substituents selected from halogen, R_{F}, —O—R_{B}, —S—R_{B}, —N(R_{B}R_{B}′), —OC(O)R_{B}, —C(O)OR_{B}, nitro, oxo, phosphonoxy, phosphono, thioxo, formyl or cyano.

A and B preferably are independently selected from C₅-C₆carbocycle (e.g., phenyl), 5- to 6-membered heterocycle (e.g., pyridinyl or thiazolyl), or 8- to 12-membered bicycles such as

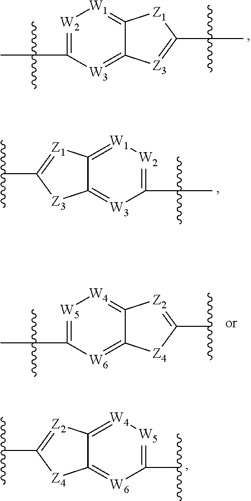

where Z₁ is independently selected at each occurrence from O, S, NH or CH₂, Z₂ is independently selected at each occurrence from N or CH, Z₃ is independently selected at each occurrence from N or CH, Z₄ is independently selected at each occurrence from O, S, NH or CH₂, and W₁, W₂, W₃, W₄, W₅ and W₆ are each independently selected at each occurrence from CH or N. A and B are each independently optionally substituted with one or more R_{A}.

More preferably, A is selected from C₅-C₆carbocycle, 5- to 6-membered heterocycle,

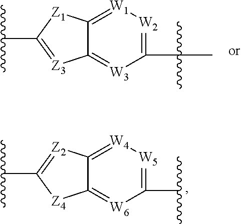

and is optionally substituted with one or more R_{A}; B is selected from C₅-C₆carbocycle, 5- to 6-membered heterocycle,

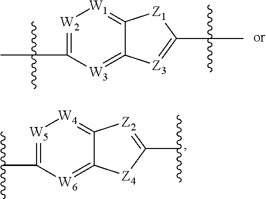

and is optionally substituted with one or more R_{A}; where Z₁, Z₂, Z₃, Z₄, W₁, W₂, W₃, W₄, W₅, W₆ are as defined above. Preferably, Z₃ is N and Z₄ is NH. For instance, A can be selected from

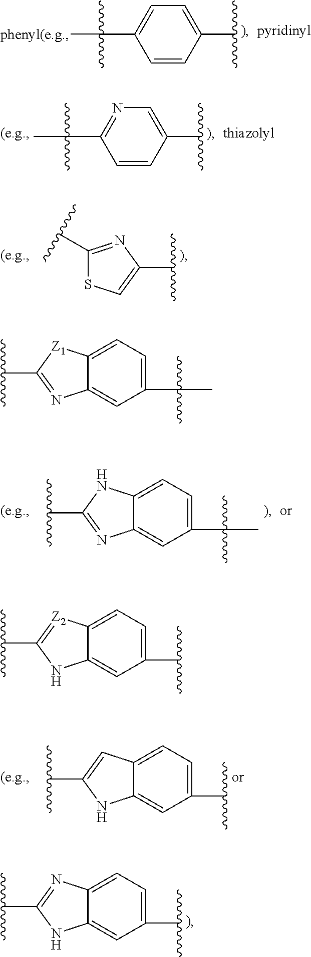

and is optionally substituted with one or more R_{A}; and B can be selected from

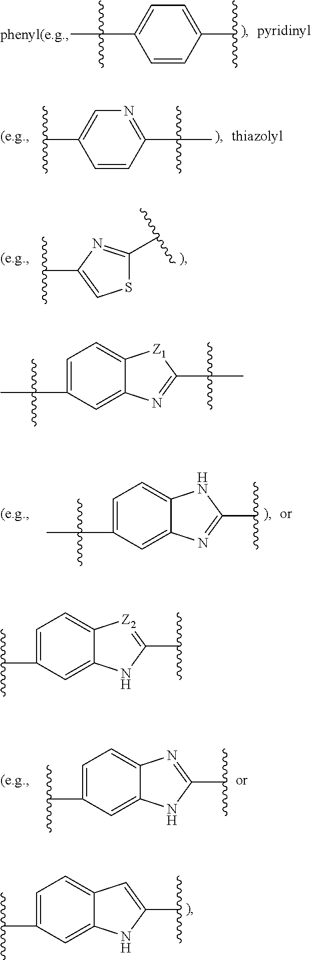

and is optionally substituted with one or more R_{A}. Highly preferably, both A and B are phenyl (e.g., both A and B are

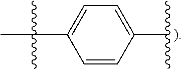

Also highly preferably,

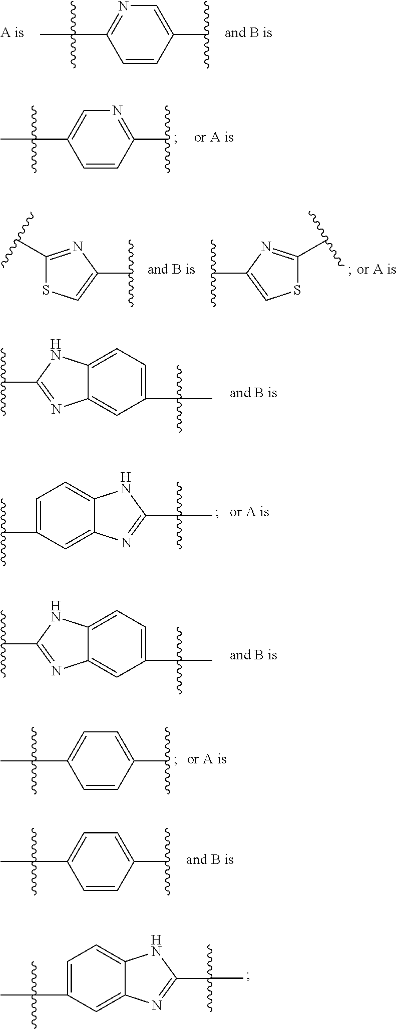

wherein each A and B is independently optionally substituted with one or more R_{A}.

D preferably is selected from C₅-C₆carbocycle, 5- to 6-membered heterocycle, or 6- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more substituents selected from R_{L}. More preferably, D is C₅-C₆carbocycle (e.g., phenyl), 5- to 6-membered heterocycle (e.g., pyridinyl, pyrimidinyl, thiazolyl), or 6- to 12-membered bicycles (e.g., indanyl, 4,5,6,7-tetrahydrobenzo[d]thiazolyl, benzo[d]thiazolyl, indazolyl, benzo[d][1,3]dioxol-5-yl), and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

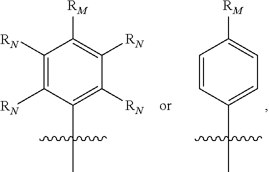

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

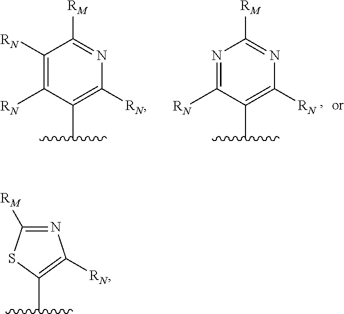

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

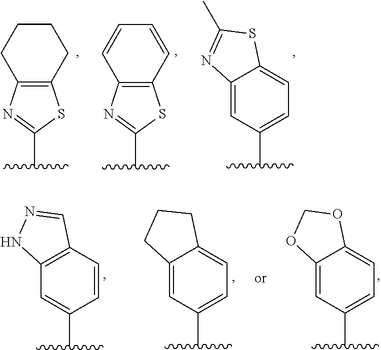

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M} is -L_{S}-R_{E} where L_{S} is a bond and R_{E} is —N(R_{S}R_{S′}), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or spiro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D is

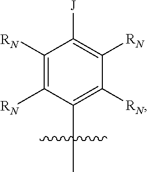

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is

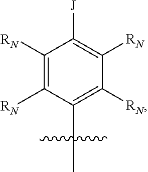

wherein each R_{N} is independently selected from R_{D} and preferably is hydrogen or halogen, and J is C₃-C₆carbocycle or 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

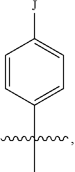

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

X preferably is C(H).

L₁ and L₂ are preferably independently bond or C₁-C₆alkylene, L₃ is preferably selected from bond, C₁-C₆alkylene or —C(O)—, and L₁, L₂, and L₃ are each independently optionally substituted with one or more R_{L}, and wherein at least one of L₁ or L₂ preferably is bond. More preferably, L₁, L₂ and L₃ are each independently bond or C₁-C₆alkylene (e.g., —CH₂— or —CH₂CH₂—), and are each independently optionally substituted with one or more R_{L}, and wherein at least one of L₁ or L₂ preferably is bond. Highly preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond.

Y is preferably selected from -L_{S}-C(R₁R₂)N(R₅)-T-R_{D}, -L_{S}-C(R₃R₄)C(R₆R₇)-T-R_{D}, -G-C(R₁R₂)N(R₅)-T-R_{D}, -G-C(R₃R₄)C(R₆R₇)-T-R_{D}, —N(R_{B})C(O)C(R₁R₂)N(R₅)-T-R_{D}, —N(R_{B})C(O)C(R₃R₄)C(R₆R₇)-T-R_{D}, —C(O)N(R_{B})C(R₁R₂)N(R₅)-T-R_{D}, —C(O)N(R_{B})C(R₃R₄)C(R₆R₇)-T-R_{D}, —N(R_{B})C(O)-L_{S}-E, or —C(O)N(R_{B})-L_{S}-E. G is C₅-C₆carbocycle or 5- to 6-membered heterocycle, such as

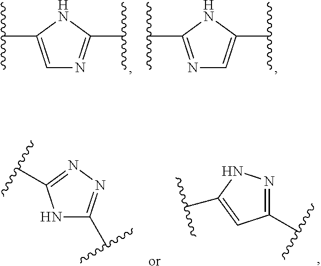

and is optionally substituted with one or more R_{A} (e.g., one or more chloro or bromo). E preferably is a 7- to 12-membered bicycle (such as

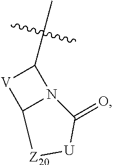

wherein U is independently selected at each occurrence from —(CH₂)— or —(NH)—; V and Z₂₀ are each independently selected from C₁-C₄alkylene, C₂-C₄alkenylene or C₂-C₄alkynylene, in which at least one carbon atom can be independently optionally replaced with O, S or N), and is independently optionally substituted with one or more R_{A}. More preferably, R₁ is R_{C}, and R₂ and R₅, taken together with the atoms to which they are attached, form a 5- to 6-membered heterocycle or 6- to 12-membered bicycle

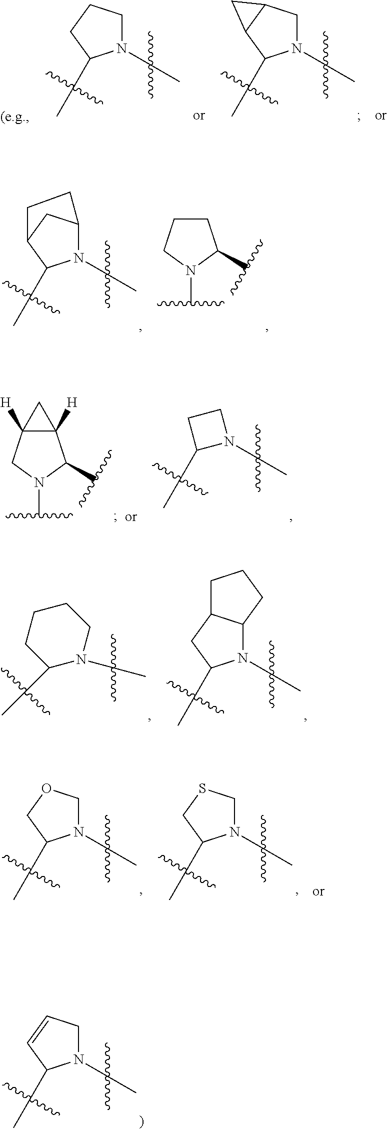

which is optionally substituted with one or more R_{A} (such as, but not limited to hydroxy, halo (e.g., fluoro), C₁-C₆alkyl (e.g., methyl), or C₂-C₆alkenyl (e.g., allyl)); and R₃ and R₆ are each independently R_{C}, and R₄ and R₇, taken together with the atoms to which they are attached, form a 5- to 6-membered carbocycle/heterocycle or 6- to 12-membered bicycle

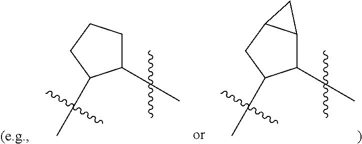

which is optionally substituted with one or more R_{A} (such as, but not limited to hydroxy, halo (e.g., fluoro), C₁-C₆alkyl (e.g., methyl), or C₂-C₆alkenyl (e.g., allyl)).

Y can also be selected from -M-C(R₁R₂)N(R₅)—C(O)-L_{Y}′-M′-R_{D}, -M-C(R₁R₂)N(R₅)-L_{Y}′-M′-R_{D}, -L_{S}-C(R₁R₂)N(R₅)—C(O)-L_{Y}′-M′-R_{D}, -L_{S}-C(R₁R₂)N(R₅)-L_{Y}′-M′-R_{D}, -M-C(R₃R₄)C(R₆R₇)—C(O)-L_{Y}′-M′-R_{D}, -M-C(R₃R₄)C(R₆R₇)-L_{Y}′-M′-R_{D}, -L_{S}-C(R₃R₄)C(R₆R₇)—C(O)-L_{Y}′-M′-R_{D}, or -L_{S}-C(R₃R₄)C(R₆R₇)-L_{Y}′-M′-R_{D}, wherein M preferably is bond, —C(O)N(R_{B})— or —N(R_{B})C(O)—, M′ preferably is bond, —C(O)N(R_{B})—, —N(R_{B})C(O)—, —N(R_{B})C(O)O—, N(R_{B})C(O)N(R_{B}′)—, —N(R_{B})S(O)— or —N(R_{B})S(O)₂—, and L_{Y}′ preferably is C₁-C₆alkylene which is optionally substituted with one or more R_{L}. L_{Y}′, for example, is a C₁-C₆alkylene such as, but not limited to,

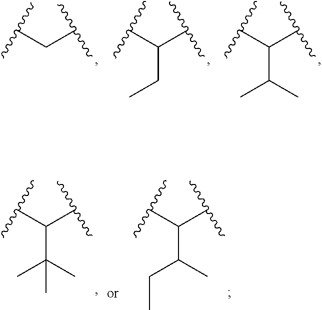

and the optional R_{L} is a substituent such as, but not limited to phenyl, —SMe, or methoxy. Any stereochemistry at a carbon within the group L_{Y}′ can be either (R) or (S). More preferably, R₁ is R_{C}, and R₂ and R₅, taken together with the atoms to which they are attached, form a 5- to 6-membered heterocycle or 6- to 12-membered bicycle

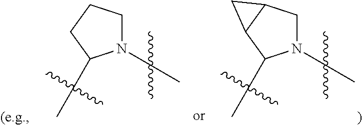

which is optionally substituted with one or more R_{A} (e.g., one or more hydroxy); and R₃ and R₆ are each independently R_{C}, and R₄ and R₇, taken together with the atoms to which they are attached, form a 5- to 6-membered carbocycle/heterocycle or 6- to 12-membered bicycle

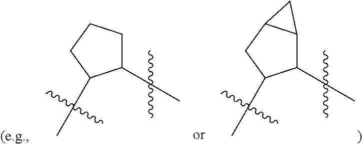

which is optionally substituted with one or more R_{A}.

Also preferably, Y is selected from —N(R_{B})CO—C(R₁R₂)N(R₅)—C(O)-L_{Y}′-N(R_{B})C(O)O—R_{D}, —N(R_{B})CO—C(R₁R₂)N(R₅)—C(O)-L_{Y}′-N(R_{B})C(O)—R_{D}, —N(R_{B})CO—C(R₁R₂)N(R₅)—C(O)-L_{Y}′-N(R_{B})S(O)₂—R_{D}, —N(R_{B})CO—C(R₁R₂)N(R₅)—C(O)-L_{Y}′-N(R_{B}R_{B}′)—R_{D}, —N(R_{B})CO—C(R₁R₂)N(R₅)—C(O)-L_{Y}′-O—R_{D}, —N(R_{B})CO—C(R₁R₂)N(R₅)—C(O)-L_{Y}′-R_{D}, —N(R_{B})CO—C(R₁R₂)N(R₅)—R_{D}, -L_{S}-C(R₁R₂)N(R₅)—C(O)-L_{Y}′-N(R_{B})C(O)O—R_{D}, -L_{S}-C(R₁R₂)N(R₅)—C(O)-L_{Y}′-N(R_{B})C(O)—R_{D}, -L_{S}-C(R₁R₂)N(R₅)—C(O)-L_{Y}′-N(R_{B})S(O)₂—R_{D}, -L_{S}-C(R₁R₂)N(R₅)—C(O)-L_{Y}′-N(R_{B}R_{B}′)—R_{D}, -L_{S}-C(R₁R₂)N(R₅)—C(O)-L_{Y}′-O—R_{D}, -L_{S}-C(R₁R₂)N(R₅)—C(O)-L_{Y}′-R_{D}, -L_{S}-C(R₁R₂)N(R₅)—R_{D}, —N(R_{B})CO—C(R₃R₄)C(R₆R₇)—C(O)-L_{Y}′-N(R_{B})C(O)O—R_{D}, —N(R_{B})CO—C(R₃R₄)C(R₆R₇)—C(O)-L_{Y}′-N(R_{B})C(O)—R_{D}, —N(R_{B})CO—C(R₃R₄)C(R₆R₇)—C(O)-L_{Y}′-N(R_{B})S(O)₂—R_{D}, —N(R_{B})CO—C(R₃R₄)C(R₆R₇)—C(O)-L_{Y}′-N(R_{B}R_{B}′)—R_{D}, —N(R_{B})CO—C(R₃R₄)C(R₆R₇)—C(O)-L_{Y}′-O—R_{D}, —N(R_{B})CO—C(R₃R₄)C(R₆R₇)—C(O)-L_{Y}′-R_{D}, —N(R_{B})CO—C(R₃R₄)C(R₆R₇)—R_{D}, -L_{S}-C(R₃R₄)C(R₆R₇)—C(O)-L_{Y}′-N(R_{B})C(O)O—R_{D}, -L_{S}-C(R₃R₄)C(R₆R₇)—C(O)-L_{Y}′-N(R_{B})C(O)—R_{D}, -L_{S}-C(R₃R₄)C(R₆R₇)—C(O)-L_{Y}′-N(R_{B})S(O)₂—R_{D}, -L_{S}-C(R₃R₄)C(R₆R₇)—C(O)-L_{Y}′-N(R_{B}R_{B}′)—R_{D}, -L_{S}-C(R₃R₄)C(R₆R₇)—C(O)-L_{Y}′-O—R_{D}, -L_{S}-C(R₃R₄)C(R₆R₇)—C(O)-L_{Y}′-R_{D}, or -L_{S}-C(R₃R₄)C(R₆R₇)—R_{D}, wherein L_{Y}′ preferably is C₁-C₆alkylene which is optionally substituted with one or more R_{L}. R₁ may be R_{C}, and R₂ and R₅, taken together with the atoms to which they are attached, may form a 5- to 6-membered heterocycle or 6- to 12-membered bicycle

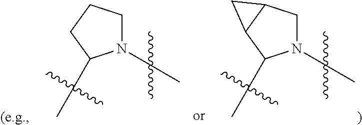

which is optionally substituted with one or more R_{A}; and R₃ and R₆ may be each independently R_{C}, and R₄ and R₇, taken together with the atoms to which they are attached, may form a 5- to 6-membered carbocycle/heterocycle or 6- to 12-membered bicycle

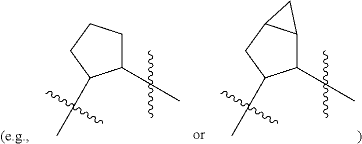

which is optionally substituted with one or more R_{A}.

Highly preferably, Y is selected from —N(R_{B}″)CO—C(R₁R₂)N(R₅)—C(O)-L_{Y}-N(R_{B}″)C(O)-L_{S}-R_{E} or —C(R₁R₂)N(R₅)—C(O)-L_{Y}-N(R_{B}″)C(O)-L_{S}-R_{E}, or Y is -G-C(R₁R₂)N(R_{S})—C(O)-L_{Y}-N(R_{B}″)C(O)-L_{S}-R_{E}, wherein L_{Y} is C₁-C₆alkylene optionally substituted with one or more R_{L}, and R_{B}″ is each independently R_{B}. R_{B}″ and R₁ are each preferably hydrogen or C₁-C₆alkyl, and R₂ and R₅, taken together with the atoms to which they are attached, preferably form a 5- to 6-membered heterocycle or 6- to 12-membered bicycle

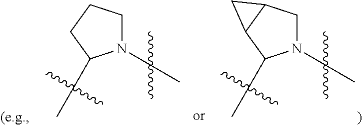

which is optionally substituted with one or more R_{A} (such as, but not limited to hydroxy, halo (e.g., fluoro), C₁-C₆alkyl (e.g., methyl), or C₂-C₆alkenyl (e.g., allyl)). Preferably, L_{Y} is C₁-C₆alkylene substituted with one or more R_{L} such as a C₃-C₆carbocycle 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, L_{Y} is a C₁-C₆alkylene such as, but not limited to,

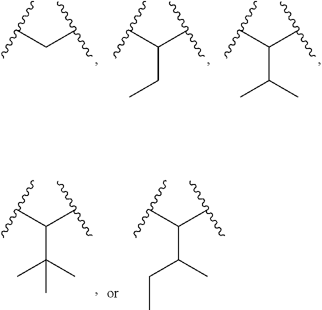

(stereochemistry at a carbon within the group L_{Y} can be either (R) or (S)), L_{Y} is independently optionally substituted with one or more R_{L}, (e.g., one or more phenyl or methoxy), G preferably is

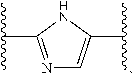

R_{B}″ is hydrogen; —C(R₇R₂)N(R_{S})— is

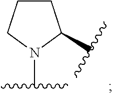

L_{S} is a bond; and R_{E} is methoxy.

Non-limiting examples of preferred Y include:

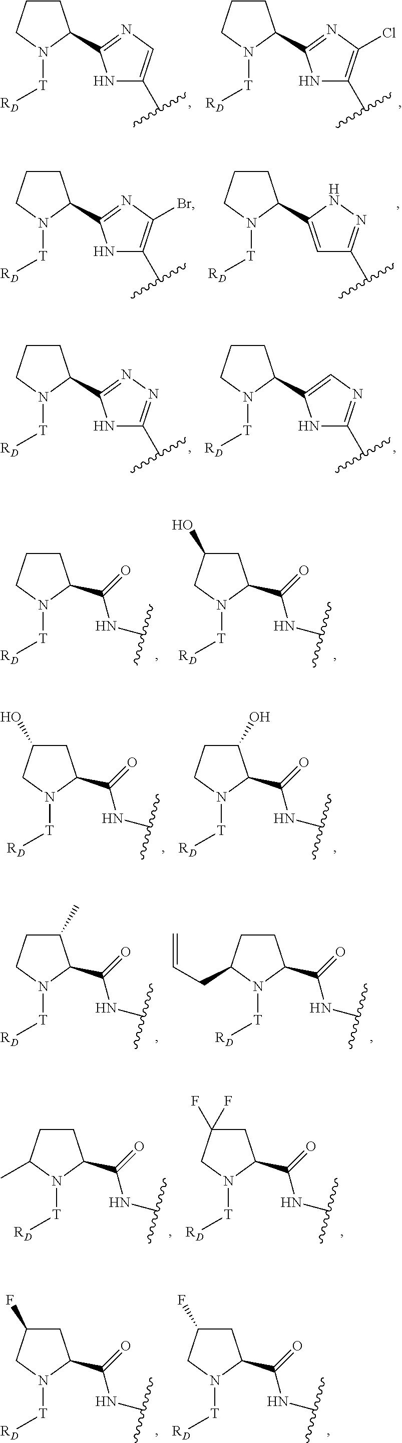

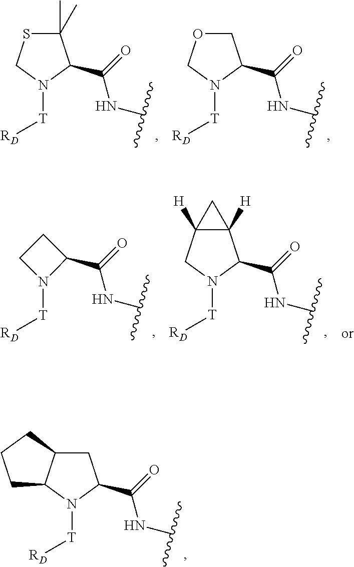

wherein T and R_{D} are as defined herein. T, for example, can be -L_{S}-M-L_{S}′-M′-L_{S}″- where L_{S} is a bond; M is C(O); L_{S}′ is C₁-C₆alkylene such as, but not limited to,

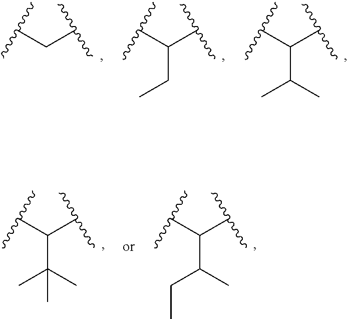

where L_{S}′ is independently optionally substituted with one or more R_{L}; R_{L} is a substituent such as, but not limited to phenyl or methoxy; M′ is —NHC(O)— or —NMeC(O)—; and L_{S}″ is a bond. Any stereochemistry at a carbon within the group L_{S}′ can be either (R) or (S). R_{D}, for example is methoxy. T-R_{D} includes, but is not limited to:

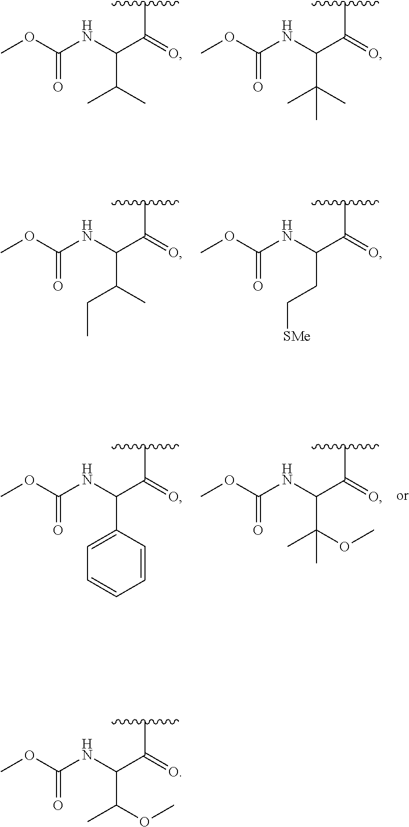

T-R_{D} may also include certain stereochemical configurations; thus T-R_{D} includes, but is not limited to:

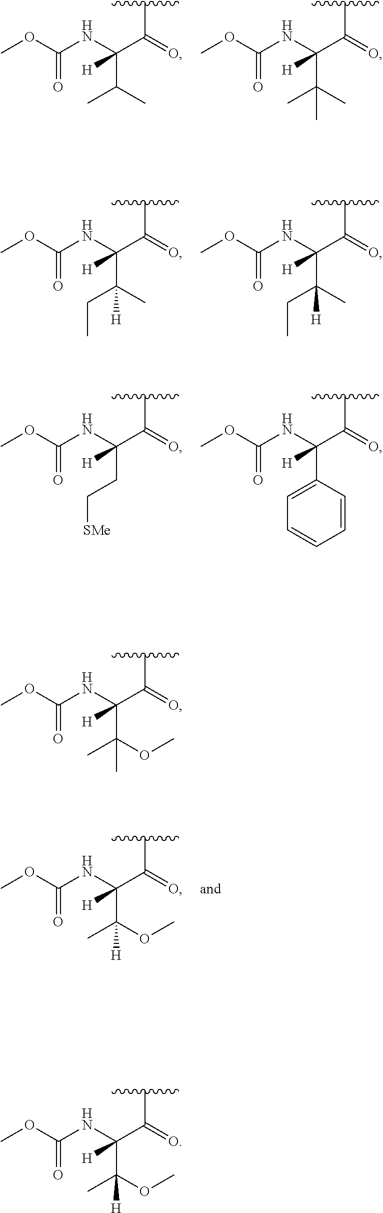

Non-limiting examples of preferred Y also include:

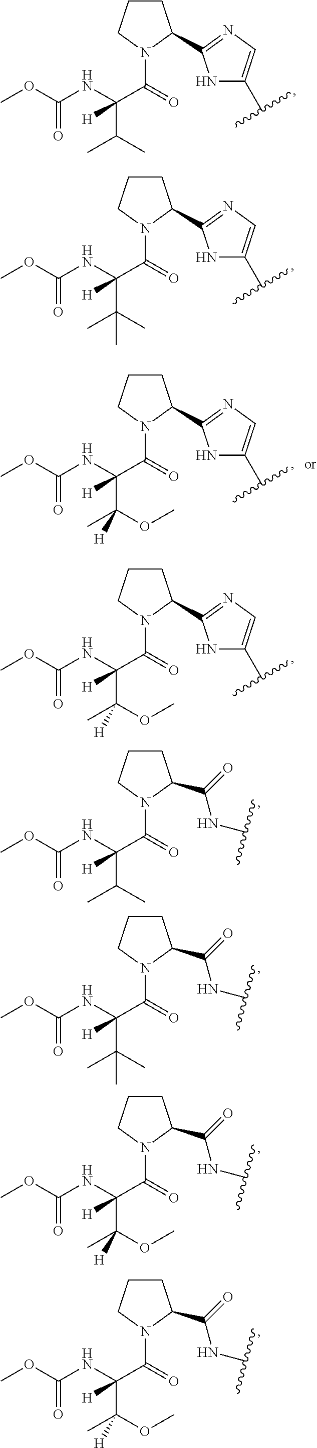

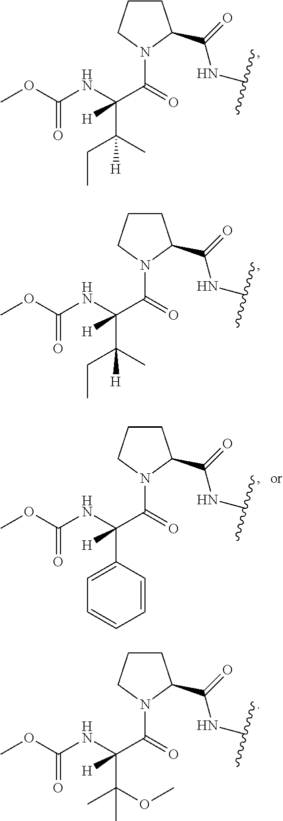

Z is preferably selected from -L_{S}-C(R₈R₉)N(R₁₂)-T-R_{D}, -L_{S}-C(R₁₀R₁₁)C(R₁₃R₁₄)-T-R_{D}, -G-C(R₈R₉)N(R₁₂)-T-R_{D}, -G-C(R₁₀R₁₁)C(R₁₃R₁₄)-T-R_{D}, —N(R_{B})C(O)C(R₈R₉)N(R₁₂)-T-R_{D}, —N(R_{B})C(O)C(R₁₀R₁₁)C(R₁₃R₁₄)-T-R_{D}, —C(O)N(R_{B})C(R₈R₉)N(R₁₂)-T-R_{D}, —C(O)N(R_{B})C(R₁₀R₁₁)C(R₁₃R₁₄)-T-R_{D}, —N(R_{B})C(O)-L_{S}-E, or —C(O)N(R_{B})-L_{S}-E. G is C₅-C₆carbocycle or 5- to 6-membered heterocycle, such as

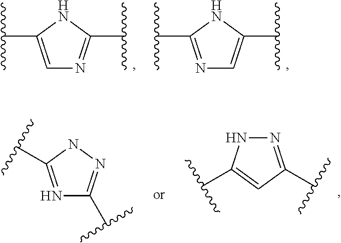

and is optionally substituted with one or more R_{A} (e.g., one or more chloro or bromo). E preferably is a 8- to 12-membered bicycle (such as

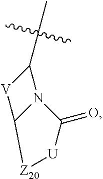

wherein U is independently selected at each occurrence from —(CH₂)— or —(NH)—; and V and Z₂₀ are each independently selected from C₁-C₄alkylene, C₂-C₄alkenylene or C₂-C₄alkynylene, in which at least one carbon atom is independently optionally replaced with O, S or N), and is independently optionally substituted with one or more R_{A}. More preferably, R₈ is R_{C}, and R₉ and R₁₂, taken together with the atoms to which they are attached, form a 5- to 6-membered heterocycle or 6- to 12-membered bicycle

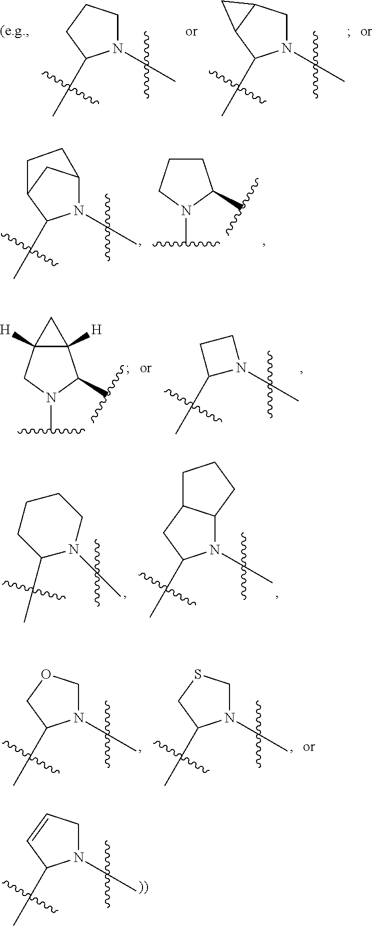

which is optionally substituted with one or more R_{A} (such as, but not limited to hydroxy, halo (e.g., fluoro), C₁-C₆alkyl (e.g., methyl), or C₂-C₆alkenyl (e.g., allyl)); and R₁₀ and R₁₃ are each independently R_{C}, and R₁₁ and R₁₄, taken together with the atoms to which they are attached, form a 5- to 6-membered carbocycle/heterocycle or 6- to 12-membered bicycle

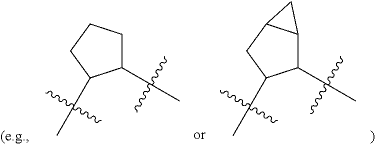

which is optionally substituted with one or more R_{A} (such as, but not limited to hydroxy, halo (e.g., fluoro), C₁-C₆alkyl (e.g., methyl), or C₂-C₆alkenyl (e.g., allyl)).

Z can also be selected from -M-C(R₈R₉)N(R₁₂)—C(O)-L_{Y}′-M′-R_{D}, -M-C(R₈R₉)N(R₁₂)-L_{Y}′-M′-R_{D}, -L_{S}-C(R₈R₉)N(R₁₂)—C(O)-L_{Y}′-M′-L_{S}-C(R₈R₉)N(R₁₂)-L_{Y}′-M′-R_{D}, -M-C(R₁₀R₁₁)C(R₁₃R₁₄)—C(O)-L_{Y}′-M′-R_{D}, -M-C(R₁₀R₁₁)C(R₁₃R₁₄)-L_{Y}′-M′-R_{D}, -L_{S}-C(R₁₀R₁₁)C(R₁₃R₁₄)—C(O)-L_{Y}′-M′-R_{D}, or -L_{S}-C(R₁₀R₁₁)C(R₁₃R₁₄)-L_{Y}′-M′-R_{D}, wherein M preferably is bond, —C(O)N(R_{B})— or —N(R_{B})C(O)—, M′ preferably is bond, —C(O)N(R_{B})—, —N(R_{B})C(O)—, —N(R_{B})C(O)O—, N(R_{B})C(O)N(R_{B}′)—, —N(R_{B})S(O)— or —N(R_{B})S(O)₂—, and L_{Y}′ preferably is C₁-C₆alkylene which is independently optionally substituted with one or more R_{L}. L_{Y}′, for example, is a C₁-C₆alkylene such as, but not limited to,

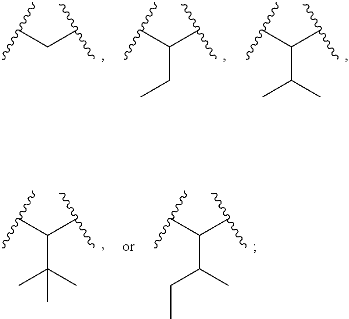

and the optional R_{L} is a substituent such as, but not limited to phenyl, —SMe, or methoxy. Any stereochemistry at a carbon within the group L_{Y}′ can be either (R) or (S). More preferably, R₈ is R_{C}, and R₉ and R₁₂, taken together with the atoms to which they are attached, form a 5- to 6-membered heterocycle or 6- to 12-membered bicycle

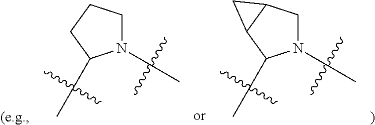

which is optionally substituted with one or more R_{A} (e.g., one or more hydroxy); and R₁₀ and R₁₃ are each independently R_{C}, and R₁₁ and R₁₄, taken together with the atoms to which they are attached, form a 5- to 6-membered carbocycle/heterocycle or 6- to 12-membered bicycle

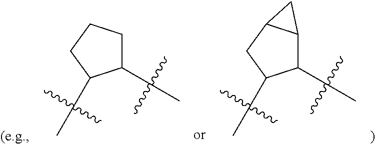

which is optionally substituted with one or more R_{A}.

Also preferably, Z is selected from —N(R_{B})CO—C(R₈R₉)N(R₁₂)—C(O)-L_{Y}′-N(R_{B})C(O)O—R_{D}, —N(R_{B})CO—C(R₈R₉)N(R₁₂)—C(O)-L_{Y′}—N(R_{B})C(O)—R_{D}, —N(R_{B})CO—C(R₈R₉)N(R₁₂)—C(O)-L_{Y}′-N(R_{B})S(O)₂—R_{D}, —N(R_{B})CO—C(R₈R₉)N(R₁₂)—C(O)-L_{Y}′-N(R_{B}R_{B})—R_{D}, —N(R_{B})CO—C(R₈R₉)N(R₁₂)—C(O)-L_{Y}-O—R_{D}, —N(R_{B})CO—C(R₈R₉)N(R₁₂)—C(O)-L_{Y}-R_{D}, —N(R_{B})CO—C(R₈R₉)N(R₁₂)—R_{D}, -L_{S}-C(R₈R₉)N(R₁₂)—C(O)-L_{Y}′-N(R_{B})C(O)O—R_{D}, -L_{S}-C(R₈R₉)N(R₁₂)—C(O)-L_{Y}′-N(R_{B})C(O)—R_{D}, -L_{S}-C(R₈R₉)N(R₁₂)—C(O)-L_{Y}′-N(R_{B})S(O)₂—R_{D}, -L_{S}-C(R₈R₉)N(R₁₂)—C(O)-L_{Y}′-N(R_{B}R_{B}′)—R_{D}, -L_{S}-C(R₈R₉)N(R₁₂)—C(O)-L_{Y}′-O—R_{D}, -L_{S}-C(R₈R₉)N(R₁₂)—C(O)-L_{Y}′-R_{D}, -L_{S}-C(R₈R₉)N(R₁₂)—R_{D}, —N(R_{B})CO—C(R₁₀R₁₁)C(R₁₃R₁₄)—C(O)-L_{Y}′-N(R_{B})C(O)O—R_{D}, —N(R_{B})CO—C(R₁₀R₁₁)C(R₁₃R₁₄)—C(O)-L_{Y}′-N(R_{B})C(O)—R_{D}, —N(R_{B})CO—C(R₁₀R₁₁)C(R₁₃R₁₄)—C(O)-L_{Y}-N(R_{B})S(O)₂—R_{D}, —N(R_{B})CO—C(R₁₀R₁₁)C(R₁₃R₁₄)—C(O)-L_{Y}′-N(R_{B}R_{B}′)—R_{D}, —N(R_{B})CO—C(R₁₀R₁₁)C(R₁₃R₁₄)—C(O)-L_{Y}-O—R_{D}, —N(R_{B})CO—C(R₁₀R₁₁)C(R₁₃R₁₄)—C(O)-L_{Y}′-N(R_{B})CO—C(R₁₀R₁₁)C(R₁₃R₁₄)—R_{D}, -L_{S}-C(R₁₀R₁₁)C(R₁₀R₁₁)C(O)-L_{Y}′-N(R_{B})C(O)O—R_{D}, -L_{S}-C(R₁₀R₁₁)C(R₁₃R₁₄)—C(O)-L_{Y}′-N(R_{B})C(O)—R_{D}, -L_{S}-C(R₁₀R₁₁)C(R₁₃R₁₄)—C(O)-L_{Y}′-N(R_{B})S(O)₂—R_{D}, -L_{S}-C(R₁₀R₁₁)C(R₁₃R₁₄)—C(O)-L_{Y}′-N(R_{B}R_{B})—R_{D}, -L_{S}-C(R₁₀R₁₁)C(R₁₃R₁₄)—C(O)-L_{Y}′O—R_{D}, -L_{S}-C(R₁₀R₁₁)C(R₁₃R₁₄)—C(O)-L_{Y}′-R_{D}, or -L_{S}-C(R₁₀R₁₁)C(R₁₃R₁₄)—R_{D}, wherein L_{Y}′ preferably is C₁-C₆alkylene which is independently optionally substituted with one or more R_{L}. R₈ may be R_{C}, and R₉ and R₁₂, taken together with the atoms to which they are attached, may form a 5- to 6-membered heterocycle or 6- to 12-membered bicycle

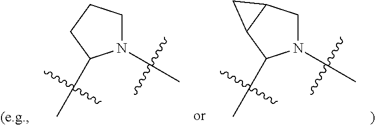

which is optionally substituted with one or more R_{A}; and R₁₀ and R₁₃ may be each independently R_{C}, and R₁₁ and R₁₄, taken together with the atoms to which they are attached, may form a 5- to 6-membered carbocycle/heterocycle or 6- to 12-membered bicycle

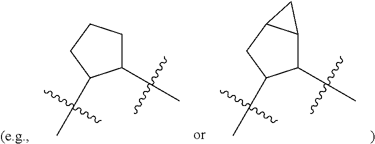

which is optionally substituted with one or more R_{A}.

Highly preferably, Z is selected from —N(R_{B}″)CO—C(R₈R₉)N(R₁₂)—C(O)-L_{Y}-N(R_{B}″)C(O)-L_{S}-R_{E} or —C(R₈R₉)N(R₁₂)—C(O)-L_{Y}-N(R_{B}″)C(O)-L_{S}-R_{E}, or Z is -G-C(R₈R₉)N(R₁₂)—C(O)-L_{Y}-N(R_{B}″)C(O)-L_{S}-R_{E}, wherein L_{Y} is C₁-C₆alkylene optionally substituted with one or more R_{L}, and R_{B}″ is each independently R_{B}. R_{B}″ and R₈ are each preferably hydrogen or C₁-C₆alkyl, and R₉ and R₁₂, taken together with the atoms to which they are attached, preferably form a 5- to 6-membered heterocycle or 6- to 12-membered bicycle

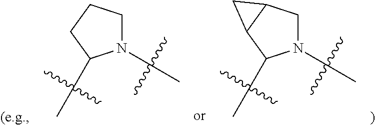

which is optionally substituted with one or more R_{A} (such as, but not limited to hydroxy, halo (e.g., fluoro), C₁-C₆alkyl (e.g., methyl), or C₂-C₆alkenyl (e.g., allyl)). Preferably, L_{Y} is C₁-C₆alkylene substituted with one or more R_{L} such as a C₃-C₆carbocycle 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, L_{Y} is a C₁-C₆alkylene such as, but not limited to,

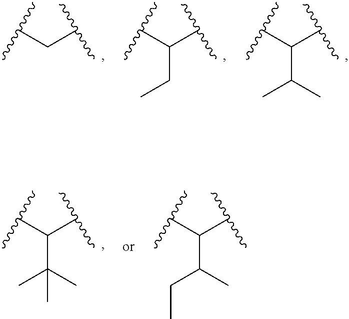

(stereochemistry at a carbon within the group L_{Y} can be either (R) or (S)); L_{Y} is independently optionally substituted with one or more R_{L} (e.g., one or more phenyl or methoxy); G preferably is

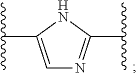

R_{B}″ is hydrogen; —C(R₈R₉)N(R₁₂)— is

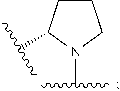

L_{S} is a bond; and R_{E} is methoxy.

Non-limiting examples of preferred Z include:

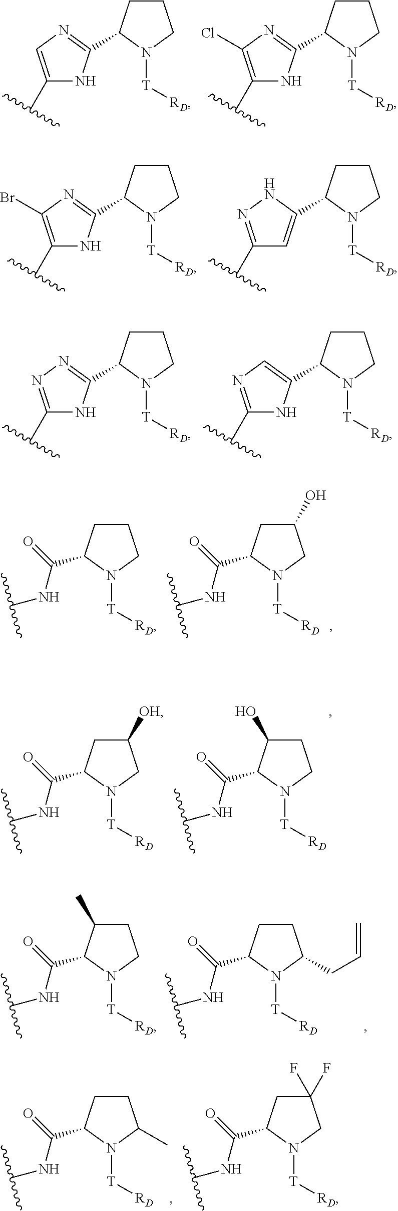

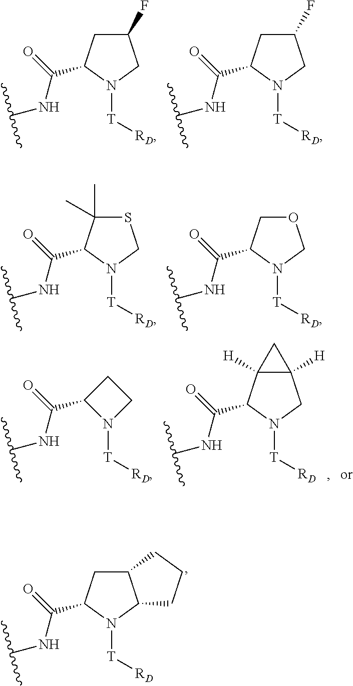

wherein T and R_{D} are as defined herein. T, for example, can be -L_{S}-M-L_{S}′-M′-L_{S}″- where L_{S} is a bond; M is C(O): L_{S}′ is C₁-C₆alkylene such as, but not limited to,

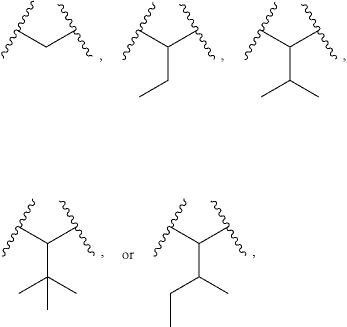

where L_{S}′ is independently optionally substituted with one or more R_{L}; the optional R_{L} is a substituent such as, but not limited to phenyl or methoxy; M′ is —NHC(O)— or —NMeC(O)—; and L_{S}″ is a bond. Any stereochemistry at a carbon within the group L_{S}′ can be either (R) or (S). R_{D}, for example is methoxy. T-R_{D} includes, but is not limited to:

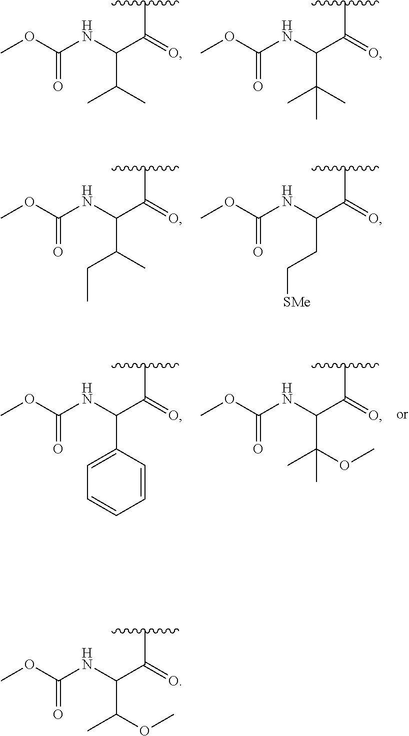

T-R_{D} may also include certain stereochemical configurations; thus T-R_{D} includes, but is not limited to:

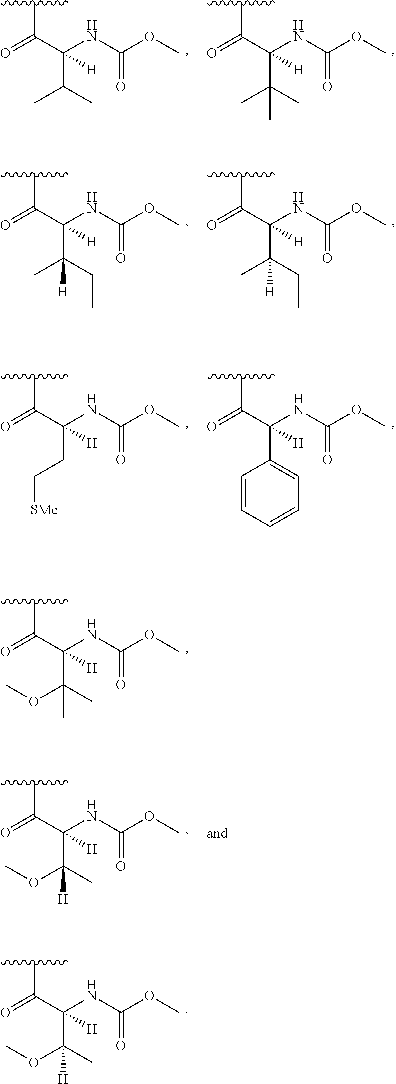

Non-limiting examples of preferred Z also include:

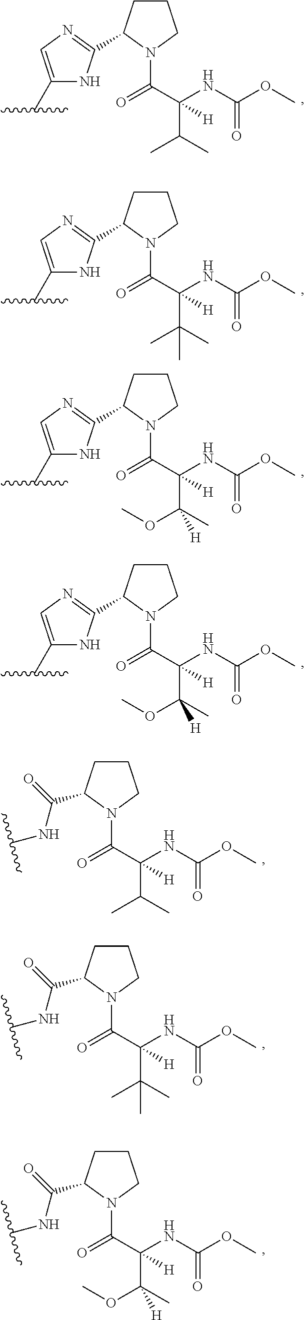

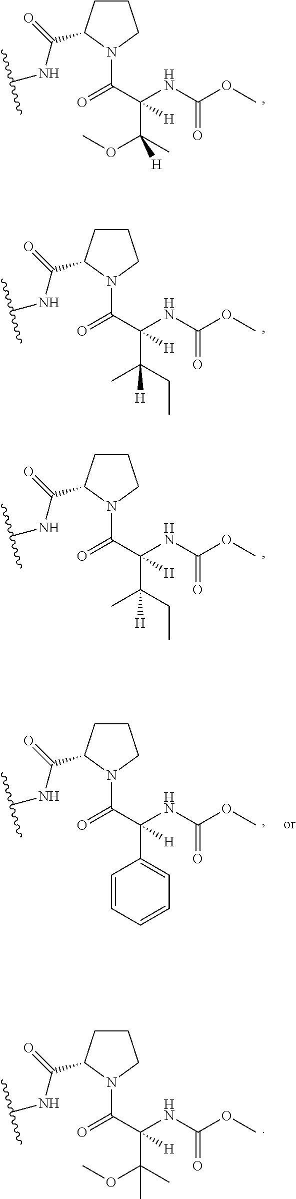

T can be, without limitation, independently selected at each occurrence from —C(O)-L_{S}′-, —C(O)O-L_{S}′-, —C(O)-L_{S}′-N(R_{B})C(O)-L_{S}″-, —C(O)-L_{S}′-N(R_{B})C(O)O-L_{S}″-, —N(R_{B})C(O)-L_{S}′-N(R_{B})C(O)-L_{S}″-, —N(R_{B})C(O)-L_{S}′-N(R_{B})C(O)O-L_{S}″-, or —N(R_{B})C(O)-L_{S}′-N(R_{B})-L_{S}″-. Preferably, T is independently selected at each occurrence from —C(O)-L_{S}′-M′-L_{S}″ or —N(R_{B})C(O)-L_{S}′-M′-L_{S}″-. More preferably, T is independently selected at each occurrence from —C(O)-L_{S}′-N(R_{B})C(O)-L_{S}″ or —C(O)-L_{S}′-N(R_{B})C(O)O-L_{S}″-.

T can also be, for example, -L_{S}-M-L_{S}′-M′-L_{S}″- where L_{S} is a bond; M is C(O); L_{S}′ is C₁-C₆alkylene

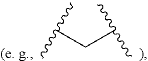

where L_{S}′ is independently optionally substituted with R_{T}; the optional R_{T} is a substituent selected from —C₁-C₆alkyl, —C₂-C₆alkenyl, —C₁-C₆alkyl-OH, —C₁-C₆alkyl-O—C₁-C₆alkyl, 3- to 6-membered heterocycle (e.g., tetrahydrofuranyl), or C₃-C₆carbocyclyl (e.g., phenyl, cyclohexyl); M′ is —NHC(O)—, —N(Et)C(O)— or —N(Me)C(O)—; and L_{S}″ is a bond. R_{D} preferably is hydrogen, —C₁-C₆alkyl (e.g., methyl), —O—C₁-C₆alkyl (e.g., methoxy, tert-butoxy), methoxymethyl, or —N(C₁-C₆alkyl)₂ (e.g., —NMe₂). T-R_{D} can be, without limitation,

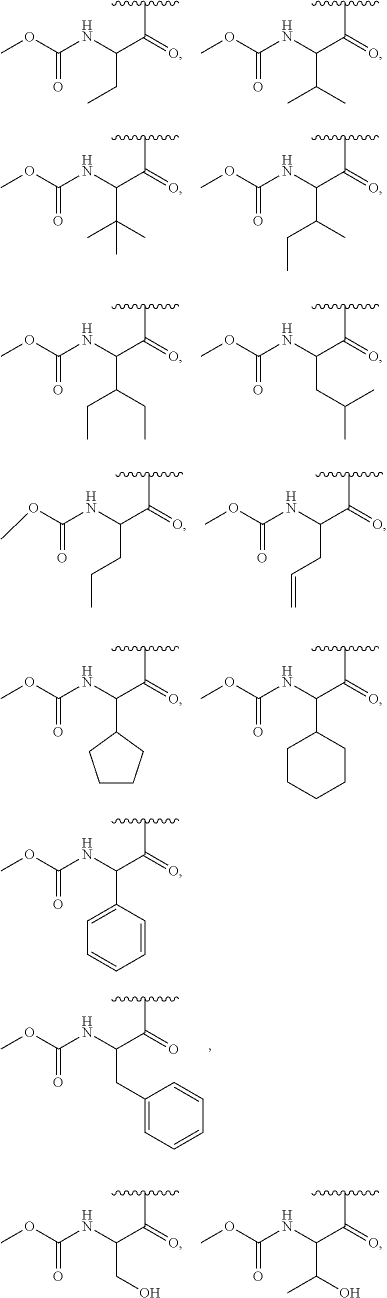

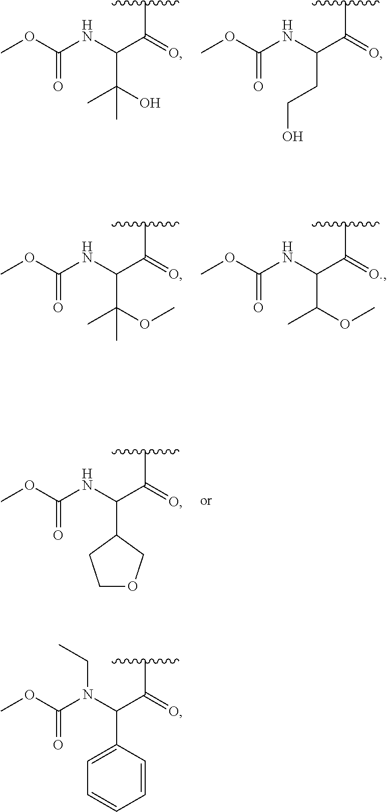

wherein the stereochemistry at a carbon within the group T-R_{D} can be either (R) or (S).

T can also be without limitation. -L_{S}-M-L_{S}′- where L_{S} is a bond; M is C(O); L_{S}′ is C₁-C₆alkylene

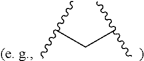

where L_{S}′ is independently optionally substituted with R_{T}; the optional R_{T} is a substituent selected from —C₁-C₆alkyl, —C₁-C₆alkyl-OH, —C₁-C₆alkyl-O—C₁-C₆alkyl, or a C₃-C₆carbocyclyl (e.g., phenyl, cyclohexyl). R_{D}, for example is —OH; —OC(O)Me; —NH(C₁-C₆alkyl) (e.g., —NHMe, —NHEt); —N(C₁-C₆alkyl)₂ (e.g., —NMe₂, —NEt₂); a 3- to 10-membered heterocyclyl (e.g., pyrrolidinyl, imidazolidinyl, hexahydropyrimidinyl, morpholinyl, piperidinyl) optionally substituted with one or more halogen, oxo; C₃-C₁₀carbocycle (e.g., cyclopentyl) optionally substituted with —OH; —C₁-C₆alkyl (e.g., isopropyl, 3-pentyl) optionally substituted with —OH; or NHR_{T} where R_{T} is a 3- to 6-membered heterocyclyl (e.g., thiazolyl, pyrimidinyl). T-R_{D} includes, but is not limited to:

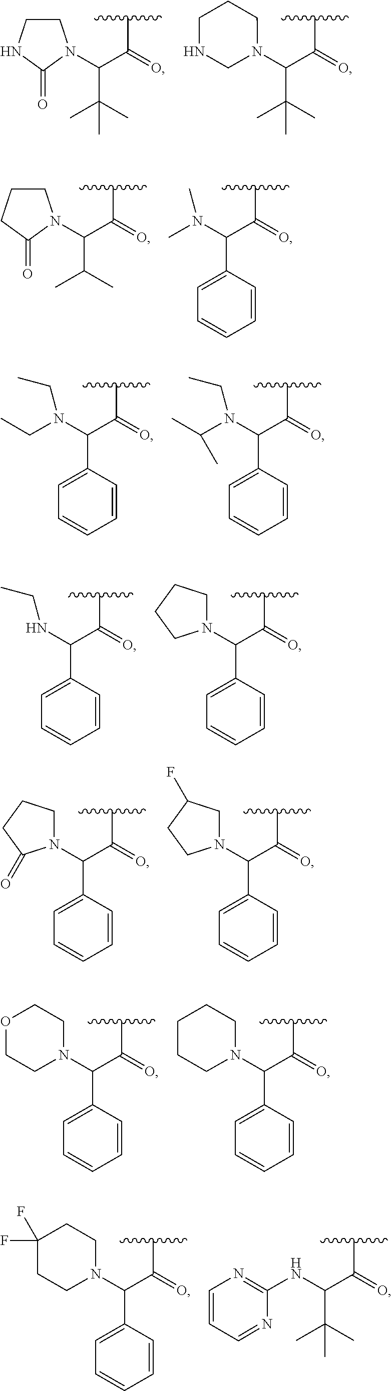

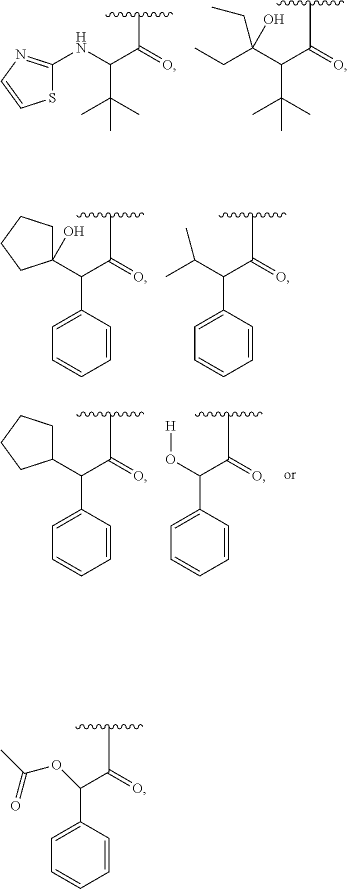

wherein the stereochemistry at a carbon within the group T-R_{D} can be either (R) or (S).

For each compound of Formula I, L_{K} can also be independently selected at each occurrence from a bond; -L_{S}′-N(R_{B})C(O)-L_{S}-; -L_{S}′-C(O)N(R_{B})-L_{S}-; or C₁-C₆alkylene, C₂-C₆alkenylene, C₂-C₆alkynylene, C₃-C₁₀carbocycle or 3- to 10-membered heterocycle, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano, wherein R_{T}, R_{B}, R_{S}, R_{S}′, L_{S} and L_{S}′ are as defined above.

For Formula I as well as Formulae I_{A}, I_{B}, I_{C}, I_{D}, I_{E}, I_{F}, I_{G}, I_{H} or I_{I} described below, including each and every embodiment described thereunder, R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}-L_{A}-N(R_{S})C(O)N(R_{S}′R_{S}″), -L_{A}-N(R_{S})SO₂R_{S}-L_{A}-SO₂N(R_{S}R_{S}), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

For Formula I as well as Formulae I_{A}, I_{B}, I_{C}, I_{D}, I_{F}, I_{F}, I_{G}, I_{H} or I_{I} described below, including each and every embodiment described thereunder, R_{F} preferably is C₁-C₁₀alkyl, C₂-C₁₀alkenyl or C₂-C₁₀alkynyl, each of which contains 0, 1, 2, 3, 4 or 5 heteroatoms selected from O, S or N and is independently optionally substituted with one or more substituents selected from halogen, hydroxy, mercapto, amino, carboxy, nitro, oxo, phosphonoxy, phosphono, thioxo, formyl or cyano. Also preferably, R_{F} is C₁-C₁₀alkyl, C₂-C₁₀alkenyl or C₂-C₁₀alkynyl, each of which contains 0, 1, 2, 3, 4 or 5 O and is independently optionally substituted with one or more R_{L}. Also preferably, R_{F} is —(R_{X}—R_{Y})_{Q}—(R_{X}—R_{Y}), wherein Q is 0, 1, 2, 3 or 4; each R_{X} is independently O, S or N(R); each R_{Y} is independently C₁-C₆alkylene, C₂-C₆alkenylene or C₂-C₆alkynylene each of which is independently optionally substituted with one or more substituents selected from halogen, hydroxy, mercapto, amino, carboxy, nitro, oxo, phosphonoxy, phosphono, thioxo, formyl or cyano; and each R_{Y}′ is independently C₁-C₆alkyl, C₂-C₆alkenyl or C₂-C₆alkynyl each of which is independently optionally substituted with one or more substituents selected from halogen, hydroxy, mercapto, amino, carboxy, nitro, oxo, phosphonoxy, phosphono, thioxo, formyl or cyano. Preferably, each R_{X} is O. More preferably, X is optionally substituted with R_{F}, each R_{F} is independently selected from C₁-C₁₀alkyl, C₂-C₁₀alkenyl or C₂-C₁₀alkynyl, each of which contains 0, 1, 2 or 3 O and is independently optionally substituted with one or more substituents selected from halogen, hydroxy, mercapto, amino, carboxy, nitro, oxo, phosphonoxy, phosphono, thioxo, formyl or cyano. Also preferably, X is optionally substituted with R_{F}, each R_{F} is independently selected from —(O—C₁-C₆alkylene)_{Q}-(O—C₁-C₆alkyl), wherein Q preferably is 0, 1, 2 or 3.

L_{S}, L_{S}′ and L_{S}″ preferably are each independently selected at each occurrence from bond; or C₁-C₆alkylene, C₂-C₆alkenylene or C₂-C₆alkynylene.

A and B can be the same or different. Likewise, L₁ and L₂, or Y and Z, or Y-A- and Z—B—, or -A-L₁- and —B-L₂-, can be the same or different. In some instances, Y-A-L₁- is identical to Z—B-L₂-. In some other instances, Y-A-L₁- is different from Z—B-L₂-.

In one embodiment, A and B are each independently 5- or 6-membered carbocycle or heterocycle (e.g., phenyl such as

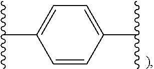

and are each independently optionally substituted with one or more R_{A}. D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

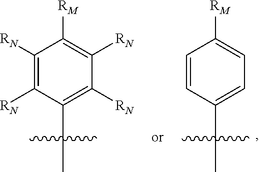

wherein R_{M} and R_{N} are as defined above. Also preferably, D is

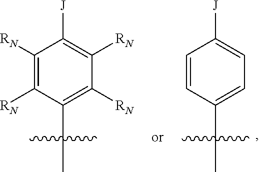

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is —N(R_{B})C(O)C(R₁R₂)N(R₅)-T-R_{D}, or —N(R_{B})C(O)C(R₃R₄)C(R₆R₇)-T-R_{D}, and Z is —N(R_{B})C(O)C(R₈R₉)N(R₁₂)-T-R_{D}, or —N(R_{B})C(O)C(R₁₀R₁₁)C(R₁₃R₁₄)-T-R_{D}. R₁ is R_{C}, and R₂ and R₅, taken together with the atoms to which they are attached, form a 5- to 6-membered heterocyclic ring

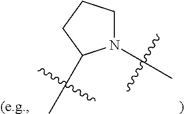

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

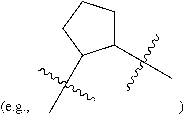

which is optionally substituted with one or more R_{A}. R₈ is R_{C}, and R₉ and R₁₂, taken together with the atoms to which they are attached, form a 5- to 6-membered heterocyclic ring

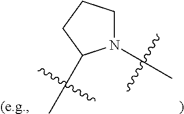

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

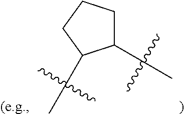

which is optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-N(R_{B})-L_{S}″-, or —C(O)-L_{Y}′-N(R_{B})S(O)₂-L_{S}″-. In some cases, at least one of Y and Z is, or both Y and Z are independently,

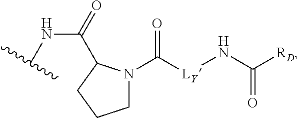

wherein non-limiting examples of R_{D} include (1) —O—C₁-C₆alkyl, —O—C₂-C₆alkenyl, —O—C₂-C₆alkynyl, C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or (2) C₃-C₆carbocycle or 3- to 6-membered heterocycle each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; and non-limiting examples of L_{Y}′ include C₁-C₆alkylene optionally substituted with halogen, hydroxy, mercapto, amino, carboxy, phosphonoxy, —O—C₂-C₆alkenyl, —O—C₂-C₆alkynyl, or 3- to 6-membered carbocycle or heterocycle, said 3- to 6-membered carbocycle or heterocycle being optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another embodiment, A is

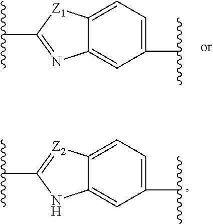

and is optionally substituted with one or more R_{A}; B is

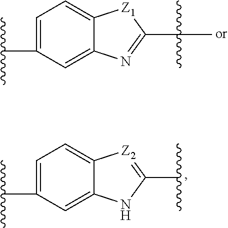

and is optionally substituted with one or more R_{A}. Z₁ is independently selected at each occurrence from O, S, NH or CH₂; and Z₂ is independently selected at each occurrence from N or CH. D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

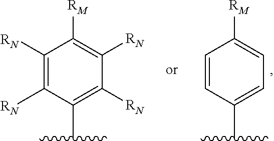

wherein R_{M} and R_{N} are as defined above. Also preferably, D is

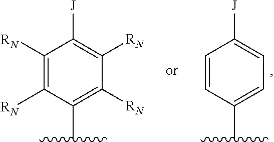

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is -L_{S}-C(R₁R₂)N(R_{S})-T-R_{D} or -L_{S}-C(R₃R₄)C(R₆R₇)-T-R_{D}, and Z is -L_{S}-C(R₈R₉)N(R₁₂)-T-R_{D} or -L_{S}-C(R₁₀R₁₁)C(R₁₃R₁₄)-T-R_{D}. R₁ is R_{C}, and R₂ and R₅, taken together with

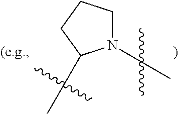

the atoms to which they are attached, form a 5- to 6-membered heterocyclic ring which is optionally substituted with one or more R_{A}; R₃ and R₆ are each independently R_{C}, and R₄ and R₇, taken together with the atoms to which they are attached, form a 5- to 6-membered carbocyclic or heterocyclic ring

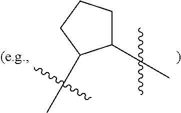

which is optionally substituted with one or more R_{A}. R₈ is R_{C}, and R₉ and R₁₂, taken together with the atoms to which they are attached, form a 5- to 6-membered heterocyclic ring

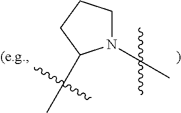

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

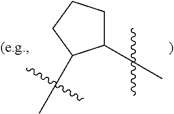

which is optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-O-L_{S}″-, —C(O)-L_{Y}′-N(R_{B})-L_{S}″-, or —C(O)-L_{Y}′-N(R_{B})S(O)₂-L_{S}″-. In some cases, at least one of Y and Z is, or both Y and Z are independently,

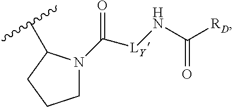

wherein non-limiting examples of R_{D} include (1) —O—C₁-C₆alkyl, —O—C₂-C₆alkenyl, —O—C₂-C₆alkynyl, C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or (2) C₃-C₆carbocycle or 3- to 6-membered heterocycle each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; and non-limiting examples of L_{Y}′ include C₁-C₆alkylene optionally substituted with halogen, hydroxy, mercapto, amino, carboxy, phosphonoxy, —O—C₁-C₆alkyl, —O—C₂-C₆alkenyl, —O—C₂-C₆alkynyl, or 3- to 6-membered carbocycle or heterocycle, said 3- to 6-membered carbocycle or heterocycle being optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In still yet another embodiment, A and B are each independently 5- or 6-membered carbocycle or heterocycle (e.g., A and B are each independently phenyl, such as

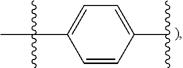

and are each independently optionally substituted with one or more R_{A}. D can be, for example, C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}.

Preferably, D is

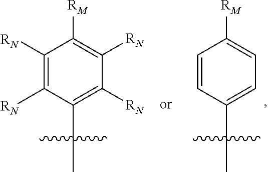

wherein R_{M} and R_{N} are as defined above. Also preferably, D is

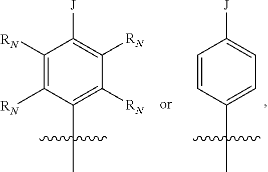

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is -G-C(R₁R₂)N(R₅)-T-R_{D} or -G-C(R₃R₄)C(R₆R₇)-T-R_{D}, and Z is -G-C(R₈R₉)N(R₁₂)-T-R_{D} or -G-C(R₁₀R₁₁)C(R₁₃R₁₄)-T-R_{D}. G is independently C₅-C₆carbocycle or 5- to 6-membered heterocycle, such as

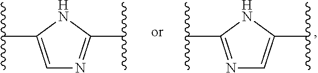

and is independently optionally substituted with one or more R_{A}. R₁ is R_{C}, and R₂ and R₅, taken together with the atoms to which they are attached, form a 5- to 6-membered heterocyclic ring

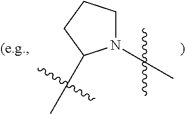

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

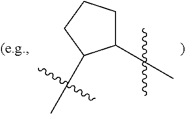

which is optionally substituted with one or more R_{A}. R₈ is R_{C}, and R₉ and R₁₂, taken together with the atoms to which they are attached, form a 5- to 6-membered heterocyclic ring

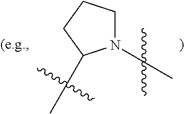

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

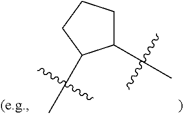

which is optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-O-L_{S}″-, —C(O)-L_{Y}′-N(R_{B})-L_{S}″-, or —C(O)-L_{Y}′-N(R_{B})S(O)₂-L_{S}″-. In some cases, at least one of Y and Z is, or both Y and Z are independently,

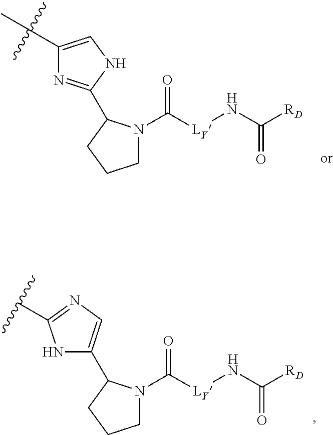

wherein non-limiting examples of R_{D} include (1) —O—C₁-C₆alkyl, —O—C₂-C₆alkenyl, —O—C₂-C₆alkynyl, C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or (2) C₃-C₆carbocycle or 3- to 6-membered heterocycle each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; and non-limiting examples of L_{Y}′ include C₁-C₆alkylene optionally substituted with halogen, hydroxy, mercapto, amino, carboxy, phosphonoxy, —O—C₁-C₆alkyl, —O—C₂-C₆alkenyl, —O—C₂-C₆alkynyl, or 3- to 6-membered carbocycle or heterocycle, said 3- to 6-membered carbocycle or heterocycle being optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In yet another embodiment, A and B are each independently 5- or 6-membered carbocycle or heterocycle (e.g., A and B are each independently phenyl, such as

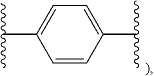

and are each independently optionally substituted with one or more R_{A}. D can be, for example, C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

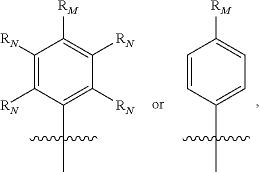

wherein R_{M} and R_{N} are as defined above. Also preferably, D is

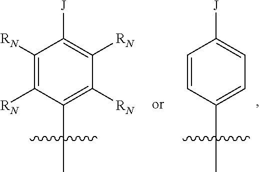

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is —N(R_{B})C(O)C(R₁R₂)N(R₅)-T-R_{D} or —N(R_{B})C(O)C(R₃R₄)C(R₆R₇)-T-R_{D}, and Z is -G-C(R₈R₉)N(R₁₂)-T-R_{D} or -G-C(R₁₀R₁₁)C(R₁₃R₁₄)-T-R_{D}; or Y is -G-C(R₁R₂)N(R₅)-T-R_{D} or -G-C(R₃R₄)C(R₆R₇)-T-R_{D}, and Z is —N(R_{B})C(O)C(R₈R₉)N(R₁₂)-T-R_{D} or —N(R_{B})C(O)C(R₁₀R₁₁)C(R₁₃R₁₄)-T-R_{D}. R₁ is R_{C}, and R₂ and R₅, taken together with the atoms to which they are attached, form a 5- to 6-membered heterocyclic ring

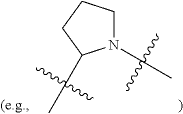

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

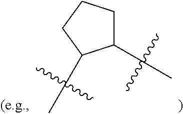

which is optionally substituted with one or more R_{A}. R₈ is R_{C}, and R₉ and R₁₂, taken together with the atoms to which they are attached, form a 5- to 6-membered heterocyclic ring

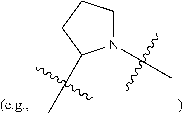

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

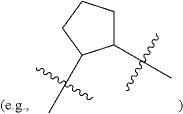

which is optionally substituted with one or more R_{A}. G is independently C₅-C₆carbocycle or 5- to 6-membered heterocycle, such as

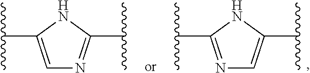

and is independently optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-O-L_{S}″-, —C(O)-L_{Y}′-N(R_{B})-L_{S}″-, or —C(O)-L_{Y}′-N(R_{B})S(O)₂-L_{S}″-. In some cases, Y is

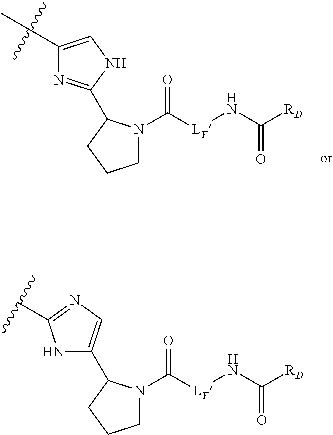

as described above, and Z is

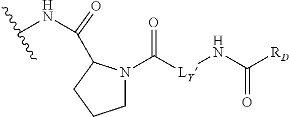

as described above. In some other cases, Y is

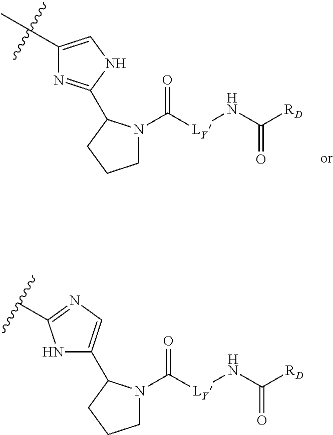

as described above, and Z is

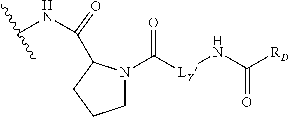

as described above.

In still another embodiment, A is 5- or 6-membered carbocycle or heterocycle (e.g., phenyl such as

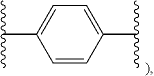

and

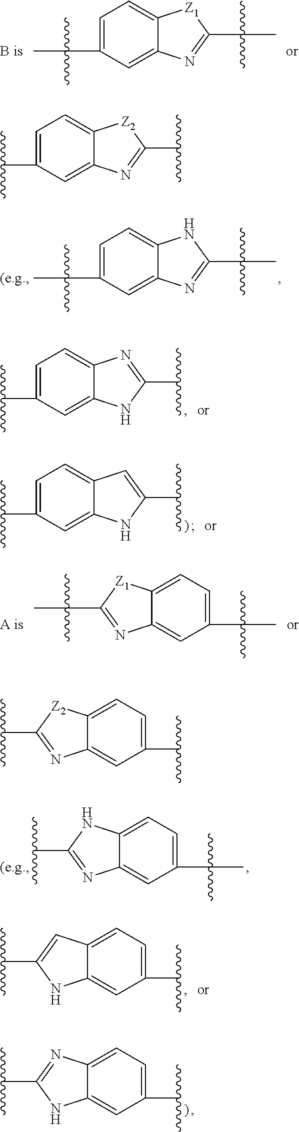

B is 5- or 6-membered carbocycle or heterocycle (e.g., phenyl such as

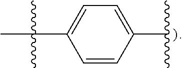

A and B are each independently optionally substituted with one or more R_{A}. Z₁ is independently selected at each occurrence from O, S, NH or CH₂; and Z₂ is independently selected at each occurrence from N or CH. D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D

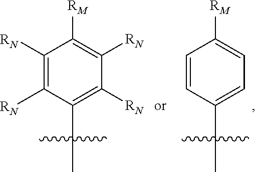

is wherein R_{M} and R_{N} are as defined above. Also preferably, D

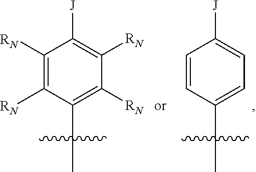

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. When A is 5- or 6-membered carbocycle or heterocycle (e.g., phenyl such as

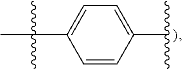

Y is —N(R_{B})C(O)C(R₁R₂)N(R₅)-T-R_{D}, —N(R_{B})C(O)C(R₃R₄)C(R₆R₇)-T-R_{D}, -G-C(R₁R₂)N(R₅)-T-R_{D} or -G-C(R₃R₄)C(R₆R₇)-T-R_{D}, and Z is -L_{S}-C(R₈R₉)N(R₁₂)-T-R_{D} or -L_{S}-C(R₁₀R₁₁)C(R₁₃R₁₄)-T-R_{D}. When B is 5- or 6-membered carbocycle or heterocycle (e.g., phenyl such as

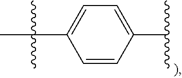

Y is -L_{S}-C(R₁R₂)N(R₅)-T-R_{D} or -L_{S}-C(R₃R₄)C(R₆R₇)-T-R_{D}, and Z is —N(R_{B})C(O)C(R₈R₉)N(R₁₂)-T-R_{D}, —N(R_{B})C(O)C(R₁₀R₁₁)C(R₁₃R₁₄)-T-R_{D}, -G-C(R₈R₉)N(R₁₂)-T-R_{D} or -G-C(R₁₀R₁₁)C(R₁₃R₁₄)-T-R_{D}. R₁ is R_{C}, and R₂ and R₅, taken together with the atoms to which they are attached, form a 5- to 6-membered heterocyclic ring

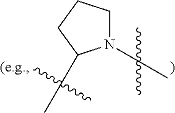

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

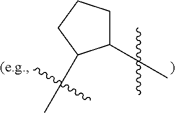

which is optionally substituted with one or more R_{A}. R₈ is R_{C}, and R₉ and R₁₂, taken together with the atoms to which they are attached, form a 5- to 6-membered heterocyclic ring

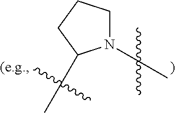

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

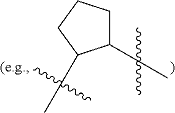

which is optionally substituted with one or more R_{A}. G is independently C₅-C₆carbocycle or 5- to 6-membered heterocycle, such as

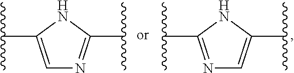

and is independently optionally substituted with one or more R_{A}. T is preferably independently selected at each occurrence from —C(O)-L_{Y}′-N(R_{B})C(O)-L_{S}″- or —C(O)-L_{Y}-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-O-L_{S}″ —C(O)-L_{Y}′-N(R_{B})-L_{S}″-, or —C(O)-L_{Y}′-N(R_{B})S(O)₂-L_{S}″-. In some cases when A is 5- or 6-membered carbocycle or heterocycle (e.g., phenyl such as

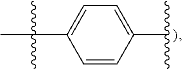

### Y is

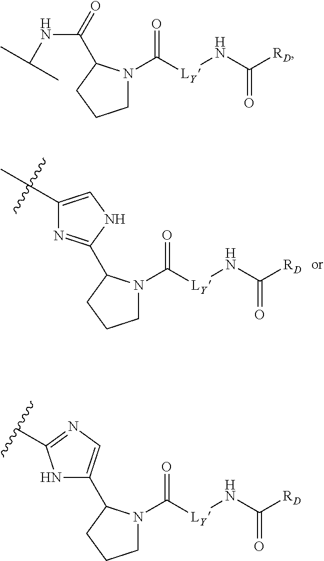

as described above, and Z is

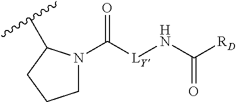

as described above. In some other cases when B is 5- or 6-membered carbocycle or heterocycle (e.g., phenyl such as

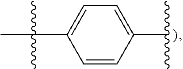

### Y is

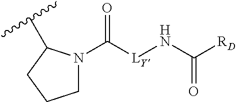

as described above, and Z is

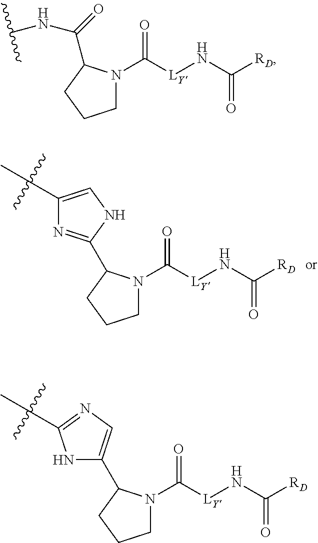

as described above.

The present invention also features compounds of Formulae I, I_{A}, I_{B}, I_{C} and I_{D} as described herein (including each embodiment described hereunder) and pharmaceutically acceptable salts thereof, wherein:
D is C₃-C₁₂carbocycle or 3- to 12-membered heterocycle, and is optionally substituted with one or more R_{A}; or D is C₃-C₁₂carbocycle or 3- to 12-membered heterocycle which is substituted with J and optionally substituted with one or more R_{A}, where J is C₃-C₁₅carbocycle or 3- to 15-membered heterocycle (e.g., a 3- to 6-membered monocycle, a 6- to 12-membered fused, bridged or spiro bicycle, a 10- to 15-membered tricycle containing fused, bridged or spiro rings, or a 13- to 15-membered carbocycle or heterocycle) and is optionally substituted with one or more R_{A}, or J is —SF₅; or D is hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, ═C(R_{S}R_{S}′), or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₁₂carbocycle or 3- to 12-membered heterocycle (e.g., 7- of 12-membered carbocycle or heterocycle), each of which is independently optionally substituted at each occurrence with one or more substituents selected from halogen, hydroxy, mercapto, amino, carboxy, nitro, oxo, phosphonoxy, phosphono, thioxo, formyl, cyano, trimethylsilyl, C₁-C₆alkyl, C₂-C₆alkenyl, C₂-C₆alkynyl, C₁-C₆haloalkyl, C₂-C₆haloalkenyl, C₂-C₆haloalkynyl, —O—R_{S}, —S—R_{S}, —C(O)R_{S}, —C(O)OR_{S}, or —N(R_{S}R_{S}′).

In one embodiment, A and B are each independently 5- or 6-membered carbocycle or heterocycle (preferably, A and B are each independently phenyl such as

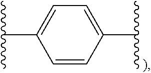

and are each independently optionally substituted with one or more R_{A} (preferably, A and B are each independently substituted with at least one halo such as F). D is a C₅-C₆carbocycle or 5- to 6-membered heterocycle (e.g., phenyl), and is substituted with J and optionally substituted with one or more R_{A}. J is C₃-C₆carbocycle, 3- to 6-membered heterocycle, 6- to 12-membered bicycle, 10- to 15-membered tricycle, or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S}, —C(O)R_{S}; and J can also be optionally substituted with one or more R_{A}. Preferably, D is

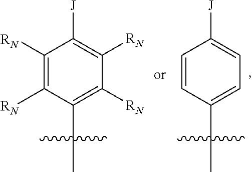

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is —N(R_{B})C(O)C(R₁R₂)N(R₅)-T-R_{D}, —N(R_{B})C(O)C(R₃R₄)C(R₆R₇)-T-R_{D}, -G-C(R₁R₂)N(R₅)-T-R_{D} or -G-C(R₃R₄)C(R₆R₇)-T-R_{D}. Z is —N(R_{B})C(O)C(R₈R₉)N(R₁₂)-T-R_{D}, —N(R_{B})C(O)C(R₁₀R₁₁)C(R₁₃R₁₄)-T-R_{D}, -G-C(R₈R₉)N(R₁₂)-T-R_{D} or -G-C(R₁₀R₁₁)C(R₁₃R₁₄)-T-R_{D}. R₁ is R_{C}; and R₂ and R₅, taken together with the atoms to which they are attached, form a 5- to 6-membered heterocyclic ring

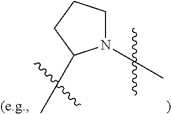

or 6- to 12-membered bicycle

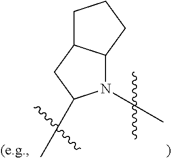

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

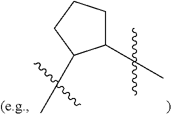

or 6- to 12-membered bicycle which is optionally substituted with one or more R_{A}. R₈ is R_{C}; and R₉ and R₁₂, taken together with the atoms to which they are attached, form a 5- to 6-membered heterocyclic ring

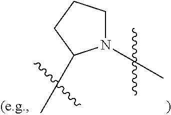

or 6- to 12-membered bicycle

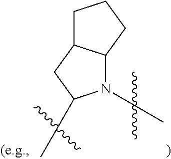

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

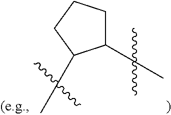

or 6- to 12-membered bicycle which is optionally substituted with one or more R_{A}. G is independently C₅-C₆carbocycle or 5- to 6-membered heterocycle, such as

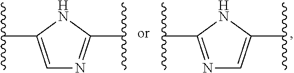

and is independently optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-N(R_{B})-L_{Y}″-, or —C(O)-L_{Y}′-N(R_{B})S(O)₂-L_{S}″-. In some cases, Y is

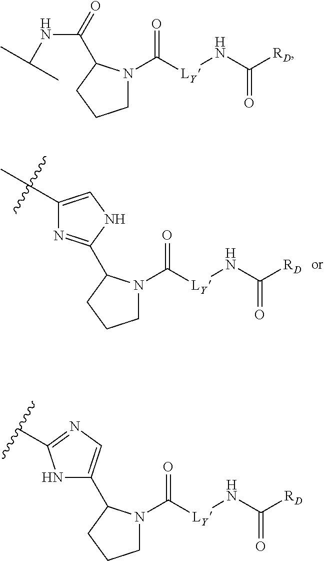

as described above, and Z is

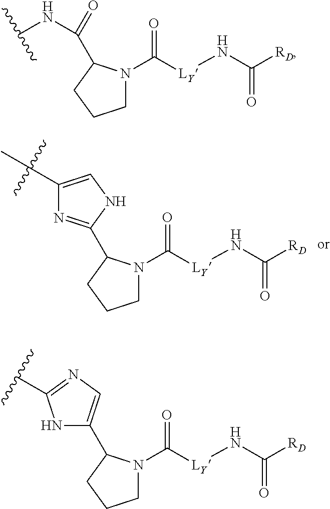

as

described above.

In another embodiment, A is

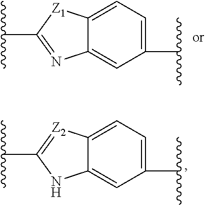

and is optionally substituted with one or more R_{A}; B is

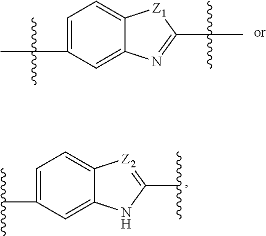

and is optionally substituted with one or more R_{A}. Z₁ is independently selected at each occurrence from O, S, NH or CH₂; and Z₂ is independently selected at each occurrence from N or CH. Preferably, A and B are each independently substituted with at least one halo such as F. D is a C₅-C₆carbocycle or 5- to 6-membered heterocycle (e.g., phenyl), and is substituted with J and optionally substituted with one or more R_{A}. J is C₃-C₆carbocycle, 3- to 6-membered heterocycle, 6- to 12-membered bicycle, 10- to 15-membered tricycle or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S}, or —C(O)R_{S}; and J can also be optionally substituted with one or more R_{A}. Preferably, D is

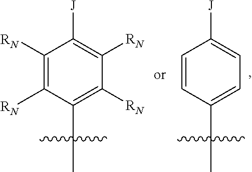

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. Y is -L_{S}-C(R₁R₂)N(R₅)-T-R_{D} or -L_{S}-C(R₃R₄)C(R₆R₇)-T-R_{D}. Z is -L_{S}-C(R₈R₉)N(R₁₂)-T-R_{D} or -L_{S}-C(R₁₀R₁₁)C(R₁₃R₁₄)-T-R_{D}. R₁ is R_{C}; and R₂ and R₅, taken together with the atoms to which they are attached, form a 5- to 6-membered heterocyclic ring

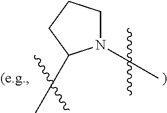

or 6- to 12-membered bicycle

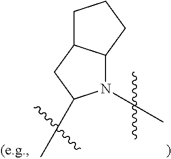

which is optionally substituted with one or more R_{A}; R₃ and R₆ are each independently R_{C}, and R₄ and R₇, taken together with the atoms to which they are attached, form a 5- to 6-membered carbocyclic or heterocyclic ring

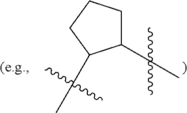

or 6- to 12-membered bicycle which is optionally substituted with one or more R_{A}. R₈ is R_{C}; and R₉ and R₁₂, taken together with the atoms to which they are attached, form a 5- to 6-membered heterocyclic ring

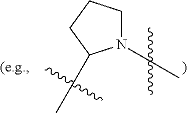

or 6- to 12-membered bicycle

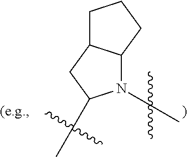

which is optionally substituted with one or more R_{A}; and R₁₀ and R₁₃ are each independently R_{C}, and R₁₁ and R₁₄, taken together with the atoms to which they are attached, form a 5- to 6-membered carbocyclic or heterocyclic ring

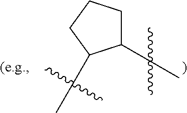

or 6- to 12-membered bicycle which is optionally substituted with one or more R_{A}. T is preferably independently selected at each occurrence from —C(O)-L_{Y}′-N(R_{B})C(O)-L_{S}″- or —C(O)-L_{Y}′-N(R_{B})C(O)O-L_{S}″-. L_{Y}′ is each independently L_{S}′ and, preferably, is each independently C₁-C₆alkylene (e.g., —CH₂—) and optionally substituted with one or more substituents selected from R_{L}. T can also be, without limitation, selected from —C(O)-L_{Y}′-L_{S}″-, —C(O)-L_{Y}′-O-L_{S}″-, —C(O)-L_{Y}′-N(R_{B})-L_{S}″-, or —C(O)-L_{Y}′-N(R_{B})S(O)₂-L_{S}″-. In some cases, Y and Z are independently

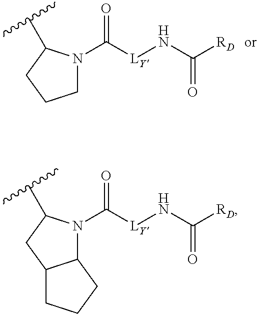

wherein non-limiting examples of R_{D} include (1) —O—C₁-C₆alkyl, —O—C₂-C₆alkenyl, —O—C₂-C₆alkynyl, C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or (2) C₃-C₆carbocycle or 3- to 6-membered heterocycle each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; and non-limiting examples of L_{Y}′ include C₁-C₆alkylene optionally substituted with halogen, hydroxy, mercapto, amino, carboxy, phosphonoxy, —O—C₁-C₆alkyl, —O—C₂-C₆alkenyl, —O—C₂-C₆alkynyl, or 3- to 6-membered carbocycle or heterocycle, said 3- to 6-membered carbocycle or heterocycle being optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another aspect, the present invention features compounds of Formula I_{A} and pharmaceutically acceptable salts thereof.

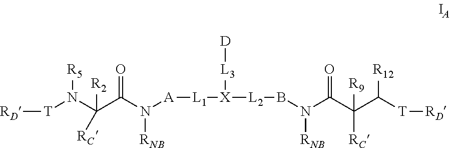

wherein:
R_{NB} is each independently selected from R_{B};
R_{C}′ is each independently selected from R_{C};
R_{D}′ is each independently selected from R_{D};
R₂ and R₅, taken together with the atoms to which they are attached, form a 3- to 12-membered heterocycle which is optionally substituted with one or more R_{A};
R₉ and R₁₂, taken together with the atoms to which they are attached, form a 3- to 12-membered heterocycle which is optionally substituted with one or more R_{A}; A, B, D, X, L₁, L₂, L₃, T, R_{A}, R_{B}, R_{C}, and R_{D} are as described above in Formula I.

In this aspect, A and B preferably are independently selected from C₅-C₆carbocycle or 5- to 6-membered heterocycle, and are each independently optionally substituted with one or more R_{A}. More preferably, at least one of A and B is phenyl

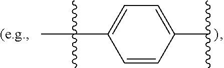

and is optionally substituted with one or more R_{A}. Highly preferably, both A and B are each independently phenyl

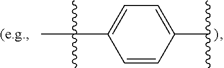

and are each independently optionally substituted with one or more R_{A}.

D preferably is selected from C₅-C₆carbocycle, 5- to 6-membered heterocycle, or 8- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more R_{L}. More preferably, D is C₅-C₆carbocycle, 5- to 6-membered heterocycle, or 6- to 12-membered bicycles, and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

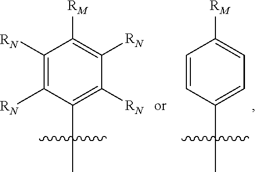

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

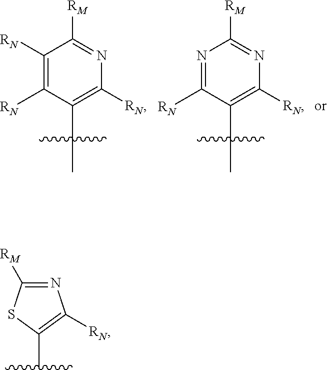

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

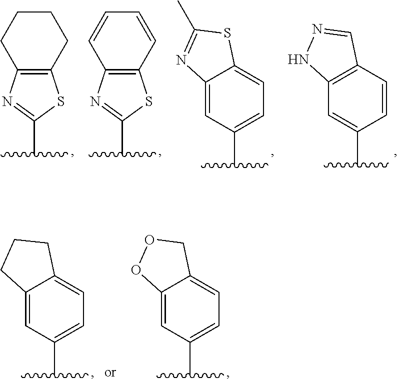

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is independently optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M is}-L_{S}-R_{E} where L_{S} is a bond and R_{E} is —N(R_{S}R_{S′}), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is independently optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or spiro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D is

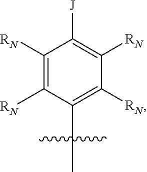

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is

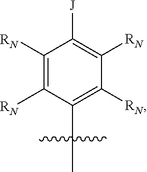

wherein each R_{N} is independently selected from R_{D} and preferably is hydrogen or halogen, and J is C₃-C₆carbocycle or 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

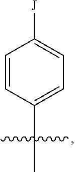

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

X preferably is C(H).

L₁ and L₂ are preferably independently bond or C₁-C₆alkylene, L₃ is preferably selected from bond, C₁-C₆alkylene or —C(O)—, and L₁, L₂, and L₃ are each independently optionally substituted with one or more R_{L}, and wherein at least one of L₁ or L₂ preferably is bond. More preferably, L₁, L₂ and L₃ are each independently bond or C₁-C₆alkylene (e.g., —CH₂— or —CH₂CH₂—), and are each independently optionally substituted with one or more R_{L}, and wherein at least one of L₁ or L₂ preferably is bond. Highly preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond.

R₂ and R₅, taken together with the atoms to which they are attached, preferably form a 5- to 6-membered heterocycle or 6- to 12-membered bicycle

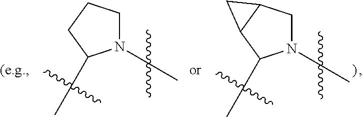

which is optionally substituted with one or more R_{A}.

R₉ and R₁₂, taken together with the atoms to which they are attached, preferably form a 5- to 6-membered heterocycle or 6- to 12-membered bicycle

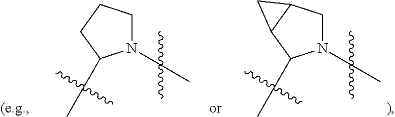

which is optionally substituted with one or more R_{A}.

-T-R_{D}′ can be, without limitation, independently selected at each occurrence from —C(O)-L_{Y}′-, —C(O)O-L_{Y}′-R_{D}′, —C(O)-L_{Y}′-N(R_{B})C(O)-L_{S}″-R_{D}′, —C(O)-L_{Y}′-N(R_{B})C(O)O-L_{S}″-R_{D}′, —N(R_{B})C(O)-L_{Y}′-N(R_{B})C(O)-L_{S}″-R_{D}′, —N(R_{B})C(O)-L_{Y}′-N(R_{B})C(O)O-L_{S}″-R_{D}′, or —N(R_{B})C(O)-L_{Y}′-N(R_{B})-L_{S}″-R_{D}′, wherein L_{Y}′ is each independently L_{S}′ and, preferably, is each independently C₁-C₆alkylene (e.g., —CH₂—) and optionally substituted with one or more substituents selected from R_{L}. Preferably, -T-R_{D}′ is independently selected at each occurrence from —C(O)-L_{Y}′-M′-L_{S}″-R_{D}′ or —N(R_{B})C(O)-L_{Y}′-M′-L_{S}″-R_{D}′. More preferably, -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′. Highly preferably, -T-R_{D}′ is independently selected at each occurrence from —C(O)-L_{Y}′-N(R_{B})C(O)—R_{D}′ or —C(O)-L_{Y}′-N(R_{B})C(O)O—R_{D}′, wherein L_{Y}′ preferably is each independently C₁-C₆alkylene (e.g., —CH₂—) and optionally substituted with one or more substituents selected from R_{L}.

R_{NB} and R_{C}′ are preferably hydrogen, and R_{D}′ preferably is independently selected at each occurrence from R_{E}. More preferably, R_{D}′ is independently selected at each occurrence from C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}′, -L_{A}-N(R_{S})C(O)N(R_{S}′R_{S}″), -L_{A}-N(R_{S})SO₂R_{S}′, -L_{A}-SO₂N(R_{S}R_{S}′), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

L_{S}, L_{S}′ and L_{S}″ preferably are each independently selected at each occurrence from bond; or C₁-C₆alkylene, C₂-C₆alkenylene or C₂-C₆alkynylene.

A and B can be the same or different. Likewise, L₁ and L₂ can be the same or different.

In one embodiment of this aspect, A and B are each independently phenyl, and are each independently optionally substituted with one or more R_{A}; D is phenyl, and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

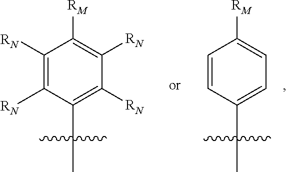

wherein R_{M} and R_{N} are as defined above. Also preferably, D is

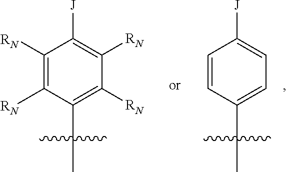

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. Preferably, R₂ and R₅, taken together with the atoms to which they are attached, form

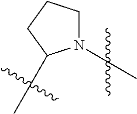

which is optionally substituted with one or more R_{A}; R₉ and R₁₂, taken together with the atoms to which they are attached, form

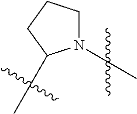

which is optionally substituted with one or more R_{A}.

In another embodiment of this aspect, A and B are each independently phenyl

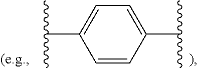

and are each independently optionally substituted with one or more R_{A} (preferably, A and B are each independently substituted with at least one halo such as F). D is phenyl, and is substituted with J and optionally substituted with one or more R_{A}. J is C₃-C₆carbocycle, 3- to 6-membered heterocycle, 6- to 12-membered bicycle, 10- to 15-membered tricycle or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S}; or —C(O)R_{S}; and J can also be optionally substituted with one or more R_{A}. Preferably, D is

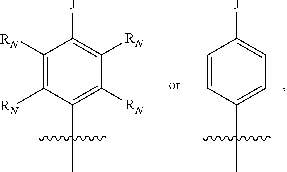

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. R₂ and R₅, taken together with the atoms to which they are attached, form a 5- to 6-membered heterocyclic ring

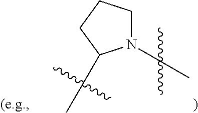

or 6- to 12-membered bicycle

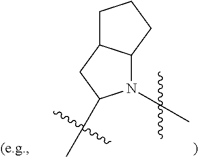

which is optionally substituted with one or more R_{A}; and R₉ and R₁₂, taken together with the atoms to which they are attached, form a 5- to 6-membered heterocyclic ring

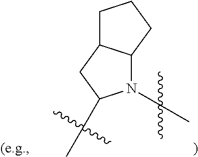

or 6- to 12-membered bicycle

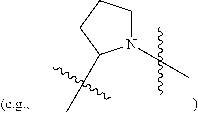

which is optionally substituted with one or more R_{A}.

In still another aspect, the present invention features compounds of Formula I_{D} and pharmaceutically acceptable salts thereof:

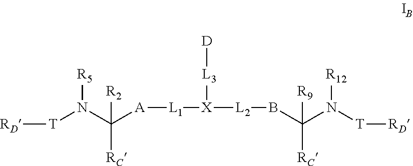

wherein:
R_{C}′ is each independently selected from R_{C};
R_{D}′ is each independently selected from R_{D};
R₂ and R₅, taken together with the atoms to which they are attached, form a 3- to 12-membered heterocycle which is optionally substituted with one or more R_{A};
R₉ and R₁₂, taken together with the atoms to which they are attached, form a 3- to 12-membered heterocycle which is optionally substituted with one or more R_{A}; A, B, D, X, L₁, L₂, L₃, T, R_{A}, R_{C}, and R_{D} are as described above in Formula I.

In this aspect, A and B preferably are independently selected from 8- to 12-membered bicycles such as

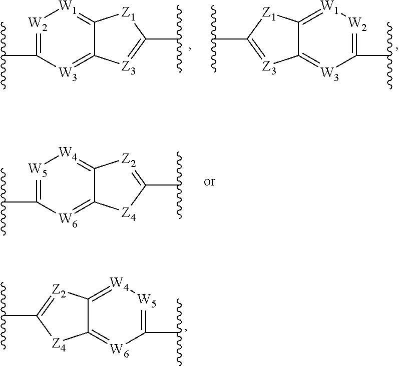

where Z₁ is independently selected at each occurrence from O, S, NH or CH₂, Z₂ is independently selected at each occurrence from N or CH, Z₃ is independently selected at each occurrence from N or CH, Z₄ is independently selected at each occurrence from O, S, NH or CH₂, and W₁, W₂, W₃, W₄, W₅ and W₆ are each independently selected at each occurrence from CH or N. A and B are each independently optionally substituted with one or more R_{A}.

More preferably, A is selected from

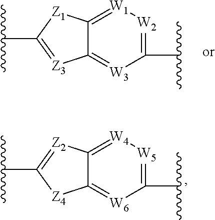

and is optionally substituted with one or more R_{A}; B is selected from

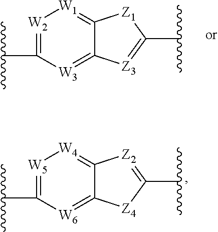

and is optionally substituted with one or more R_{A}, where Z₁, Z₂, Z₃, Z₄, W₁, W₂, W₃, W₄, W₅, W₆ are as defined above. Preferably. Z₃ is N and Z₄ is NH. For instance. A can be selected from

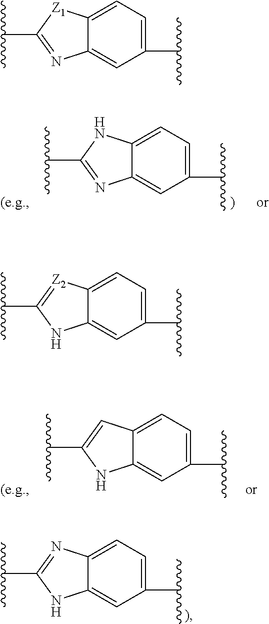

and is optionally substituted with one or more R_{A}; and B can be selected from

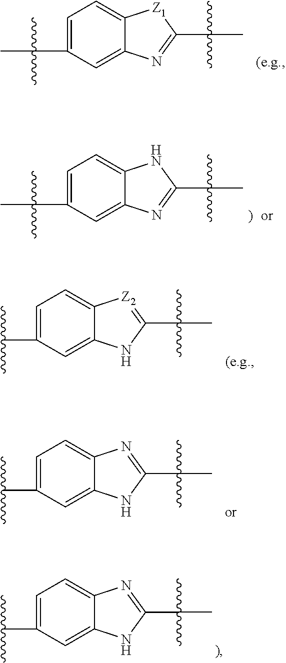

and is optionally substituted with one or more R_{A}.

Also preferably, A is

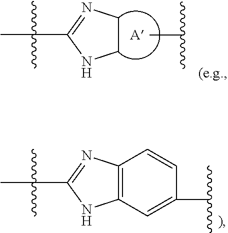

### and B is

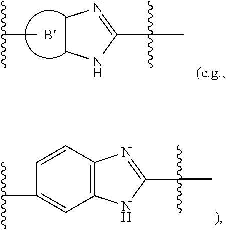

wherein A′ and B′ are independently selected from C₅-C₆carbocycle or 5- to 6-membered heterocycle, and A and B are independently optionally substituted with one or more R_{A}.

D preferably is selected from C₅-C₆carbocycle, 5- to 6-membered heterocycle, or 6- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more substituents selected from R_{L}. More preferably, D is C₅-C₆carbocycle, 5- to 6-membered heterocycle, or 6- to 12-membered bicycles, and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

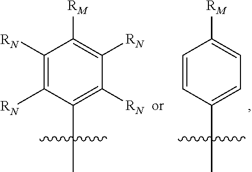

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

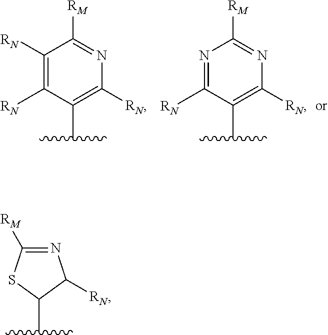

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

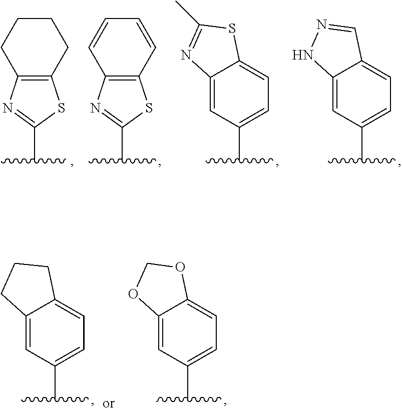

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M} is -L_{S}-R_{E} where L_{S} is a bond and R_{E} is —N(R_{S}R_{S′}), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or spiro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D is

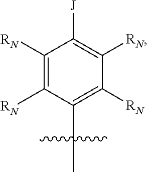

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₉-C₆haloalkenyl, C₂-C₆haloalkynyl, C(O)OR_{S} or —N(R_{S}R_{S}′). Also preferably, D is

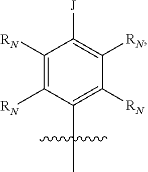

wherein each R_{N} is independently selected from R_{D} and preferably is hydrogen or halogen, and J is C₃-C₆carbocycle or 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

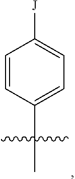

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

X preferably is C(H).

L₁ and L₂ are preferably independently bond or C₁-C₆alkylene, L₃ is preferably selected from bond, C₁-C₆alkylene or —C(O)—, and L₁, L₂, and L₃ are each independently optionally substituted with one or more R_{L}, and wherein at least one of L₁ or L₂ preferably is bond. More preferably, L₁, L₂ and L₃ are each independently bond or C₁-C₆alkylene (e.g., —CH₂— or —CH₂CH₂—), and are each independently optionally substituted with one or more R_{L}, and wherein at least one of L₁ or L₂ preferably is bond. Highly preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond.

R₂ and R₅, taken together with the atoms to which they are attached, preferably form a 5- to 6-membered heterocycle or 6- to 12-membered bicycle

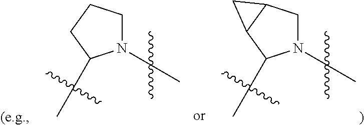

which is optionally substituted with one or more R_{A}. R₉ and R₁₂, taken together with the atoms to which they are attached, preferably form a 5- to 6-membered heterocycle or 6- to 12-membered bicycle

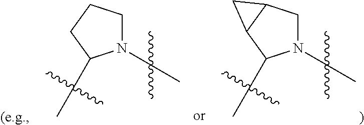

which is optionally substituted with one or more R_{A}.

-T-R_{D}′ can be, without limitation, independently selected at each occurrence from —C(O)-L_{Y}′-R_{D}′, —C(O)O-L_{Y}′-R_{D}′, —C(O)-L_{Y}′-N(R_{B})C(O)-L_{S}″-R_{D}′, —C(O)-L_{Y}′-N(R_{B})C(O)O-L_{S}″-R_{D}′, —N(R_{B})C(O)-L_{Y}′-N(R_{B})C(O)-L_{S}″-R_{D}′, —N(R_{B})C(O)-L_{Y}′-N(R_{B})C(O)O-L_{S}″-R_{D}′, or N(R_{B})C(O)-L_{Y}′-N(R_{B})-L_{S}″-R_{D}′, wherein L_{Y}′ is each independently L_{S}′ and, preferably, is each independently C₁-C₆alkylene (e.g., —CH₂—) and optionally substituted with one or more substituents selected from R_{L}. Preferably, -T-R_{D}′ is independently selected at each occurrence from —C(O)-L_{Y}′-M′-L_{S}″-R_{D}′ or —N(R_{B})C(O)-L_{Y}′-M′-L_{S}″-R_{D}′. More preferably, -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′. Highly preferably, -T-R_{D}′ is independently selected at each occurrence from —C(O)-L_{Y}′-N(R_{B})C(O)—R_{D}′ or —C(O)-L_{Y}′-N(R_{B})C(O)O—R_{D}′, wherein L_{Y}′ preferably is each independently C₁-C₆alkylene (e.g., —CH₂—) and optionally substituted with one or more substituents selected from R_{L}.

R_{C}′ is preferably hydrogen, and R_{D}′ preferably is independently selected at each occurrence from R_{E}. More preferably, R_{D}′ is independently selected at each occurrence from C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}′, -L_{A}-N(R_{S})C(O)N(R_{S}′R_{S}″), -L_{A}-N(R_{S})SO₂R_{S}′, -L_{A}-SO₂N(R_{S}R_{S}′), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

L_{S}, L_{S}′ and L_{S}″ preferably are each independently selected at each occurrence from bond; or C₁-C₆alkylene, C₂-C₆alkenylene or C₂-C₆alkynylene.

A and B can be the same or different. Likewise, L₁ and L₂ can be the same or different.

In one embodiment of this aspect, A is

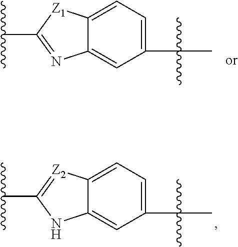

and is optionally substituted with one or more R_{A}; B is

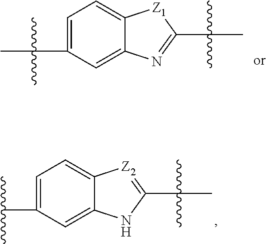

and is optionally substituted with one or more R_{A}; and D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′) and J can also be optionally substituted with one or more R_{A}. Preferably, D is

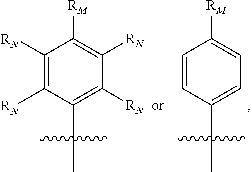

wherein R_{M} and R_{N} are as defined above. Also preferably, D is

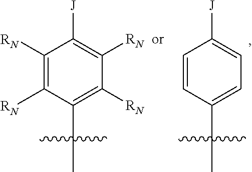

wherein J and R_{N} are as defined above. Z₁ is independently selected at each occurrence from O, S, NH or CH₂; and Z₂ is independently selected at each occurrence from N or CH. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′.

In another embodiment of this aspect, A is

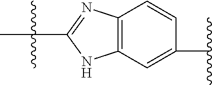

and optionally substituted with one or more R_{A} (e.g., halogen); B is

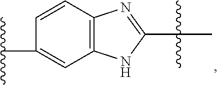

and is optionally substituted with one or more R_{A} (e.g., halogen); and D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

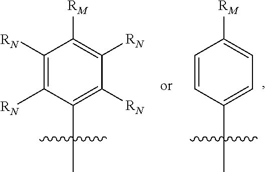

wherein R_{M} and R_{N} are as defined above. Also preferably, D is

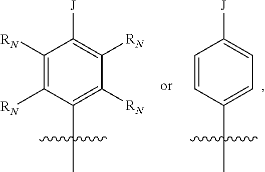

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. R₂ and R₅, taken together with the atoms to which they are attached, preferably form a 5- to 6-membered heterocycle or 6- to 12-membered bicycle

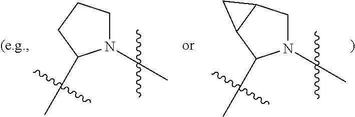

which is optionally substituted with one or more R_{A}. R₉ and R₁₂, taken together with the atoms to which they are attached, preferably form a 5- to 6-membered heterocycle or 6- to 12-membered bicycle

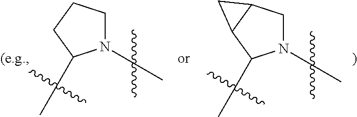

which is optionally substituted with one or more R_{A}. More preferably, R₂ and R₅, taken together with the atoms to which they are attached, form

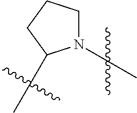

which is optionally substituted with one or more R_{A}; R₉ and R₁₂, taken together with the atoms to which they are attached, form

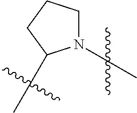

which is optionally substituted with one or more R_{A}.

In still another embodiment of this aspect, A is

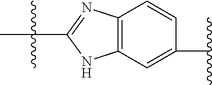

and optionally substituted with one or more R_{A} (preferably, A is substituted with at least one halogen such as F); B is

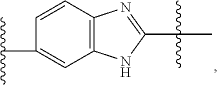

and is optionally substituted with one or more R_{A} (preferably, B is substituted with at least one halogen such as F). D is phenyl, and is substituted with J and optionally substituted with one or more R_{A}. J is C₃-C₆carbocycle, 3- to 6-membered heterocycle, 6- to 12-membered bicycle, 10- to 15-membered tricycle or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S} or —C(O)R_{S}; and J can also be optionally substituted with one or more R_{A}. Preferably, D is

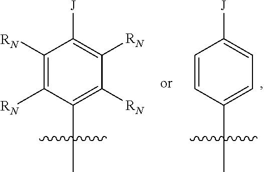

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. R₂ and R₅, taken together with the atoms to which they are attached, preferably form a 5- to 6-membered heterocycle or 6- to 12-membered bicycle

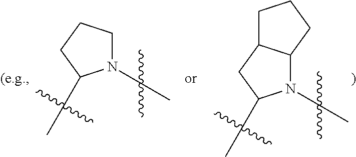

which is optionally substituted with one or more R_{A}. R₉ and R₁₂, taken together with the atoms to which they are attached, preferably form a 5- to 6-membered heterocycle or 6- to 12-membered bicycle

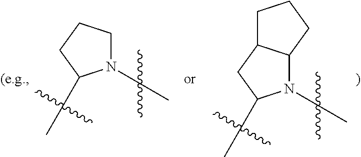

which is optionally substituted with one or more R_{A}. More preferably, R₂ and R₅, taken together with the atoms to which they are attached, form

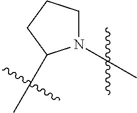

which is optionally substituted with one or more R_{A}; R₉ and R₁₂, taken together with the atoms to which they are attached, form

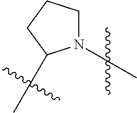

which is optionally substituted with one or more R_{A}.

In yet another aspect, the present invention further features compounds of Formula I_{C} and pharmaceutically acceptable salts thereof.

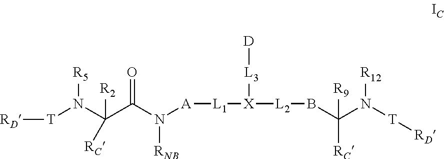

wherein:
R_{NB} is R_{B};
R_{C}′ is each independently selected from R_{C};
R_{D}′ is each independently selected from R_{D};
R₂ and R₅, taken together with the atoms to which they are attached, form a 3- to 12-membered heterocycle which is optionally substituted with one or more R_{A};
R₉ and R₁₂, taken together with the atoms to which they are attached, form a 3- to 12-membered heterocycle which is optionally substituted with one or more R_{A};
A, B, D, X, L₁, L₂, L₃, T, R_{A}, R_{B}, R_{C}, and R_{D} are as described above in Formula I.

In this aspect, A preferably is C₅-C₆carbocycle or 5- to 6-membered heterocycle, and is optionally substituted with one or more R_{A}; and B preferably is 8- to 12-membered bicycle (such as

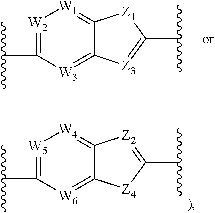

and is optionally substituted with one or more R_{A}. Z₁ is O, S, NH or CH₂; Z₂ is N or CH; Z₃ is N or CH; Z₄ is O, S, NH or CH₂; and W₁, W₂, W₃, W₄, W₅ and W₆ are each independently selected from CH or N.

More preferably, A is phenyl

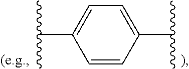

and is optionally substituted with one or more R_{A}; and B is

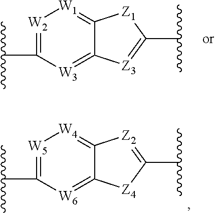

and is optionally substituted with one or more R_{A}, where Z₁, Z₂, Z₃, Z₄, W₁, W₂, W₃, W₄, W₅, W₆ are as defined above. Preferably, Z₃ is N and Z₄ is NH. For instance, B can be

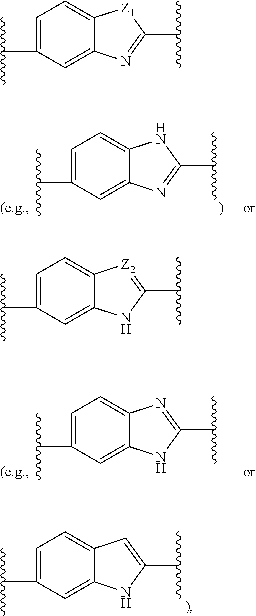

and is optionally substituted with one or more R_{A}.

Also preferably, A is C₅-C₆carbocycle (e.g., phenyl such as

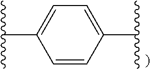

or 5- to 6-membered heterocycle; and B is

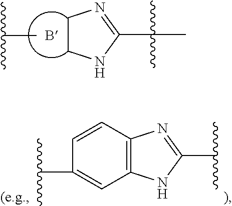

wherein B′ is selected from C₅-C₆carbocycle or 5- to 6-membered heterocycle. A and B are independently optionally substituted with one or more R_{A}.

D preferably is selected from C₅-C₆carbocycle, 5- to 6-membered heterocycle, or 6- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more substituents selected from R_{L}. More preferably, D is C₅-C₆carbocycle, 5- to 6-membered heterocycle, or 6- to 12-membered bicycles, and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

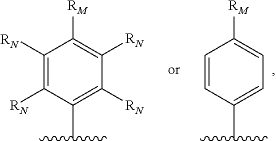

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

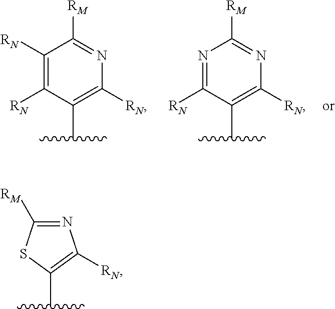

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

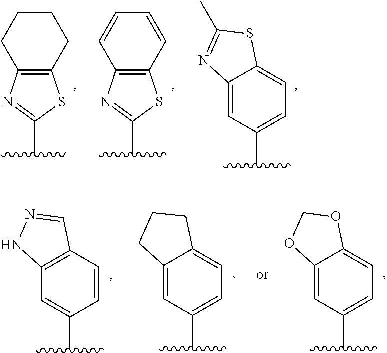

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M is}-L_{S}-R_{E} where L_{S} is a bond and R_{E} is —N(R_{S}R_{S}′), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or spiro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D is

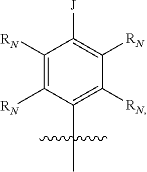

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is

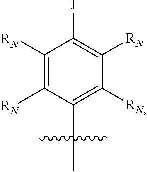

wherein each R_{N} is independently selected from R_{D} and preferably is hydrogen or halogen, and J is C₃-C₆carbocycle or 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

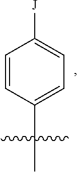

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

X preferably is C(H).

L₁ and L₂ are preferably independently bond or C₁-C₆alkylene, L₃ is preferably selected from bond, C₁-C₆alkylene or —C(O)—, and L₁, L₂, and L₃ are each independently optionally substituted with one or more R_{L}, and wherein at least one of L₁ or L₂ preferably is bond. More preferably, L₁, L₂ and L₃ are each independently bond or C₁-C₆alkylene (e.g., —CH₂— or —CH₂CH₂—), and are each independently optionally substituted with one or more R_{L}, and wherein at least one of L₁ or L₂ preferably is bond. Highly preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond.

R₂ and R₅, taken together with the atoms to which they are attached, preferably form a 5- to 6-membered heterocycle or 6- to 12-membered bicycle

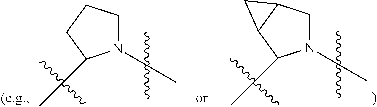

which is optionally substituted with one or more R_{A}. R₉ and R₁₂, taken together with the atoms to which they are attached, preferably form a 5- to 6-membered heterocycle or 6- to 12-membered bicycle

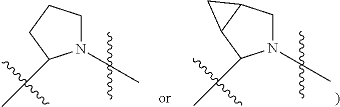

which is optionally substituted with one or more R_{A}.

-T-R_{D}′ can be, without limitation, independently selected at each occurrence from —C(O)-L_{Y}′-R_{D}′, —C(O)O-L_{Y}′-R_{D}′, —C(O)-L_{Y}′-N(R_{B})C(O)-L_{S}″-R_{D}′, —C(O)-L_{Y}′-N(R_{B})C(O)O-L_{S}″-R_{D}′, N(R_{B})C(O)-L_{Y}′-N(R_{B})C(O)-L_{S}″-R_{D}′, —N(R_{B})C(O)-L_{Y}′-N(R_{B})C(O)O-L_{S}″-R_{D}′, or N(R_{B})C(O)-L_{Y}′-N(R_{B})-L_{S}″-R_{D}′, wherein L_{Y}′ is each independently L_{S}′ and, preferably, is each independently C₁-C₆alkylene (e.g., —CH₂—) and optionally substituted with one or more substituents selected from R_{L}. Preferably, -T-R_{D}′ is independently selected at each occurrence from —C(O)-L_{Y}′-M′-L_{S}″-R_{D}′ or —N(R_{B})C(O)-L_{Y}′-M′-L_{S}″-R_{D}′. More preferably, -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′. Highly preferably, -T-R_{D}′ is independently selected at each occurrence from —C(O)-L_{Y}′-N(R_{B})C(O)—R_{D}′ or —C(O)-L_{Y}′-N(R_{B})C(O)O—R_{D}′, wherein L_{Y}′ preferably is each independently C₁-C₆alkylene (e.g., —CH₂—) and optionally substituted with one or more substituents selected from R_{L}.

R_{NB} and R_{C}′ are preferably hydrogen, and R_{D}′ preferably is independently selected at each occurrence from R_{E}. More preferably, R_{D}′ is independently selected at each occurrence from C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}′, -L_{A}-N(R_{S})C(O)N(R_{S}′R_{S}″), -L_{A}-N(R_{S})SO₂R_{S}′, -L_{A}-SO₂N(R_{S}R_{S}′), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

L_{S}, L_{S}′ and L_{S}″ preferably are each independently selected at each occurrence from bond; or C₁-C₆alkylene, C₂-C₆alkenylene or C₂-C₆alkynylene.

In one embodiment of this aspect, A is phenyl, and is optionally substituted with one or more R_{A}; and B is

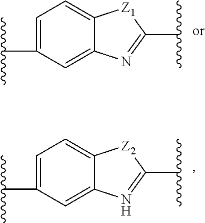

and is optionally substituted with one or more R_{A}, wherein Z₁ is O, S, NH or CH₂; and Z₂ is N or CH. D is C₅-C₆carbocycle or 5- to 6-membered heterocycle (e.g., phenyl), and is optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Preferably, D is

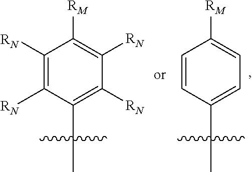

wherein R_{M} and R_{N} are as defined above. Also preferably, D is

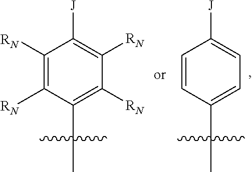

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. Preferably, R₂ and R₅, taken together with the atoms to which they are attached, form

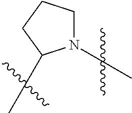

which is optionally substituted with one or more R_{A}; R₉ and R₁₂, taken together with the atoms to which they are attached, form

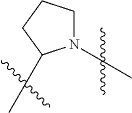

which is optionally substituted with one or more R_{A}.

In another embodiment of this aspect, A is phenyl

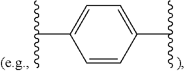

and is optionally substituted with one or more R_{A} (preferably, A is substituted with at least one halogen such as F); and B is

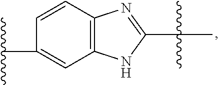

and is optionally substituted with one or more R_{A} (preferably, B is substituted with at least one halogen such as F). D is phenyl, and is substituted with J and optionally substituted with one or more R_{A}. J is C₃-C₆carbocycle, 3- to 6-membered heterocycle, 6- to 12-membered bicycle, 10- to 15-membered tricycle or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S} or —C(O)R_{S}; and J can also be optionally substituted with one or more R_{A}. Preferably, D is

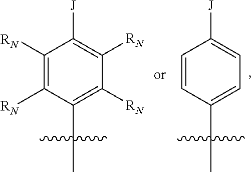

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B}) S(O)₂-L_{S}″-R_{D}′. Preferably, R₂ and R₅, taken together with the atoms to which they are attached, form a 5- to 6-membered heterocyclic ring

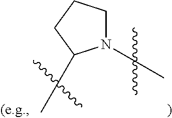

or 6- to 12-membered bicycle

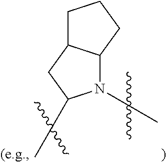

which is optionally substituted with one or more R_{A}; R₉ and R₁₂, taken together with the atoms to which they are attached, form a 5- to 6-membered heterocyclic ring

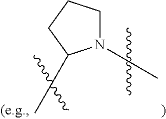

or 6- to 12-membered bicycle

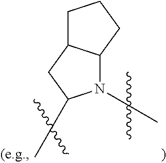

which is optionally substituted with one or more R_{A}.

In yet another aspect, the present invention features compounds of Formula I_{D} and pharmaceutically acceptable salts thereof.

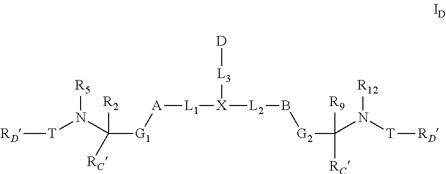

wherein:
G₁ and G₂ are each independently selected from C₅-C₆carbocycle or 5- to 6-membered heterocycle, and are each independently optionally substituted with one or more R_{A};
R_{C}′ is each independently selected from R_{C};
R_{D}′ is each independently selected from R_{D};
R₂ and R₅, taken together with the atoms to which they are attached, form a 3- to 12-membered heterocycle which is optionally substituted with one or more R_{A};
R₉ and R₁₂, taken together with the atoms to which they are attached, form a 3- to 12-membered heterocycle which is optionally substituted with one or more R_{A};
A, B, D, X, L₁, L₂, L₃, T, R_{A}, R_{C}, and R_{D} are as described above in Formula I.

In this aspect, A and B preferably are independently selected from C₅-C₆carbocycle or 5- to 6-membered heterocycle, and are each independently optionally substituted with one or more R_{A}. More preferably, at least one of A and B is phenyl

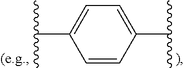

and is optionally substituted with one or more R_{A}. Highly preferably, both A and B are each independently phenyl

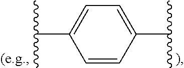

and are each independently optionally substituted with one or more R_{A}.

D preferably is selected from C₅-C₆carbocycle, 5- to 6-membered heterocycle, or 8- to 12-membered bicycles, and is optionally substituted with one or more R_{A}. D can also be preferably selected from C₁-C₆alkyl, C₂-C₆alkenyl or C₂-C₆alkynyl, and is optionally substituted with one or more R_{L}. More preferably, D is C₅-C₆carbocycle, 5- to 6-membered heterocycle, or 6- to 12-membered bicycles, and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

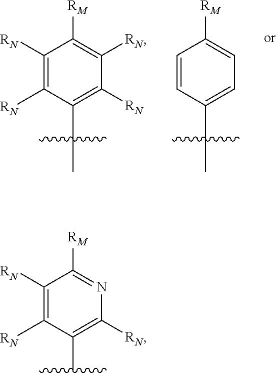

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

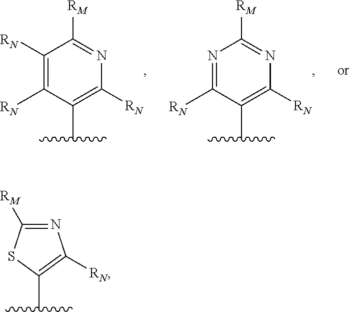

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl, and is substituted with one or more R_{M}. Highly preferably, D is

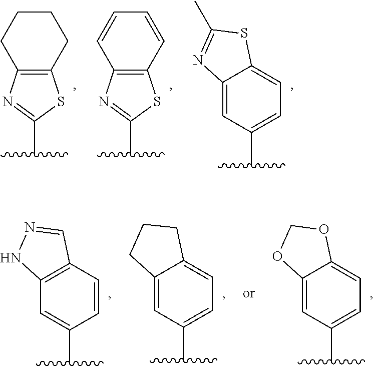

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is halogen, hydroxy, mercapto, amino, carboxy; or C₁-C₆alkyl, C₂-C₆alkenyl or C₂-C₆alkynyl, each of which is independently optionally substituted at each occurrence with one or more substituents selected from halogen, hydroxy, mercapto, amino or carboxy. Highly preferably, R_{M} is C₁-C₆alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR_{S}, or —P(O)(OR_{S})₂, wherein R_{S} and R_{S}′ can be, for example, each independently selected at each occurrence from (1) hydrogen or (2) C₁-C₆alkyl optionally substituted at each occurrence with one or more halogen, hydroxy, —O—C₁-C₆alkyl or 3- to 6-membered heterocycle; or R_{M} is C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example R_{M} is CF₃, —C(CF₃)₂—OH, —C(CH₃)₂—CN, —C(CH₃)₂—CH₂OH, or —C(CH₃)₂—CH₂NH₂. Also preferably R_{M} is -L_{S}-R_{E} where L_{S} is a bond and R_{E} is —N(R_{S}R_{S′}), —O—R_{S}, —N(R_{S})C(O)OR_{S}′, —N(R_{S})SO₂R_{S}′, —SO₂R_{S}, or —SR_{S}. For example where L_{S} is a bond, R_{E} is —N(C₁-C₆alkyl)₂ (e.g., —NMe₂); —N(C₁-C₆alkylene-O—C₁-C₆alkyl)₂ (e.g. —N(CH₂CH₂OMe)₂); —N(C₁-C₆alkyl)(C₁-C₆alkylene-O—C₁-C₆alkyl) (e.g. —N(CH₃)(CH₂CH₂OMe)); —O—C₁-C₆alkyl (e.g., —O-Me, —O-Et, —O-isopropyl, —O-tert-butyl, —O-n-hexyl); —O—C₁-C₆haloalkyl (e.g., —OCF₃, —OCH₂CF₃); —O—C₁-C₆alkylene-piperidine (e.g., —O—CH₂CH₂-1-piperidyl); —N(C₁-C₆alkyl)C(O)OC₁-C₆alkyl (e.g., —N(CH₃)C(O)O—CH₂CH(CH₃)₂), —N(C₁-C₆alkyl)SO₂C₁-C₆alkyl (e.g., —N(CH₃)SO₂CH₃); —SO₂C₁-C₆alkyl (e.g., —SO₂Me); —SO₂C₁-C₆haloalkyl (e.g., —SO₂CF₃); or —S—C₁-C₆haloalkyl (e.g., SCF₃). Also preferably R_{M} is -L_{S}-R_{E} where L_{S} is C₁-C₆alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆alkylene-O—R_{S} (e.g., —C(CH₃)₂—CH₂—OMe); —C₁-C₆alkylene-C(O)OR_{S} (e.g., —C(CH₃)₂—C(O)OMe); —C₁-C₆alkylene-N(R_{S})C(O)OR_{S}′ (e.g., —C(CH₃)₂—CH₂—NHC(O)OCH₃); or —C₁-C₆alkylene-P(O)(OR_{S})₂ (e.g., —CH₂—P(O)(OEt)₂). Also more preferably R_{M} is C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or spiro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Highly preferably, D is

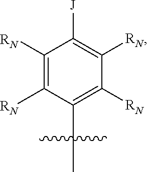

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′). Also preferably, D is

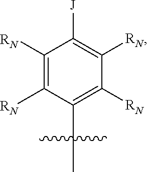

wherein each R_{N} is independently selected from R_{D} and preferably is hydrogen or halogen, and J is C₃-C₆carbocycle or 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}. Also preferably, D is

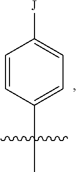

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′).

X preferably is C(H).

L₁ and L₂ are preferably independently bond or C₁-C₆alkylene, L₃ is preferably selected from bond, C₁-C₆alkylene or —C(O)—, and L₁, L₂, and L₃ are each independently optionally substituted with one or more R_{L}, and wherein at least one of L₁ or L₂ preferably is bond. More preferably, L₁, L₂ and L₃ are each independently bond or C₁-C₆alkylene (e.g., —CH₂— or —CH₂CH₂—), and are each independently optionally substituted with one or more R_{L}, and wherein at least one of L₁ or L₂ preferably is bond. Highly preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond.

R₂ and R₅, taken together with the atoms to which they are attached, preferably form a 5- to 6-membered heterocycle or 6- to 12-membered bicycle

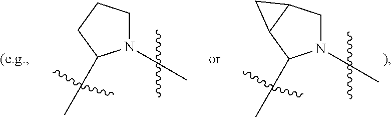

which is optionally substituted with one or more R_{A}.

R₉ and R₁₂, taken together with the atoms to which they are attached, preferably form a 5- to 6-membered heterocycle or 6- to 12-membered bicycle

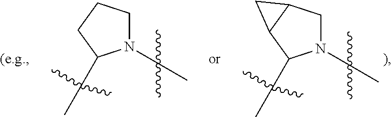

which is optionally substituted with one or more R_{A}.

G₁ and G₂ preferably are each independently selected from

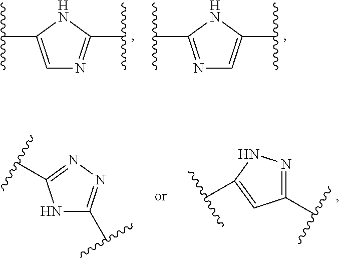

and are each independently optionally substituted with one or more R_{A} (e.g., one or more chloro or bromo). More preferably, G₁ is

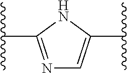

(including any tautomer thereof), and G₂ is

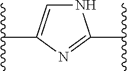

(including any tautomer thereof), and each G₁ and G₂ is independently optionally substituted with one or more R_{A} (e.g., one or more chloro or bromo).

-T-R_{D}′ can be, without limitation, independently selected at each occurrence from —C(O)-L_{T}′-, —C(O)O-L_{Y}′-R_{D}′, —C(O)-L_{Y}′-N(R_{B})C(O)-L_{S}″-R_{D}′, —C(O)-L_{Y}′-N(R_{B})C(O)O-L_{S}″-R_{D}′, —N(R_{B})C(O)-L_{Y}′-N(R_{B})C(O)-L_{S}″-R_{D}′, —N(R_{B})C(O)-L_{Y}′-N(R_{B})C(O)O-L_{S}″-R_{D}′, or N(R_{B})C(O)-L_{Y}′-N(R_{B})-L_{S}″-R_{D}′, wherein L_{Y}′ is each independently L_{S}′ and, preferably, is each independently C₁-C₆alkylene (e.g., —CH₂—) and optionally substituted with one or more substituents selected from R_{L}. Preferably, -T-R_{D}′ is independently selected at each occurrence from —C(O)-L_{Y}′-M′-L_{S}″-R_{D}′ or —N(R_{B})C(O)-L_{Y}′-M′-L_{S}″-R_{D}′. More preferably, -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′. Highly preferably, -T-R_{D}′ is independently selected at each occurrence from —C(O)-L_{Y}′-N(R_{B})C(O)—R_{D}′ or —C(O)-L_{Y}′-N(R_{B})C(O)O—R_{D}′, wherein L_{Y}′ preferably is each independently C₁-C₆alkylene (e.g., —CH₂—) and optionally substituted with one or more substituents selected from R_{L}.

R_{C}′ is preferably hydrogen, and R_{D}′ preferably is independently selected at each occurrence from R_{E}. More preferably, R_{D}′ is independently selected at each occurrence from C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆carbocycle or 3- to 6-membered heterocycle; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}′, -L_{A}-N(R_{S})C(O)N(R_{S}′R_{S}″), -L_{A}-N(R_{S})SO₂R_{S}′, -L_{A}-SO₂N(R_{S}R_{S}′), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

L_{S}, L_{S}′ and L_{S}″ preferably are each independently selected at each occurrence from bond; or C₁-C₆alkylene, C₂-C₆alkenylene or C₂-C₆alkynylene.

A and B can be the same or different. Likewise, L₁ and L₂ can be the same or different.

In one embodiment of this aspect, A and B are each independently phenyl, and are each independently optionally substituted with one or more R_{A}; D is phenyl, and is independently optionally substituted with one or more R_{A}, or is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, C(O)OR_{S} or —N(R_{S}R_{S}′), and J can also be optionally substituted with one or more R_{A}; and G₁ is

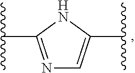

### G₂ is

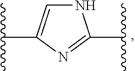

and each G₁ and G₂ is independently optionally substituted with one or more R_{A} (e.g., one or more chloro or bromo). Preferably, D is

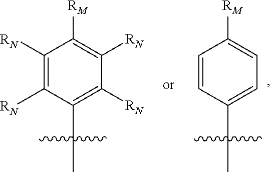

wherein R_{M} and R_{N} are as defined above. Also preferably, D is

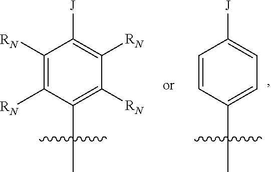

wherein J and R_{N} are as defined above. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. Preferably, R₂ and R₅, taken together with the atoms to which they are attached, form

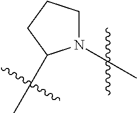

which is optionally substituted with one or more R_{A}; R₉ and R₁₂, taken together with the atoms to which they are attached, form

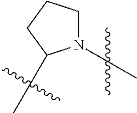

which is optionally substituted with one or more R_{A}.

In another embodiment of this aspect, A and B are each independently phenyl

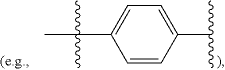

and are each independently optionally substituted with one or more R_{A} (preferably, A and B are each independently substituted with at least one halogen such as F). D is phenyl, and is substituted with J and optionally substituted with one or more R_{A}. J is C₃-C₆carbocycle, 3- to 6-membered heterocycle, 6- to 12-membered bicycle, 10- to 15-membered tricycle or 13- to 15-membered carbocycle/heterocycle, and J is optionally substituted with one or more R_{A}. Preferably, J is substituted with a C₃-C₆carbocycle, 3- to 6-membered -heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S} or —C(O)R_{S}; and J can also be optionally substituted with one or more R_{A}. Preferably, D is

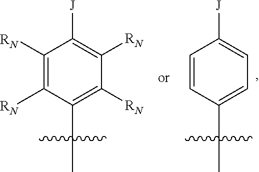

wherein J is as defined above, and each R_{N} is independently selected from R_{D} and preferably is hydrogen or halo such as F. G₁ is

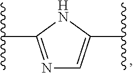

### G₂ is

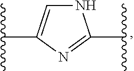

and each G₁ and G₂ is independently optionally substituted with one or more R_{A} (e.g., one or more chloro or bromo). L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁ is bond, L₂ is C₁-C₆alkylene (e.g., —CH₂— or —CH₂CH₂—) and is optionally substituted with one or more R_{L}, and L₃ are bond; or L₂ is bond, L₁ is C₁-C₆alkylene (e.g., —CH₂— or —CH₂CH₂—) and is optionally substituted with one or more R_{L}, and L₃ are bond. -T-R_{D}′ is independently selected at each occurrence from —C(O)-L_{Y}′-N(R_{B})C(O)-L_{S}″-R_{D}′ or —C(O)-L_{Y}′-N(R_{B})C(O)O-L_{S}″-R_{D}′, wherein L_{Y}′ is C₁-C₆alkylene (e.g., —CH₂—) and optionally substituted with one or more substituents selected from R_{L}, and L_{S}″ preferably is bond. -T-R_{D}′ can also be, without limitation, selected from —C(O)-L_{Y}′-L_{S}″-R_{D}′, —C(O)-L_{Y}′-O-L_{S}″-R_{D}′, —C(O)-L_{Y}′-N(R_{B})-L_{S}″-R_{D}′, or —C(O)-L_{Y}′-N(R_{B})S(O)₂-L_{S}″-R_{D}′. Preferably, R₂ and R₅, taken together with the atoms to which they are attached, form a 5- to 6-membered heterocyclic ring

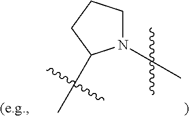

or 6- to 12-membered bicycle

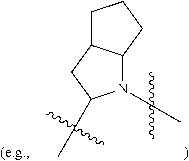

which is optionally substituted with one or more R_{A}; R₉ and R₁₂, taken together with the atoms to which they are attached, form a 5- to 6-membered heterocyclic ring

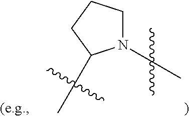

or 6- to 12-membered bicycle

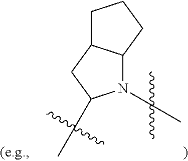

which is optionally substituted with one or more R_{A}.

In another aspect, the present invention features compounds having Formula I_{E} and pharmaceutically acceptable salts thereof,

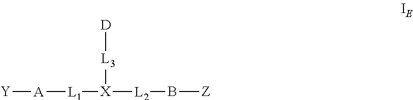

wherein:
X is C(H) and is substituted with one or more R_{A};
L₁ and L₂ are each independently selected from bond or C₁-C₆alkylene which is independently optionally substituted at each occurrence with one or more halo, hydroxy, —O—C₁-C₆alkyl, or —O—C₁-C₆haloalkyl; (preferably, L₁ is bond, and L₂ is C₁-C₆alkylene (e.g., —CH₂— or —CH₂CH₂—) and is optionally substituted with one or more R_{L}; or L₂ is bond, L₁ is C₁-C₆alkylene (e.g., —CH₂— or —CH₂CH₂—) and is optionally substituted with one or more R_{L});
L₃ is bond or C₁-C₆alkylene;
A and B are each independently phenyl, pyridinyl, thiazolyl, or

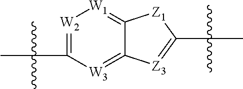

where Z₁ is independently selected at each occurrence from O, S, NH or CH₂, Z₃ is independently selected at each occurrence from N or CH, and W₁, W₂, and W₃ are each independently selected at each occurrence from CH or N; A and B are each independently optionally substituted with one or more R_{A}.
D is C₆-C₁₀carbocycle or 5- to 12-membered heterocycle, each of which is optionally substituted with one or more R_{M};
Y is -T′-C(R₁R₂)N(R₅)-T-R_{D};
Z is -T′-C(R₈R₉)N(R₁₂)-T-R_{D};
R₁ is hydrogen, C₁-C₆alkyl, C₁-C₆haloalkyl, or 3- to 6-membered carbocycle or heterocycle, wherein each said 3- to 6-membered carbocycle or heterocycle is independently optionally substituted at each occurrence with one or more substituents selected from halogen, C₁-C₆alkyl, C₁-C₆haloalkyl, —O—C₁-C₆alkyl or —O—C₁-C₆haloalkyl;
R₂ and R₅ are each independently hydrogen, C₁-C₆alkyl, C₁-C₆haloalkyl, or 3- to 6-membered carbocycle or heterocycle, wherein each said 3- to 6-membered carbocycle or heterocycle is independently optionally substituted at each occurrence with one or more substituents selected from halogen, C₁-C₆alkyl, C₁-C₆haloalkyl, —O—C₁-C₆alkyl or —O—C₁-C₆haloalkyl; or R₂ and R₅, taken together with the atoms to which they are attached, form a 3- to 12-membered heterocycle which is optionally substituted with one or more R_{A} (e.g., 1, 2, 3, or 4 R_{A});
R₈ is hydrogen, C₁-C₆alkyl, C₁-C₆haloalkyl, or 3- to 6-membered carbocycle or heterocycle, wherein each said 3- to 6-membered carbocycle or heterocycle is independently optionally substituted at each occurrence with one or more substituents selected from halogen, C₁-C₆alkyl, C₁-C₆haloalkyl, —O—C₁-C₆alkyl or —O—C₁-C₆haloalkyl;
R₉ and R₁₂ are each independently hydrogen, C₁-C₆alkyl, C₁-C₆haloalkyl, or 3- to 6-membered carbocycle or heterocycle, wherein each said 3- to 6-membered carbocycle or heterocycle is independently optionally substituted at each occurrence with one or more substituents selected from halogen, C₁-C₆alkyl, C₁-C₆haloalkyl, —O—C₁-C₆alkyl or —O—C₁-C₆haloalkyl; or R₉ and R₁₂, taken together with the atoms to which they are attached, form a 3- to 12-membered heterocycle which is optionally substituted with one or more R_{A} (e.g., 1, 2, 3, or 4 R_{A});
T is independently selected at each occurrence from bond or —C(O)-L_{S}′-;
T′ is independently selected at each occurrence from bond, —C(O)N(R_{B})—, —N(R_{B})C(O)—, or 3- to 12-membered heterocycle, wherein said 3- to 12-membered heterocycle is independently optionally substituted at each occurrence with one or more R_{A};
R_{D} is each independently selected at each occurrence from hydrogen or R_{A};
R_{A} is independently selected at each occurrence from halogen, nitro, oxo, phosphonoxy, phosphono, thioxo, cyano, or -L_{S}-R_{E};
R_{B} and R_{B}′ are each independently selected at each occurrence from hydrogen; or C₁-C₆alkyl which is independently optionally substituted at each occurrence with one or more substituents selected from halogen or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, C₁-C₆alkyl, C₁-C₆haloalkyl, —O—C₁-C₆alkyl, or —O—C₁-C₆haloalkyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, or ═C(R_{S}R_{S}′); or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₁₂carbocycle or 3- to 12-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), or —N(R_{S})C(O)R_{S}′; or C₃-C₁₂carbocycle or 3- to 12-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
L_{S} is independently selected at each occurrence from bond; or C₁-C₆alkylene, C₂-C₆alkenylene or C₂-C₆alkynylene, each independently optionally substituted with halogen;
L_{S}′ is independently selected at each occurrence from bond; or C₁-C₆alkylene, C₂-C₆alkenylene or C₂-C₆alkynylene, each of which is independently optionally substituted at each occurrence with one or more R_{L};
R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆haloalkyl, or 3- to 12-membered carbocycle or heterocycle; or 3- to 12-membered carbocycle or heterocycle; wherein each 3- to 12-membered carbocycle or heterocycle in R_{S}, R_{S}′ or R_{S}″ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{M} is independently selected at each occurrence from:
   halogen, hydroxy, mercapto, amino, carboxy, nitro, oxo, phosphonoxy, phosphono, thioxo, cyano, SF₅, —N(R_{S}R_{S}′), —O—R_{S}, —OC(O)R_{S}, —OC(O)OR_{S}, —OC(O)N(R_{S}R_{S}′), —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —S(O)R_{S}, —SO₂R_{S}, —S(O)N(R_{S}R_{S}′), —SR_{S}, —Si(R_{S})₃, or —P(O)(OR_{S})₂;
   C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —N(R_{S}R_{S}′), —O—R_{S}, —OC(O)R_{S}, —OC(O)OR_{S}, —OC(O)N(R_{S}R_{S}′), —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —S(O)R_{S}, —SO₂R_{S}, —S(O)N(R_{S}R_{S}′), —SR_{S}, or —P(O)(OR_{S})₂; or
   G₂, wherein G₂ is a C₃-C₁₂carbocycle or 3- to 12-membered heterocycle, each of which is independently optionally substituted at each occurrence with one or more R_{G2}, and each R_{G2} is independently selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —O—R_{S}, —C(O)OR_{S}, —C(O)R_{S}, —N(R_{S}R_{S}′), or -L₄-G₃;
L₄ is a bond, C₁-C₆alkylene, C₂-C₆alkenylene, C₂-C₆alkynylene, —O—, —S—, —N(R_{B})—, —C(O)—, —S(O)₂—, —S(O)—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —N(R_{B})C(O)N(R_{B}′)—, —N(R_{B})SO₂N(R_{B}′)—, or —N(R_{B})S(O)N(R_{B}′)—;
G₃ is a C₃-C₁₂carbocycle or 3- to 12-membered heterocycle, and is optionally substituted with one or more R_{G3}; and
R_{G3} is each independently, at each occurrence, halogen, —C₁-C₆alkyl, —C(O)C₁-C₆alkyl, —C₁-C₆haloalkyl, —O—C₁-C₆alkyl, —O—C₁-C₆haloalkyl, C₃-C₆carbocycle, or 3- to 6-membered heterocycle.

As described hereinabove for compounds of Formula I_{E} A and B are each phenyl, pyridinyl, thiazolyl, or

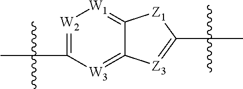

where Z₁ is independently selected at each occurrence from O, S, NH or CH₂, Z₃ is independently selected at each occurrence from N or CH, and W₁, W₂, and W₃ are each independently selected at each occurrence from CH or N; A and B are each independently optionally substituted with one or more R_{A}.

Preferably, A is selected from

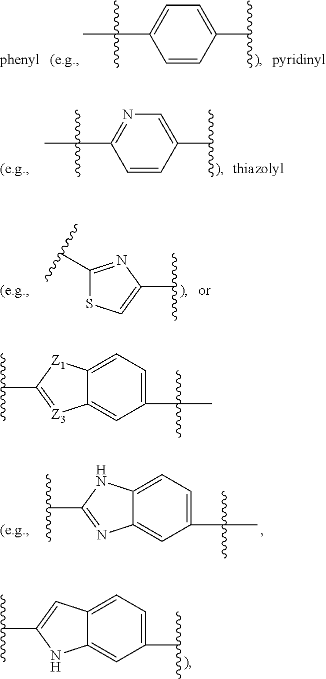

and is optionally substituted with one or more R_{A}.

Preferably, B is selected from

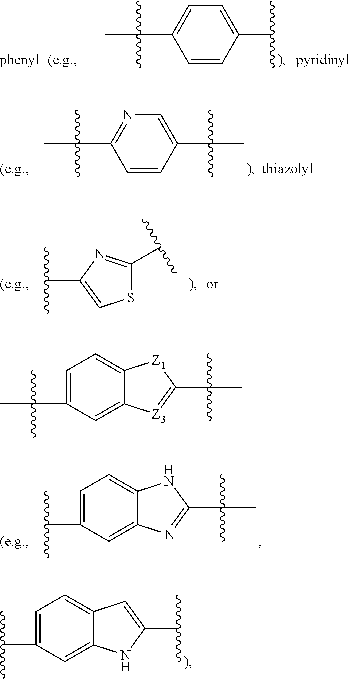

and is optionally substituted with one or more R_{A}.

Highly preferably, both A and B are phenyl (e.g., both A and B are

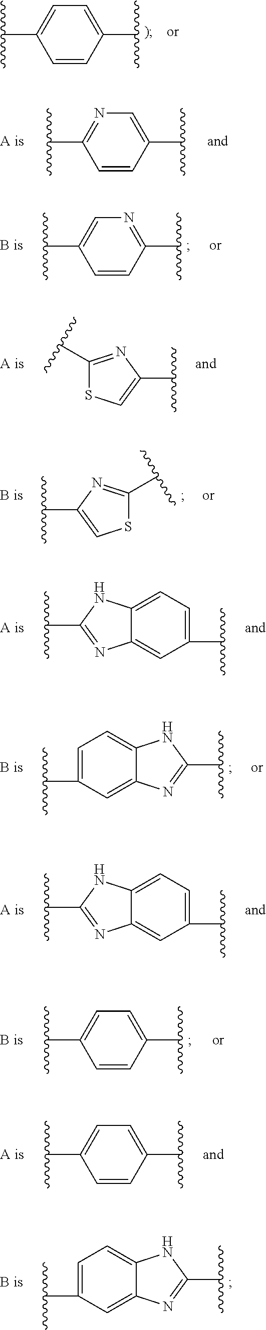

wherein each A and B is independently optionally substituted with one or more R_{A}.

In certain embodiments of this aspect of the invention, A and B are substituted by one or more R_{A}, wherein each R_{A} is independently selected from halogen (e.g., fluoro, chloro), L_{S}-R_{E} (where L_{S} is bond and R_{E} is —C₁-C₆alkyl (e.g., methyl), —O—R_{S} (e.g., —O—C₁-C₆alkyl, —OCH₃), or —C₁-C₆alkyl optionally substituted with one or more halogen (e.g., —CF₃)), or L_{S}-R_{E} (where L_{S} is C₁-C₆alkylene and R_{E} is —O—R_{S} (e.g., —C₁-C₆alkyl-O—C₁-C₆alkyl, —CH₂OCH₃)). For example, in certain embodiments A is

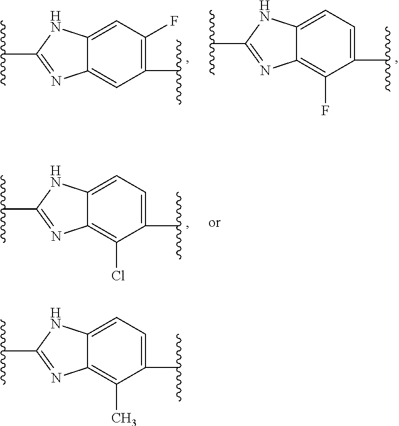

and B is as defined hereinabove. In certain other embodiments B is

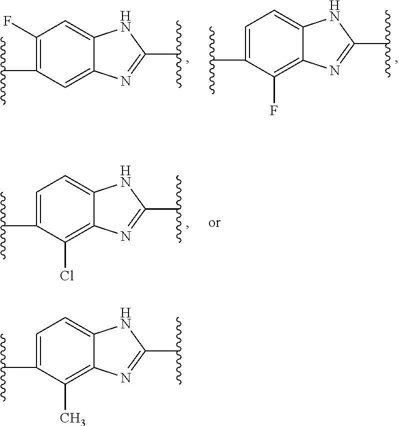

and A is as defined hereinabove. In still other embodiments A is

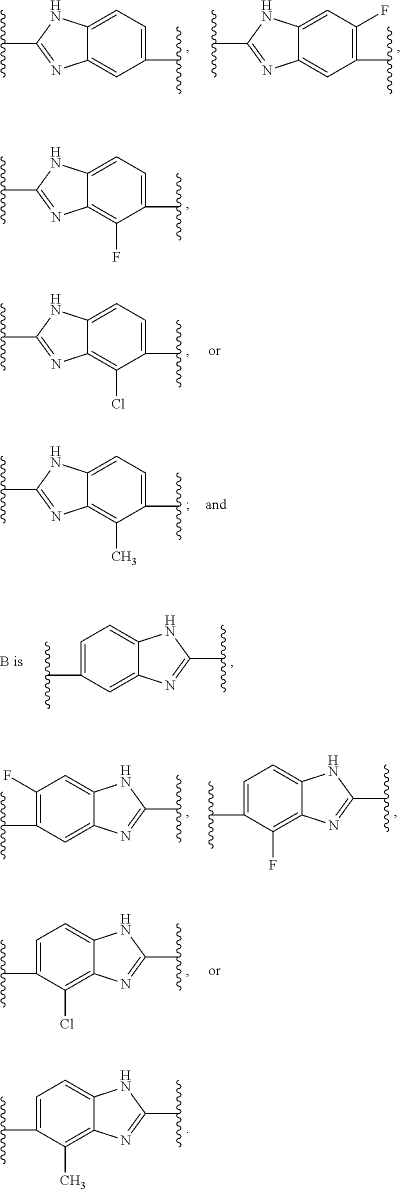

As described hereinabove for compounds of Formula I_{E} D is C₆-C₁₀carbocycle or 3- to 12-membered heterocycle optionally substituted by one or more R_{M}. Preferably, D is C₆-C₁₀aryl (e.g., phenyl, naphthyl, indanyl), or 5- to 10-membered heteroaryl (pyridinyl, thiazolyl, 4,5,6,7-tetrahydrobenzo[d]thiazolyl, benzo[d]thiazolyl, indazolyl, benzo[d][1,3]dioxol-5-yl), and D is substituted with one or more R_{M}. For example, in certain embodiments D is preferably phenyl substituted by one or more R_{M}, wherein each R_{M} is independently halogen (e.g., fluoro, chloro, bromo); C₁-C₆alkyl (e.g., tert-butyl); C₁-C₆alkyl substituted with one or more halogen (e.g., CF₃); —O—R_{S} such as —O—C₁-C₆alkyl (e.g., —O—CH₂CH₃); or —O—C₁-C₆alkyl substituted at each occurrence with one or more halogen (e.g., —O—CF₃, —O—CH₂CHF₂) or —O—C₁-C₆alkyl (e.g., —O—CH₂CH₂OCH₃); —O—R_{S} (e.g., —O—C₁-C₆alkyl, such as —O—CH₂) substituted with 3- to 12-membered heterocycle (e.g., 3-ethyloxetan-3-yl, 1,3-dioxolan-4-yl); —O—R_{S} where R_{S} is an optionally substituted 3- to 12-membered carbocycle or heterocycle (e.g., cyclopentyl, cyclohexyl, phenyl, 1,3-dioxan-5-yl); —N(R_{S})C(O)R_{S}′ wherein R_{S} and R_{S}′ are each independently C₁-C₆alkyl (e.g., —N(t-Bu)C(O)Me); SF₅; —SO₂R_{S} wherein R_{S} is C₁-C₆alkyl (e.g., —SO₂Me); or C₃-C₁₂carbocycle (e.g., cyclopropyl, cyclohexyl, phenyl).

In certain embodiments of this aspect of the invention, D is preferably phenyl or pyridyl and is substituted by one or more R_{M} where one R_{M} is G₂. In certain embodiments where D is phenyl or pyridyl, D is substituted by G₂, G₂ is 3- to 12-membered heterocycle (e.g., pyridinyl, piperidinyl, pyrrolidinyl, azetidinyl, oxazolyl) and is optionally substituted with one or more halogen (e.g., fluoro, chloro), hydroxy, oxo, cyano, C₁-C₆alkyl (e.g., methyl), C₂-C₆alkenyl, C₂-C₆alkynyl, C₁-C₆haloalkyl (e.g., CF₃), C₂-C₆haloalkenyl, C₂-C₆haloalkynyl, —O—C₁-C₆alkyl (e.g., —O—CH₃), —C(O)OR_{S} (e.g., —C(O)OCH₃), —C(O)R_{S} (e.g., —C(O)CH₃), or —N(R_{S}R_{S}′); and D is further optionally substituted by one or more R_{M} where R_{M} is halogen (e.g., fluoro, chloro), C₁-C₆alkyl (e.g., methyl), C₁-C₆haloalkyl (e.g., —CF₃), or —O—C₁-C₆alkyl (e.g., —O—CH₃). In certain other embodiments D is phenyl or pyridyl and G₂ is, for example, a monocyclic 3-8 membered carbocycle or monocyclic 4-8 membered heterocycle substituted with L₄-G₃ and optionally substituted with one or more R_{G2} wherein L₄, G₃ and R_{G2} are as defined herein. L₄, for example is a bond, a C₁-C₆ alkylene (e.g., —CH₂—, —CH₂CH₂—, —CH₂CH₂CH₂—, etc.), —O—, or —S(O)₂—. G₃ is for example a C₃-C₁₂carbocycle optionally substituted with one or more R_{G3}. R_{G2} and R_{G3} are each independently at each occurrence halogen, —C(O)C₁-C₆alkyl, —C₁-C₆alkyl, —C₁-C₆haloalkyl, —O—C₁-C₆alkyl, or —O—C₁-C₆haloalkyl. In certain embodiments G₂ is

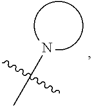

wherein

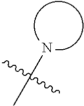

is a monocyclic 4-8 membered nitrogen-containing heterocycle (e.g., azetidinyl, pyrrolidinyl, piperidinyl, piperazinyl) attached to the parent molecular moiety through a nitrogen atom and substituted with one or two L₄-G₃ and optionally substituted with one or more R_{G2}. Thus, in certain embodiments where L₄ is a bond G₂ is

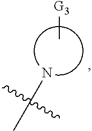

where

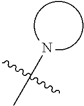

is optionally substituted with R_{G2} and G₃ is optionally substituted with R_{G3}. Thus,

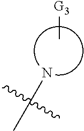

can be, for example, 3-phenylazetidin-1-yl, 3-phenylpyrrolidin-1-yl, 4-phenylpiperazin-1-yl, 4-phenylpiperidin-1-yl, 4-phenyl-3,6-dihydropyridin-1(2H)-yl, 4,4-diphenylpiperidin-1-yl, 4-acetyl-4-phenylpiperidin-1-yl, 4-(4-methoxyphenyl)piperidin-1-yl, 4-(4-fluorophenyl)piperidin-1-yl, or 3-phenylpiperidin-1-yl, and wherein D can be further optionally substituted with one or more R_{M} (e.g., fluoro, chloro, methyl, methoxy).

In certain other embodiments of this aspect of the invention, L₄ is a C₁-C₆ alkylene, —O—, or —S(O)₂—, and G₂ is

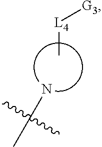

where

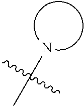

is as defined above and is optionally substituted with R_{G2} and G₃ is as defined above and is optionally substituted with R_{G3}. Thus,

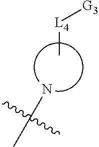

can be, for example, 4-tosylpiperazin-1-yl, 4-phenoxypiperidin-1-yl, 3-phenoxypyrrolidin-1-yl, 4-benzylpiperidin-1-yl, 4-phenethylpiperidin-1-yl, or 3-phenylpropyl)piperidin-1-yl.

In certain other embodiments of this aspect of the invention, D is phenyl or pyridyl, D is substituted by G₂ and G₂ is a spiro, bridged, or fused bicyclic carbocycle or heterocycle optionally substituted with L₄-G₃ and one or more R_{G2}, wherein D is optionally substituted with one or more R_{M} and R_{M}, L₄, G₃, and R_{G2} are as defined herein. In certain embodiments G₂ is

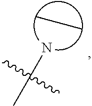

wherein

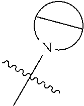

is a spiro, bridged, or fused bicyclic nitrogen-containing heterocycle (e.g., 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, 1,4-dioxa-8-azaspiro[4.5]dec-8-yl) attached to the parent molecular moiety through a nitrogen atom and optionally substituted with G₃ and one or more R_{G2}. Thus, G₂ is 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, or 1,4-dioxa-8-azaspiro[4.5]dec-8-yl; L₄ is a bond and D is optionally substituted with one or more R_{M} (e.g., fluoro, chloro, methyl, methoxy).

In certain embodiments of this aspect of the invention, D is

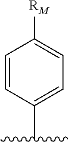

wherein R_{M} is as defined above in connection with Formula I_{E}, and D is optionally substituted by one or more additional R_{M}. For instance, where D is

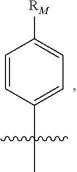

R_{M} can be fluoro, chloro, tert-butyl, —O—CH₂CH₃, —O—CF₃, —O—CH₂CHF₂, —O—CH₂CH₂OCH₃, —O—CH₂—(3-ethyloxetan-3-yl), —O—CH₂—(1,3-dioxolan-4-yl), —O-cyclopentyl, —O-cyclohexyl, —O-phenyl, —O-(1,3-dioxan-5-yl), cyclopropyl, cyclohexyl, phenyl, SF_{S}, —SO₂Me, or —N(t-Bu)C(O)Me and D can be optionally substituted by one or more additional R_{M} selected from the group consisting of halogen (e.g., fluoro, chloro) and C₁-C₆alkyl (e.g., methyl).

In certain embodiments, D is

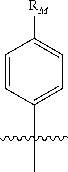

wherein R_{M} is fluoro, chloro, tert-butyl, —O—CH₂CH₃, —O—CF₃, —O—CH₂CHF₂, —O—CH₂CH₂OCH₃, SF₅, —SO₂Me, or —N(t-Bu)C(O)Me and D is optionally substituted by one or more additional R_{M} selected from the group consisting of halogen (e.g., fluoro, chloro) and C₁-C₆alkyl (e.g., methyl).

In certain embodiments, D is

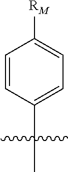

wherein R_{M} is cyclopropyl, cyclohexyl, or phenyl and D is optionally substituted by one or more additional R_{M} selected from the group consisting of halogen (e.g., fluoro, chloro) and C₁-C₆alkyl (e.g., methyl).

In certain embodiments, D is

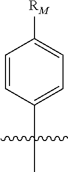

wherein R_{M} is —O—CH₂-(3-ethyloxetan-3-yl), —O—CH₂-(1,3-dioxolan-4-yl), —O-cyclopentyl, —O-cyclohexyl, —O-phenyl, or —O-(1,3-dioxan-5-yl) and D is optionally substituted by one or more additional R_{M} selected from the group consisting of halogen (e.g., fluoro, chloro) and C₁-C₆alkyl (e.g., methyl).

In certain embodiments, D is

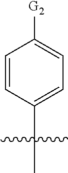

wherein G₂ is pyridinyl (e.g., pyridin-2-yl), piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, 3,3-dimethylazetidin-1-yl, or oxazolyl (e.g., 1,3-oxazol-2-yl) and D is optionally substituted by one or more additional R_{M} selected from the group consisting of halogen (e.g., fluoro, chloro) and C₁-C₆alkyl (e.g., methyl).

In another embodiment of this aspect of the invention, D is

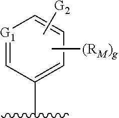

wherein G₁ is N, C—H, or C—R_{M}; G₂ is

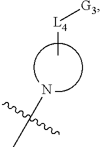

wherein

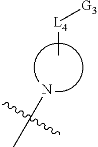

is a monocyclic 4-8 membered nitrogen-containing heterocycle (e.g., azetidinyl, pyrrolidinyl, piperidinyl) attached to the parent molecular moiety through a nitrogen atom and substituted by L₄-G₃ and optionally substituted with one or more R_{G2}; L₄ is a bond, C₁-C₆ alkylene, —O—, or —S(O)₂—; G₃ is aryl (e.g., phenyl), cycloalkyl (e.g., cyclohexyl), or heterocycle (e.g., thienyl) wherein each G₃ is optionally substituted with one or more R_{G3}; R_{G2} and R_{G3} at each occurrence are each independently halogen, —C(O)C₁-C₆alkyl, —C₁-C₆alkyl, —C₁-C₆haloalkyl, —O—C₁-C₆alkyl, or —O—C₁-C₆haloalkyl; g is 0, 1, 2, or 3; and R_{M} is as defined above in connection with Formula I_{E}. In one group of compounds according to this embodiment, D is

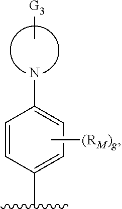

wherein G₃ is phenyl optionally substituted with one or two R_{G3}; g is 0, 1, or 2; R_{M} is each independently fluoro, chloro, methyl, methoxy, trifluoromethyl, or trifluoromethoxy; and

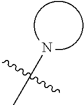

R_{G3} are as defined above. In a further subgroup of compounds of this embodiment, D is

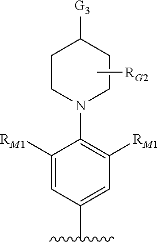

wherein G₃ is phenyl optionally substituted with one or two R_{G3}; R_{M1} is each independently hydrogen, fluoro, chloro, or methyl; and R_{G2} is an optional substituent as described herein. In another group of compounds according to this embodiment, D is

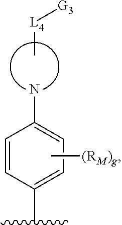

wherein L₄ is C₁-C₆ alkylene, —O—, or —S(O)₂—; G₃ is phenyl optionally substituted with one or two R_{G3}; g is 0, 1, or 2; R_{M} is each independently fluoro, chloro, methyl, methoxy, trifluoromethyl, or trifluoromethoxy; and

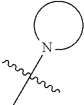

and R_{G3} are as defined above.

In yet another embodiment of this aspect of the invention, D is

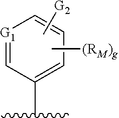

wherein G₁ is N, C—H, or C—R_{M}; G₂ is

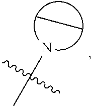

wherein

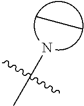

is a spiro, bridged, or fused bicyclic nitrogen-containing heterocycle (e.g., 3-azabicyclo[3.2.0]hept-3-yl, 2-azabicyclo[2.2.2]oct-2-yl, 6-azaspiro[2.5]oct-6 octahydro-2H-isoindol-2-yl, 3-azaspiro[5.5]undec-3-yl, 1,3-dihydro-2H-isoindol-2-yl, 1,4-dioxa-8-azaspiro[4.5]dec-8-yl) attached to the parent molecular moiety through a nitrogen atom and optionally substituted with L₄-G₃ and one or more R_{G2}; L₄ is a bond, C₁-C₆ alkylene, —O—, or —S(O)₂—; G₃ is aryl (e.g., phenyl), cycloalkyl (e.g., cyclohexyl), or heterocycle (e.g., thienyl) wherein each G₃ is optionally substituted with one or more R_{G3}, R_{G2} and R_{G3} at each occurrence are each independently halogen, —C(O)C₁-C₆alkyl, —C₁-C₆alkyl, —C₁-C₆haloalkyl, —O—C₁-C₆alkyl, or —O—C₁-C₆haloalkyl; g is 0, 1, 2, or 3; and R_{M} is as defined above in connection with Formula I_{E}. In one group of compounds according to this embodiment, D is

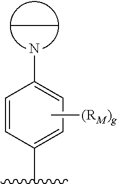

wherein g is 0, 1, or 2; R_{M} is each independently fluoro, chloro, methyl, methoxy, trifluoromethyl, or trifluoromethoxy; and

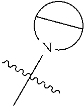

is as defined above. In a further subgroup of compounds D is

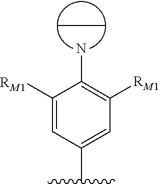

wherein R_{M1} is each independently hydrogen, fluoro, chloro, or methyl, and

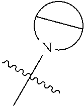

is as defined above (e.g., 3-azabicyclo[3.2.0]hept-3-yl, octahydro-2H-isoindol-2-yl, 2-azabicyclo[2.2.2]oct-2-yl, 6-azaspiro[2.5]oct-6-yl, 3-azaspiro[5.5]undec-3-yl, 1,3-dihydro -2H -isoindol-2-yl, 1,4-dioxa-8-azaspiro[4.5]dec-8-yl).

In still another embodiment of this aspect of the invention, D is

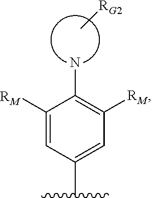

wherein

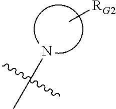

is a monocyclic 4-8 membered nitrogen-containing heterocycle (e.g., azetidinyl, pyrrolidinyl, piperidinyl) substituted with one or more R_{G2}, wherein R_{G2} at each occurrence is each independently halogen, —C(O)C₁-C₆alkyl, —C₁-C₆alkyl, —C₁-C₆haloalkyl, —O—C₁-C₆alkyl, or —O—C₁-C₆haloalkyl; and R_{M} is each independently halogen, —C₁-C₆alkyl, —C₁-C₆haloalkyl, —O—C₁-C₆alkyl, or —O—C₁-C₆haloalkyl. In one group of compounds according to this embodiment,

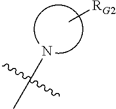

is azetidinyl, pyrrolidinyl, or piperidinyl substituted with one or two R_{G2}, wherein R_{G2} at each occurrence is each independently methyl, ethyl, isopropyl, tert-butyl, fluoro, chloro, or trifluoromethyl; and R_{M} is each independently fluoro, chloro, or methyl. For example

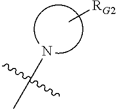

is 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl.

In compounds of Formula I_{E}, Y is -T′-C(R₁R₂)N(R₅)-T-R_{D} and Z is -T′-C(R₈R₉)N(R₁₂)-T-R_{D}; wherein T′, R₁, R₂, R₅, R₈, R₉, R₁₂, T, and R_{D} are as defined herein.

Preferably R₁, R₂, R₅, R₈, R₉, and R₁₂ are each independently hydrogen; C₁-C₆alkyl; or 3- to 6-membered carbocycle or heterocycle, wherein each 3- to 6-membered carbocycle or heterocycle is independently optionally substituted at each occurrence with one or more substituents selected from halogen or C₁-C₆alkyl; wherein R₂ and R₅, taken together with the atoms to which they are attached, optionally form a 3- to 12-membered heterocycle which is substituted with 0, 1, 2, 3, or 4 R_{A}, and R₉ and R₁₂ taken together with the atoms to which they are attached, optionally form a 3- to 12-membered heterocycle which is substituted with 0, 1, 2, 3, or 4 R_{A} wherein R_{A} is as defined herein.

In certain embodiments of this aspect of the invention, R₁ is hydrogen and R₂ and R₅, taken together with the atoms to which they are attached form a 3- to 12-membered heterocycle

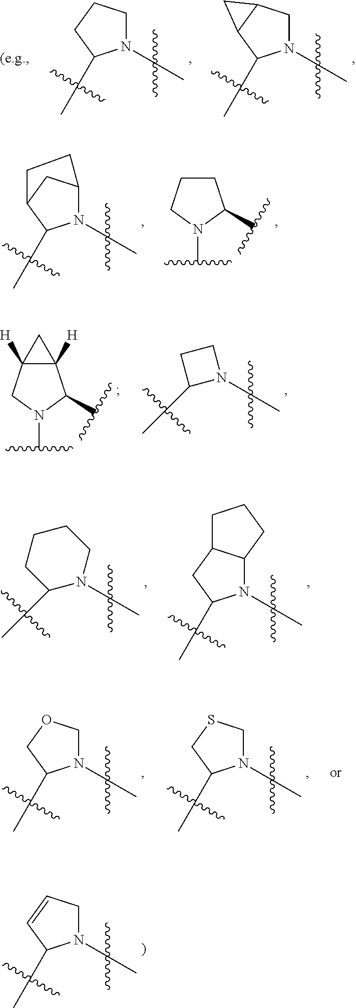

substituted with 0, 1, 2, 3, or 4 R_{A} wherein R_{A} is halogen (e.g., fluoro, chloro); cyano; L_{S}-R_{E} where L_{S} is a single bond and R_{E} is C₁-C₆alkyl (e.g., methyl, ethyl), —O—C₁-C₆alkyl (e.g., methoxy), or —O—C₁-C₆haloalkyl (e.g., trifluoromethoxy); or L_{S}-R_{E} where L_{S} is a double bond and R_{E} is ═C(R_{S}R_{S}′)

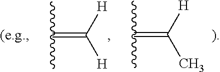

In a preferred embodiment R₂ and R₅, taken together with the atoms to which they are attached form a pyrrolidine ring

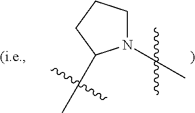

substituted with 0 or 1 R_{A} wherein R_{A} is fluoro, methoxy, methyl, ethyl, or cyano. In another preferred embodiment R₂ and R₅, taken together with the atoms to which they are attached form a pyrrolidine ring

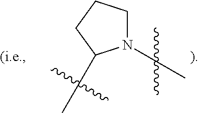

In certain other embodiments of this aspect of the invention, R₈ is hydrogen and R₉ and R₁₂, taken together with the atoms to which they are attached form a 3- to 12-membered heterocycle (e.g.,

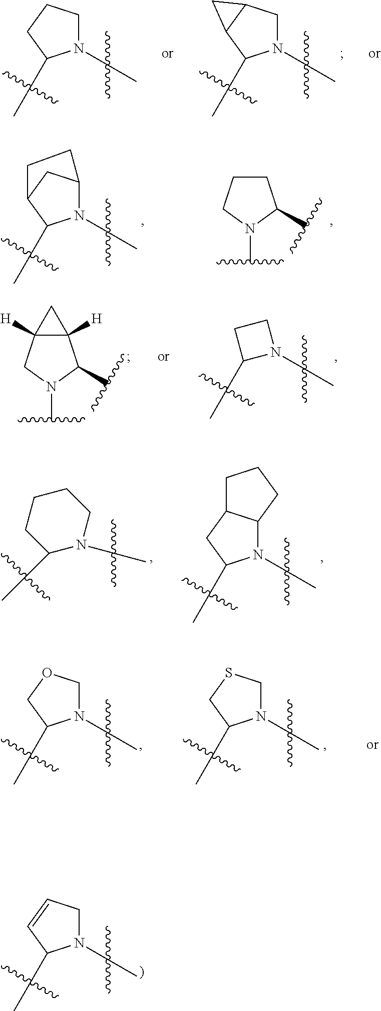

substituted with 0, 1, 2, 3, or 4 R_{A} wherein R_{A} is halogen (e.g., fluoro, chloro); cyano; L_{S}-R_{E} where L_{S} is a single bond and R_{E} is C₁-C₆alkyl (e.g., methyl, ethyl), —O—C₁-C₆alkyl (e.g., methoxy), or —O—C₁-C₆haloalkyl (e.g., trifluoromethoxy); or L_{S}-R_{E} where L_{S} is a double bond and R_{E} is ═C(R_{S}R_{S}′)

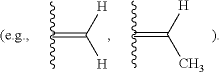

In a preferred embodiment, R₉ and R₁₂, taken together with the atoms to which they are attached form a pyrrolidine ring

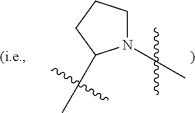

substituted with 0 or 1 R_{A} wherein R_{A} is fluoro, methoxy, methyl, ethyl, or cyano. In another preferred embodiment R₉ and R₁₂, taken together with the atoms to which they are attached form a pyrrolidine ring

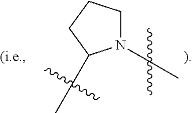

As used herein, a chiral carbon in any rings formed by joining R₂ and R₅ or R₉ and R₁₂ may possess either (R) or (S) stereochemistry. A pyrrolidine ring

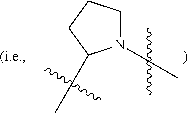

formed from either R₂ and R₅ or R₉ and R₁₂ preferably possesses the (S) stereochemistry

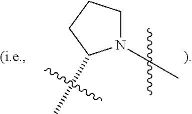

In this aspect of the invention, T′ is independently selected at each occurrence from a bond, —C(O)N(R_{B})—, —N(R_{B})C(O)—, or 3- to 12-membered heterocycle, and wherein said 3- to 12-membered heterocycle is each independently optionally substituted at each occurrence with one or more R_{A}, and R_{A} and R_{B} are as described herein. In particular, where T′ is —C(O)N(R_{B})—, R_{B} can be hydrogen (i.e., T′ is —C(O)N(H)—). In certain embodiments, T′ is imidazolyl

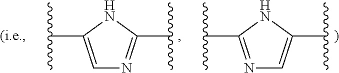

optionally substituted at each occurrence with one or more R_{A} wherein R_{A} is halogen (e.g., fluoro, chloro), C₁-C₆alkyl (e.g., methyl, ethyl), or C₁-C₆haloalkyl (e.g., trifluoromethyl). In certain embodiments, T′ is imidazolyl

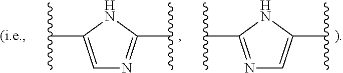

This aspect of the invention contemplates particular combinations of A with Y and B with Z. Non-limiting examples of preferred Y when A is C₅-C₆carbocycle (e.g., phenyl) or 5- to 6-membered heterocycle (e.g., pyridinyl or thiazolyl) and preferred Z when B is C₅-C₆carbocycle (e.g., phenyl) or 5- to 6-membered heterocycle (e.g., pyridinyl or thiazolyl) include:

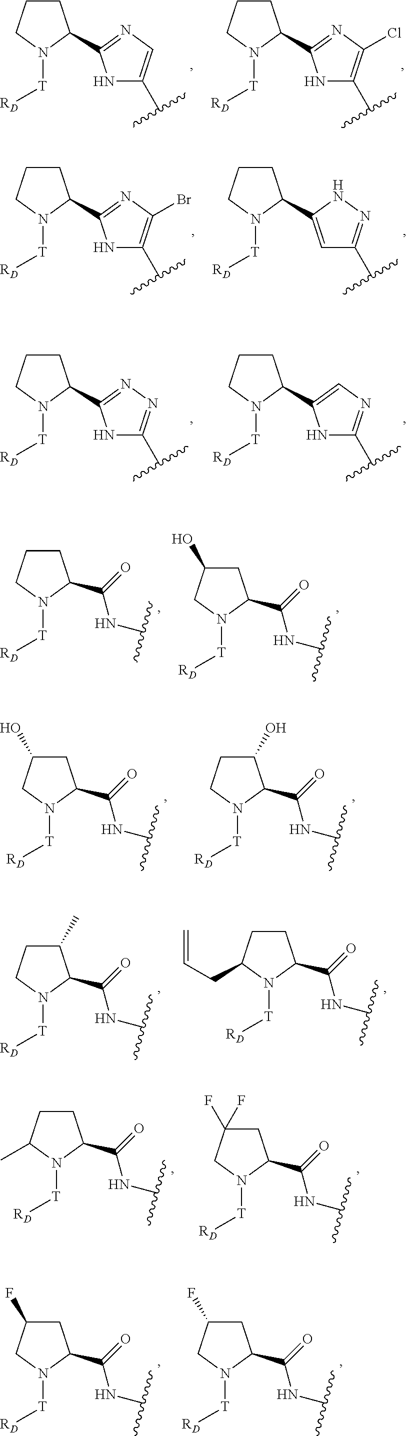

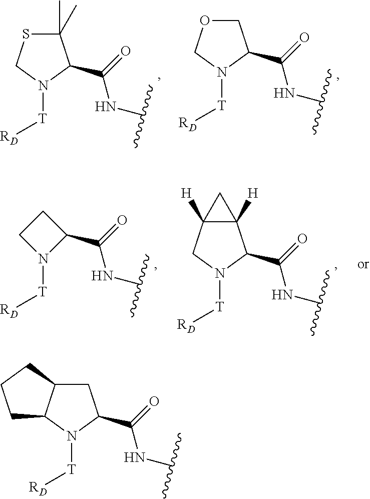

wherein T and R_{D} are as defined herein.

In certain embodiments of this aspect of the invention, A is

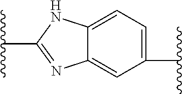

optionally substituted with one or more R_{A} as described herein, or Y-A is

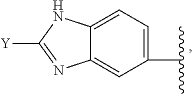

and non-limiting examples of preferred Y, where T′ is a bond, include:

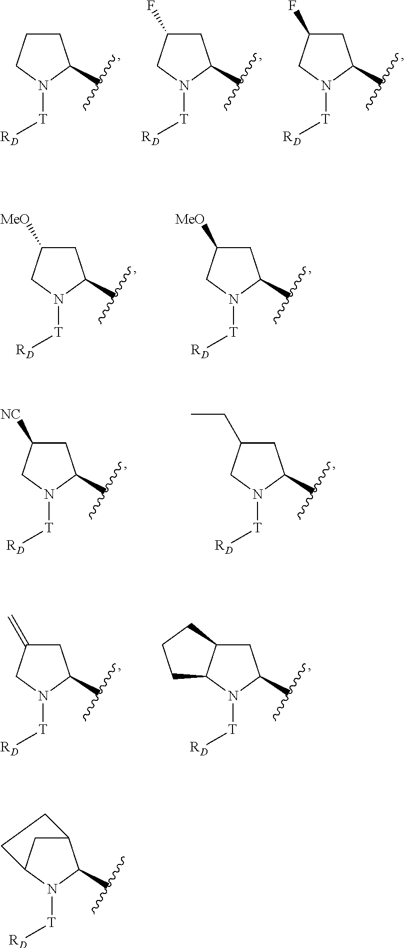

wherein T and R_{D} are as defined herein.

In certain embodiments of this aspect of the invention, B is

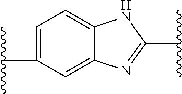

optionally substituted with one or more R_{A} as described herein, or B—Z is

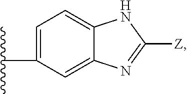

and non-limiting examples of preferred Z, where T′ is a bond, include:

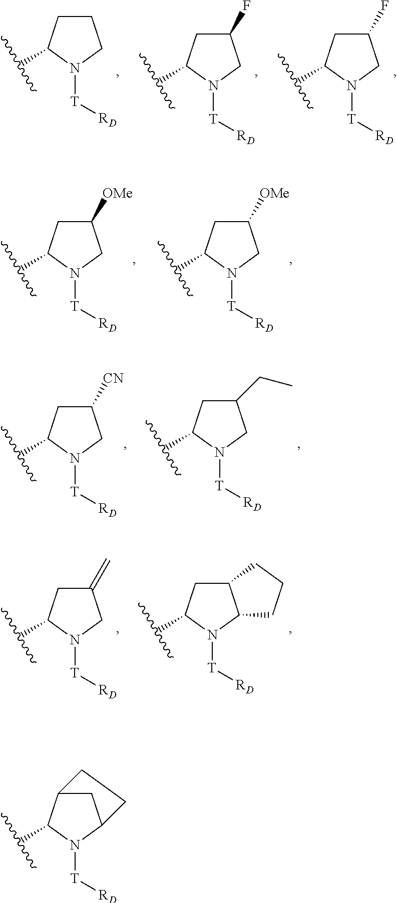

wherein T and R_{D} are as defined herein.

T at each occurrence is independently a bond or —C(O)-L_{S}′-, wherein L_{S}′ is as defined herein. L_{S}′ includes, but is not limited to,

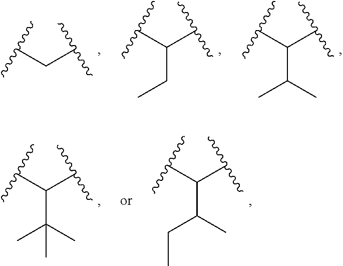

where L_{S}′ is optionally substituted with one or more R_{L}; and R_{L} is a substituent such as, but not limited to carbocycle (e.g., cyclohexyl, cyclopentyl, cyclobutyl, cyclopropyl, phenyl), methoxy, or heterocycle (e.g., tetrahydropyranyl, tetrahydropyranyl).

R_{D} is hydrogen or R_{A} wherein R_{A} is as defined herein. Thus R_{D} includes, but is not limited to, R_{A} wherein R_{A} is L_{S}-R_{E}, and L_{S} and R_{E} are as defined herein. Thus R_{D} includes, but is not limited to, L_{S}-R_{E} wherein L_{S} is a bond and R_{E} is —N(R_{S}R_{S}′), —N(R_{S})C(O)R_{S}′, —N(R_{S})C(O)N(R_{S}′R_{S}″), —N(R_{S})SO₂R_{S}′, —N(R_{S})SO₂N(R_{S}′R_{S}″), —N(R_{S})S(O)N(R_{S}′R_{S}″), —N(R_{S})C(O)OR_{S}′, or N(R_{S})S(O)—R_{S}′; or C₃-C₁₂carbocycle or 3- to 12-membered heterocycle, each of which is independently optionally substituted at each occurrence with one or more substituents selected from halogen, hydroxy, cyano, C₁-C₆alkyl, C₂-C₆alkenyl, C₂-C₆alkynyl, or C₁-C₆haloalkyl.

In one embodiment of this aspect of the invention, R_{D} is L_{S}-R_{E} wherein L_{S} is a bond and R_{E} is N(R_{S})C(O)OR_{S}′ or 3- to 12-membered heterocycle (e.g., pyrrolidine, piperidine, azepanyl) wherein R_{S} and R_{S}′ are as defined herein. For example R_{D} is preferably L_{S}-R_{E} wherein L_{S} is a bond and R_{E} is —N(H)C(O)OMe.

Thus according to the foregoing description T-R_{D} includes, but is not limited to:

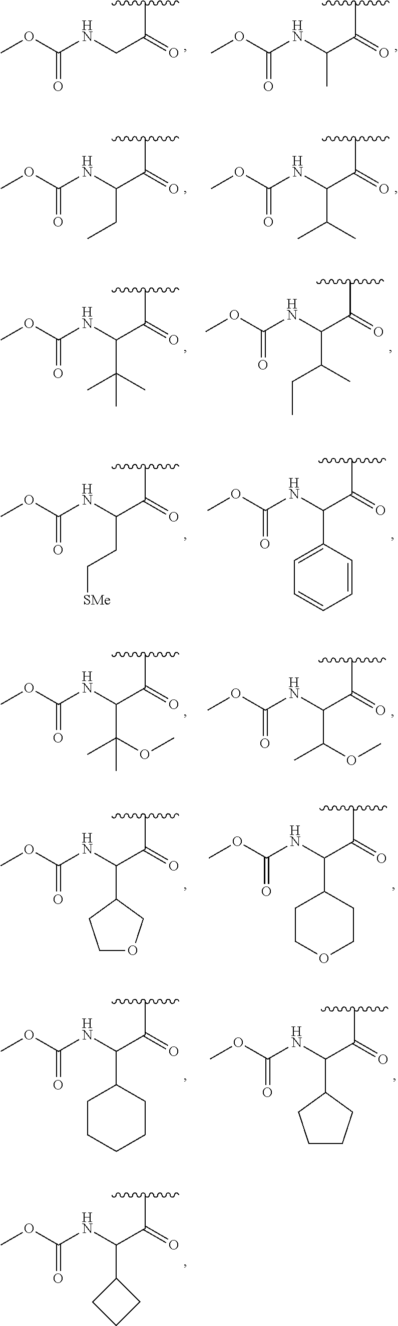

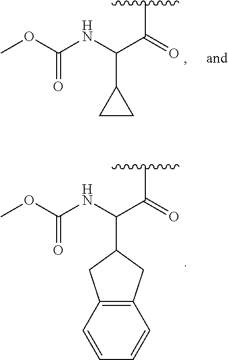

T-R_{D} may also include particular stereochemical configurations; thus T-R_{D} includes, but is not limited to:

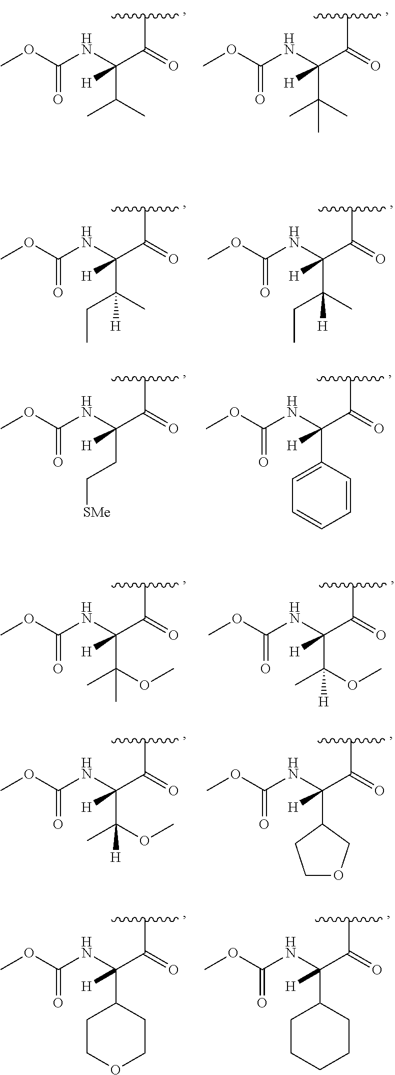

etc.

According to this aspect of the invention, non-limiting examples of preferred Y when A is C₅-C₆carbocycle (e.g., phenyl) or 5- to 6-membered heterocycle (e.g., pyridinyl or thiazolyl) and preferred Z when B is C₅-C₆carbocycle (e.g., phenyl) or 5- to 6-membered heterocycle (e.g., pyridinyl or thiazolyl) include:

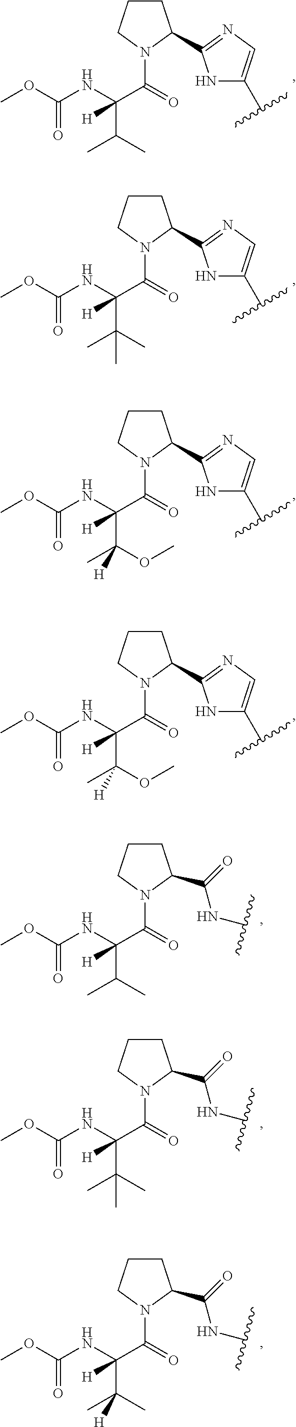

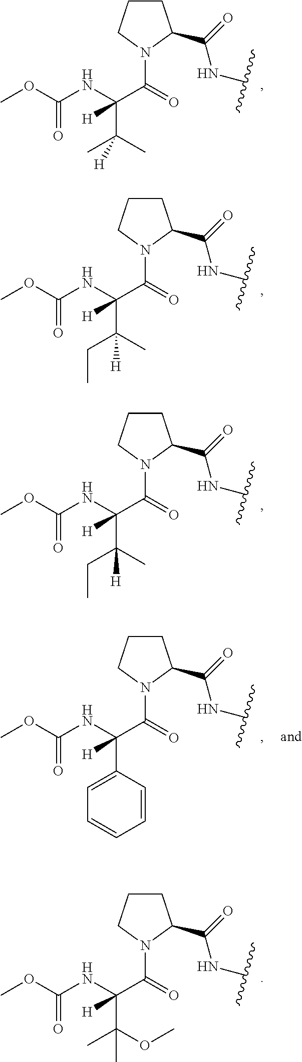

Non-limiting examples of preferred Y when A is

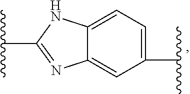

optionally substituted with one or more R_{A} as described herein, and Y-A is

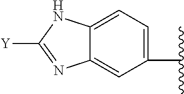

include:

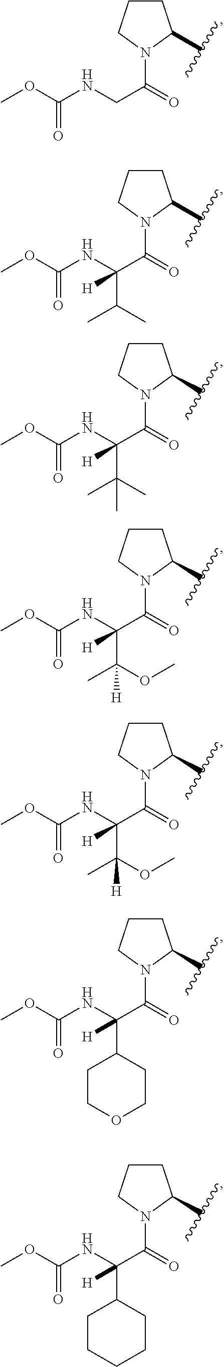

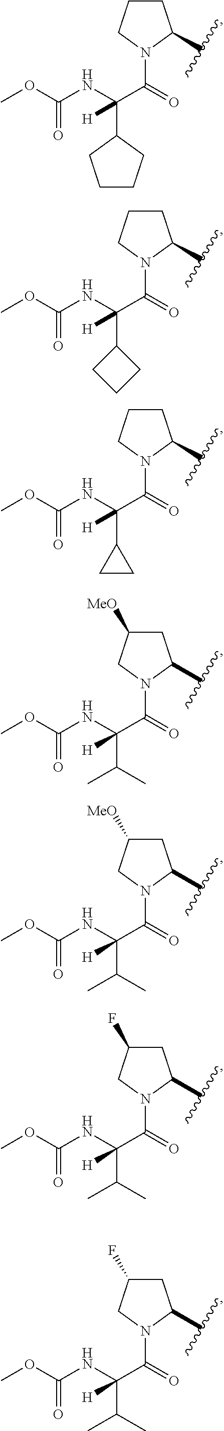

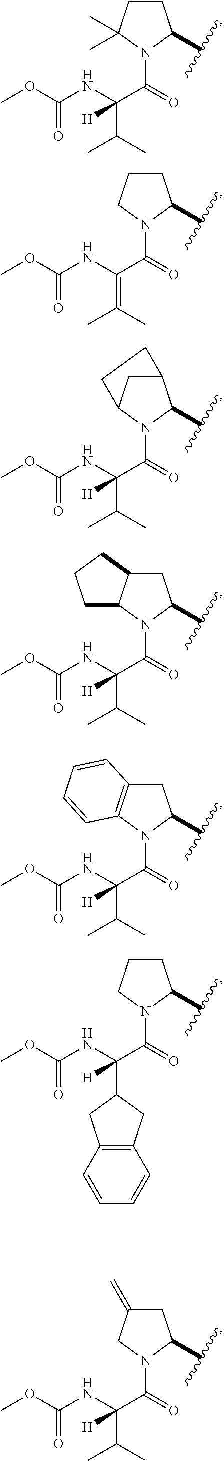

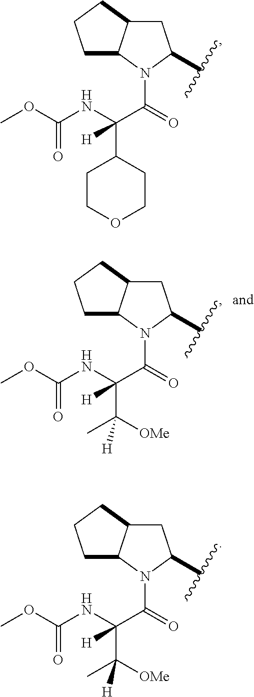

Non-limiting examples of preferred Z where B is

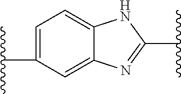

optionally substituted with one or more R_{A} as described herein, and B—Z is

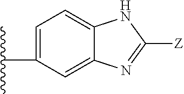

include:

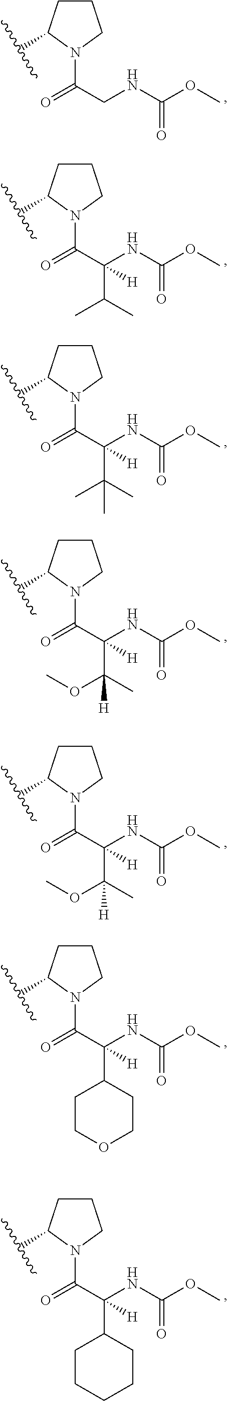

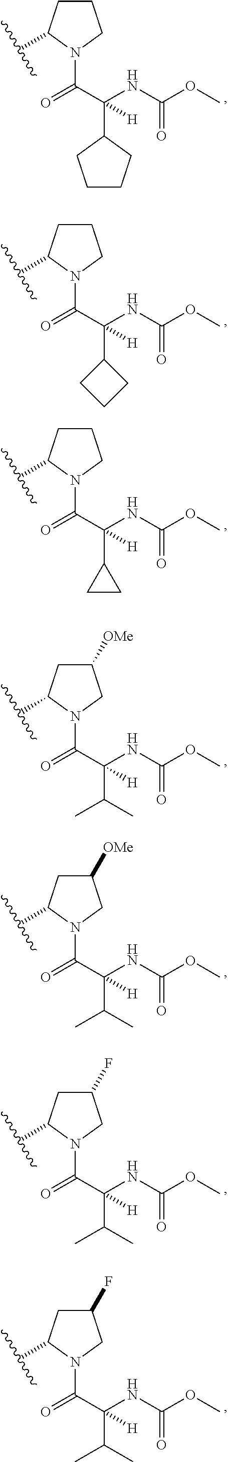

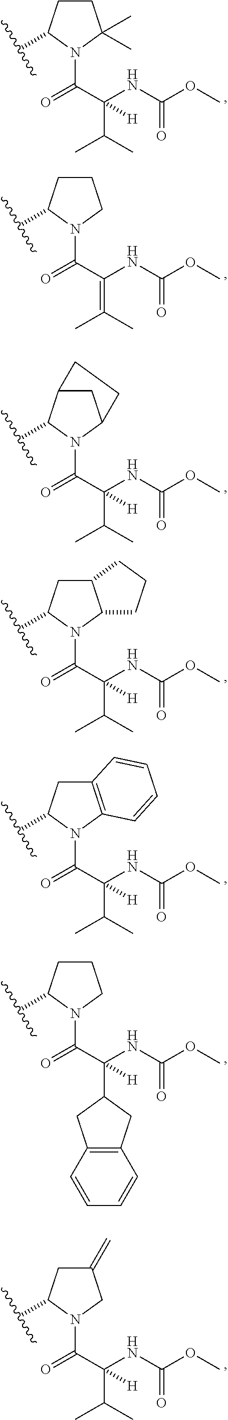

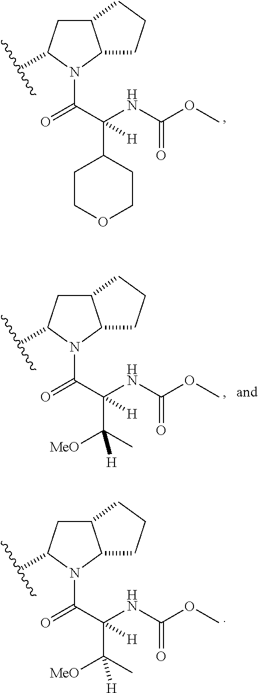

In still another aspect, the present invention features compounds of Formula I_{F} and pharmaceutically acceptable salts thereof:

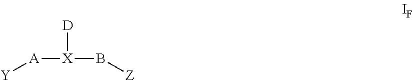

wherein:
X is CH₂CH, CHCH₂, C═C(H) or C(H)═C, and is optionally substituted with one or more R_{A}
A is

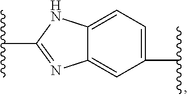

wherein A is optionally substituted with one or more R_{A};
B is

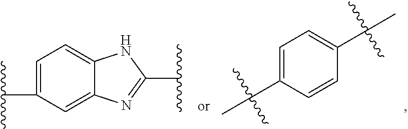

wherein B is optionally substituted with one or more R_{A}; and
Y, Z, R_{A}, and D are as described hereinabove (e.g., Y, Z, R_{A}, and D as described for Formula I, I_{A}, I_{B}, I_{C}, I_{D}, or I_{E}, preferably as described for Formula I_{E}).

In one embodiment of this aspect of the invention, A is

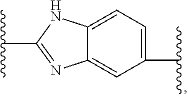

wherein A is optionally substituted with one or more R_{A}; B is

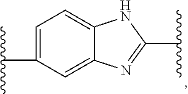

wherein B is optionally substituted with one or more R_{A}; Y is

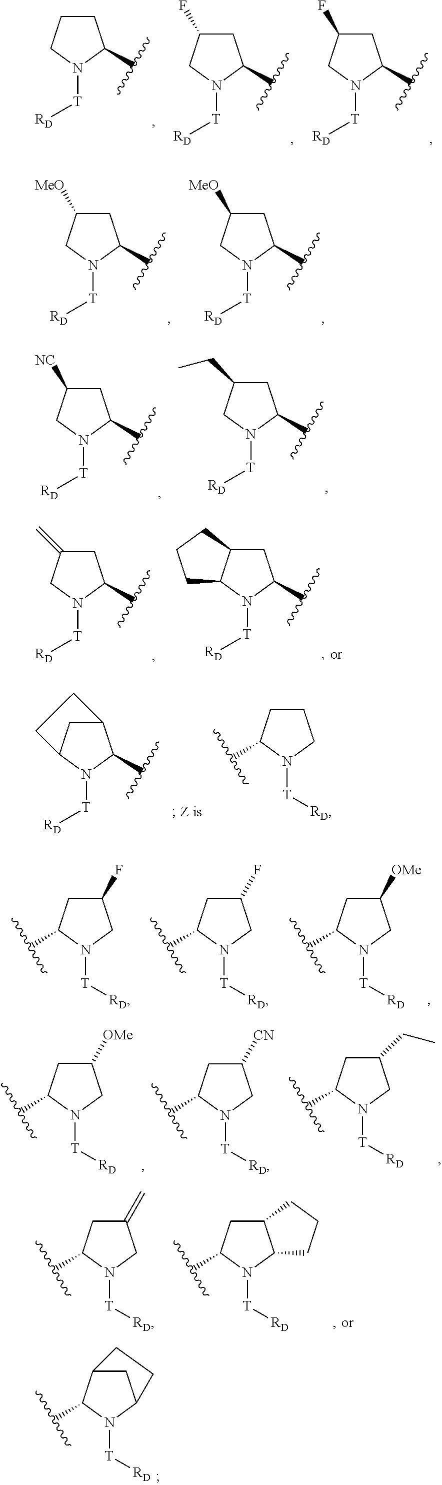

and D, R_{A}, T and R_{D} are as defined hereinabove (e.g., as described for Formula I, I_{A}, I_{B}, I_{C}, I_{D} or I_{E}, preferably as described for Formula I_{E}).

In another embodiment according to this aspect of the invention, A or B are optionally substituted with one or more substituents selected from: R_{A} wherein R_{A} is each independently halogen (e.g., fluoro, chloro); L_{S}-R_{E} where L_{S} is a single bond, and R_{E} is —C₁-C₆alkyl (e.g., methyl), —O—R_{S} (e.g., —O—C₁-C₆alkyl, —OCH₃), or —C₁-C₆alkyl optionally substituted with one or more halogen (e.g., —CF₃); or L_{S}-R_{E} where L_{S} is a C₁-C₆alkylene and R_{E} is —O—R_{S} (e.g., —C₁-C₆alkyl-O—C₁-C₆alkyl, —CH₂OCH₃). This embodiment includes compounds where A and B are both substituted by one R_{A}; compounds where A and B are both substituted by zero R_{A}; compounds where A is substituted by one R_{A} and B is substituted by zero R_{A}; and compounds where A is substituted by zero R_{A} and B is substituted by one R_{A}. Preferably,

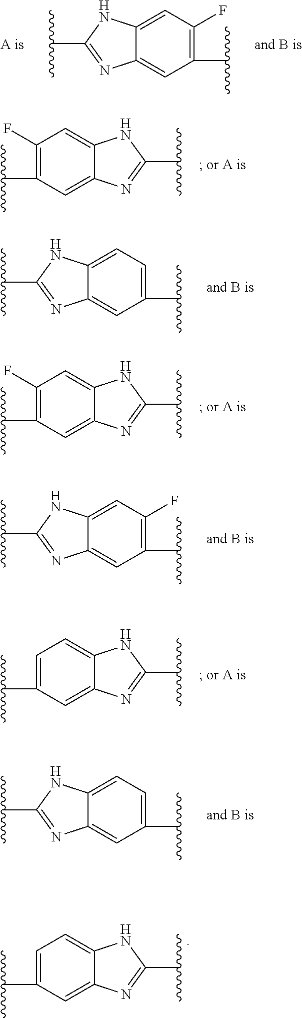

In a further embodiment of this aspect of the invention, T-R_{D} is independently selected at each occurrence from the group consisting of

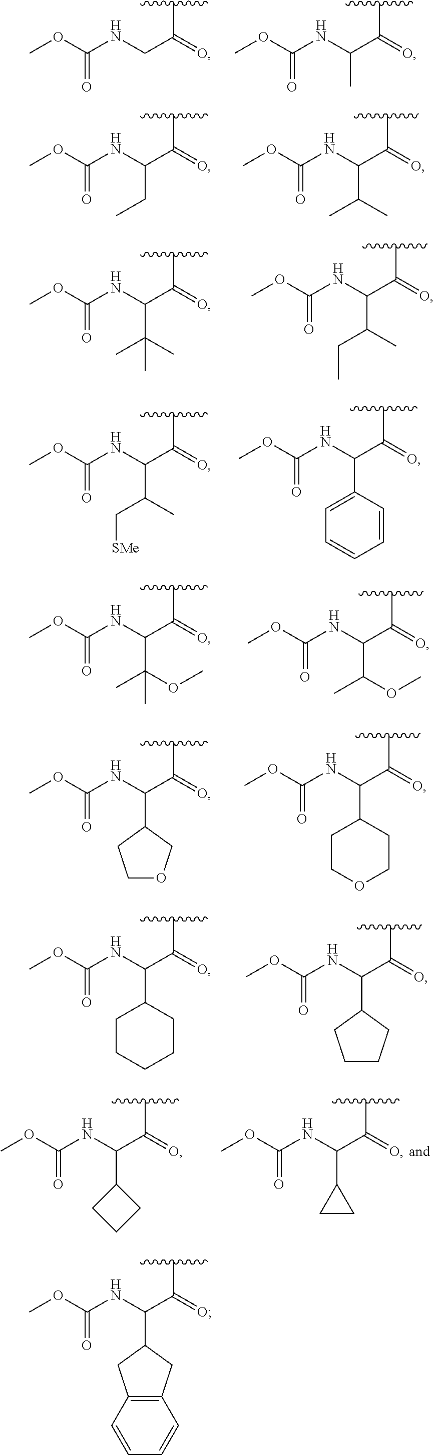

wherein compounds having (S) stereochemistry

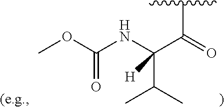

are preferred and wherein D is as defined hereinabove.

In another embodiment, this aspect of the invention features compound of Formula I_{F} and pharmaceutically acceptable salts thereof, wherein:

A is

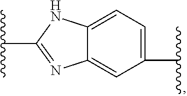

wherein A is optionally substituted with one or more R_{A}; B is

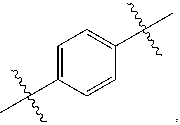

wherein B is optionally substituted with one or more R_{A}; Y is

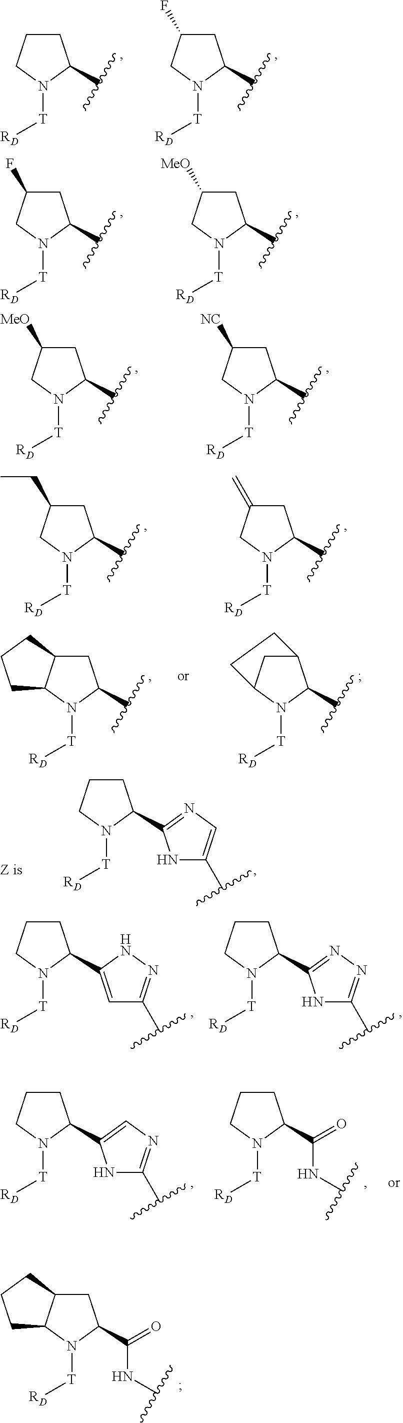

and D, R_{A}, T and R_{D} are as defined hereinabove. A particular subgroup according to this embodiment includes compounds where

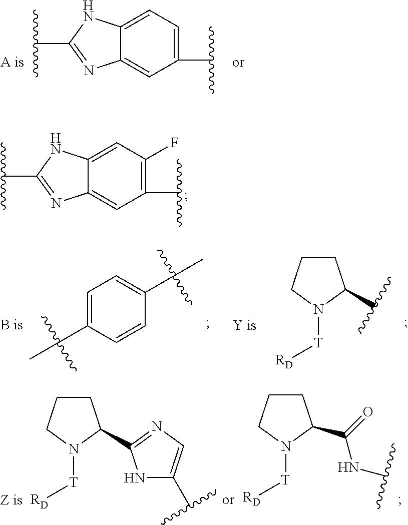

T-R_{D} is each independently

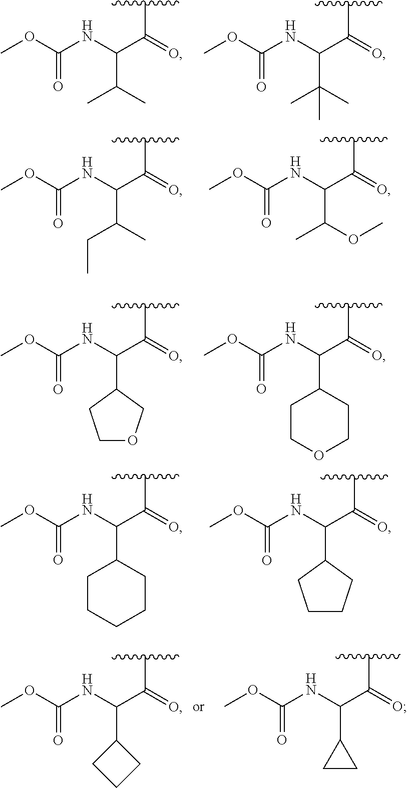

and D is as defined hereinabove.

In yet another embodiment, this aspect of the invention features compounds of Formula I_{F} and pharmaceutically acceptable salts thereof, wherein: A and B are each

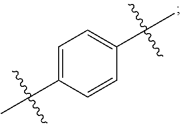

Y and Z are each independently

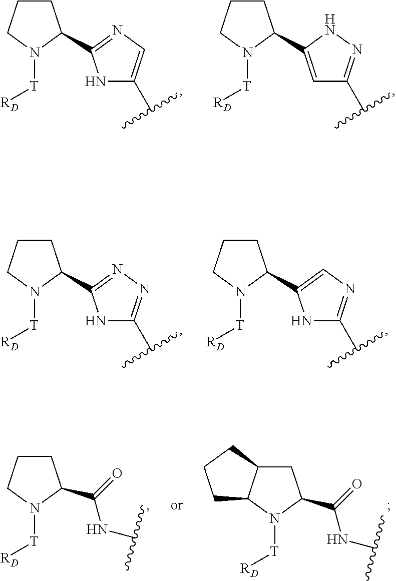

and D, T and R_{D} are as defined hereinabove. A particular subgroup according to this embodiment includes compounds where T-R_{D} is each independently selected from

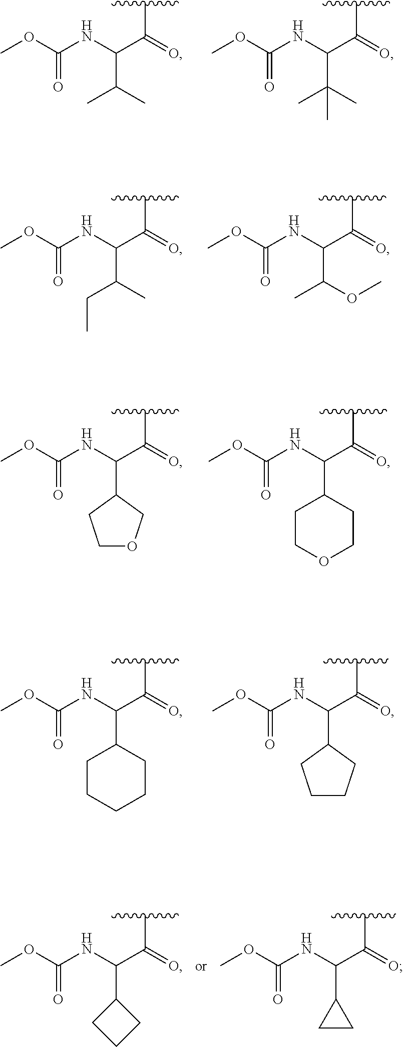

and D is as defined hereinabove.

According to each of the foregoing embodiments and description of this aspect of the invention of Formula I_{F} are groups and subgroups of compounds having particular values for D. Included in each of the foregoing embodiments are groups and subgroups of compounds with the following particular values for D:

In certain groups of compounds according to Formula I_{F} and the foregoing embodiments and description of this aspect of the invention, D is

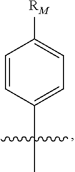

where R_{M} is fluoro, chloro, tert-butyl, —O—CH₂CH₃, —O—CF₃, —O—CH₂CHF₂, —O—CH₂CH₂OCH₃, —O—CH₂-(3-ethyloxetan-3-yl), —O—CH₂-(1,3-dioxolan-4-yl), —O-cyclopentyl, —O-cyclohexyl, —O-phenyl, —O-(1,3-dioxan-5-yl), cyclopropyl, cyclohexyl, phenyl, SF₅, —SO₂Me, or —N(t-Bu)C(O)Me and D is optionally substituted by one or more additional R_{M}, selected from the group consisting of halogen (e.g., fluoro, chloro) or C₁-C₆alkyl (e.g., methyl).

In other groups of compounds according Formula I_{F} and the foregoing embodiments and description of this aspect of the invention, D is

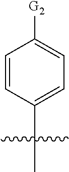

wherein G₂ is pyridinyl (e.g., pyridin-2-yl), piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, yl, 3,3-dimethylazetidin-1-yl, or oxazolyl (e.g., 1,3-oxazol-2-yl) and D is optionally substituted by one or more additional R_{M} selected from the group consisting of halogen (e.g., fluoro, chloro), or C₁-C₆allyl (e.g., methyl). In particular according to these groups are compounds where D is

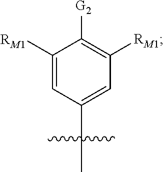

G₂ is piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl; and R_{M1} is each independently hydrogen, fluoro, chloro, or methyl.

In other groups of compounds according Formula I_{F} and the foregoing embodiments and description of this aspect of the invention, D is

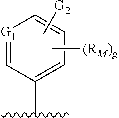

wherein G₁ is N, C—H, or C—R_{M}; G₂ is

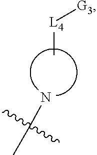

wherein

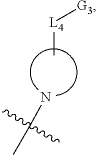

R_{M}, and g are as defined hereinabove. In particular according to these groups, R_{M} is each independently fluoro, chloro, methyl, methoxy, trifluoromethyl, or trifluoromethoxy; g is 0, 1, or 2; and

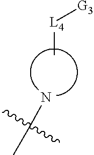

is as defined hereinabove. In further subgroups L₄ is a bond; G₂ is

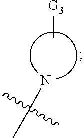

R_{M} is each independently fluoro, chloro, methyl, methoxy, trifluoromethyl, or trifluoromethoxy; and g is 0, 1, or 2. In particular subgroups,

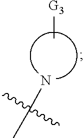

is 3-phenylazetidin-1-yl, 3-phenylpyrrolidin-1-yl, 4-phenylpiperazin-1-yl, 4-phenylpiperidin-1-yl, 4-phenyl-3,6-dihydropyridin-1(2H)-yl, 4,4-diphenylpiperidin-1-yl, 4-acetyl-4-phenylpiperidin-1-yl, 4-(4-methoxyphenyl)piperidin-1-yl, 4-(4-fluorophenyl)piperidin-1-yl, or 3-phenylpiperidin-1-yl; R_{M} is each independently fluoro, chloro, methyl, methoxy, trifluoromethyl, or trifluoromethoxy; and g is 0, 1, or 2. In other subgroups L₄ is C₁-C₆ alkylene, —O—, or —S(O)₂—; G₂ is

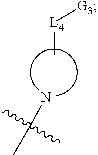

R_{M} is each independently fluoro, chloro, methyl, methoxy, trifluoromethyl, or trifluoromethoxy; and g is 0, 1, or 2. In particular subgroups,

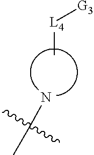

is 4-tosylpiperazin-1-yl, 4-phenoxypiperidin-1-yl, 3-phenoxypyrrolidin-1-yl, 4-benzylpiperidin-1-yl, 4-phenethylpiperidin-1-yl, or 3-phenylpropyl)piperidin-1-yl; R_{M} is each independently fluoro, chloro, methyl, methoxy, trifluoromethyl, or trifluoromethoxy; and g is 0, 1, or 2. In further subgroups of compounds D is

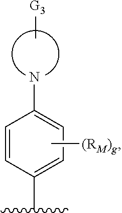

wherein G₃ is phenyl optionally substituted with one or two R_{G3}; g is 0, 1, or 2; R_{M} is each independently fluoro, chloro, methyl, methoxy, trifluoromethyl, or trifluoromethoxy; and

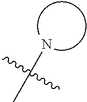

and R_{G3} are as defined above. In other groups of compounds D is

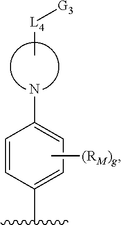

wherein L₄ is C₁-C₆ alkylene, —O—, or —S(O)₂—; G₃ is phenyl optionally substituted with one or two R_{G3}; g is 0, 1, or 2; R_{M} is each independently fluoro, chloro, methyl, methoxy, trifluoromethyl, or trifluoromethoxy; and

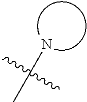

and R_{G3} are as defined above. In further subgroups of compounds D is

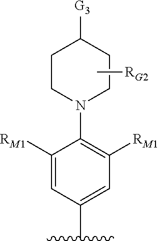

wherein G₃ is phenyl optionally substituted with one or two R_{G3} as defined hereinabove; R_{M1} is each independently hydrogen, fluoro, chloro, or methyl; and R_{G2} is an optional substituent, as described above, selected from the group consisting of —C(O)C₁-C₆alkyl, —C₁-C₆alkyl, —C₁-C₆haloalkyl, —O—C₁-C₆alkyl, and —O—C₁-C₆haloalkyl.

In other groups of compounds according Formula I_{F} and the foregoing embodiments and description of this aspect of the invention, D is

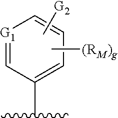

wherein G₁ is N, C—H, or C—R_{M}; G₂ is

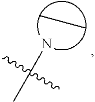

wherein

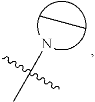

R_{M}, and g are as defined hereinabove. In particular according to these subgroups, R_{M} is each independently fluoro, chloro, methyl, methoxy, trifluoromethyl, or trifluoromethoxy; g is 0, 1, or 2; and

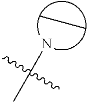

is 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, or 1,4-dioxa-8-azaspiro[4.5]dec-8-yl. In further subgroups of compounds D is

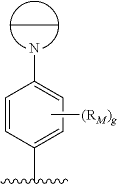

wherein g is 0, 1, or 2; R_{M} is each independently fluoro, chloro, methyl, methoxy, trifluoromethyl, or trifluoromethoxy; and

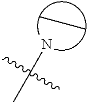

is as defined above. In further subgroups of compounds D is

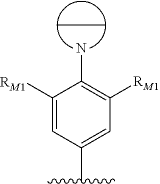

wherein R_{M1} is each independently hydrogen, fluoro, chloro, or methyl and

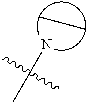

is as defined above (e.g., 3-azabicyclo[3.2.0]hept-3-yl, octahydro-2H-isoindol-2-yl, 2-azabicyclo[2.2.2]oct-2-yl, 6-azaspiro[2.5]oct-6-yl, 3-azaspiro[5.5]undec-3-yl, 1,3-dihydro-2H-isoindol-2-yl, 1,4-dioxa-8-azaspiro[4.5]dec-8-yl).

In other groups of compounds according Formula I_{F} and the foregoing embodiments and description of this aspect of the invention, D is

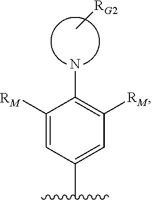

wherein

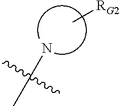

is a monocyclic 4-8 membered nitrogen-containing heterocycle (e.g., azetidinyl, pyrrolidinyl, piperidinyl) substituted with one or more R_{G2}, wherein R_{G2} at each occurrence is each independently halogen, —C(O)C₁-C₆alkyl, —C₁-C₆alkyl, —C₁-C₆haloalkyl, —O—C₁-C₆alkyl, or —O—C₁-C₆haloalkyl; and R_{M} is each independently halogen, —C₁-C₆alkyl, —C₁-C₆haloalkyl, —O—C₁-C₆alkyl, or —O—C₁-C₆haloalkyl. In each group of compounds according to the foregoing embodiments

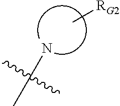

is azetidinyl, pyrrolidinyl, or piperidinyl substituted with one or two R_{G2}, wherein R_{G2} at each occurrence is each methyl, ethyl, isopropyl, tert-butyl, fluoro, chloro, or trifluoromethyl; and R_{M} is each independently fluoro, chloro, or methyl. For example

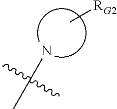

is 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl.

In still another aspect, the present invention features compounds of Formula I_{G} and pharmaceutically acceptable salts thereof,

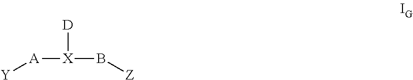

wherein:
wherein X is CH₂CH, CHCH₂, C═C(H) or C(H)═C, and is optionally substituted with one or more R_{A}
A is

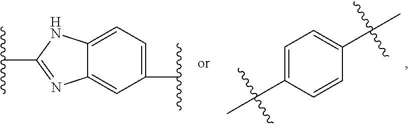

wherein A is optionally substituted with one or more R_{A};
B is

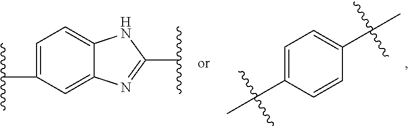

wherein B is optionally substituted with one or more R_{A}; and
Y, Z, R_{A}, and D are as described hereinabove (e.g., as described for Formula I, I_{A}, I_{B}, I_{C}, I_{D}, I_{E} or I_{F}, preferably as described for Formula I_{E}).

In one embodiment, this aspect of the invention features compounds of Formula I_{G} and pharmaceutically acceptable salts thereof, wherein: A is

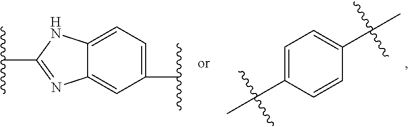

wherein A is optionally substituted with one R_{A}; B is

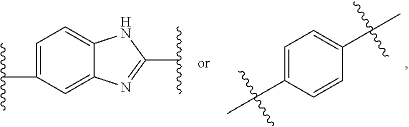

wherein B is optionally substituted with one R_{A}; R_{A} is halogen (e.g., fluoro, chloro); L_{S}-R_{E} where L_{S} is a single bond and R_{E} is —C₁-C₆alkyl (e.g., methyl), —O—R_{S} (e.g., —O—C₁-C₆alkyl, —OCH₃), or —C₁-C₆alkyl optionally substituted with one or more halogen (e.g., —CF₃); or L_{S}-R_{E} where L_{S} is a C₁-C₆alkylene and R_{E} is —O—R_{S} (e.g., —C₁-C₆alkyl-O—C₁-C₆alkyl, —CH₂OCH₃); Y and Z are each independently

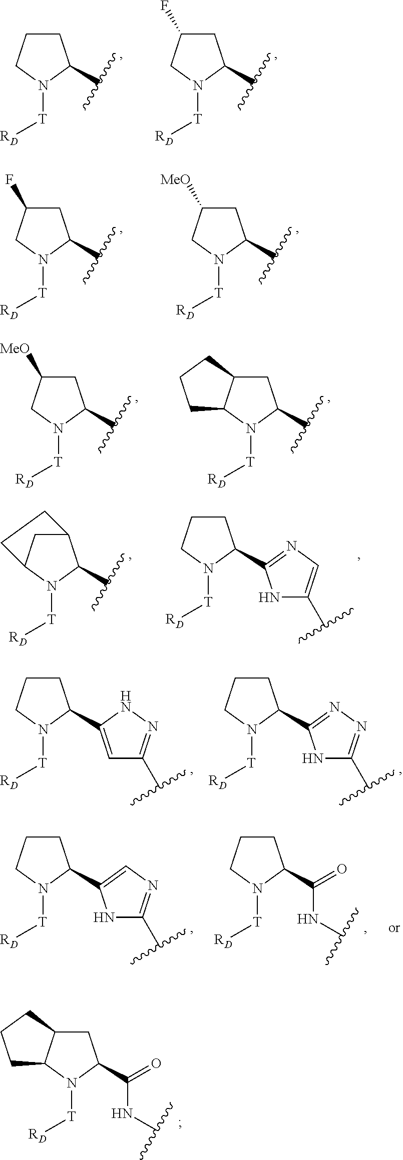

T-R_{D} is each independently

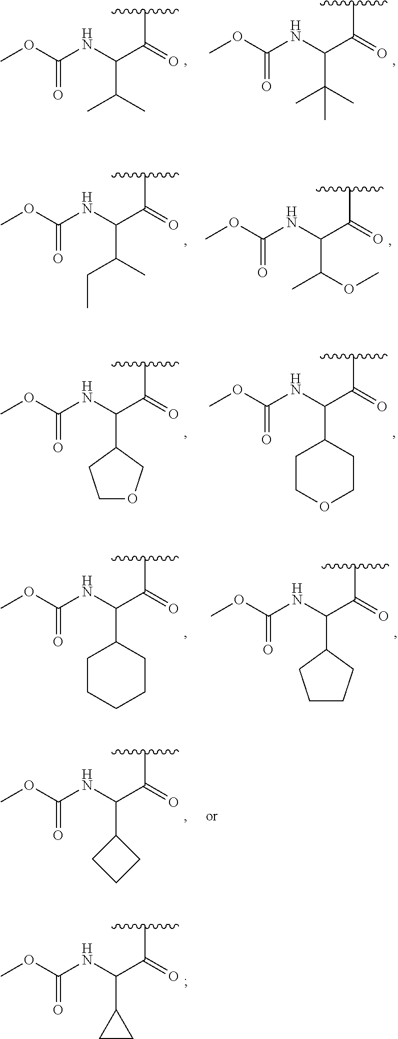

and D is as defined hereinabove.

In another embodiment, this aspect of the invention features compounds of Formula I_{G} and pharmaceutically acceptable salts thereof, wherein A is

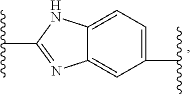

wherein A is optionally substituted with one R_{A}; B is

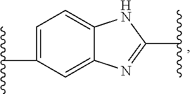

wherein B is optionally substituted with one R_{A}; R_{A} is halogen (e.g., fluoro, chloro); L_{S}-R_{E} where L_{S} is a single bond and R_{E} is —C₁-C₆alkyl (e.g., methyl), —O—R_{S} (e.g., —O—C₁-C₆alkyl, —OCH₃), or —C₁-C₆alkyl optionally substituted with one or more halogen (e.g., —CF₃); or L_{S}-R_{E} where L_{S} is a C₁-C₆alkylene and R_{E} is —O—R_{S} (e.g., —C₁-C₆alkyl-O—C₁-C₆alkyl, —CH₂OCH₃); Y and Z are each independently

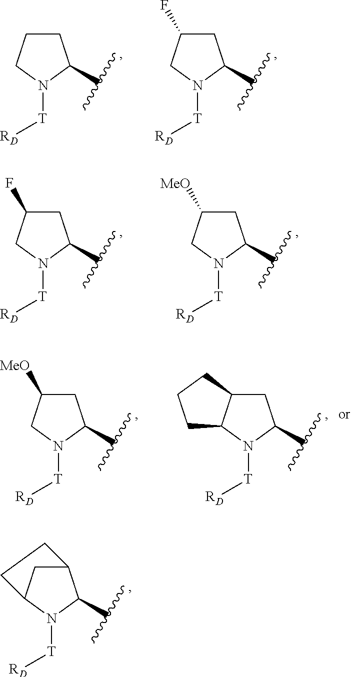

T-R_{D} is each independently

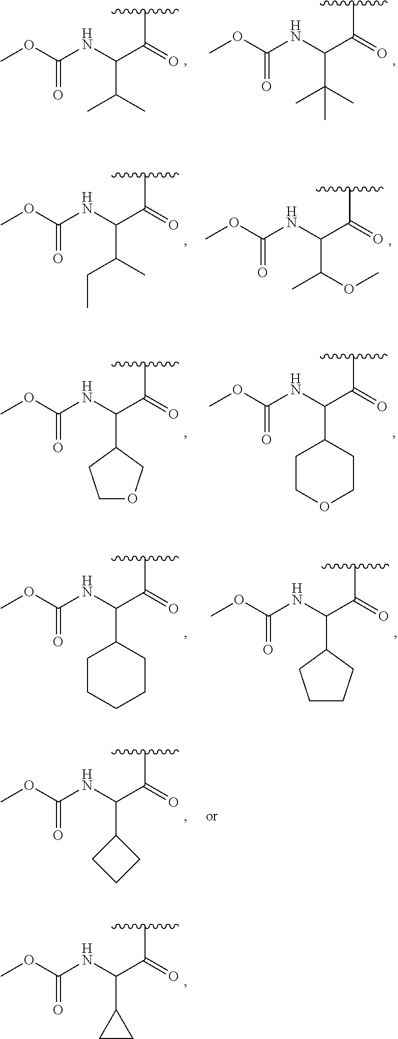

wherein compounds having (S) stereochemistry

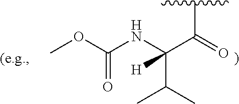

are particularly contemplated; and D is as defined hereinabove. This subgroup includes compounds where A and B are both substituted by one R_{A}; compounds where A and B are both substituted by zero R_{A}; compounds where A is substituted by one R_{A} and B is substituted by zero R_{A}; and compounds where A is substituted by zero R_{A} and B is substituted by one R_{A}. In particular, according to this subgroup are included compounds where

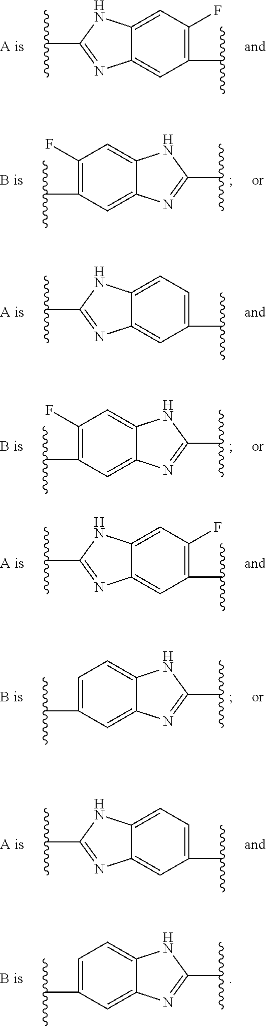

According to each of the foregoing embodiments and description of this aspect of the invention of Formula I_{G} are groups and subgroups of compounds having particular values for D. Included in each of the foregoing embodiments are groups and subgroups of compounds with the following particular values for D:

Groups of compounds according to this aspect of the invention include compounds where D is C₆-C₁₀aryl (e.g., phenyl, naphthyl, indanyl), or 5- to 10-membered heteroaryl (pyridinyl, thiazolyl, 4,5,6,7-tetrahydrobenzo[d]thiazolyl, benzo[d]thiazolyl, indazolyl, benzo[d][1,3]dioxol-5-yl), and D is substituted with one or more R_{M}. Particular subgroups according to this aspect and these embodiments include compounds wherein R_{M} is halogen (e.g., fluoro, chloro, bromo); C₁-C₆alkyl (e.g., tert-butyl); C₁-C₆alkyl substituted with one or more halogen (e.g., CF₃); —O—C₁-C₆alkyl (e.g., —O—CH₂CH₃); —O—C₁-C₆alkyl substituted at each occurrence with one or more halogen (e.g., —O—CF₃, —O—CH₂CHF₂) or —O—C₁-C₆alkyl (—O—CH₂CH₂OCH₃); —O—C₁-C₆alkyl (e.g., —O—CH₂) substituted with an optionally substituted 3- to 12-membered heterocycle (e.g., 3-ethyloxetan-3-yl, 1,3-dioxolan-4-yl); —O—R_{S} where R_{S} is an optionally substituted 3- to 12-membered carbocycle or heterocycle (e.g., cyclopentyl, cyclohexyl, phenyl, 1,3-dioxan-5-yl); —N(R_{S})C(O)R_{S}′ wherein R_{S} and R_{S}′ are each independently C₁-C₆alkyl (e.g., —N(t-Bu)C(O)Me); SF₅; —SO₂R_{S} wherein R_{S} is C₁-C₆alkyl (e.g., —SO₂Me); or C₃-C₁₂carbocycle (e.g., cyclopropyl, cyclohexyl, phenyl). Other subgroups according to this embodiment include compounds wherein D is phenyl substituted by G₂ and optionally substituted by one or more R_{M}, wherein G₂ is a 3- to 12-membered heterocycle (e.g., pyridinyl, piperidinyl, pyrrolidinyl, azetidinyl, oxazolyl) wherein the heterocycle is optionally substituted with one or more substituents selected from halogen, hydroxy, oxo, cyano, C₁-C₆alkyl (e.g., methyl), C₂-C₆alkenyl, C₂-C₆alkynyl, C₁-C₆haloalkyl (e.g., CF₃), C₂-C₆haloalkenyl, C₂-C₆haloalkynyl, —O—C₁-C₆alkyl (e.g., —O—CH₃), —C(O)OR_{S} (e.g., —C(O)OCH₃), —C(O)R_{S} (e.g., —C(O)CH₃), —N(R_{S}R_{S}′), or L₄-G₃; R_{M} is halogen (e.g., fluoro, chloro), alkyl (e.g., methyl), haloalkyl (e.g., CF₃), or —O—C₁-C₆alkyl (e.g., —O—CH₃); and L₄, G₃, R_{S}, and R_{S}′ are as defined hereinabove.

In certain groups of compounds according to Formula I_{G} and the foregoing embodiments and description of this aspect of the invention, D is

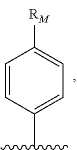

where R_{M} is fluoro, chloro, tert-butyl, —O—CH₂CH₃, —O—CF₃, —O—CH₂CHF₂, —O—CH₂CH₂OCH₃, —O—CH₂-(3-ethyloxetan-3-yl), —O—CH₂-(1,3-dioxolan-4-yl), —O-cyclopentyl, —O-cyclohexyl, —O-phenyl, —O-(1,3-dioxan-5-yl), cyclopropyl, cyclohexyl, phenyl, SF₅, —SO₂Me, or —N(t-Bu)C(O)Me and D is optionally substituted by one or more additional R_{M}, selected from the group consisting of halogen (e.g., fluoro, chloro) or C₁-C₆alkyl (e.g., methyl).

In other groups of compounds according Formula I_{G} and the foregoing embodiments and description of this aspect of the invention, D is

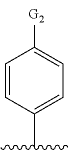

wherein G₂ is pyridinyl (e.g., pyridin-2-yl), piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, 3,3-dimethylazetidin-1-yl, or oxazolyl (e.g., 1,3-oxazol-2-yl) and D is optionally substituted by one or more additional R_{M} selected from the group consisting of halogen (e.g., fluoro, chloro), or C₁-C₆alkyl (e.g., methyl). In particular according to these groups are compounds where D is

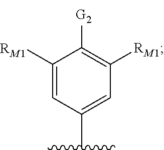

G₂ is piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl; and R_{M1} is each independently hydrogen, fluoro, chloro, or methyl.

In other groups of compounds according Formula I_{G} and the foregoing embodiments and description of this aspect of the invention, D is

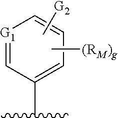

wherein G₁ is N, C—H, or C—R_{M}; G₂ is

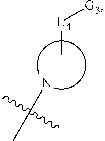

wherein

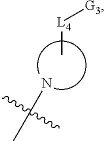

R_{M}, and g are as defined hereinabove. In particular according to these groups, R_{M} is each independently fluoro, chloro, methyl, methoxy, trifluoromethyl, or trifluoromethoxy; g is 0, 1, or 2; and

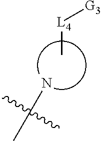

is as defined hereinabove. In further subgroups L₄ is a bond; G₂ is

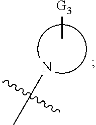

R_{M} is each independently fluoro, chloro, methyl, methoxy, trifluoromethyl, or trifluoromethoxy; and g is 0, 1, or 2. In particular subgroups,

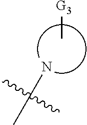

is 3-phenylazetidin-1-yl, 3-phenylpyrrolidin-1-yl, 4-phenylpiperazin-1-yl, 4-phenylpiperidin-1-yl, 4-phenyl-3,6-dihydropyridin-1(2H)-yl, 4,4-diphenylpiperidin-1-yl, 4-acetyl-4-phenylpiperidin-1-yl, 4-(4-methoxyphenyl)piperidin-1-yl, 4-(4-fluorophenyl)piperidin-1-yl, or 3-phenylpiperidin-1-yl; R_{M} is each independently fluoro, chloro, methyl, methoxy, trifluoromethyl, or trifluoromethoxy; and g is 0, 1, or 2. In other subgroups L₄ is C₁-C₆ alkylene, —O—, or —S(O)₂—; G₂ is

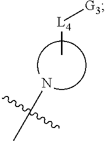

R_{M} is each independently fluoro, chloro, methyl, methoxy, trifluoromethyl, or trifluoromethoxy; and g is 0, 1, or 2. In particular subgroups,

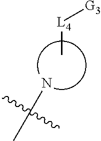

is 4-tosylpiperazin-1-yl, 4-phenoxypiperidin-1-yl, 3-phenoxypyrrolidin-1-yl, 4-benzylpiperidin-1-yl, 4-phenethylpiperidin-1-yl, or 3-phenylpropyl)piperidin-1-yl; R_{M} is each independently fluoro, chloro, methyl, methoxy, trifluoromethyl, or trifluoromethoxy; and g is 0, 1, or 2. In further subgroups of compounds D is

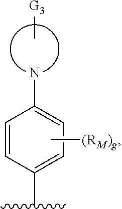

wherein G₃ is phenyl optionally substituted with one or two R_{G3}; g is 0, 1, or 2; R_{M} is each independently fluoro, chloro, methyl, methoxy, trifluoromethyl, or trifluoromethoxy; and

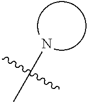

and R_{G3} are as defined above. In other groups of compounds D is

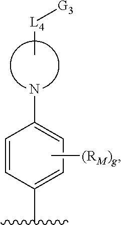

wherein L₄ is C₁-C₆ alkylene, —O—, or —S(O)₂—; G₃ is phenyl optionally substituted with one or two R_{G3}; g is 0, 1, or 2; R_{M} is each independently fluoro, chloro, methyl, methoxy, trifluoromethyl, or trifluoromethoxy; and

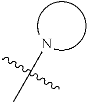

and R_{G3} are as defined above. In further subgroups of compounds D is

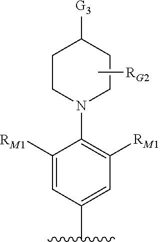

wherein G₃ is phenyl optionally substituted with one or two R_{G3} as defined hereinabove; R_{M1} is each independently hydrogen, fluoro, chloro, or methyl; and R_{G2} is an optional substituent, as described above, selected from the group consisting of —C(O)C₁-C₆alkyl, —C₁-C₆haloalkyl, —O—C₁-C₆alkyl, and —O—C₁-C₆haloalkyl.

In other groups of compounds according Formula I_{G} and the foregoing embodiments and description of this aspect of the invention, D is

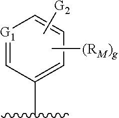

wherein G₁ is N, C—H, or C—R_{M}; G₂ is

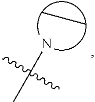

wherein

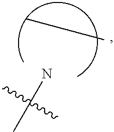

R_{M}, and g are as defined hereinabove. In particular according to these subgroups, R_{M} is each independently fluoro, chloro, methyl, methoxy, trifluoromethyl, or trifluoromethoxy; g is 0, 1, or 2; and

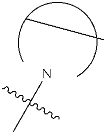

is 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, or 1,4-dioxa-8-azaspiro[4.5]dec-8-yl. In further subgroups of compounds D is

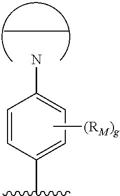

wherein g is 0, 1, or 2; R_{M} is each independently fluoro, chloro, methyl, methoxy, trifluoromethyl, or trifluoromethoxy; and

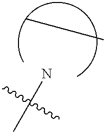

is as defined above. In further subgroups of compounds D is

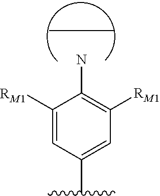

wherein R_{M1} is each independently hydrogen, fluoro, chloro, or methyl and

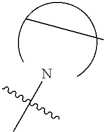

is as defined above (e.g., 3-azabicyclo[3.2.0]hept-3-yl, octahydro-2H-isoindol-2-yl, 2-azabicyclo[2.2.2]oct-2-yl, 6-azaspiro[2.5]oct-6-yl, 3-azaspiro[5.5]undec-3-yl, 1,3-dihydro-2H-isoindol-2-yl, 1,4-dioxa-8-azaspiro[4.5]dec-8-yl).

In other groups of compounds according Formula I_{G} and the foregoing embodiments and description of this aspect of the invention, D is

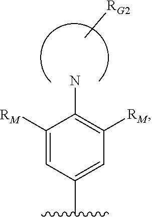

wherein

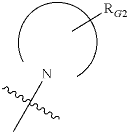

is a monocyclic 4-8 membered nitrogen-containing heterocycle (e.g., azetidinyl, pyrrolidinyl, piperidinyl) substituted with one or more R_{G2}, wherein R_{G2} at each occurrence is each independently halogen, —C(O)C₁-C₆allyl, —C₁-C₆alkyl, —C₁-C₆haloalkyl, —O—C₁-C₆alkyl, or —O—C₁-C₆haloalkyl; and R_{M} is each independently halogen, —C₁-C₆alkyl, —C₁-C₆haloalkyl, —O—C₁-C₆alkyl, or —O—C₁-C₆haloalkyl. In each group of compounds according to the foregoing embodiments

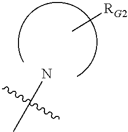

is azetidinyl, pyrrolidinyl, or piperidinyl substituted with one or two R_{G2}, wherein R_{G2} at each occurrence is each methyl, ethyl, isopropyl, tert-butyl, fluoro, chloro, or trifluoromethyl, and R_{M} is each independently fluoro, chloro, or methyl. For example

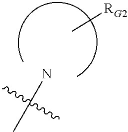

is 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl.

The present invention also features compounds of Formulae I_{F}, I_{F} and I_{G} as described herein (including each embodiment described hereunder) and pharmaceutically acceptable salts thereof, wherein:
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, ═C(R_{S}R_{S}′), or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₁₂carbocycle or 3- to 12-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, trimethylsilyl, C₁-C₆alkyl, C₂-C₆alkenyl, C₂-C₆alkynyl, C₁-C₆haloalkyl, C₂-C₆haloalkenyl, C₂-C₆haloalkynyl, —O—R_{S}, —S—R_{S}, —C(O)R_{S}, —C(O)OR_{S}, or —N(R_{S}R_{S}′).

In yet another aspect, the present invention further features compounds of Formula I_{H} and pharmaceutically acceptable salts thereof;

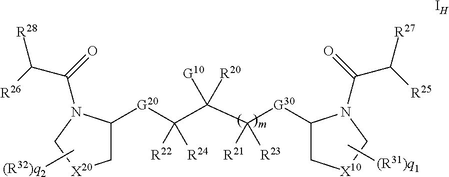

wherein:

G¹⁰ is C₃-C₁₂carbocycle or 3- to 12-membered heterocycle, and is optionally substituted with one or more R_{A}; or G¹⁰ is C₃-C₁₂carbocycle or 3- to 12-membered heterocycle, and is optionally substituted with one ore more R_{A}, and is substituted with J¹, J¹-J², J¹-J²-J³, or J¹-J²-J³-J⁴;

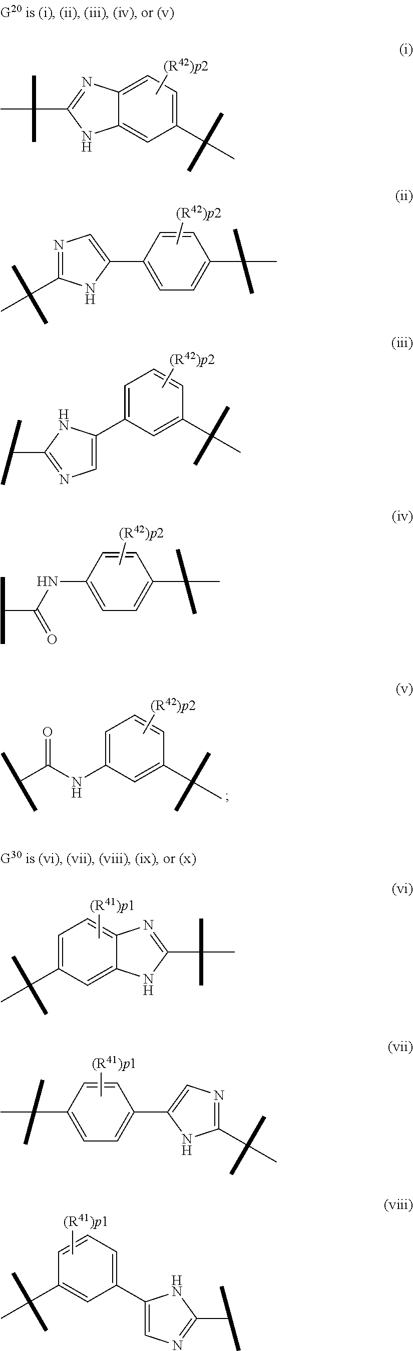

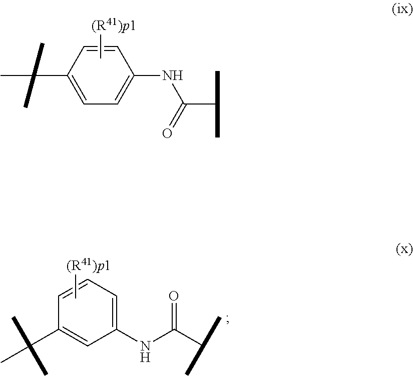

J¹, J², J³ or J⁴ are each independently a C₃-C₁₂carbocycle or 3- to 12-membered heterocycle each of which is optionally and independently substituted with one or more R_{A}.

R²⁰ is hydrogen, alkyl, or haloalkyl;

R²¹, R²², R²³, R²⁴ are each independently hydrogen, alkyl, haloalkyl, or halo;

R²⁵ and R²⁶ are each independently hydrogen, alkyl, alkenyl, alkynyl, cycloalkyl, phenyl, cycloalkylalkyl, haloalkyl, alkoxyalkyl, hydroxyalkyl, heteroaryl, or heterocycle;

R²⁷ and R²⁸ are each independently —N(R^{2a})C(O)R^{2b}, —N(R^{2a})S(O)₂R^{2b}, —N(R^{2a})C(O)O(R²), N(R^{2a})₂, NR^{2a}G^{2a}, or -G^{2a};

R^{2a} at each occurrence is each hydrogen, alkyl, or haloalkyl;

R^{2b} at each occurrence is each, hydrogen, alkyl, haloalkyl, cycloalkyl, alkoxyalkyl, or cycloalkylalkyl;

R³¹ and R³² at each occurrence are each independently halo, alkyl, hydroxy, alkoxy, or haloalkyl;

R⁴¹ and R⁴² at each occurrence are each independently halo, alkoxy, nitro, alkyl, cyano, or haloalkyl;

G^{2a} at each occurrence is each independently aryl, heteroaryl, or heterocycle wherein each G^{2a} is independently unsubstituted or substituted with 1, 2, or 3 substituents selected from the group consisting of halo, oxo, alkyl, alkoxy, and haloalkyl;

X¹⁰ is —O—, —S—, or —(CH₂)ₘ₁—;

X²⁰ is —O—, —S—, or —(CH₂)ₘ₂—;

p1 and p2 are each independently 0, 1, 2, 3, or 4;

m is 0 or 1;

q1 and q2 are each independently 0, 1, 2, or 3; and

m1 and m2 are each independently 0, 1, 2, or 3.

Particular values of variable groups in compounds of Formula (I_{H}) are described hereinbelow. Such values may be used where appropriate with any of the other values, definitions, claims or embodiments defined hereinbefore or hereinafter. Combinations of substituents are permissible only if such combinations result in stable compounds (i.e., compounds that can be isolated from a reaction mixture).

In various embodiments, the present invention provides at least one variable that occurs more than one time in any substituent or in the compound of the invention or any other formulae herein. Definition of a variable on each occurrence is independent of its definition at another occurrence.

As described generally above, for compounds of Formula (I_{H}), G¹⁰ is optionally substituted C₃-C₁₂carbocycle or 3- to 12-membered heterocycle. In certain embodiments, G¹⁰ is optionally substituted phenyl. In other embodiments, G¹⁰ is optionally substituted heteroaryl (e.g., pyridin-3-yl, pyrimidin-5-yl, thiazolyl, benzothiazolyl).

As described generally above, for compounds of Formula (I_{H}), G¹⁰ is optionally substituted with one or more R_{A}; or G¹⁰ is C₃-C₁₂carbocycle or 3- to 12-membered heterocycle, and is optionally substituted with one or more R_{A}, and is substituted with J¹, J¹-J², J¹-J²-J³, or J¹-J²-J³-J⁴.

In certain embodiments, G¹⁰ is substituted with an alkyl (e.g., t-butyl, isopropyl), halogen (e.g., fluoro, chloro), or haloalkyl (e.g., trifluoromethyl).

In other embodiments G¹⁰ may be substituted with R_{A}, wherein R_{A} is L_{S}-R_{E}, and L_{S} is a bond or C₁-C₆alkylene and R_{E} is O—R_{S}, wherein R_{S} is hydrogen or C₁-C₆alkyl.

In certain embodiments, G¹⁰ is substituted with R_{A}, wherein R_{A} is L_{S}-R_{E}, and L_{S} is C₁-C₆alkylene and R_{E} is C₃-C₆carbocyclyl which is optionally substituted with C₁-C₆alkyl which in turn is optionally substituted with one or more halogen.

In certain embodiments, G¹⁰ is phenyl substituted in the 4-position with alkyl (e.g., 4-tert-butyl, 4-isopropyl), halo (e.g., 4-fluoro, 4-chloro), haloalkyl (e.g., 4-trifluoromethyl), —O-alkyl (e.g., 4-isopropoxy), heterocycle (e.g., 4-morpholin-4-yl), cycloalkyl (e.g., 4-cyclohexyl). In other embodiments, G¹⁰ is phenyl substituted in the 3-position with alkyl, halo, haloalkyl, —O-alkyl or cycloalkyl. In other embodiments, G¹⁰ is phenyl substituted in the 3- and 4-positions with combinations of alkyl, halo, haloalkyl, —O-alkyl, or cycloalkyl.

As described generally above, for compounds of Formula (I_{H}), G²⁰ is (i), (ii), (iii), (iv), or (v)

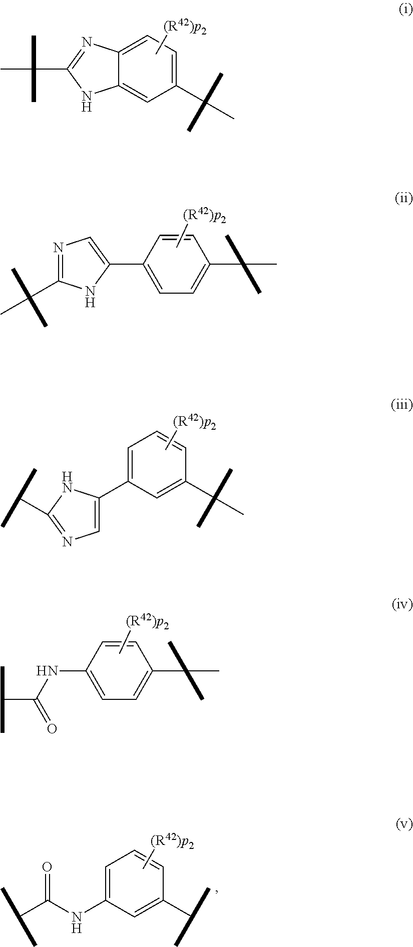

wherein R⁴² and p2 are as described generally above. For example R⁴², at each occurrence, is each independently halo (e.g., fluoro, chloro), alkoxy (e.g., methoxy), nitro, alkyl (e.g., methyl, ethyl), cyano, or haloalkyl (e.g., trifluoromethyl). In certain embodiments, G²⁰ lacks an R⁴² substituent (i.e., p2 is 0). In other embodiments G²⁰ has one or two R⁴² substituents (i.e., p2 is 1 or 2).

As described generally above, for compounds of Formula (I_{H}), G³⁰ is (vi), (vii), (viii), (ix), or (x)

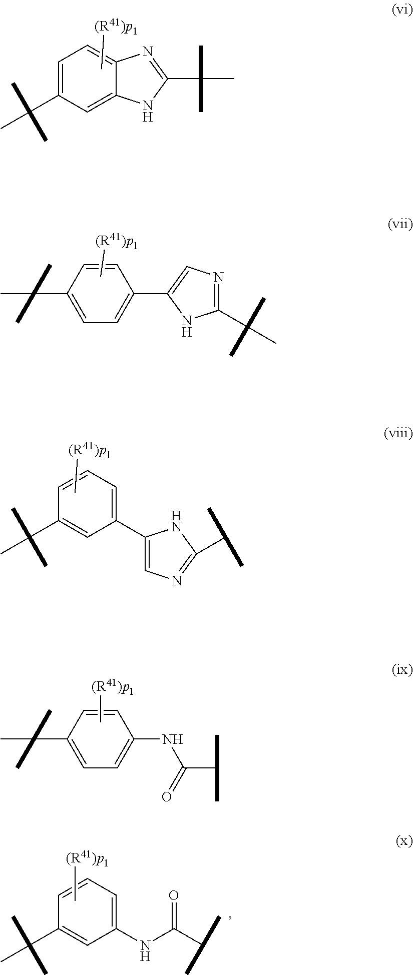

wherein R⁴¹ and p1 are as described generally in the Summary. For example R⁴¹, at each occurrence, is each independently halo (e.g., fluoro, chloro), alkoxy (e.g., methoxy), nitro, alkyl (e.g., methyl, ethyl), cyano, or haloalkyl (e.g., trifluoromethyl). In certain embodiments, G³⁰ lacks an R⁴¹ substituent (i.e., p1 is 0). In other embodiments G³⁰ has one or two R⁴¹ substituents (i.e., p1 is 1 or 2).

The structures (i), (ii), (iii), (vi), (vii), and (viii) each show a single tautomeric form for the groups G²⁰ and G³⁰. It is understood by those skilled in the art that other tautomeric forms may be drawn to depict the actual chemical structures. It is understood that the instant invention embraces the actual chemical structures, including all possible distinct tautomeric structures that may be drawn to depict the chemical structure.

It is understood that the instant invention includes embodiments having particular combinations of G²⁰ and G³⁰. Thus, each of (i), (ii), (iii), (iv), or (v) may be individually incorporated into compounds of the invention in conjunction with any of (vi), (vii), (viii), (ix), or (x).

R²⁰ is as described generally in Formula (I_{H}) above. For example, R²⁰ is hydrogen, alkyl (e.g., methyl), or haloalkyl (e.g., trifluoromethyl). In certain embodiments, R²⁰ is hydrogen.

R²¹, R²², R²³, and R²⁴ are as described generally in Formula (I_{H}) above. For example, R²¹, R²², R²³, and R²⁴, are each independently hydrogen, alkyl (e.g., methyl), haloalkyl (e.g., trifluoromethyl), or halo (e.g., fluoro). In certain embodiments, R²¹, R²², R²³, and R²⁴, are each hydrogen. In certain embodiments, m is 0. When m is 0, the group G³⁰ is bonded directly to the carbon atom to which G¹⁰ and R²⁰ are bonded, and thus R²¹ and R²³ are not part of the structure. In other embodiments, m is 1. When m is 1, G³⁰ is bonded directly to the carbon atom to which R²¹ and R²³ are bonded. When m is 1, certain embodiments of the invention include compounds where R²¹ and R²³ are hydrogen or alkyl (i.e., methyl).

R²⁵ and R²⁶ and other variable groups contained therein are as described generally in Formula (I_{H}) above, as further described in the Definitions above, and the description hereinbelow. For example, in certain embodiments R²⁵ and R²⁶ are each independently hydrogen, alkyl (e.g., methyl, ethyl, isopropyl, tert-butyl, isobutyl, sec-butyl, neopentyl), cycloalkyl (e.g., cyclopentyl, cyclohexyl), phenyl, cycloalkylalkyl (e.g., cyclopropylmethyl), haloalkyl (e.g., trifluoromethyl, trifluoroethyl), alkoxyalkyl (e.g., —CH(CH₃)—OCH₃, having either (R) or (S) stereochemistry), hydroxyalkyl (e.g., —CH₂—OH), or heterocycle (e.g. tetrahydrofuranyl such as tetrahydrofuran-3-yl having either (R) or (S) stereochemistry).

R²⁷ and R²⁸ and other variable groups contained therein are as described generally in Formula (I_{H}) above, as further described herein. For example, in certain embodiments R²⁷ and R²⁸ are each independently —N(R^{2a})C(O)R^{2b} (e.g., —N(H)C(O)CH₃); —N(R^{2a})S(O)₂R^{2b} (e.g., —N(H)S(O)₂CH₃); —N(R^{2a})C(O)O(R^{2b}) (e.g., —N(H)C(O)OCH₃); N(R^{2a})₂ (e.g., —N(CH₃)₂); NR^{2a}G^{2a} (e.g., —N(H)-pyrimidinyl); or -G^{2a} (e.g., piperidinyl, morpholinyl).

X¹⁰ and X²⁰ are as described generally in Formula (I_{H}) above. In certain embodiments, X¹⁰ and X²⁰ are the same. In other embodiments X¹⁰ and X²⁰ are different. For example, in certain embodiments, X¹⁰ and X²⁰ are both —(CH₂)— (i.e., both m1 and m2 are 1). In certain other embodiments, one of X¹⁰ and X²⁰ may be —(CH₂)— and the other of X¹⁰ and X²⁰ may be —O—, —S—, —(CH₂)₂—, —(CH₂)₃—, or a bond (i.e., m1 or m2 is 0). Certain embodiments of the invention comprise compounds containing other combinations of —O—, —S—, —(CH₂)ₘ₁—, and —(CH₂)ₘ₂— for X¹⁰ and X²⁰.

R³¹ and R³² and other variable groups contained therein are as described generally in Formula (I_{H}) above, as further described in the Definitions above, and the description hereinbelow. For example, in certain embodiments R³¹ and R³² are each independently halo (e.g., fluoro), alkyl (e.g., methyl), hydroxy, alkoxy (e.g., methoxy), or haloalkyl (e.g., trifluoromethyl).

As described in Formula (I_{H}) above, q1 and q2 are each independently 0, 1, 2, or 3. In certain embodiments where q1 or q2 is 0, R³¹ or R³², respectively, is absent. In embodiments where q1 and q2 are both 0, R³¹ and R³² are both absent. When either q1 or q2 is 1, 2, or 3, then, respectively, 1, 2, or 3 groups R³¹ or R³² is bonded to the parent molecular structure as indicated in Formula (I_{H}).

It is appreciated that the present invention contemplates separate groups of compounds of Formula (I_{H}) derived from combinations of the above embodiments. As illustrative examples, Formulae (I_{H1}), (I_{H2}), (I_{H3}), (I_{H4}), (I_{H5}), (I_{H6}), (I_{H7}), (I_{H8}), (I_{H9}), or (I_{H10}) each represent a particular embodiment of the invention, wherein G¹⁰, X¹⁰, X²⁰, R²⁵, R²⁶, R²⁷, R²⁸, R³¹, R³², R⁴¹, R⁴², p1, p2, q1, and q2 are as defined in Formula (I_{H}) and as further described hereinabove and hereinbelow.

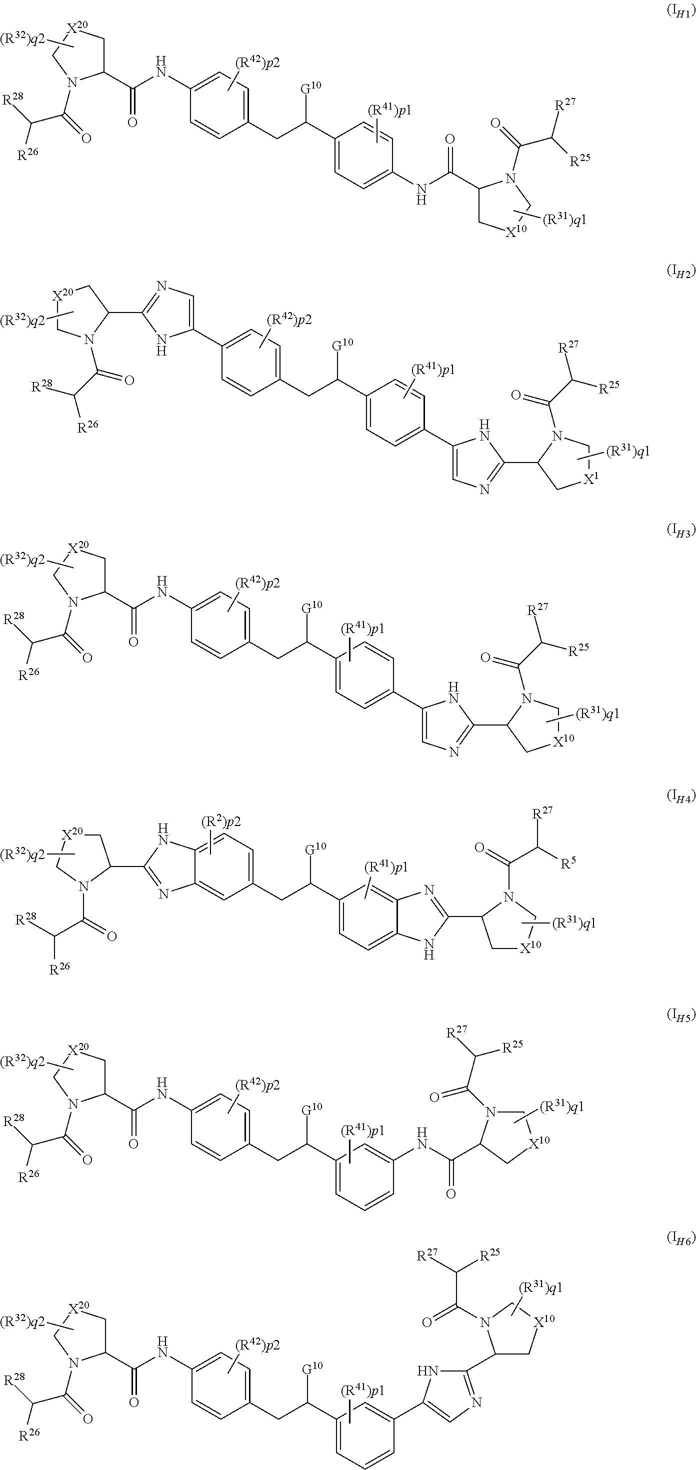

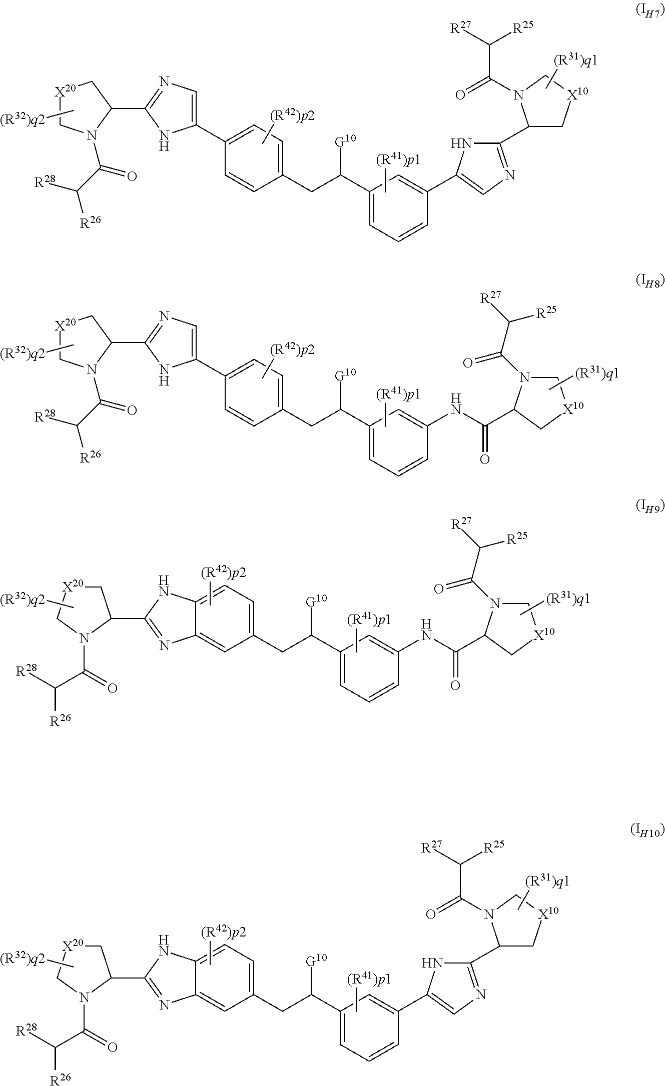

In one embodiment of the invention, separate groups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: q1 and q2 are 0; X¹⁰ and X²⁰ are each —CH₂—; and G¹⁰, R²⁵, R²⁶, R²⁷, R²⁸, R⁴¹, R⁴², p1, and p2 are as defined in Formula (I_{H}) and as further described herein in the Detailed Description.

In another embodiment of the invention, separate groups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: R²⁵ and R²⁶ are alkyl (e.g., ethyl, isopropyl, tert-butyl) or alkoxyalkyl (e.g., —CH(CH₃)—OCH₃, having either (R) or (S) stereochemistry); R²⁷ and R²⁸ are —N(R^{2a})C(O)O(R^{2b}) (e.g., —N(H)C(O)OCH₃); and G¹⁰, X¹⁰, X²⁰, R³¹, R³², R⁴¹, R⁴², p1, p2, q1, and q2 are as defined in Formula (I_{H}) and as further described herein in the Detailed Description.

In another embodiment of the invention, separate groups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: p1 and p2 are 0; and G¹⁰, X¹⁰, X²⁰, R²⁵, R²⁶, R²⁷, R²⁸, R³¹, R³², R⁴¹, R⁴², q1, and q2 are as defined in Formula (I_{H}) and as further described herein in the Detailed Description. Alternatively, in the foregoing formulae and description, one or both of R⁴¹ and R⁴² are fluoro and one or both of p1 and p2, respectively, are 1.

In another embodiment of the invention, separate groups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: G¹⁰ is phenyl optionally substituted with alkyl (e.g., t-butyl, isopropyl), halogen (e.g., fluoro, chloro), haloalkyl (e.g., trifluoromethyl), or J¹ wherein J¹ is heterocycle (e.g., morpholin-4-yl, piperidin-1-yl, tetrahydropyran-4-yl), or cycloalkyl (e.g., cyclopropyl, cyclobutyl, cyclopentyl, cyclohexyl); and X¹⁰, X²⁰, R²⁵, R²⁶, R²⁷, R²⁸, R³¹, R³², R⁴¹, R⁴², p1, p2, q1, q1 and q2 are as defined in Formula (I_{H}) and as further described herein in the Detailed Description.

In another embodiment of the invention, separate groups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: q1 and q2 are 0; X¹⁰ and X²⁰ are each —CH₂—; R²⁵ and R²⁶ are alkyl (e.g., ethyl, isopropyl, tert-butyl) or alkoxyalkyl (e.g., —CH(CH₃)—OCH₃, having either (R) or (S) stereochemistry); R²⁷ and R²⁸ are —N(R^{2a})C(O)O(R^{2b}) (e.g., —N(H)C(O)OCH₃); p1 and p2 are 0; and G¹⁰ is phenyl, pyridinyl, pyrimidinyl, or thiazolyl each optionally substituted as described hereinabove.

The present invention contemplates subgroups of compounds of Formulae ((I_{H1}), (I_{H2}), (I_{H3}), (I_{H4}), (I_{H5}), (I_{H6}), (I_{H7}), (I_{H8}), (I_{H9}), or (I_{H10}) with combinations of the above embodiments.

Separate subgroups of compounds are represented by Formulae (I_{H1}), (I_{H2}), (I_{H3}), (I_{H4}), (I_{H5}), (I_{H6}), (I_{H7}), (I_{H8}), (I_{H9}), or (I_{H10}) wherein: q1 and q2 are 0; X¹⁰ and X²⁰ are each —CH₂—; R²⁵ and R²⁶ are ethyl, isopropyl, tert-butyl or —CH(CH₃)—OCH₃ (having either (R) or (S) stereochemistry); R²⁷ and R²⁸ are —N(H)C(O)OCH₃; p1 and p2 are 0; and G¹⁰ is phenyl substituted at the 3- or 4-position with substituents as described hereinabove. Particular subgroups include those of the foregoing Formulae wherein G¹⁰ is 4-tert-butylphenyl, 4-isopropylphenyl, 4-trifluoromethylphenyl, 4-isopropoxyphenyl, 4-morpholin-4-ylphenyl, or 4-cyclohexylphenyl.

Compounds of the invention of Formulae (I_{H1}), (I_{H2}), (I_{H3}), (I_{H4}), (I_{H5}), (I_{H6}), (I_{H7}), (I_{H8}), (I_{H9}), or (I_{H10}) contain carbon atoms that may be in either (R) or (S) stereochemistry. The present invention contemplates stereoisomers and mixtures thereof and these are specifically included within the scope of this invention. Stereoisomers include enantiomers and diastereomers, and mixtures of enantiomers or diastereomers.

One embodiment of the invention includes compounds possessing the stereochemical configurations shown in Formula (I_{H}). Included in each foregoing embodiment and description of the separate groups and subgroups of compounds having Formulae (I_{H1}), (I_{H2}), (I_{H5}), (I_{H4}), (I_{H5}), (I_{H6}), (I_{H7}), (I_{H8}), (I_{H9}), or (I_{H10}), are further groups and subgroups having the stereochemical configuration shown in Formula (I_{I}).

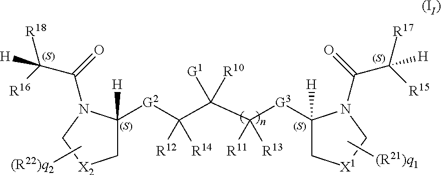

Individual stereoisomers of compounds of the present application may be prepared synthetically from commercially available starting materials which contain asymmetric or chiral centers or by preparation of racemic mixtures followed by resolution which is well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Within the present invention it is to be understood that compounds disclosed herein may exhibit the phenomenon of tautomerism.

Thus, the formulae drawings within this specification can represent only one of the possible tautomeric or stereoisomeric forms. It is to be understood that the invention encompasses any tautomeric or stereoisomeric form, and mixtures thereof, and is not to be limited merely to any one tautomeric or stereoisomeric form utilized within the naming of the compounds or formulae drawings.

Specific embodiments of compounds of the invention include, but are not limited to:
dimethyl [(1-phenylethane-1,2-diyl)bis{benzene-4,1-diylcarbamoyl(2S)pyrrolidine-2,1-diyl [(2S)-3-methyl-1-oxobutane-1,2-diyl]}]biscarbamate
dimethyl [(1-phenylethane-1,2-diyl)bis{benzene-4,1-diylcarbamoyl(2S)pyrrolidine-2,1-diyl[(2S)-3,3-dimethyl-1-oxobutane-1,2-diyl]}]biscarbamate
dimethyl [(1-phenylethane-1,2-diyl)bis{benzene-4,1-diylcarbamoyl(2S)pyrrolidine-2,1-diyl[(2S)-1-oxobutane-1,2-diyl]}]biscarbamate
N-(methoxycarbonyl)-L-valyl-N-(4-{2-[4-(2-{(2S)-1-[N-(methoxycarbonyl)-L -valyl]pyrrolidin-2-yl}-1-1H-imidazol-5-yl)phenyl]-2-phenylethyl}-phenyl)-L-prolinamide
N-(methoxycarbonyl)-3-methyl-L-valyl-N-(4-{2-[4-(2-{(2S)-1-[N-(methoxycarbonyl)-3-methyl-L -valyl]pyrrolidin-2-yl}-1H-imidazol-5-yl)phenyl]-2-phenylethyl}-phenyl)-L-prolinamide
1-{(2S)-2-[(methoxycarbonyl)amino]butanoyl}-N-{4-[2-(4-{2-[(2S)-1-{(2S)-2-[(methoxycarbonyl)amino]butanoyl}pyrrolidin-2-yl]-1H-imidazol-5-yl}phenyl)-2-phenylethyl]phenyl}-1-L-prolinamide
methyl [(2S)-1-{(2S)-2-[5-(4-{2(4-{2-[(2S)-1-{(2S)-2-[(methoxycarbonyl)amino]-3-methylbutanoyl}pyrrolidin-2-yl]-1H-imidazol-5-yl}phenyl)-2-[4-(trifluoromethyl)phenyl]ethyl}phenyl)-1H-imidazol-2-yl]pyrrolidin-1-yl}-3-methyl-1-oxobutan-2-yl]carbamate
methyl {(2S)-1-[(2S)-2-(5-{4-[2-(4-tert-butylphenyl)-2-(4-{2-[(2S)-1-{(2S)-2-[(methoxycarbonyl)amino]-3-methylbutanoyl}pyrrolidin-2-yl]-1H-imidazol-4-yl}phenyl)ethyl]phenyl}-1H-imidazol-2-yl)pyrrolidin-1-yl]-3-methyl-1-oxobutan-2-yl}carbamate
methyl {(2S)-1-[(2S)-2-{6-[1-(4-tert-butylphenyl)-2-{2-[(2S)-1-{(2S)-2-[(methoxycarbonyl)amino]-3-methylbutanoyl}pyrrolidin-2-yl]-1H-benzimidazol-5-yl}ethyl]-1H-benzimidazol-2-yl}pyrrolidin-1-yl]-3-methyl-1-oxobutan-2-yl}carbamate

The compounds of the present invention can be used in the form of salts. Depending on the particular compound, a salt of a compound may be advantageous due to one or more of the salt's physical properties, such as enhanced pharmaceutical stability under certain conditions or desired solubility in water or oil. In some instances, a salt of a compound may be useful for the isolation or purification of the compound.

Where a salt is intended to be administered to a patient, the salt preferably is pharmaceutically acceptable. Pharmaceutically acceptable salts include, but are not limited to, acid addition salts, base addition salts, and alkali metal salts.

Pharmaceutically acceptable acid addition salts may be prepared from inorganic or organic acids. Examples of suitable inorganic acids include, but are not limited to, hydrochloric, hydrobromic, hydroionic, nitric, carbonic, sulfuric, and phosphoric acid. Examples of suitable organic acids include, but are not limited to, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically acceptable base addition salts include, but are not limited to, metallic salts and organic salts. Non-limiting examples of suitable metallic salts include alkali metal (group Ia) salts, alkaline earth metal (group IIa) salts, and other pharmaceutically acceptable metal salts. Such salts may be made, without limitation, from aluminum, calcium, lithium, magnesium, potassium, sodium, or zinc. Non-limiting examples of suitable organic salts can be made from tertiary amines and quaternary amine, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as alkyl halides (e.g., methyl, ethyl, propyl, butyl, decyl, lauryl, myristyl, and stearyl chlorides/bromides/iodides), dialkyl sulfates (e.g., dimethyl, diethyl, dibuytl, and diamyl sulfates), aralkyl halides (e.g., benzyl and phenethyl bromides), and others.

The compounds or salts of the present invention may exist in the form of solvates, such as with water (i.e., hydrates), or with organic solvents (e.g., with methanol, ethanol or acetonitrile to form, respectively, methanolate, ethanolate or acetonitrilate).

The compounds or salts of the present invention may also be used in the form of prodrugs. Some prodrugs are aliphatic or aromatic esters derived from acidic groups on the compounds of the invention. Others are aliphatic or aromatic esters of hydroxyl or amino groups on the compounds of the invention. Phosphate prodrugs of hydroxyl groups are preferred prodrugs.

The compounds of the invention may comprise asymmetrically substituted carbon atoms known as chiral centers. These compounds may exist, without limitation, as single stereoisomers (e.g., single enantiomers or single diastereomer), mixtures of stereoisomers (e.g. a mixture of enantiomers or diastereomers), or racemic mixtures. Compounds identified herein as single stereoisomers are meant to describe compounds that are present in a form that is substantially free from other stereoisomers (e.g., substantially free from other enantiomers or diastereomers). By “substantially free,” it means that at least 80% of the compound in a composition is the described stereoisomer; preferably, at least 90% of the compound in a composition is the described stereoisomer; and more preferably, at least 95%, 96%, 97%, 98% or 99% of the compound in a composition is the described stereoisomer. Where the stereochemistry of a chiral carbon is not specified in the chemical structure of a compound, the chemical structure is intended to encompass compounds containing either stereoisomer of the chiral center.

Individual stereoisomers of the compounds of this invention can be prepared using a variety of methods known in the art. These methods include, but are not limited to, stereospecific synthesis, chromatographic separation of diastereomers, chromatographic resolution of enantiomers, conversion of enantiomers in an enantiomeric mixture to diastereomers followed by chromatographically separation of the diastereomers and regeneration of the individual enantiomers, and enzymatic resolution.

Stereospecific synthesis typically involves the use of appropriate optically pure (enantiomerically pure) or substantial optically pure materials and synthetic reactions that do not cause racemization or inversion of stereochemistry at the chiral centers. Mixtures of stereoisomers of compounds, including racemic mixtures, resulting from a synthetic reaction may be separated, for example, by chromatographic techniques as appreciated by those of ordinary skill in the art. Chromatographic resolution of enantiomers can be accomplished by using chiral chromatography resins, many of which are commercially available. In a non-limiting example, racemate is placed in solution and loaded onto the column containing a chiral stationary phase. Enantiomers can then be separated by HPLC.

Resolution of enantiomers can also be accomplished by converting enantiomers in a mixture to diastereomers by reaction with chiral auxiliaries. The resulting diastereomers can be separated by column chromatography or crystallization/re-crystallization. This technique is useful when the compounds to be separated contain a carboxyl, amino or hydroxyl group that will form a salt or covalent bond with the chiral auxiliary. Non-limiting examples of suitable chiral auxiliaries include chirally pure amino acids, organic carboxylic acids or organosulfonic acids. Once the diastereomers are separated by chromatography, the individual enantiomers can be regenerated. Frequently, the chiral auxiliary can be recovered and used again.

Enzymes, such as esterases, phosphatases or lipases, can be useful for the resolution of derivatives of enantiomers in an enantiomeric mixture. For example, an ester derivative of a carboxyl group in the compounds to be separated can be treated with an enzyme which selectively hydrolyzes only one of the enantiomers in the mixture. The resulting enantiomerically pure acid can then be separated from the unhydrolyzed ester.

Alternatively, salts of enantiomers in a mixture can be prepared using any suitable method known in the art, including treatment of the carboxylic acid with a suitable optically pure base such as alkaloids or phenethylamine, followed by precipitation or crystallization/re-crystallization of the enantiomerically pure salts. Methods suitable for the resolution/separation of a mixture of stereoisomers, including racemic mixtures, can be found in ENANTIOMERS, RACEMATES, AND RESOLUTIONS (Jacques et al., 1981, John Wiley and Sons, New York, N.Y.).

A compound of this invention may possess one or more unsaturated carbon-carbon double bonds. All double bond isomers, such as the cis (Z) and trans (E) isomers, and mixtures thereof are intended to be encompassed within the scope of a recited compound unless otherwise specified. In addition, where a compound exists in various tautomeric forms, a recited compound is not limited to any one specific tautomer, but rather is intended to encompass all tautomeric forms.

Certain compounds of the invention may exist in different stable conformational forms which may be separable. Torsional asymmetry due to restricted rotations about an asymmetric single bond, for example because of steric hindrance or ring strain, may permit separation of different conformers. The invention encompasses each conformational isomer of these compounds and mixtures thereof.

Certain compounds of the invention may also exist in zwitterionic form and the invention encompasses each zwitterionic form of these compounds and mixtures thereof.

The compounds of the present invention are generally described herein using standard nomenclature. For a recited compound having asymmetric center(s), it should be understood that all of the stereoisomers of the compound and mixtures thereof are encompassed in the present invention unless otherwise specified. Non-limiting examples of stereoisomers include enantiomers, diastereomers, and cis-transisomers. Where a recited compound exists in various tautomeric forms, the compound is intended to encompass all tautomeric forms. Certain compounds are described herein using general formulas that include variables (e.g., A, B, D, X, L₁, L₂, L₃, Y, Z, T, R_{A} or R_{B}). Unless otherwise specified, each variable within such a formula is defined independently of any other variable, and any variable that occurs more than one time in a formula is defined independently at each occurrence. If moieties are described as being “independently” selected from a group, each moiety is selected independently from the other. Each moiety therefore can be identical to or different from the other moiety or moieties.

The number of carbon atoms in a hydrocarbyl moiety can be indicated by the prefix “Cₓ-C_{y},” where x is the minimum and y is the maximum number of carbon atoms in the moiety. Thus, for example, “C₁-C₆alkyl” refers to an alkyl substituent containing from 1 to 6 carbon atoms. Illustrating further, C₃-C₆cycloalkyl means a saturated hydrocarbyl ring containing from 3 to 6 carbon ring atoms. A prefix attached to a multiple-component substituent only applies to the first component that immediately follows the prefix. To illustrate, the term “carbocyclylalkyl” contains two components: carbocyclyl and alkyl. Thus, for example, C₃-C₆carbocyclylC₁-C₆alkyl refers to a C₃-C₆carbocyclyl appended to the parent molecular moiety through a C₁-C₆alkyl group.

Unless otherwise specified, when a linking element links two other elements in a depicted chemical structure, the leftmost-described component of the linking element is bound to the left element in the depicted structure, and the rightmost-described component of the linking element is bound to the right element in the depicted structure. To illustrate, if the chemical structure is -L_{S}-M-L_{S}′- and M is —N(R_{B})S(O)—, then the chemical structure is -L_{S}-N(R_{B})S(O)-L_{S}′-.

If a linking element in a depicted structure is a bond, then the element left to the linking element is joined directly to the element right to the linking element via a covalent bond. For example, if a chemical structure is depicted as -L_{S}-M-L_{S}′- and M is selected as bond, then the chemical structure will be -L_{S}-L_{S}′-. If two or more adjacent linking elements in a depicted structure are bonds, then the element left to these linking elements is joined directly to the element right to these linking elements via a covalent bond. For instance, if a chemical structure is depicted as -L_{S}-M-L_{S}′-M′-L_{S}″-, and M and L_{S}′ are selected as bonds, then the chemical structure will be -L_{S}-M′-L_{S}″-. Likewise, if a chemical structure is depicted as -L_{S}-M-L_{S}′-M′-L_{S}″-, and M, L_{S}′ and M′ are bonds, then the chemical structure will be -L_{S}-L_{S}″-.

When a chemical formula is used to describe a moiety, the dash(s) indicates the portion of the moiety that has the free valence(s).

If a moiety is described as being “optionally substituted”, the moiety may be either substituted or unsubstituted. If a moiety is described as being optionally substituted with up to a particular number of non-hydrogen radicals, that moiety may be either unsubstituted, or substituted by up to that particular number of non-hydrogen radicals or by up to the maximum number of substitutable positions on the moiety, whichever is less. Thus, for example, if a moiety is described as a heterocycle optionally substituted with up to three non-hydrogen radicals, then any heterocycle with less than three substitutable positions will be optionally substituted by up to only as many non-hydrogen radicals as the heterocycle has substitutable positions. To illustrate, tetrazolyl (which has only one substitutable position) will be optionally substituted with up to one non-hydrogen radical. To illustrate further, if an amino nitrogen is described as being optionally substituted with up to two non-hydrogen radicals, then a primary amino nitrogen will be optionally substituted with up to two non-hydrogen radicals, whereas a secondary amino nitrogen will be optionally substituted with up to only one non-hydrogen radical.

The term “alkenyl” means a straight or branched hydrocarbyl chain containing one or more double bonds. Each carbon-carbon double bond may have either cis or trans geometry within the alkenyl moiety, relative to groups substituted on the double bond carbons. Non-limiting examples of alkenyl groups include ethenyl (vinyl), 2-propenyl, 3-propenyl, 1,4-pentadienyl, 1,4-butadienyl, 1-butenyl, 2-butenyl, and 3-butenyl.

The term “alkenylene” refers to a divalent unsaturated hydrocarbyl chain which may be linear or branched and which has at least one carbon-carbon double bond. Non-limiting examples of alkenylene groups include —C(H)═C(H)—, —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkyl” means a straight or branched saturated hydrocarbyl chain. Non-limiting examples of alkyl groups include methyl, ethyl, n-propyl, isopropyl, n-butyl, isobutyl, sec-butyl, t-butyl, pentyl, iso-amyl, and hexyl.

The term “alkylene” denotes a divalent saturated hydrocarbyl chain which may be linear or branched. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” means a straight or branched hydrocarbyl chain containing one or more triple bonds. Non-limiting examples of alkynyl include ethynyl, 1-propynyl, 2-propynyl, 3-propynyl, decynyl, 1-butynyl, 2-butynyl, and 3-butynyl.

The term “alkynylene” refers to a divalent unsaturated hydrocarbon group which may be linear or branched and which has at least one carbon-carbon triple bonds. Representative alkynylene groups include, by way of example, —C≡C—, —C≡C—CH₂—, —C≡C—CH₂—CH₂—, —CH₂—C≡C—CH₂—, —C≡C—CH(CH₃)—, and —CH₂—C≡C—CH(CH₂CH₃)—.

The term “carbocycle” or “carbocyclic” or “carbocyclyl” refers to a saturated (e.g., “cycloalkyl”), partially saturated (e.g., “cycloalkenyl” or “cycloalkynyl”) or completely unsaturated (e.g., “aryl”) ring system containing zero heteroatom ring atom. “Ring atoms” or “ring members” are the atoms bound together to form the ring or rings. A carbocyclyl may be, without limitation, a single ring, two fused rings, or bridged or spiro rings. A substituted carbocyclyl may have either cis or trans geometry. Representative examples of carbocyclyl groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, indanyl, 1,2,3,4-tetrahydro-naphthyl, indenyl, isoindenyl, decalinyl, and norpinanyl. A carbocycle group can be attached to the parent molecular moiety through any substitutable carbon ring atom. Where a carbocycle group is a divalent moiety linking two other elements in a depicted chemical structure (such as A in Formula I), the carbocycle group can be attached to the two other elements through any two substitutable ring atoms.

The term “carbocyclylalkyl” refers to a carbocyclyl group appended to the parent molecular moiety through an alkylene group. For instance, C₃-C₆carbocyclylC₁-C₆alkyl refers to a C₃-C₆carbocyclyl group appended to the parent molecular moiety through C₁-C₆alkylene.

The term “cyano” means —CN.

The term “cyanoalkyl” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” refers to a non-aromatic, partially unsaturated carbocyclyl moiety having zero heteroatom ring member. Representative examples of cycloalkenyl groups include, but are not limited to, cyclobutenyl, cyclopentenyl, cyclohexenyl, and octahydronaphthalenyl.

The term “cycloalkyl” or “cycloalkane” refers to a saturated carbocyclyl group containing zero heteroatom ring member. Non-limiting examples of cycloalkyls include cyclopropyl, cyclobutyl, cyclopentyl, cyclohexyl, cycloheptyl, cyclooctyl, decalinyl and norpinanyl.

The prefix “halo” indicates that the substituent to which the prefix is attached is substituted with one or more independently selected halogen radicals. For example, “C₁-C₆haloalkyl” means a C₁-C₆alkyl substituent wherein one or more hydrogen atoms are replaced with independently selected halogen radicals. Non-limiting examples of C₁-C₆haloalkyl include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated). Likewise, “C₁-C₆haloalkoxy” means a C₁-C₆alkoxy substituent wherein one or more hydrogen atoms are replaced with independently selected halogen radicals. Representative examples of haloalkoxy include, but are not limited to, 2-fluoroethoxy, 2,2,2-trifluoroethoxy, trifluoromethoxy, and difluoromethoxy.

The term “heterocycle” or “heterocyclo” or “heterocyclyl” refers to a saturated (e.g., “heterocycloalkyl”), partially unsaturated (e.g., “heterocycloalkenyl” or “heterocycloalkynyl”) or completely unsaturated (e.g., “heteroaryl”) ring system where at least one of the ring atoms is a heteroatom (i.e., nitrogen, oxygen or sulfur), with the remaining ring atoms being independently selected from the group consisting of carbon, nitrogen, oxygen and sulfur. A heterocycle may be, without limitation, a single ring, two fused rings, or bridged or spiro rings. A heterocycle group can be linked to the parent molecular moiety via any substitutable carbon or nitrogen atom(s) in the group. Where a heterocycle group is a divalent moiety linking two other elements in a depicted chemical structure (such as A in Formula I), the heterocycle group can be attached to the two other elements through any two substitutable ring atoms.

A heterocyclyl may be, without limitation, a monocycle which contains a single ring. Non-limiting examples of monocycles include furanyl, dihydrofuranyl, tetrahydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl and 1,3,5,2-oxadiazinyl), morpholinyl, azepinyl, oxepinyl, thiepinyl, and diazepinyl.

A heterocyclyl may also be, without limitation, a bicycle containing two fused rings, such as, for example, naphthyridinyl (including [1,8] naphthyridinyl, and [1,6] naphthyridinyl), thiazolpyrimidinyl, thienopyrimidinyl, pyrimidopyrimidinyl, pyridopyrimidinyl, pyrazolopyrimidinyl, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, and pyrido[4,3-b]-pyridinyl), pyridopyrimidine, and pteridinyl. Other non-limiting examples of fused-ring heterocycles include benzo-fused heterocyclyls, such as indolyl, isoindolyl, indoleninyl (also known as “pseudoindolyl”), isoindazolyl (also known as “benzpyrazolyl”), benzazinyl (including quinolinyl (also known as “1-benzazinyl”) and isoquinolinyl (also known as “2-benzazinyl”)), benzimidazolyl, phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including “chromenyl” and “isochromenyl”), benzothiopyranyl (also known as “thiochromenyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), and tetrahydroisoquinolinyl.

A heterocyclyl may comprise one or more sulfur atoms as ring members; and in some cases, the sulfur atom(s) is oxidized to SO or SO₂. The nitrogen heteroatom(s) in a heterocyclyl may or may not be quaternized, and may or may not be oxidized to N-oxide. In addition, the nitrogen heteroatom(s) may or may not be N-protected.

The term “hydroxyalkyl” as used herein, means at least one hydroxy group, as defined herein, is appended to the parent molecular moiety through an alkylene group, as defined herein. Representative examples of hydroxyalkyl include, but are not limited to, hydroxymethyl, 2-hydroxyethyl, 3-hydroxypropyl, 2,3-dihydroxypentyl, 2-hydroxy-2-methylpropyl, 1-hydroxy-1-methylethyl, and 2-ethyl-4-hydroxyheptyl.

The term “oxo” as used herein, means an oxygen atom appended to the parent molecular moiety through a double bond.

[symbol] in a chemical formula refers to a single or double bond.

The term “pharmaceutically acceptable” is used adjectivally to mean that the modified noun is appropriate for use as a pharmaceutical product or as a part of a pharmaceutical product.

The term “therapeutically effective amount” refers to the total amount of each active substance that is sufficient to show a meaningful patient benefit, e.g. a reduction in viral load.

The term “prodrug” refers to derivatives of the compounds of the invention which have chemically or metabolically cleavable groups and become, by solvolysis or under physiological conditions, the compounds of the invention which are pharmaceutically active in vivo. A prodrug of a compound may be formed in a conventional manner by reaction of a functional group of the compound (such as an amino, hydroxy or carboxy group). Prodrugs often offer advantages of solubility, tissue compatibility, or delayed release in mammals (see, Bungard, H., DESIGN OF PRODRUGS, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine Examples of prodrugs include, but are not limited to, acetate, formate, benzoate or other acylated derivatives of alcohol or amine functional groups within the compounds of the invention.

The term “solvate” refers to the physical association of a compound of this invention with one or more solvent molecules, whether organic or inorganic. This physical association often includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include, but are not limited to, hydrates, ethanolates, and methanolates.

The term “N-protecting group” or “N-protected” refers to those groups capable of protecting an amino group against undesirable reactions. Commonly used N-protecting groups are described in Greene and Wuts, PROTECTIVE GROUPS IN ORGANIC SYNTHESIS (3^{rd} ed., John Wiley & Sons, NY (1999). Non-limiting examples of N-protecting groups include acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, benzoyl, 4-chlorobenzoyl, 4-bromobenzoyl, or 4-nitrobenzoyl; sulfonyl groups such as benzenesulfonyl or p-toluenesulfonyl; sulfenyl groups such as phenylsulfenyl (phenyl-S—) or triphenylmethylsulfenyl (trityl-S—); sulfinyl groups such as p-methylphenylsulfinyl (p-methylphenyl-S(O)—) or t-butylsulfinyl (t-Bu-S(O)—); carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxy carbonyl, 2-nitro-4,5-dimethoxybenzyloxycarbonyl, 3,4,5-trimethoxybenzyloxycarbonyl, 1-(p-biphenylyl)-1-methylethoxycarbonyl, dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloro-ethoxy-carbonyl, phenoxycarbonyl, 4-nitro-phenoxycarbonyl, cyclopentyloxycarbonyl, adamantyloxycarbonyl, cyclohexyloxycarbonyl, or phenylthiocarbonyl; alkyl groups such as benzyl, p-methoxybenzyl, triphenylmethyl, or benzyloxymethyl; p-methoxyphenyl; and silyl groups such as trimethylsilyl. Preferred N-protecting groups include formyl, acetyl, benzoyl, pivaloyl, t-butylacetyl, phenylsulfonyl, benzyl, t-butyloxycarbonyl (Boc) and benzyloxycarbonyl (Cbz).

The compounds of the present invention can be prepared using a variety of methods. For example, certain compounds of the invention (40) wherein G¹⁰ is optionally substituted phenyl and R²⁵, R²⁶, R²⁷, and R²⁸ are as described above, can be prepared according to the general method illustrated in Scheme V.

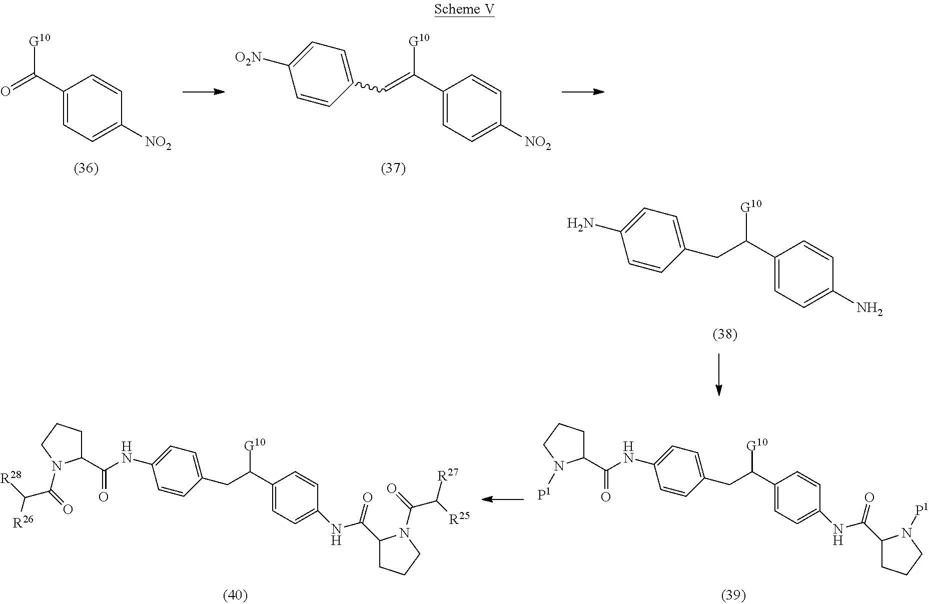

Ketones (71) can be subjected to Wittig, Horner-Wadworth-Emmons, or like reaction to produce alkenes of general formula (37). These general alkene forming reactions are well known to those of skill in the art and are described in J. March, Advanced Organic Chemistry: Reactions, Mechanisms, and Structure 4th Ed. p956-963, and references cited therein. In particular, ketones (71) can be reacted with diethyl 4-nitrobenzylphosphonate in the presence of a base such as, but not limited to, sodium hydride or sodium bistrimethylsilylamide (NaHMDS) at temperatures from about 0° C. to about 110° C. in solvents such as, but not limited to, dimethylsulfoxide, tetrahydrofuran, or dimethylformamide to afford alkenes (37). The reaction may also be conducted in the presence of 15-crown-5 (Chempartner selection).

Alkenes (37) can be transformed to the diaminoalkanes (38) by catalytic hydrogenation. Typical catalysts include palladium on carbon, platinum, or platinum oxide. Solvents for this reaction include, but are not limited to, ethyl acetate, methanol, or ethanol.

The diaminoalkanes (38) can be transformed to the amides (39) by reaction with a suitably protected proline acid wherein P¹ represents a protecting group such as, but not limited to, t-butoxycarbonyl (Boc), benzyloxycarbonyl (Cbz), 2,2,2-trichloroethoxycarbonyl (Troc), 9-fluorenylmethoxycarbonyl (Fmoc) and the like. Additional protecting groups suitable for N-protection can be found in T. W. Greene and P. G. M. Wuts, Protective Groups in Organic Synthesis. The coupling of (38) with a protected proline acid is conducted with a peptide coupling reagent such N-(3-dimethylaminopropyl)-N′-ethylcarbodiimide hydro chloride/1-hydroxybenzotriazole (EDAC/HOBt), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), O-(7-azabenzotriazol-1-yl)-N,N,N′,N′-tetramethyluronium hexafluorophosphate (HATU), or 3-(diethoxyphosphoryloxy)-1,2,3-benzotriazin-4(310-one (DEPBT); in solvents such as, but not limited to tetrahydrofuran or dimethylformamide; with bases such as, but not limited to, diisopropylethylamine, pyridine, 2,6-lutidine, or triethylamine at temperatures from about room temperature to about 60° C. to give compounds of general formula (39).

Compounds of general formula (39) can be converted to compounds of the invention of general formula (40) by removal of the P¹ protecting group followed by reaction with an acid such as, but not limited to, (S)-2-(methoxycarbonylamino)-3-methylbutanoic acid (methyl carbamate of L-valine), (S)-2-(methoxycarbonylamino)-3,3-dimethylbutanoic acid (methyl carbamate of L-tert-leucine), or (2S,3R)-3-methoxy-2-(methoxycarbonylamino)butanoic acid (methyl carbamate of O-methyl-L-threonine). Removal of the P¹ group can be effected with conditions well known to those of skill in the art to be suitable for a particular protecting group. In particular, where P¹ is Boc, the Boc group can be removed by treatment with trifluoroacetic acid (TFA) in CH₂Cl₂. Coupling of the deprotected intermediate can be accomplished using the conditions for transforming (38) to (39) to give compounds of the invention (40).

Certain compounds of the invention (48) wherein G¹⁰ is optionally substituted phenyl and R²⁵, R²⁶, R²⁷, and R²⁸ are as described above can be prepared according to the general method illustrated in Scheme VI.

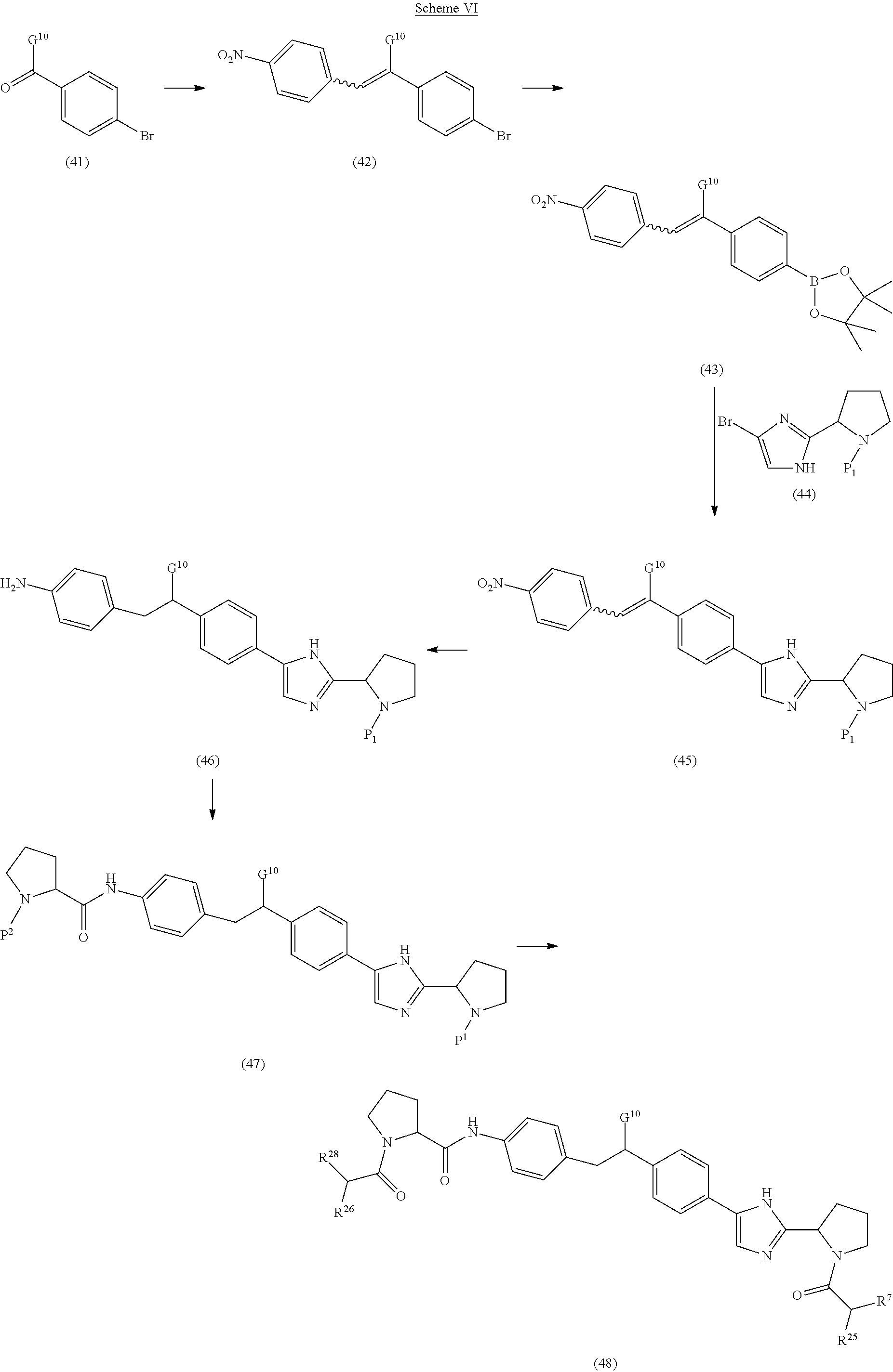

Ketones (41) can be converted to bromophenylalkenes (42) using the methods of Scheme V to convert (36) to (37). The bromophenylalkenes (42) can be reacted with bis(pinacolato)diboron with potassium acetate in solvents such as, but not limited to, toluene at temperatures from about 80° C. to about 120° C. to give the pinacolboranes (43). The pinacolboranes (43) can be reacted with bromoimidazoles (44), wherein P¹ is a nitrogen-protecting group, using Suzuki reaction conditions to give the phenylimidazole (45). A variety of reaction conditions are well known to those of skill in the art to be effective in mediating the Suzuki reaction. In particular, the reaction of (43) with (44) to produce (45) can be performed with [1,1′-bis(diphenylphosphino)ferrocene]dichloropalladium(II) (Pd(dppf)Cl₂) catalyst and potassium carbonate in a mixture of toluene and water and with heating to about 100° C. The phenylimidazole (45) can be converted to (46) by catalytic hydrogenation as described for the conversion of (37) to (38) in Scheme V. Compounds (46) can be transformed to compounds (47) using the coupling conditions as described in Scheme V for the conversion of (38) to (39). The P² substituent in compounds (47) represents a nitrogen protecting group that may be the same as or different from P¹, but P² is generally chosen independently from the same protecting groups as P¹ that were described in Scheme V. When P¹ and P² are the same, they may be removed simultaneously from (47) to produce a bis-deprotected intermediate which can be coupled with a carboxylic acid such as, but not limited to, those described above in the synthesis of compounds of formula (40) to produce compounds of the invention (48) wherein R²⁵ is the same as R²⁶ and R²⁷ is the same as R²⁸. When P¹ and P² are different, one of P¹ or P² may be independently removed and the deprotected product coupled with a first acid, followed by removal of the other of P¹ or P² and the resultant product coupled with a second acid to give compounds of the invention (48) wherein R²⁵ may be different from R²⁶ and may be different from R²⁸. The order of removal of P¹ and P² is determined by design considerations involving the reactivity of the particular protecting group and the chemical composition of the groups R²⁵, R²⁶, R²⁷, and R²⁸.

Certain compounds of the invention (53) wherein G¹⁰ is optionally substituted phenyl and R²⁵, R²⁶, R²⁷, and R²⁸ are as described above can be prepared according to the general method illustrated in Scheme VII.

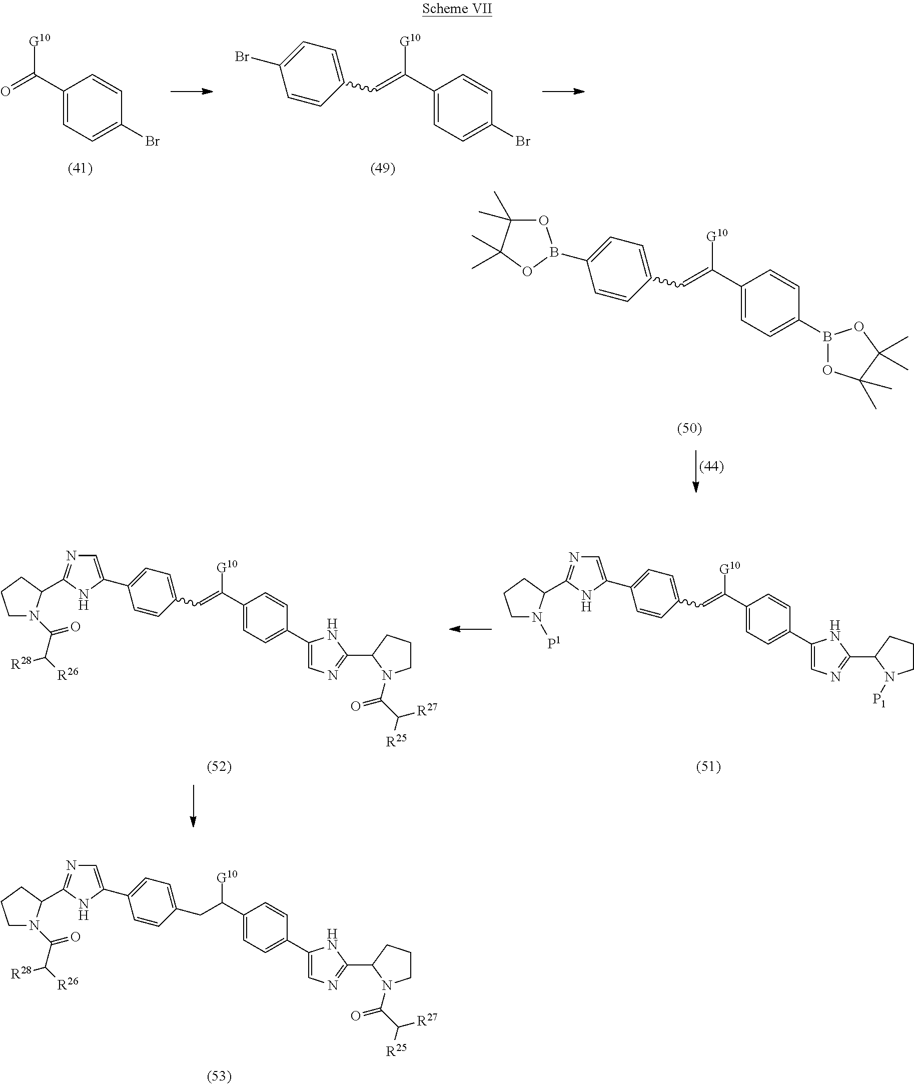

Ketones (41) can be converted to dibromodiphenylalkenes (49) using the methods of Scheme V to convert (36) to (37). In particular, ketones (41) can be reacted with diethyl 4-bromobenzylphosphonate in the presence of a base such as, but not limited to, sodium hydride or sodium bistrimethylsilylamide (NaHMDS) at temperatures from about 0° C. to about 110° C. in solvents such as, but not limited to, dimethylsulfoxide, tetrahydrofuran, or dimethylformamide to afford dibromodiphenylalkenes (49). Dibromodiphenylalkenes (49) can be converted to bispinacolboranes (50) using the methods of Scheme VI to convert (42) to (43). Likewise (50) can be converted to (51) by reaction with (44) using the method of Scheme VI to convert (43) to (45). Compounds (52) can be formed from (51) using the reagents and methods described for Scheme V to convert (39) to (40). Compounds (52) can be reduced by catalytic hydrogenation, such as used to convert (37) to (38), to provide compounds of formula (53).

Certain of the starting materials of general structures (36) and (41) can be purchased from commercial sources (e.g, 4-nitrobenzophenone, 4-bromo-4′-tert-butylbenzophenone, 4-bromo-4′-isopropylbenzophenone). These and others can also be prepared according to published procedures such as those found in the following references: Kagaku to Kogyo (1986) 60, 112-117 (4-tert-butyl-4′-nitrobenzophenone); Tetrahedron Lett. (2008) 49, 6715-6719 (4-isopropyl-4′nitrobenzophenone); J. Am. Chem. Soc. (2004) 126, 6608-6626 (4-bromo-4′-tert-butylbenzophenone). Illustrated in Scheme VIII is a general method of preparing compounds of general formula (41) wherein G¹⁰ is optionally substituted phenyl.

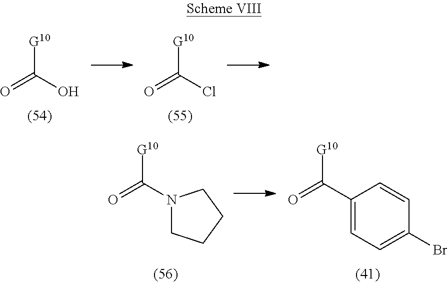

Carboxylic acids (54) can be converted to the corresponding acid chlorides (55) using standard procedures well known to those of skill in the art. For example, reaction of (54) with oxalyl chloride in dichloromethane with catalytic dimethylformamide at temperatures from 0° C. to room temperature gives the acid chlorides (55). The acid chlorides (55) can be converted to the pyrrolidine amides (56) by reaction with pyrrolidine in the presence of a base such as, but not limited to, triethylamine or diisopropylethylamine to provide the amides (56). Compounds of general formula (41) can be prepared by reaction of (56) with (4-bromophenyl)lithium in diethylether and hexanes at −78° C.

Other benzophenone starting materials with various substitutions on the aromatic rings may be substituted for those specifically shown in the foregoing schemes. These alternate benzophenones provide access to compounds of the invention with various substitutions off the rings G¹⁰ or G³⁰; or with regiochemistries on G³⁰ other than that shown in the foregoing schemes. Another general method of preparing a variety of benzophenones involves use of the Friedel-Crafts reaction as shown in Scheme IX, wherein X¹⁰⁰ and X¹⁰¹ are optional aromatic substituents of G¹⁰ or G³⁰, chemical precursors of said optional substituents, or suitable functional groups (e.g., a halogen or nitro) that enable further elaboration of the benzophenone to the compounds of the invention.

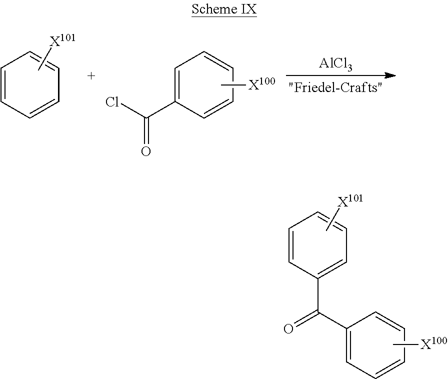

The intermediate of general formula (44), wherein P¹ is a nitrogen protecting group as described hereinabove, can be prepared using the general method in Scheme X.

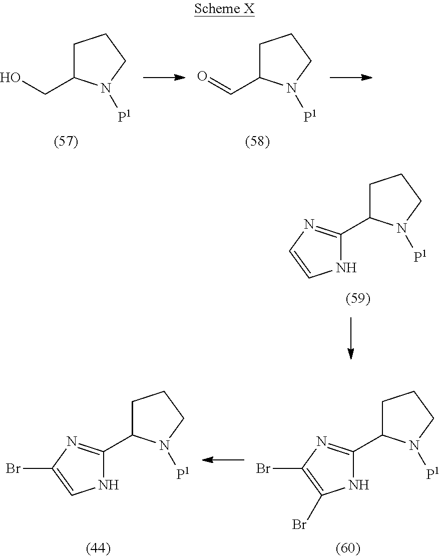

Alcohols (57) can be oxidized to aldehydes (58) using well-known methods such as, for example, reacting the alcohols (57) with Dess-Martin periodinane in the presence of sodium bicarbonate in a solvent such as, but not limited to, dichloromethane. Swern oxidation conditions (oxalyl chloride, dimethyl sulfoxide, triethylamine, dichloromethane) are an alternative for the conversion of alcohols (57) to aldehydes (58). Compounds (58) can be reacted with glyoxal and ammonium hydroxide in methanol/water to give (59). Compounds (59), in turn can be brominated using N-bromosuccinimide in solvents such as, but not limited to, dichloromethane at temperatures from 0° C. to room temperature to give (60). Compounds (60) can be mono-debrominated by reaction with sodium sulfite (Na₂SO₃) in a mixture of dioxane and water with heating to reflux to give intermediates (44). Although no particular stereochemistry is designated for intermediate (44), the foregoing chemical methods can be used to prepare (44) as a racemate or a single enantiomer (R or S stereochemistry). The choice of (R) or (S) stereochemistry in the starting alcohol (57) will lead to compounds of the invention having a single absolute stereochemistry at the corresponding carbon of the final compound.

Certain compounds of the invention (69) wherein R²⁵, R²⁶, R²⁷, and R²⁸ are as described above and X¹⁰⁰ is an optional substituent of G¹⁰, where G¹⁰ is phenyl, can be prepared according to the general method illustrated in Scheme XI.

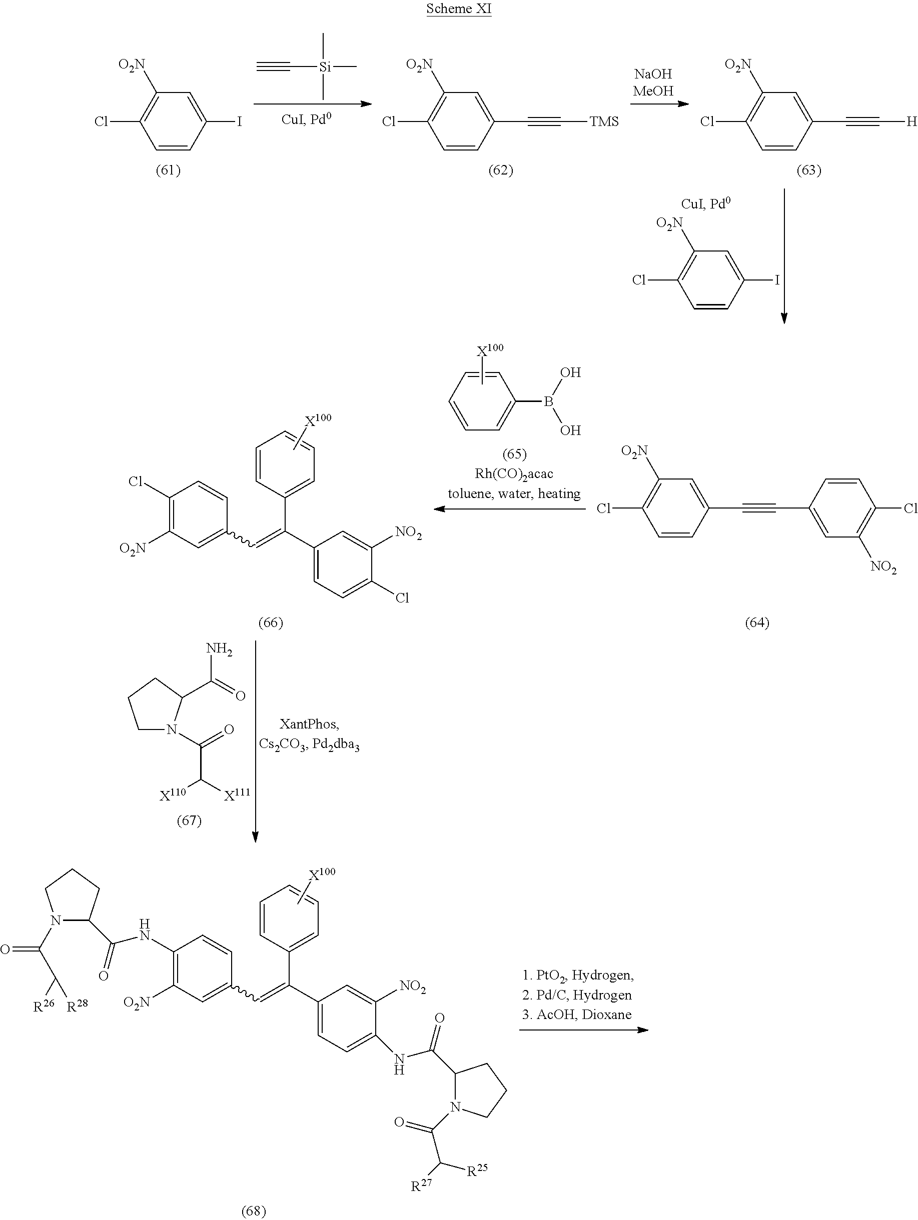

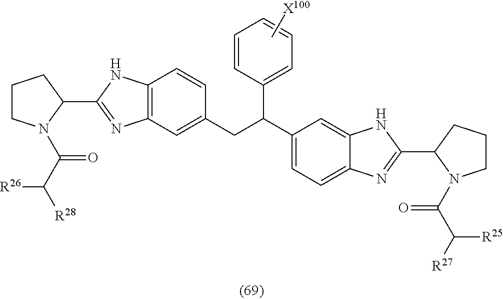

Sonogashira coupling of (61) with trimethylsilylacetylene in the presence of CuI and palladium catalyst such as, but not limited to, dichlorobis(triphenylphosphine)palladium(II), in solvents such as, but not limited to, triethylamine or mixtures of triethylamine and tetrahydrofuran, gives the intermediate (62). Removal of the trimethylsilyl group by reaction with sodium hydroxide in methanol or potassium carbonate in aqueous methanol at room temperature gives the intermediate (63). Compound (63) can be subjected to a second Sonogashira reaction using the same conditions as used to convert (61) to (62) to give (64). Compound (64) can be converted to compound (66) by reaction with an aryl boronic acid (65) in the presence of (acetylacetonato)dicarbonylrhodium(I) (Rh(CO)₂acac) with heating to between 80-120° C. in water and toluene. A Buchwald coupling of (66) with (67), in the presence of a palladium reagent such as but not limited to, tris(dibenzylideneacetone)dipalladium(0) (Pd₂dba₃), a base such as, but not limited to, Cs₂CO₃, and a bis-phosphine ligand such as, but not limited to, 4,5-bis(diphenylphosphino)-9,9-dimethylxanthene (Xantphos), in solvents such as dioxane or tetrahydrofuran and temperatures from about 80 to about 100° C. can give the intermediate (68). In formula (67), X¹¹⁰ represents R¹⁵ and R¹⁶ where R¹⁵ and R¹⁶ are the same and X¹¹¹ represents R¹⁷ and R¹⁸ where R¹⁷ and R¹⁸ are the same. Compounds (68) can be transformed to compounds of the invention (69) by sequential catalytic hydrogenation of the nitro group using PtO₂ in ethanol and/or tetrahydrofuran, hydrogenation of the double bond using Pd/C in ethanol or ethanol/tetrahydrofuran mixtures, and cyclization with acetic acid in dioxane with heating to about 70° C.

Compounds of the invention (69) can also be prepared by the alternate route illustrated in Scheme XII.

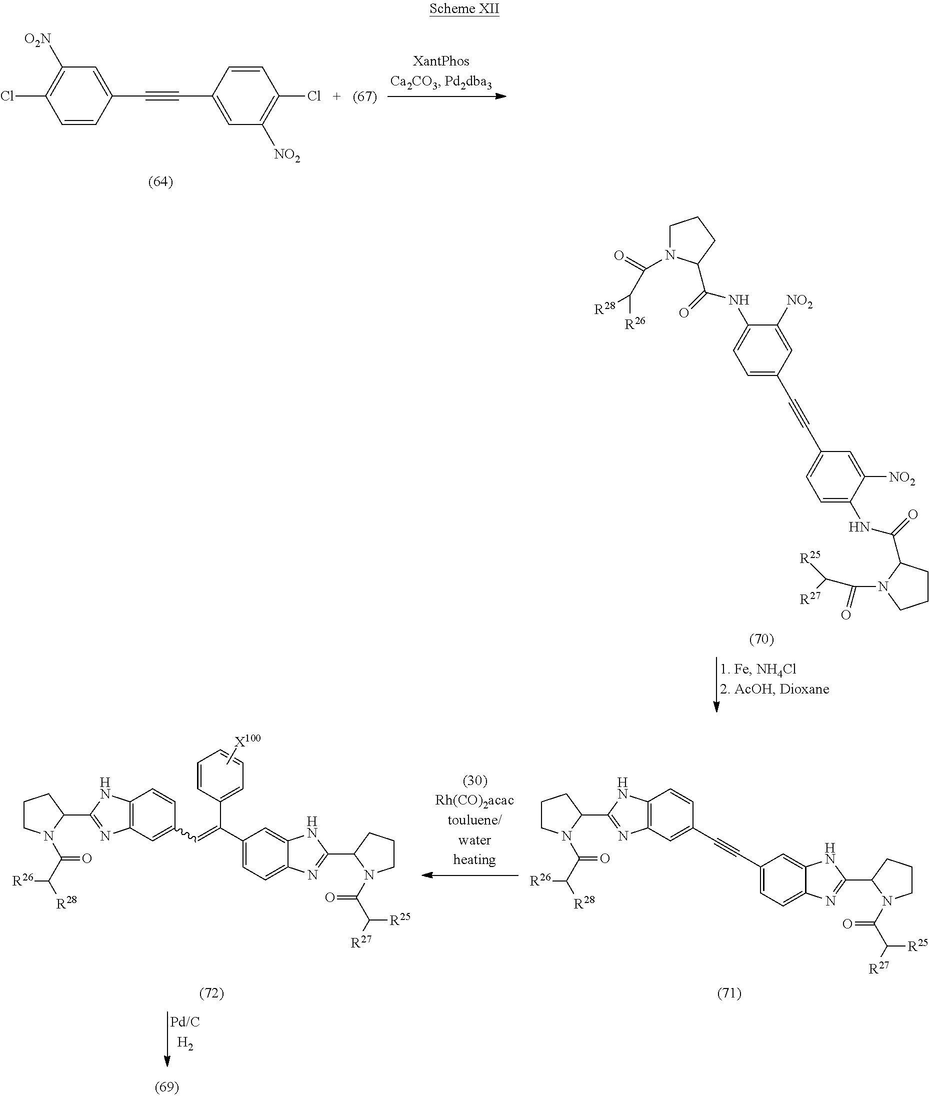

The compound of formula (64) can be reacted with compounds of formula (67) to give compounds of formula (70), using the method from Scheme XI to convert (66) to (68). Compounds of formula (70) can be converted to compounds of formula (71) by a two-step method involving reduction of the nitro group with Fe/NH₄Cl in solvent mixtures of water/tetrahydrofuran/ethanol at around 90° C., followed by cyclization using acetic acid in dioxane at about 70° C. Compounds of formula (71) can be reacted with boronic acids of formula (65) to give compounds of formula (72) using the general method used to convert (64) to (66) in Scheme XI and using 0.5 to 1.0 equiv of Rh(CO)₂acac. Compounds of formula (72) can be converted to compounds of formula (69) using catalytic hydrogenation over Pd/C in ethanol or ethanol/tetrahydrofuran mixtures as described generally above.

If a moiety described herein (e.g., —NH₂ or —OH) is not compatible with the synthetic methods, the moiety may be protected with a suitable protecting group that is stable to the reaction conditions used in the methods. The protecting group may be removed at a suitable point in the reaction sequence to provide a desired intermediate or target compound. Suitable protecting groups and methods for protecting or deprotecting moieties are well know in the art, examples of which can be found in Greene and Wuts, supra. Optimum reaction conditions and reaction times for each individual step may vary depending on the particular reactants employed and substituents present in the reactants used. Solvents, temperatures and other reaction conditions may be readily selected by one of ordinary skill in the art based on the present invention.

Other compounds of the invention can be similarly prepared according to the above-described schemes as well as the procedures described in the following examples, as appreciated by those skilled in the art. It should be understood that the above-described embodiments and schemes and the following examples are given by way of illustration, not limitation. Various changes and modifications within the scope of the present invention will become apparent to those skilled in the art from the present description.

Example compounds below were named using either ChemDraw version 9.0 or ACD Name version 10 or 12 (ACD v10, or ACD v12). Final compounds for Examples 1-9 were named using ACD Name v12. Intermediates for Examples 1-1 were named using ChemDraw, unless otherwise indicated.

LC/MS measurements for Examples 1-9 were run on an Agilent 1200 HPLC/6100 SQ System using the follow condition: Mobile Phase: A: Water (0.05% trifluoroacetic acid), B: acetonitirle (0.05% trifluoroacetic acid); Gradient Phase: 5%-95% in 1.7 minutes; Flow rate: 1.6 mL/minute; Column: XBridge; Oven Temp. 50° C. Some intermediates were monitored with a run of 1.5 minutes.

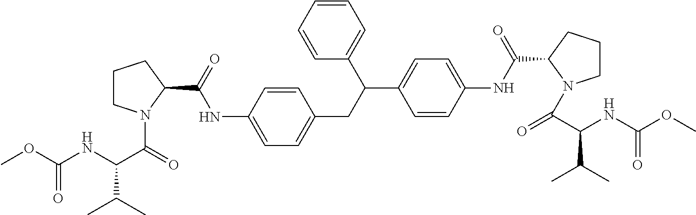

### Example 1

### dimethyl [(1-phenylethane-1,2-diyl)bis{benzene-4,1-diylcarbamoyl(2S)pyrrolidine-2,1-diyl[(2S)-3-methyl-1-oxobutane-1,2-diyl]}]biscarbamate

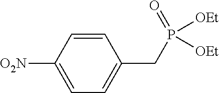

### Example 1A

### diethyl 4-nitrobenzylphosphonate

A mixture of 4-nitrobenzyl bromide (4.1 g, 1.9 mmol) and triethylphosphite (4.46 g, 2.68 mmol) was heated at 160° C. under a nitrogen atmosphere for 2 hours. Excess triethylphosphite was removed in vacuo to give the title compound as brown oil (5 g, 18.3 mmol, 96%) which was used directly without further purification.

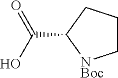

### Example 1B

### (S)-1-(tert-butoxycarbonyl)pyrrolidine-2-carboxylic acid

A solution of sodium carbonate (1.83 g, 17.2 mmol), 1 M NaOH (33 mL, 33 mmol) and (S)-pyrrolidine-2-carboxylic acid (3.83 g, 33.3 mmol) was cooled to OC and treated with di-tert-butyl dicarbonate (7.88 g, 36.1 mmol). The reaction solution allowed to warm to room temperature and stirred for 3-4 hours. The solution was acidified to a pH of about 1-2 with concentrated HCl and extracted with CH₂Cl₂ (50 mL×3). The organic layer was dried (Na₂SO₄), filtered, and concentrated in vacuo to provide the title compound that was used without further purification.

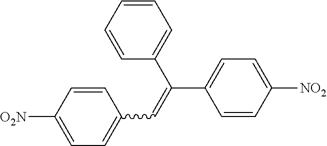

### Example 1C

### 4,4′-(1-phenylethene-1,2-diyl)bis(nitrobenzene)

To a solution of (4-nitrophenyl)(phenyl)methanone (391 mg, 1.72 mmol), and Example 1A in anhydrous dimethyl sulfoxide (15 mL) under N₂ atmosphere was added NaH (194 mg, 1.72 mmol) at room temperature, and the mixture was stirred for 3 hours at 100° C. After the reaction was completed, the reaction mixture was partitioned between water (30 mL) and dichloromethane (30 mL). The organic layer was separated, washed twice with brine, dried over Na₂SO₄, filtered and concentrated to provide the title compound as a yellow solid (405 mg) that consisted of a mixture of double bond geometries (E and Z).

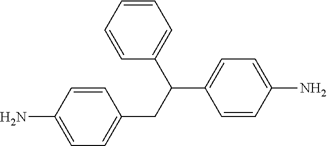

### Example 1D

### 4,4′-(1-phenylethane-1,2-diyl)dianiline

To a solution of Example 1C (400 mg, 1.12 mmol) in ethyl acetate (4 mL) was added Pd/C (40 mg) in portions under H₂. The reaction was stirred overnight, the solution filtered and concentrated to provide the title compound (226 mg).

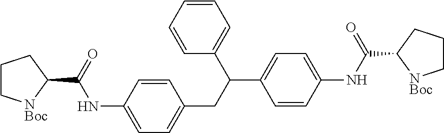

### Example 1E

### di-tert-butyl (2S,2′S)-2,2′-[(1-phenylethane-1,2-diyl)bis(4,1-phenylene carbamoyl)]dipyrrolidine-1-carboxylate (ACD v12)

To a solution of Example 1D (100 mg, 0.35 mmol), (7-azabenzotriazol-1-yloxy)tripyrrolidinophosphonium hexafluorophosphate (PyAOP, 365 mg, 0.7 mmol), and diisopropylethylamine (181 mg, 1.4 mmol) in 3 mL of N,N-dimethylformamide was added Example 1B (166 mg, 0.77 mmol). The reaction mixture was stirred at room temperature overnight, treated with saturated NH₄Cl and partitioned between CH₂Cl₂ and water. The organic layer was dried (Na₂SO₄), filtered and evaporated to provide the title compound (189 mg).

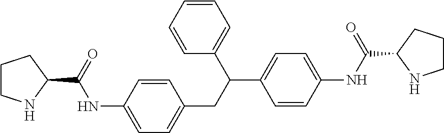

### Example 1F

### (2S,2′S)—N,N′-[(1-phenylethane-1,2-diyl)di-4,1-phenylene]dipyrrolidine-2-carboxamide (ACD v12)

To a solution of Example 1E (218 mg, 0.32 mmol) in 3 mL of CH₂Cl₂ was added trifluoroacetic acid (3 mL). The resulting mixture was stirred at room temperature for 2 hours and then concentrated to give the title compound which was directly used without further purification.

### Example 1G

### dimethyl [(1-phenylethane-1,2-diyl)bis{benzene-4,1-diylcarbamoyl(2S)pyrrolidine-2,1-diyl[(2S)-3-methyl-1-oxobutane-1,2-diyl]}]biscarbamate

To a solution of Example 1F (168 mg, 0.35 mmol), (7-azabenzotriazol-1-yloxy)tripyrrolidinophosphonium hexafluorophosphate (PyAOP, 365 mg, 0.7 mmol), and diisopropylethylamine (181 mg, 1.4 mmol) in 3 mL of N,N-dimethylformamide was added (S)-2-(methoxycarbonylamino)-3-methylbutanoic acid (0.77 mmol). The reaction mixture was stirred at room temperature overnight, treated with saturated NH₄Cl, and then partitioned between CH₂Cl₂ and water. The organic layer was dried (Na₂SO₄), filtered, and concentrated. The crude product was purified by prep-HPLC, using a Waters-X-Bridge column (19×150 mm, 5 μm) and a mobile phase of acetonitrile (35-80%)/water (10 ppm NH₄HCO₃), to give the title compound. ¹HNMR (CDCl₃, 400 MHz) δ ppm 0.88 (m, 12H), 1.73-2.49 (m, 11H), 3.23 (d, J=6.8 Hz, 2H), 3.64 (m, 2H), 3.67 (s, 6H), 3.76 (m, 2H), 4.11 (t, J=7.6 Hz, 1H), 4.33 (t, J=7.6 Hz, 2H), 4.75 (d, J=6 Hz, 1H), 5.45 (s, 2H), 6.87 (d, J=8.0 Hz, 2H), 7.06-7.14 (m, 5H), 7.19-7.25 (m, 4H), 7.31 (d, J=8.0 Hz, 2H), 9.15 (s, 1H), 9.20 (s, 1H); LC/MS m/z 779 [M+H]⁺.

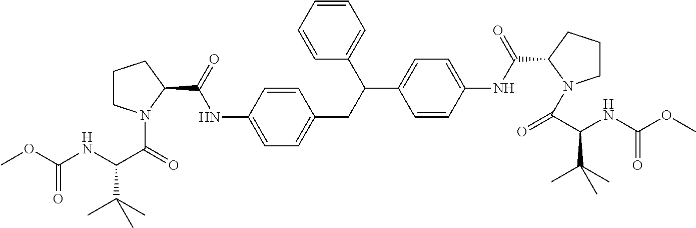

### Example 2

### dimethyl [(1-phenylethane-1,2-diyl)bis{benzene-4,1-diylcarbamoyl(2S)pyrrolidine-2,1-diyl[(2S)-3,3-dimethyl-1-oxobutane-1,2-diyl]}]biscarbamate

To a solution of Example 1F (168 mg, 0.35 mmol), (7-azabenzotriazol-1-yloxy)tripyrrolidinophosphonium hexafluorophosphate (PyAOP, 365 mg, 0.7 mmol), and diisopropylethylamine (181 mg, 1.4 mmol) in 3 mL of N,N-dimethylformamide was added (S)-2-(methoxycarbonylamino)-3,3-dimethylbutanoic acid (0.77 mmol). The reaction mixture was stirred at room temperature overnight, treated with saturated NH₄Cl and then partitioned between CH₂Cl₂ and water. The organic layer was dried (Na₂SO₄), filtered, and concentrated. The crude product was purified by prep-HPLC, using a Waters-X-Bridge column (19×150 mm, 5 μm) and a mobile phase of acetonitrile (40-80%)/water (10 ppm NH₄HCO₃), to give the title compound. ¹HNMR (CDCl₃, 400 MHz) δ ppm 1.00 (s, 18H), 1.88-2.46 (m, 9H), 3.21 (d, J=7.6 Hz, 2H), 3.68 (s, 8H), 3.80 (m, 2H), 4.10 (m, 1H), 4.37 (d, J=8.4 Hz, 2H), 4.74 (s, 1H), 5.55 (s, J=8.4 Hz, 2H), 6.86 (d, J=8.4 Hz, 2H), 7.06-7.14 (m, 5H), 7.19-7.25 (m, 4H), 7.31 (d, J=8.4 Hz, 2H), 9.19 (s, 1H), 9.26 (d, J=5.2 Hz, 1H); LC/MS m/z 825 [M+H]⁺.

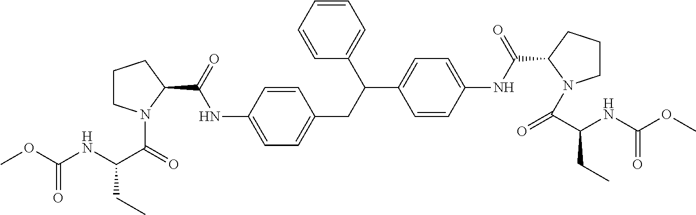

### Example 3

### dimethyl [(1-phenylethane-1,2-diyl)bis{benzene-4,1-diylcarbamoyl(2S)pyrrolidine-2,1-diyl[(2S)-1-oxobutane-1,2-diyl]}]biscarbamate

To a solution of Example 1F (168 mg, 0.35 mmol), (7-azabenzotriazol-1-yloxy)tripyrrolidinophosphonium hexafluorophosphate (PyAOP, 365 mg, 0.7 mmol), and diisopropylethylamine (181 mg, 1.4 mmol) in 3 mL of N,N-dimethylformamide was added (S)-2-(methoxycarbonylamino)butanoic acid (0.77 mmol). The reaction mixture was stirred at room temperature overnight, treated with saturated NH₄Cl, and then partitioned between CH₂Cl₂ and water. The organic layer was dried (Na₂SO₄), filtered, and concentrated. The crude product was purified by prep-HPLC, using a Waters-X-Bridge column (19×150 mm, 5 μm) and a mobile phase of acetonitrile (30-70%)/water (10 ppm NH₄HCO₃), to give the title compound. ¹HNMR (CDCl₃, 400 MHz) δ ppm 0.93 (m, 6H), 1.58-2.20 (m, 12H), 2.40 (m, 2H), 3.19 (m, 2H), 3.68 (s, 6H), 3.80 (m, 2H), 4.10 (m, 1H), 4.43 (m, 2H), 4.72 (m, 2H), 5.68 (d, J=8.0 Hz, 2H), 6.85 (d, J=8.4 Hz, 2H), 7.03-7.16 (m, 5H), 7.18-7.24 (m, 4H), 7.29 (d, J=8.4 Hz, 2H), 9.16 (d, J=3.6 Hz, 1H), 9.23 (s, 1H); LC/MS m/z 769 [M+H]⁺.

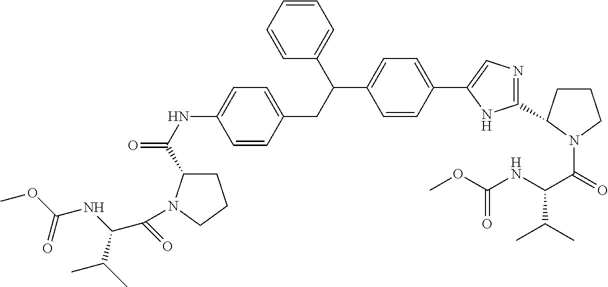

### Example 4

### N-(methoxycarbonyl)-L-valyl-N-(4-{2-[4-(2-{(2S)-1-[N-(methoxycarbonyl)-L-valyl]pyrrolidin-2-yl}-1H-imidazol-5-yl)phenyl]-2-phenylethyl}phenyl)-L-prolinamide

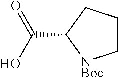

### Example 4A

### (S)-tert-butyl 2-formylpyrrolidine-1-carboxylate

To an oven-dried 500-mL 3-neck flask purged with nitrogen was added oxalyl chloride (5.32 mL, 60.8 mmol) and anhydrous dichloromethane (125 mL), and the solution was cooled to −78° C. A solution of anhydrous dimethyl sulfoxide (7.30 mL, 103 mmol) in anhydrous dichloromethane (25 mL) was added dropwise from a constant-pressure addition funnel over 20 minutes. A solution of (S)-tert-butyl 2-(hydroxymethyl)pyrrolidine-1-carboxylate (9.41 g, 46.8 mmol) in anhydrous dichloromethane (50 mL) was added dropwise from a constant-pressure addition funnel over 20 minutes, and the reaction mixture was stirred at −78° C. for 30 minutes. Triethylamine (32.6 mL, 234 mmol) was added dropwise via syringe over 5 minutes, and the thick white mixture was stirred at 0° C. for 30 minutes. The reaction was quenched with 10% (w/v) aqueous citric acid (30 mL), poured into a separatory funnel with diethyl ether (550 mL) and 10% (w/v) aqueous citric acid, the layers were separated, and the organic phase was washed with water and brine. The organic phase was dried over anhydrous Na₂SO₄, filtered, and concentrated to afford the title compound as a yellow oil (9.4 g) which was used directly in the next reaction.

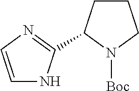

### Example 4B

### (S)-tert-butyl 2-(1H-imidazol-2-yl)pyrrolidine-1-carboxylate

The product from Example 4A (20 g, 100 mmol) was dissolved in methanol (50.2 mL) and ammonium hydroxide (50.2 mL) was added. To this solution, glyoxal (40% in water; 24.08 mL, 211 mmol) was added dropwise over 10 minutes. The reaction was stirred at room temperature overnight. The reaction was concentrated under reduced pressure, diluted with 50 mL of water, and then extracted with ethyl acetate. The organic layer was washed with brine, dried (Na₂SO₄) and concentrated to give a tan solid that was treated with ether and concentrated. The solid was then triturated with 2:1 diethyl ether:hexanes (150 mL) to afford 17 g of the title compound as a solid, which was used directly in the next reaction. ¹HNMR (DMSO-d₆, 400 MHz) δ ppm 1.14/1.40 (s, 9H), 1.81-2.12 (m, 4H), 3.32-3.33 (m, 1H), 3.35-3.50 (m, 1H), 4.72-4.81 (m, 1H), 6.84 (s, 1H), 11.68 (s, 1 H); LC/MS m/z 238 [M+H]⁺.

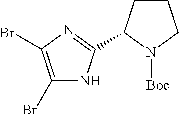

### Example 4C

### (S)-tert-butyl 2-(4,5-dibromo-1H-imidazol-2-yl)pyrrolidine-1-carboxylate

N-Bromosuccinimide (108 mmol) was added to a cold (0° C.) solution of the product from Example 4B (12.05 g, 50.8 mmol) in dichloromethane (200 mL). The reaction was stirred at 0° C. for 2 hours and then concentrated. The residue was dissolved in ethyl acetate (250 mL), washed with water (3×150 mL) and brine (1×100 mL), dried (MgSO₄) and concentrated to give a dark residue. The residue was dissolved in dichloromethane, diluted with an equal volume of hexanes and concentrated to give a brown solid (˜19 g). The solid was triturated with diethyl ether (˜100 mL), filtered, and concentrated to give a tan solid (13.23 g). ¹H NMR (400 MHz, CDCl₃) δ ppm 1.49 (s, 9 H), 1.86-2.17 (m, 3H), 2.80-2.95 (m, 1H), 3.30-3.44 (m, 2H), 4.85 (dd, J=7.54, 2.55 Hz, 1H), 10.82 (s, 1H); MS (DCI+) m/z 394/396/398 [M+H]⁺.

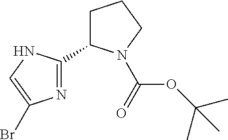

### Example 4D

### (S)-tert-butyl 2-(4-bromo -1H-imidazol-2-yl)pyrrolidine-1-carboxylate

Example 4C (6.25 g, 15.82 mmol) was dissolved in dioxane (200 mL) and water (200 mL), treated with a solution of sodium sulfite (22.38 g, 174 mmol) in water (200 mL), and heated at reflux for 16 hours. The reaction was cooled to room temperature and concentrated in vacuo. The residue was extracted with dichloromethane. The combined organic extracts were washed with brine (50 mL), dried over anhydrous Na₂SO₄, filtered, and concentrated by rotary evaporation. Addition and evaporation of 2:1 hexanes/dichloromethane (100 mL) gave a beige foam (4.38 g) that was purified by gradient silica gel flash chromatography eluting with 30% to 80% ethyl acetate/hexanes to afford the title compound as a white solid (3.48 g). ¹H NMR (400 MHz, CDCl₃) δ ppm 1.48 (s, 9H), 1.83-2.33 (m, 3H), 2.79-3.02 (m, 1H), 3.37 (dd, J=7.10, 5.37 Hz, 2H), 4.88 (dd, J=7.59, 2.49 Hz, 1H), 6.92 (s, 1H), 10.70 (br s, 1H); MS (ESI+) m/z 316/318 (M+H)⁺.

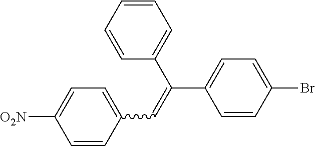

### Example 4E

### 1-bromo-4-(2-(4-nitrophenyl)-1-phenylvinyl)benzene

To a stirred solution of NaH (805 mg, 20.1 mmol) and 15-crown-5 (442 mg, 2.01 mmol) in tetrahydrofuran (40 mL) was added Example 1A (5 g, 18.3 mmol) at 0° C. in batches over 10 minutes, and the mixture was stirred at 0° C. for 0.5 hour. 4-Bromobenzophenone (4.7 g, 18.3 mmol) was added into the reaction mixture with continued stirring for 12 hours. The reaction mixture was then poured into water (50 mL), the aqueous layer was extracted with dichloromethane, and the organic layer was dried and concentrated. The residue was purified by reverse phase flash chromatography (20%˜95% CH₃OH/H₂O, 0.1% trifluoroacetic acid) to afford the title compound as a mixture of geometric isomers (E and Z) (613 mg).

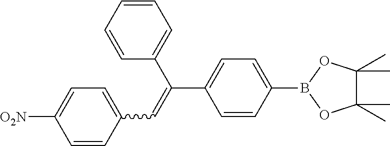

### Example 4F

### 4,4,5,5-tetramethyl-2-(4-(2-(4-nitrophenyl)-1-phenylvinyl)phenyl)-1,3,2-dioxaborolane

A mixture of Example 4E (4.3 g, 11.3 mmol), bis(pinacolato)diboron (3.1 g, 12.4 mmol), potassium acetate (3.3 g, 33.9 mmol), and [1,1′-bis(diphenylphosphino)ferrocene]dichloropalladium(II) (Pd(dppf)Cl₂, 1.3 g, 1.62 mmol) in dioxane (80 mL) was stirred at 100° C. for 2 hours. The solvent was removed in vacuo, and the residue was washed with water (40 mL) and extracted with dichloromethane. The combined organic layer was dried, concentrated, and purified by gradient silica gel column chromatography (petroleum ether to petroleum ether:ethyl acetate=5:1) to afford 4.1 g of the title compound.

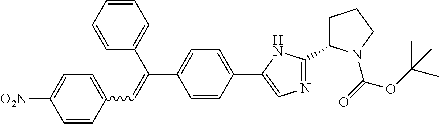

### Example 4G

### tert-butyl (2S)-2-(5-{4-[2-(4-nitrophenyl)-1-phenylvinyl]phenyl}-1H-imidazol-2-yl)pyrrolidine-1-carboxylate (ACD v12)

A mixture of Example 4F (4.1 g, 9.7 mmol), Example 4D (3.1 g, 9.7 mmol), K₂CO₃ (2.0 g, 14.5 mmol), and [1,1′-bis(diphenylphosphino)ferrocene]dichloropalladium(II) (Pd(dppf)Cl₂, 1.03 g, 1.26 mmol) in toluene (80 mL) and water (40 mL) was stirred at 100° C. for 2 hours. The aqueous phase was extracted with ethyl acetate, and the combined organic layers were dried and concentrated. The residue was purified by gradient silica gel column chromatography (petroleum ether to petroleum ether:ethyl acetate=3:1) to afford 4.1 g of the title compound.

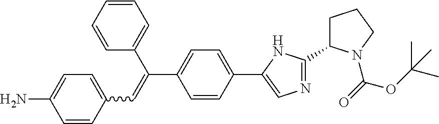

### Example 4H

### ACD v12

### tert-butyl (2S)-2-(5-{4-[2-(4-aminophenyl)-1-phenylethyl]phenyl}-1H-imidazol-2-yl)pyrrolidine-1-carboxylate

A mixture of Example 4G (4.1 g, 7.7 mmol) and Pd/C (200 mg) in CH₃OH (150 mL) was stirred under a hydrogen atmosphere at room temperature for 12 hours. The Pd/C was removed by filtration, and the solution was concentrated. The residue was purified by gradient silica gel column chromatography (petroleum ether to petroleum ether:ethyl acetate=1:1) to afford 3.1 g of the title compound.

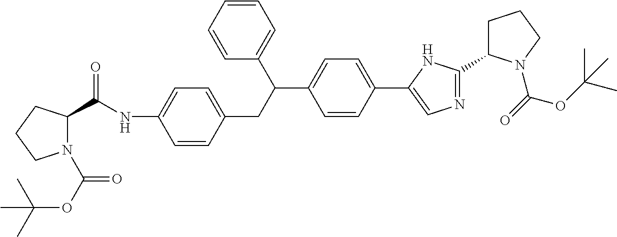

### Example 4I

### tert-butyl (2S)-2-(5-{4-[2-(4-{[1-(tert-butoxycarbonyl)-L-prolyl]amino}phenyl)-1-phenylethyl]phenyl}-1H-imidazol-2-yl)pyrrolidine-1-carboxylate (ACD v12)

A mixture of Example 4H (3.1 g, 6.2 mmol), Example 1B (1.3 g, 6.2 mmol), diisopropylethylamine (3.2 g, 24.8 mmol) and (benzotriazol-1-yloxy)tripyrrolidinophosphonium hexafluorophosphate (PyBOP®, 6.4 g, 12.4 mmol) in N,N-dimethylformamide (80 mL) was stirred at room temperature for 12 hours. The solvent was removed in vacuo, and the residue was washed with water (40 mL) and extracted with dichloromethane. The combined organic layer was dried, concentrated and purified by gradient silica gel column chromatography (petroleum ether to petroleum ether:ethyl acetate=1:1) to afford 3.2 g of the title compound.

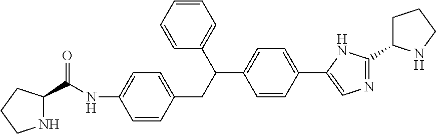

### Example 4J

### N-{4-[2-phenyl-2-(4-{2-[(2S)-pyrrolidin-2-yl]-1H-imidazol-5-yl}phenyl)ethyl]phenyl}-L-prolinamide (ACD v12)

To a solution of Example 4I (3.2 g, 4.6 mmol) in 15 mL of dichloromethane was added trifluoroacetic acid (15 mL), and the mixture was stirred at room temperature for 2 hours. The solvent was removed in vacuo, the residue was washed with aqueous NaHCO₃ (20 mL) and extracted with dichloromethane. The combined organic layer was dried, concentrated, and purified by gradient silica gel column chromatography (dichloromethane to dichloromethane:ethyl acetate=3:1) to afford 1.6 g of the title compound.

### Example 4K

### N-(methoxycarbonyl)-L-valyl-N-(4-{2-[4-(2-{(2S)-1-[N-(methoxycarbonyl)-L -valyl]pyrrolidin-2-yl}-1H-imidazol-5-yl)phenyl]-2-phenylethyl}-phenyl)-L-prolinamide

A mixture of Example 4J (300 mg, 0.59 mmol), (S)-2-(methoxycarbonyl-amino)-3-methylbutanoic acid (206 mg, 1.18 mmol), diisopropylethylamine (619 mg, 4.8 mmol) and O-benzotriazol-1-yl-N,N,N′,N′-tetramethyluronium tetrafluoroborate (TBTU, 770 mg, 2.4 mmol) in N,N-dimethylformamide (5 mL) was stirred at room temperature for 12 hours. The solvent was removed in vacuo, and the residue was washed with aqueous NH₄Cl (10 mL) and extracted with dichloromethane. The combined organic layer was dried, concentrated, and purified by preparative thin layer chromatography (dichloromethane:ethyl acetate=1:1) to afford 70 mg of the title compound. ¹HNMR (DMSO-d₆, 400 MHz) δ ppm 0.87-0.95 (m, 12H), 1.78-2.12 (m, 10H), 3.28-3.30 (m, 2H), 3.57 (s, 6H), 3.58-3.61 (m, 1H), 3.77-3.81 (m, 2H), 3.99-4.05 (m, 2H), 4.22-4.40 (m, 2H), 5.04-5.05 (m, 1H), 7.03-7.11 (m, 3H), 7.24-7.36 (m, 11H), 7.53-7.54 (m, 2H), 9.85 (s, 1H), 11.68 (brs, 1H); LC/MS m/z 820 [M+H]⁺.

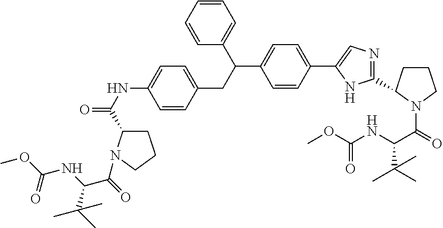

### Example 5

### N-(methoxycarbonyl)-3-methyl-L-valyl-N-(4-{2-[4-(2-{(2S)-1-[N-(methoxycarbonyl)-3-methyl-L-valyl]pyrrolidin-2-yl}-1H-imidazol-5-yl)phenyl]-2-phenylethyl}phenyl)-L-prolinamide

A mixture of Example 4J (300 mg, 0.59 mmol), (S)-2-(methoxycarbonyl-amino)-3,3-dimethylbutanoic acid (223 mg, 1.18 mmol), diisopropylethylamine (619 mg, 4.8 mmol) and O-benzotriazol-1-yl-N,N,N′,N′-tetramethyluronium tetrafluoroborate (TBTU, 770 mg, 2.4 mmol) in N,N-dimethylformamide (5 mL) was stirred at room temperature for 12 hours. The solvent was removed in vacuo, and the residue was washed with aqueous NH₄Cl (10 mL) and extracted with dichloromethane. The combined organic layer was dried, concentrated, and purified by preparative thin layer chromatography (dichloromethane:ethyl acetate=1:1) to afford 65 mg of the title compound. ¹HNMR (CDCl₃), 400 MHz: δ 0.85 (s, 9H), 0.89 (s, 9H), 1.81-2.15 (m, 7H), 2.34-2.36 (m, 1H), 3.53-3.79 (m, 12H), 4.16-4.18 (m, 2H), 4.35-4.39 (m, 2H), 5.05-5.09 (m, 1H), 7.05-7.36 (m, 15H), 7.95 (s, 1H), 9.89 (s, 1H), 14.46 (brs. 1H); LC/MS m/z 848 [M+H]⁺.

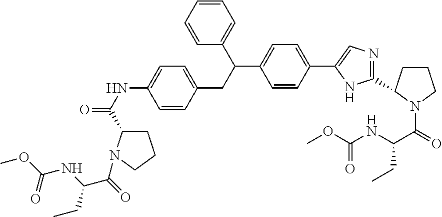

### Example 6

### 1-{(2S)-2-[(methoxycarbonyl)amino]butanoyl}-N-{4-[2-(4-{2-[(2S)-1-{(2S)-2-[(methoxycarbonyl)amino]butanoyl}pyrrolidin-2-yl]-1H-imidazol-5-yl}phenyl)-2-phenylethyl]phenyl}-L-prolinamide

A mixture of Example 4J (300 mg, 0.59 mmol), (S)-2-(methoxycarbonyl amino)butanoic acid (190 mg, 1.18 mmol), diisopropylethylamine (619 mg, 4.8 mmol) and O-benzotriazol-1-yl-N,N,N′,N′-tetramethyluronium tetrafluoroborate (TBTU, 770 mg, 2.4 mmol) in N,N-dimethylformamide (5 mL) was stirred at room temperature for 12 hours. The solvent was removed in vacuo, and the residue washed with aqueous NH₄Cl (10 mL) and extracted with dichloromethane. The combined organic layer was dried, concentrated, and purified by preparative thin layer chromatography (dichloromethane:ethyl acetate=1:1) to afford 60 mg of the title compound. ¹HNMR (CDCl₃, 400 MHz) δ ppm 0.90-0.94 (m, 6H), 1.52-2.14 (m, 12H), 2.30-2.49 (m, 2H), 2.92-2.95 (m, 1H), 3.27-2.29 (m, 2H), 3.52-3.80 (m, 10H), 4.16-4.18 (m, 1H), 4.42-4.49 (m, 2H), 4.72-4.75 (m, 1H), 5.22-5.23 (m, 1H), 5.57-5.64 (m, 2H), 6.89-6.91 (m, 2H), 6.91-7.62 (m, 12H), 9.09 (s, 1H); LC/MS m/z 792 [M+H]⁺.

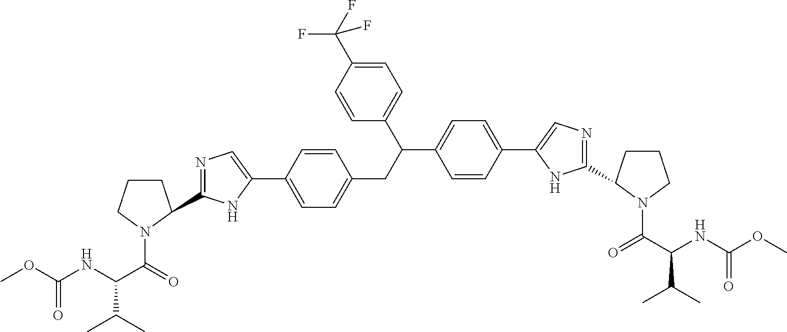

### Example 7

### methyl [(2S)-1-{(2S)-2-[5-(4-{2-(4-{2-[(2S)-1-{(2S)-2-[(methoxycarbonyl)amino]-3-methylbutanoyl}pyrrolidin-2-yl]-1H-imidazol-5-yl}phenyl)-2-[4-(trifluoromethyl)phenyl]ethyl}phenyl)-1H-imidazol-2-yl]pyrrolidin-1-yl}-3-methyl-1-oxobutan-2-yl]carbamate

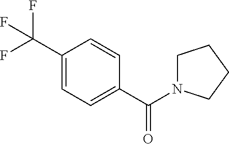

### Example 7A

### pyrrolidin-1-yl(4-(trifluoromethyl)phenyl)methanone

4-(Trifluoromethyl)benzoic acid (5.0 g, 26.3 mmol) was dissolved in 50 mL of CH₂Cl₂ and consecutively treated with 2 drops of N,N-dimethylformamide then oxalyl chloride (4.0 g, 31.5 mmol). The mixture was stirred at 40° C. for 4 hours and then the mixture was added dropwise to pyrrolidine (2.2 g, 31.5 mmol) and diisopropylethylamine (6.7 g, 52.6 mmol). The reaction was stirred at 30° C. overnight. The reaction was washed with water. The organic phase was dried over Na₂SO₄, filtered and concentrated to provide the crude title compound. LC/MS m/z 244 [M+H]⁺.

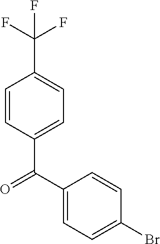

### Example 7B

### (4-bromophenyl)(4-(trifluoromethyl)phenyl)methanone

To 30 mL of diethyl ether at −78° C. was added n-butyllithium (16 mL, 25.12 mmol, 1.6 M in hexanes). After the reaction temperature equilibrated (˜15 minutes), a solution of 1,4-dibromobenzene (5.8 g, 24.7 mmol in 30 mL of diethyl ether) was added dropwise over a 20 minutes. The resulting mixture was stirred for 1 hour, Example 7A (6.0 g, 24.7 mmol) was added, and the reaction mixture was stirred for 2 hours at −78° C. under N₂. The reaction was warmed to room temperature and allowed to stir for 1 day. The reaction was quenched by the dropwise addition of 1 N cold HCl followed by extraction with diethyl ether. The layers were separated; the combined organic layers were dried over MgSO₄, filtered, and concentrated under vacuum. Purification by column chromatography over silica gel (eluent: petroleum ether/ethyl acetate=50:1) afforded the title compound. LC/MS m/z 329 [M+H]⁺.

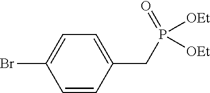

### Example 7C

### diethyl 4-bromobenzylphosphonate

A mixture of 4-bromobenzyl bromide (10 g, 4 mmol) and triethylphosphite (9.3 g, 5.6 mmol) was heated at 160° C. under a nitrogen atmosphere for 2 hours. The excess triethylphosphite was removed in vacuo to give the title compound as a colorless oil (12 g). The compound was used directly without further purification. LC/MS m/z 307 [M+H]⁺.

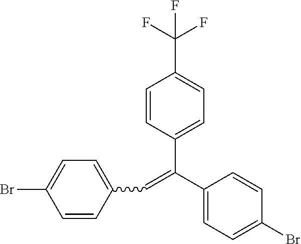

### Example 7D

### 4,4′-(1-(4-(trifluoromethyl)phenyl)ethene-1,2-diyl)bis(bromobenzene)

Sodium hexamethyldisilazane (2 M in tetrahydrofuran) was added to a solution of Example 7C (1.8 g, 6.09 mmol) in tetrahydrofuran (20 mL) at 0° C. After 2 hours, Example 7B (1.0 g, 3.05 mmol) in 20 mL of tetrahydrofuran was added into the reaction. The mixture was stirred at 30° C. for 12 hours, poured into H₂O (80 mL) and extracted with ethyl acetate. The combined organic phase was dried, concentrated, and purified by gradient silica gel column chromatography (petroleum ether to petroleum ether: ethyl acetate=100:1) to afford 0.56 g the title compound. LC/MS m/z 483 [M+H]⁺.

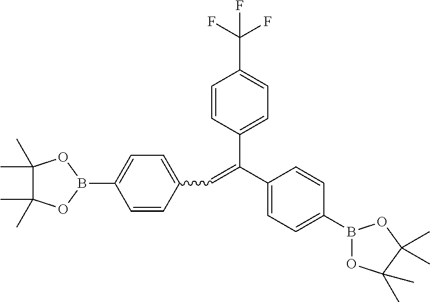

### Example 7E

### 2,2′-(4,4′-(1-(4-(trifluoromethyl)phenyl)ethene-1,2-diyl)bis(4,1-phenylene))bis(4,4,5,5-tetramethyl-1,3,2-dioxaborolane)

A mixture of Example 7D (0.5 g, 1.03 mmol), bis(pinacolato)diboron (395 mg, 1.55 mmol), [1,1′-bis(diphenylphosphino)ferrocene]dichloropalladium(II) (Pd(dppf)Cl₂, 108 mg, 0.13 mmol), and potassium acetate (202 mg, 2.06 mmol) in N,N-dimethylformamide (30 mL) was stirred at 100° C. for 2 hours. The mixture was poured into water (50 mL) and extracted with dichloromethane. The combined organic layer was dried, concentrated, and purified by gradient silica gel column chromatography (petroleum ether to petroleum ether:ethyl acetate=30:1) to afford 270 mg of the title compound. LC/MS m/z 576 [M+H]⁺.

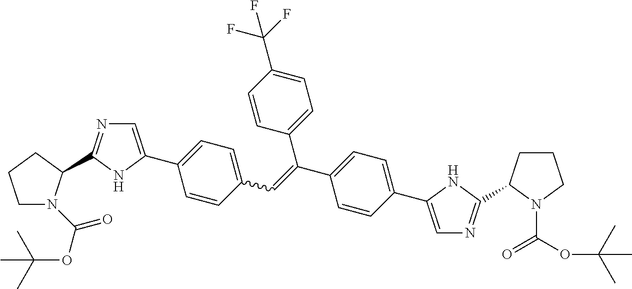

### Example 7F

### di-tert-butyl (2S,2′S)-2,2′-[{1-[4-(trifluoromethyl)phenyl]ethene-1,2-diyl}bis(4,1-phenylene-1H-imidazole-5,2-diyl)]dipyrrolidine-1-carboxylate (ACD v12)

A mixture of Example 7E (300 mg, 0.52 mmol), Example 4D (330 mg, 1.04 mmol), [1,1′-bis(diphenylphosphino)ferrocene]dichloropalladium(II) (Pd(dppf)Cl₂, 40 mg, 0.05 mmol), and K₂CO₃ (110 mg, 0.78 mmol) in N,N-dimethylformamide (8 mL) and water (2 mL) was stirred at 100° C. for 2 hours. The aqueous phase was extracted with ethyl acetate. The mixture was poured into water (50 mL) and extracted with dichloromethane. The combined organic layer was dried and concentrated. The residue was purified by silica gel column chromatography (petroleum ether:ethyl acetate=1:2) to afford 250 mg of a Z/E mixture of the title compound.

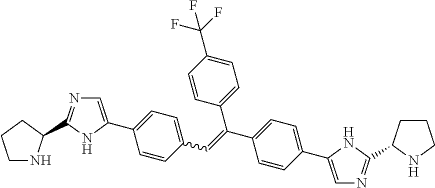

### Example 7G

### 5,5′-({(1-[4-(trifluoromethyl)phenyl]ethene-1,2-diyl}di-4,1-phenylene)bis{2-[(2S)-pyrrolidin-2-yl]-1H-imidazole} (ACD v12)

To a solution of Example 7F (200 mg, 0.25 mmol) in 3 mL of dichloromethane was added trifluoroacetic acid (3 mL), and the mixture was stirred at room temperature for 2 hours. The solvent was removed in vacuo. The residue was washed with aqueous NaHCO₃ (20 mL) and extracted with dichloromethane. The combined organic layer was dried and concentrated to afford 120 mg the title compound, that was used directly without purification. LC/MS m/z 595 [M+H]⁺.

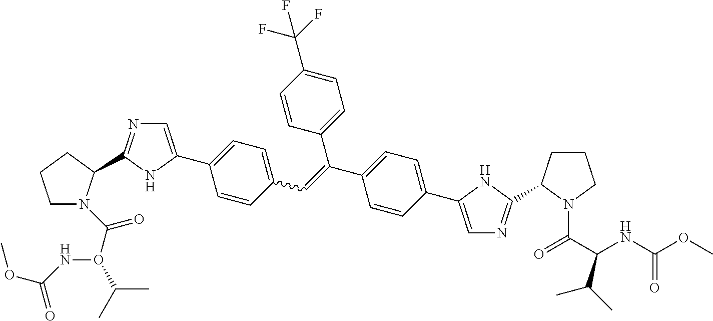

### Example 7H

### methyl [(2S)-1-{(2S)-2-[5-(4-{2-(4-{2-[(2S)-1-{(2S)-2-[(methoxycarbonyl)amino]-3-methylbutanoyl}pyrrolidin-2-yl]-1H-imidazol-5-yl}phenyl)-2-[4-(trifluoromethyl)phenyl]vinyl}phenyl)-1H-imidazol-2-yl]pyrrolidin-1-yl}-3-methyl-1-oxobutan-2-yl]carbamate (ACD v12)

A mixture of Example 7G (160 mg, 0.27 mmol), (S)-2-(methoxycarbonyl-amino)-3-methylbutanoic acid (94 mg, 0.54 mmol), diisopropylethylamine (139 mg, 1.07 mmol) and (benzotriazol-1-yloxy)tripyrrolidinophosphonium hexafluorophosphate (PyBOP®, 280 mg, 0.54 mmol) in N,N-dimethylformamide (5 mL) was stirred at room temperature for 3 hours. The solvent was removed in vacuo. The residue was washed with water (40 mL), and the aqueous phase was extracted by dichloromethane. The combined organic layer was dried and concentrated. The residue was purified by prep-HPLC (20%-95% acetonitrile/0.1% NH₄HCO₃ in H₂O) to afford 40 mg of the title compound. ¹HNMR (CDCl₃, 400 MHz) δ ppm 0.85-0.86 (m, 11H), 1.00-1.07 (m, 3H), 1.94-2.35 (m, 10H), 2.94-3.07 (m, 2H), 3.56-3.83 (m, 10H), 4.31-4.35 (m, 2H), 5.20-5.25 (m, 2H), 6.90-7.24 (m, 7H), 7.29-7.42 (m, 7H); LC/MS m/z 908 [M+H]⁺.

### Example 7I

### methyl [(2S)-1-{(2S)-2-[5-(4-{2-(4-{2-[(2S)-1-{(2S)-2-[(methoxycarbonyl)amino]-3-methylbutanoyl}pyrrolidin-2-yl]-1H-imidazol-5-yl}phenyl)-2-[4-(trifluoromethyl)phenyl]ethyl}phenyl)-1H-imidazol-2-yl]pyrrolidin-1-yl}-3-methyl-1-oxobutan-2-yl]carbamate

A mixture of Example 7H (100 mg, 0.11 mmol), Pd/C (10 mg) in CH₃OH (10 mL) was stirred under H₂ at 60° C. for 36 hours. The Pd/C was removed by filtration, and the solution was concentrated and the residue was purified by prep-HPLC (20%-95% acetonitrile/0.1% NH₄HCO₃ in H₂O) to afford 20 mg of the title compound. ¹HNMR (CDCl₃, 400 MHz) δ ppm 0.84-0.85 (m, 12H), 1.01-1.06 (m, 2H), 1.93-2.35 (m, 8H), 2.91-3.04 (m, 2H), 3.30-3.34 (m, 2H), 3.62-3.84 (m, 10H), 4.18-4.33 (m, 3H), 5.21-5.23 (m, 2H), 4.48-4.50 (m, 2H), 6.90-7.26 (m, 9H), 7.28-7.60 (m, 5H); LC/MS m/z 911 [M+H]⁺.

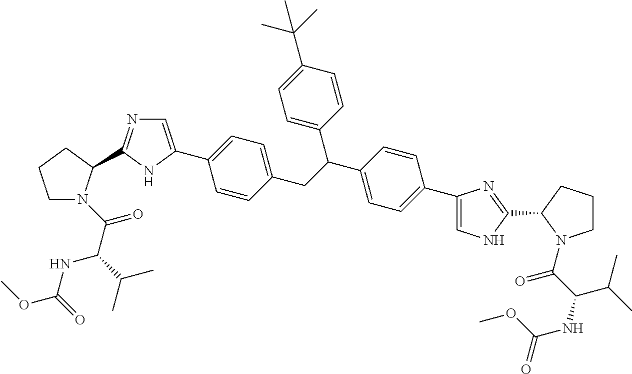

### Example 8

### methyl {(2S)-1-[(2S)-2-(5-{4-[2-(4-tert-butylphenyl)-2-(4-{2-[(2S)-1-{(2S)-2-[(methoxycarbonyl)amino]-3-methylbutanoyl}pyrrolidin-2-yl]-1H-imidazol-4-yl}phenyl)ethyl]phenyl}-1H-imidazol-2-yl)pyrrolidin-1-yl]-3-methyl-1-oxobutan-2-yl}carbamate

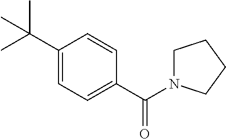

### Example 8A

### (4-tert-butylphenyl)(pyrrolidin-1-yl)methanone

A mixture of 4-tert-butylbenzoic acid (10 g, 56.2 mmol), oxalyl chloride (21.4 g, 168.5 mmol), and 0.5 mL of N,N-dimethylformamide in dichloromethane (100 mL) was stirred at 0° C. for 2 hours. The solvent was removed under reduced pressure to afford an intermediate acid chloride that was combined with pyrrolidine (4.4 g, 61.8 mmol) and triethylamine (6.2 g, 61.8 mmol) in dichloromethane (100 mL) and stirred at 0° C. for 0.5 hour then room temperature for 12 hours. The mixture was washed with water (50 mL). The organic layer was dried and concentrated. The residue was purified by gradient silica gel column chromatography (petroleum ether to petroleum ether: ethyl acetate=10:1) to afford 10.9 g of the title compound. LC/MS m/z 232 [M+H]⁺.

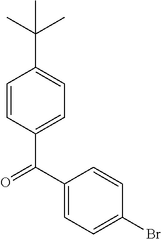

### Example 8B

### (4-bromophenyl)(4-tert-butylphenyl)methanone

n-Butyllithium (33.6 mL of a 1.6 M in hexane) was added to a solution of 1,4-dibromobenzene (12.7 g, 53.7 mmol) in diethyl ether (250 mL) at −78° C. After stirring for 2 hours at −78° C., Example 8A was added into the reaction mixture as a solid. The reaction mixture was allowed to warm up to room temperature for 12 hours. Water (100 mL) was added into the mixture. The aqueous layer was extracted with ethyl acetate. The combined organic phase was dried and concentrated. The residue was purified by gradient silica gel column chromatography (petroleum ether to petroleum ether: ethyl acetate=20:1) to afford 15.2 g of the title compound. LC/MS m/z 317 [M+H]⁺.

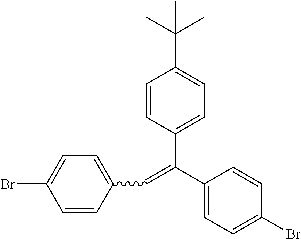

### Example 8C

### 4,4′-(1-(4-tert-butylphenyl)ethene-1,2-diyl)bis(bromobenzene)

Sodium hexamethyldisilazane (2 M in tetrahydrofuran) was added to a solution of Example 7C (4.3 g, 15.8 mmol) in tetrahydrofuran (50 mL) at 0° C. After 2 hours, Example 8B (5 g, 15.8 mmol) in 30 mL of tetrahydrofuran was added into the reaction. The mixture was stirred at 30° C. for 12 hours. The reaction mixture was poured into H₂O (80 mL) and extracted with ethyl acetate. The combined organic phase was dried and concentrated. The residue was purified by gradient silica gel column chromatography (petroleum ether to petroleum ether: ethyl acetate=10:1) to afford 6.4 g of the title compound. ¹HNMR (CDCl₃, 400 MHz) δ ppm 1.31/1.34 (s, 9H), 6.80-6.87 (m, 3H), 7.04 (d, 2H, J=8.4 Hz), 7.15-7.26 (m, 4H), 7.31-7.45 (m, 4H).

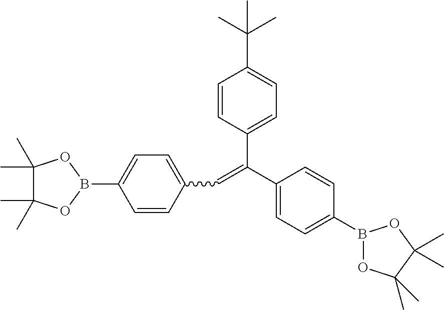

### Example 8D

### 2,2′-(4,4′-(1-(4-tert-butylphenyl)ethene-1,2-diyl)bis(4,1-phenylene))bis(4,4,5,5-tetramethyl-1,3,2-dioxaborolane)

A mixture of Example 8C (2 g, 4.25 mmol), bis(pinacolato)diboron (2.37 g, 9.36 mmol), [1,1′-bis(diphenylphosphino)ferrocene]dichloropalladium(II) (Pd(dppf)Cl₂, 903 mg, 1.105 mmol), and potassium acetate (2.5 g, 25.5 mmol) in dioxane (20 mL) was stirred at 100° C. for 2 hours. The mixture was poured into water (50 mL) and extracted with dichloromethane. The combined organic layer was dried and concentrated. The residue was purified by gradient silica gel column chromatography (petroleum ether to petroleum ether: ethyl acetate=10:1) to afford 2.35 g of the title compound. LC/MS m/z 565 [M+H]⁺.

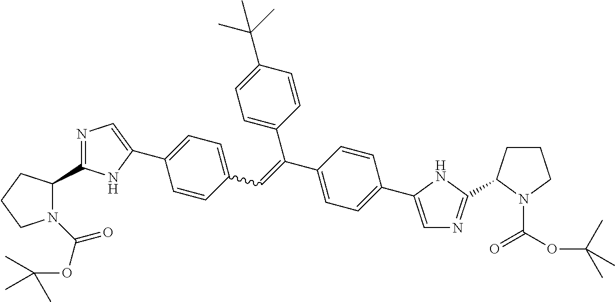

### Example 8E

### di-tert-butyl (2S,2′S)-2,2′-{[1-(4-tert-butylphenyl)ethene-1,2-diyl]bis(4,1-phenylene-1H-imidazole-5,2-diyl)}dipyrrolidine-1-carboxylate (ACD v12)

A mixture of Example 8D (895 mg, 1.58 mmol), Example 4D (1 g, 3.17 mmol), [1,1′-bis(diphenylphosphino)ferrocene]dichloropalladium(II) (Pd(dppf)Cl₂, 258 mg, 0.316 mmol), and K₂CO₃ (1.3 g, 9.48 mmol) in dioxane (30 mL) and water (10 mL) was stirred at 100° C. for 2 hours. The mixture was poured into water (50 mL) and extracted with dichloromethane. The combined organic layer was dried and concentrated. The residue was purified by gradient silica gel column chromatography (petroleum ether to petroleum ether:ethyl acetate=5:1 to petroleum ether:ethyl acetate=1:1) to afford 860 mg of the title compound as an E/Z mixture. LC/MS m/z 783 [M+H]⁺.

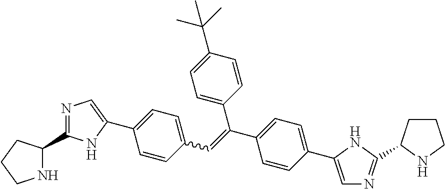

### Example 8F

### 5,5′-{[1-(4-tert-butylphenyl)ethene-1,2-diyl]di-4,1-phenylene}bis{2-[(2S)-pyrrolidin-2-yl]-1H-imidazole} (ACD v12)

To a solution of Example 8E (860 mg, 1.1 mmol) in 10 mL of dichloromethane was added trifluoroacetic acid (10 mL), and the mixture was stirred at room temperature for 2 hours. The solvent was removed in vacuo. The residue was washed with aqueous NaHCO₃ (20 mL) and extracted with dichloromethane. The combined organic layer was dried and concentrated to afford 610 mg of the title compound that was used directly without purification. LC/MS m/z 583 [M+H]⁺.

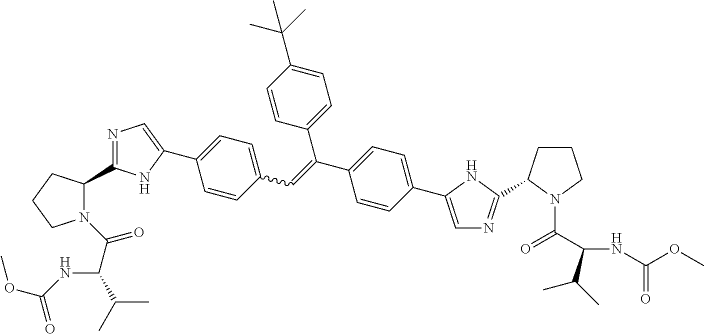

### Example 8G

### methyl {(2S)-1-[(2S)-2-(5-{4-[(E)-2-(4-tert-butylphenyl)-2-(4-{2-[(2S)-1-{(2S)-2-[(methoxycarbonyl)amino]-3-methylbutanoyl}pyrrolidin-2-yl]-1H-imidazol-5-yl}phenyl)vinyl]phenyl}-1H-imidazol-2-yl)pyrrolidin-1-yl]-3-methyl-1-oxobutan-2-yl}carbamate (ACD V12)

A mixture of Example 8F (600 mg, 1.03 mmol), (S)-2-(methoxycarbonyl-amino)-3-methylbutanoic acid (360 mg, 2.06 mmol), diisopropylethylamine (1.1 g, 8.24 mmol) and (benzotriazol-1-yloxy)tripyrrolidinophosphonium hexafluorophosphate (PyBOP®, 2.1 g, 4.12 mmol) in N,N-dimethylformamide (10 mL) was stirred at room temperature for 24 hours. The solvent was removed in vacuo. The residue was washed with water (40 mL) and the aqueous phase was extracted with dichloromethane. The combined organic layer was dried and concentrated. The residue was purified by prep-HPLC (20%-95% acetonitrile/0.1% NH₄HCO₃ in H₂O) to afford 170 mg of the title compound. ¹HNMR (CDCl₃, 400 MHz) δ ppm 0.85-0.86 (m, 12H), 1.00-1.07 (m, 2H), 1.32/1.35 (s, 9H), 1.94-2.35 (m, 10H), 2.88-2.95 (m, 2H), 3.67-3.68 (m, 6H), 3.82-3.84 (m, 2H), 4.31-4.35 (m, 2H), 5.20-5.25 (m, 2H), 5.58-5.63 (m, 2H), 6.90-7.24 (m, 6H), 7.29-7.42 (m, 5H); LC/MS m/z 897 [M+H]⁺.

### Example 8H

### methyl {(2S)-1-[(2S)-2-(5-{4-[2-(4-tert-butylphenyl)-2-(4-{2-[(2S)-1-{(2S)-2-[(methoxycarbonyl)amino]-3-methylbutanoyl}pyrrolidin-2-yl]-1H-imidazol-4-yl}phenyl)ethyl]phenyl}-1H-imidazol-2-yl)pyrrolidin-1-yl]-3-methyl-1-oxobutan-2-yl}carbamate

A mixture of Example 8G (90 mg, 0.1 mmol), Pd/C (20 mg) and a drop of acetic acid in CH₃OH (10 mL) was stirred under H₂ at 60° C. for 36 hours. The Pd/C was removed by filtration, and the solution was concentrated and the residue was purified by prep-HPLC (20%-95% acetonitrile/0.1% NH₄HCO₃ in H₂O) to afford 50 mg of the title compound. ¹H NMR (CDCl₃, 400 MHz) δ ppm 0.84-0.85 (m, 12H), 1.01-1.06 (m, 2H), 1.28 (s, 9H), 1.93-2.35 (m, 9H), 2.91-3.04 (m, 2H), 3.30-3.34 (m, 2H), 3.62-3.84 (m, 10H), 4.18-4.33 (m, 3H), 5.21-5.23 (m, 2H), 4.48-4.50 (m, 2H), 6.90-7.26 (m, 9H), 7.28-7.60 (m, 4H); LC/MS m/z 899 [M+H]⁺.

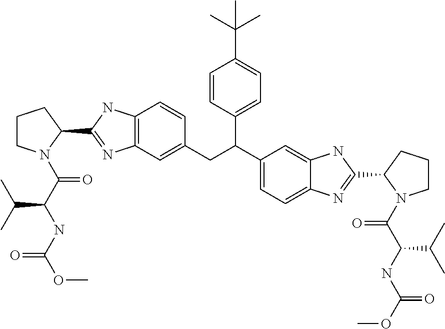

### Example 9

### methyl {(2S)-1-[(2S)-2-{6-[1-(4-tert-butylphenyl)-2-{2-[(2S)-1-{(2S)-2-[(methoxycarbonyl)amino]-3-methylbutanoyl}pyrrolidin-2-yl]-1H-benzimidazol-5-yl}ethyl]-1H-benzimidazol-2-yl}pyrrolidin-1-yl]-3-methyl-1-oxobutan-2-yl}carbamate

### Example 9A

### ((4-chloro-3-nitrophenyl)ethynyl)trimethylsilane

To a solution of 1-chloro-4-iodo-2-nitrobenzene (10 g, 35.6 mmol), and dichlorobis(triphenylphosphine)palladium (II) (0.495 g, 0.706 mmol) in triethylamine (130 mL) was added ethynyltrimethylsilane (6.35 mL, 45.9 mmol), and then the mixture was stirred for 10 minutes at room temperature. Copper(I) iodide (1.075 g, 5.64 mmol) was then added, and the solution was stirred at room temperature for 18 hours. The solution was then diluted with dichloromethane and filtered. The filtrate was concentrated, and then the residue was purified by chromatography (silica gel, ethyl acetate in hexanes) which afforded 7.77 g, (87%) of the title compound.

### Example 9B

### 1-chloro-4-ethynyl-2-nitrobenzene

To a solution of the product from Example 9A (7.77 g, 30.6 mmol) in methanol (200 mL) was added an aqueous solution of potassium carbonate (1.0 M, 136 mL, 136 mmol) and the mixture was stirred for 18 hours at room temperature. The solution was then concentrated. The residue was diluted water and extracted with dichloromethane. The organic extract was then dried, filtered, and concentrated to afford 4.13 g (74%) of the title compound.

### Example 9C

### 1,2-bis(4-chloro-3-nitrophenyl)ethyne

The product from Example 9B (4.13 g, 22.75 mmol) was processed using the method described in Example 9A substituting Example 9B for ethynyltrimethylsilaneto afford the title compound. MS (ESI) m/z 338 [M+H]⁺.

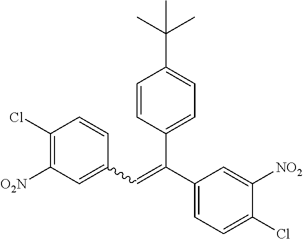

### Example 9D

### 4,4′-(1-(4-tert-butylphenyl)ethene-1,2-diyl)bis(1-chloro-2-nitrobenzene)

To a solution of the product from Example 9C (500 mg, 1.483 mmol), 4-tert-butylphenylboronic acid (396 mg, 2.23 mmol) and (acetylacetonato)dicarbonylrhodium(I) (19.1 mg, 0.074 mmol) in toluene (20 mL) and water (2 mL) was heated to 110° C. for 2 hours. The reaction mixture was diluted with ethyl acetate, and the mixture was extracted with water. The organic extract was then dried, filtered, concentrated and purified by chromatography (silica gel, 0-30% ethyl acetate in hexanes) to afford 300 mg (43%) of the title compound as a mixture of alkene isomers. MS (ESI) m/z 472 [M+H]⁺.

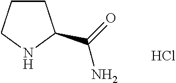

### Example 9E

### (S)-pyrrolidine-2-carboxamide hydrochloride salt

To (S)-tert-butyl 2-carbamoylpyrrolidine-1-carboxylate (29.8 g, 139 mmol) was added a solution of 4 N HCl in dioxane (209 mL, 836 mmol) and the resultant mixture was stirred at room temperature for 18 hours. The mixture was then concentrated and triturated with diethyl ether. The solid was collected by vacuum filtration and dried under vacuum to provide 21.6 g (104%) of the title compound as a colorless solid.

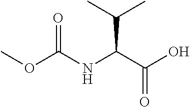

### Example 9F

### (S)-2-(methoxycarbonylamino)-3-methylbutanoic acid

To (S)-2-amino-3-methylbutanoic acid (57 g, 487 mmol) dissolved in dioxane (277 mL) was added a 2 N aqueous sodium hydroxide solution (803 mL, 1606 mmol) followed by the dropwise addition of methyl chloroformate (75 mL, 973 mmol) over 1 hour which caused warming of the solution to occur. After the addition, the mixture was heated at 60° C. for 22 hours, then cooled and extracted with dichloromethane (400 mL). The resultant aqueous layer was cooled in an ice bath then 12 N hydrochloric acid was added dropwise until the pH was 2. The resultant mixture was stirred at 0° C. for 2 hours, then the resultant solid was vacuum filtered and dried in a vacuum oven to provide 80 g (94%) of the title compound as a colorless solid. ¹H NMR (400 MHz, DMSO-d₆) δ ppm 12.50 (bs, 1H), 7.34 (d, J=8.6 Hz, 1H), 3.84 (dd, J=8.6, 6.0 Hz, 1H), 3.54 (s, 3H), 2.03 (m, 1H), 0.86 (t, J=7.0 Hz, 6H).

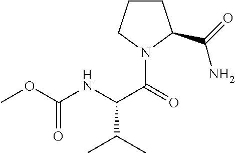

### Example 9G

### methyl (S)-14(S)-2-carbamoylpyrrolidin-1-yl)-3-methyl-1-oxobutan-2-ylcarbamate

The product of Example 9E (21.6 g, 144 mmol), the product of Example 9F (29.1 g, 166 mmol), 1H-benzo[d][1,2,3]triazol-1-ol hydrate (27.6 g, 180 mmol), N¹-((ethylimino)methylene)-N³,N³-dimethylpropane-1,3-diamine hydrochloride (34.6 g, 180 mmol) and 4-methylmorpholine (63.5 mL, 578 mmol) were dissolved in dichloromethane (960 mL) and stirred at room temperature for 18 hours. The resultant solution was then concentrated to a residue, water was then added and the solution was extracted with a 25% isopropanol in chloroform solution (2×2000 mL). The organic layer was washed with brine, and then the organic extract was dried over MgSO₄ and concentrated to a yellow oil which was purified by column chromatography eluting with a gradient of 0-10% methanol in dichloromethane to provide 25 g (64%) of the title compound as a colorless solid. ¹H NMR (400 MHz, DMSO-d₆) δ ppm 7.28 (m, 2H), 6.81 (s, 1H), 4.24 (dd, J=8.1, 4.4 Hz, 1H), 4.00 (t, J=8.4 Hz, 1H), 3.75 (m, 1H), 3.55 (m, 1H), 3.50 (s, 3H), 2.02 (m, 1H), 1.97 (m, 2H), 1.80 (m, 2H), 0.92 (d, J=6.7 Hz, 3H), 0.86 (d, J=8.6 Hz, 3H).

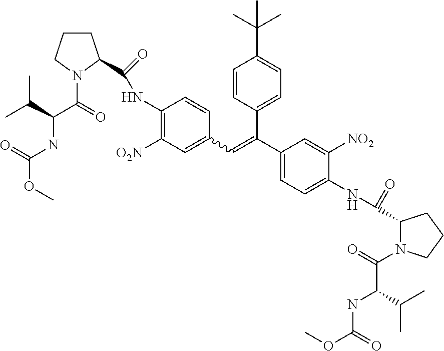

### Example 9H

### dimethyl ([1-(4-tert-butylphenyl)ethene-1,2-diyl]bis {(2-nitro-4,1-phenylene)carbamoyl(2S)pyrrolidine-2,1-diyl[(2S)-3-methyl-1-oxobutane-1,2-diyl]})biscarbamate (ACD V12)

A solution of the product from Example 9D (275 mg, 0.583 mmol), the product from Example 9G (396 mg, 1.459 mmol), tris(dibenzylideneacetone)dipalladium(0) (42.7 mg, 0.047 mmol), 4,5-bis(diphenylphosphino)-9,9-dimethylxanthene (40.5 mg, 0.070 mmol) and cesium carbonate (532 mg, 1.634 mmol) in dioxane (10 mL) was sparged with nitrogen gas for 15 minutes, and then the mixture was heated at 100° C. for 3 hours. After cooling, ethyl acetate was added, and the mixture was extracted with water. The organic extract was then dried, filtered, concentrated and purified by chromatography (silica gel, 30-100% ethyl acetate in hexanes) to afford 370 mg (67%) of the title compound as a mixture of alkene isomers. MS (ESI) m/z 942 [M+H]⁺.

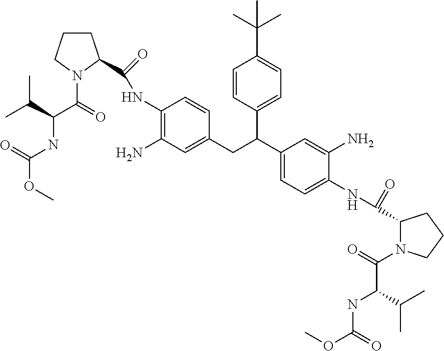

### Example 9I

### dimethyl ([1-(4-tert-butylphenyl)ethane-1,2-diyl]bis {(2-amino-4,1-phenylene)carbamoyl(2S)pyrrolidine-2,1-diyl[(2S)-3-methyl-1-oxobutane-1,2-diyl]})biscarbamate (ACD v12)

A mixture of Example 9H (350 mg, 0.372 mmol) and platinum(IV) oxide (25.3 mg, 0.112 mmol) in tetrahydrofuran (5 mL) and ethanol (5 mL) was evacuated and placed under hydrogen (balloon pressure) to reduce the nitro groups. Then 10% palladium on carbon (50 mg) was added and the hydrogenation resumed to reduce the double bond (˜4 days). The solids were removed by filtration, and the filtrate was concentrated to provide 190 mg (58%) of the title compound. MS (ESI) m/z 884 [M+H]⁺.

### Example 9J

### methyl {(2S)-1-[(2S)-2-{6-[1-(4-tert-butylphenyl)-2-{2-[(2S)-1-{(2S)-2-[(methoxycarbonyl)amino]-3-methylbutanoyl}pyrrolidin-2-yl]-1H-benzimidazol-5-yl}ethyl]-1H-benzimidazol-2-yl}pyrrolidin-1-yl]-3-methyl-1-oxobutan-2-yl}carbamate

A solution of the product from Example 9I (190 mg, 0.215 mmol) and acetic acid (0.062 mL, 1.076 mmol) in dioxane (4.5 mL) was heated at 70° C. for 23 hours. After cooling the mixture was concentrated and the resultant residue was diluted with acetonitrile and water (0.1% trifluoroacetic acid) and purified by reversed phase chromatography (C18), eluting with 10-100% acetonitrile in water (0.1% trifluoroacetic acid). The combined desired fractions were concentrated under vacuum to remove acetonitrile, then dichloromethane and aqueous sodium bicarbonate added. The organic layer was separated, dried, filtered and concentrated to afford 54 mg (30%) of the title compound as a mixture of diastereoisomers. ¹H NMR (400 MHz, DMSO-d₆) δ ppm 11.97 (m, 2H), 7.20 (m, 12H), 5.18 (m, 2H), 4.45 (m, 2H), 4.11 (m, 2H), 3.86 (m, 4H), 3.59 (s, 6H), 3.55 (m, 2H), 2.21 (m, 4H), 2.02 (m, 6H), 1.27 (s, 9H), 0.88 (m, 12H); MS (ESI) m/z 848 [M+H]⁺.

The title compounds of Examples 2, 4, 5, 7, 8, and 9 showed an EC50 of less than 1 nM in HCV 1b-Con-1 replicon assay; and the title compounds of Examples 1, 3, and 6 showed an EC50 of from 1 nM to 10 nM in HCV 1b-Con-1 replicon assay. Each compound's anti-HCV activity was determined by measuring the activity of the luciferase reporter gene in the replicon in the presence of 5% FBS. The luciferase reporter gene was placed under the translational control of the poliovirus IRES instead of the HCV IRES, and HuH-7 cells were used to support the replication of the replicon.

Moreover, the following compounds of Formula I can be similarly prepared according to the present invention,

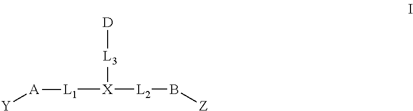

wherein A is selected from Table 1a, B is selected from Table 1b, D is selected from Tablet 2, Y and Z are each independently selected from Table 3, and A, B, and D are each independently optionally substituted with one or more R_{A}, and wherein X, L₁, L₂, L₃ and R_{A} are as described above. Preferably, X is C(H), L₁ is bond, L₂ is C₁-C₆alkylene (e.g., —(CH₂)—), and L₃ is a bond; or X is C(H), L₂ is bond, L₁ is C₁-C₆alkylene (e.g., —(CH₂)—), and L₃ is a bond; wherein said C₁-C₆alkylene is optionally substituted with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano, and R_{T}, R_{S}, and R_{S}′ are as defined above.

TABLE 1a
A

| | |
|---|---|
| 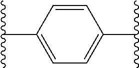 | 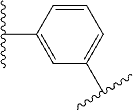 |
| 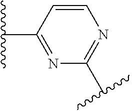 | 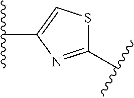 |
| 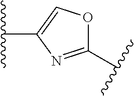 | 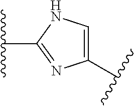 |
| 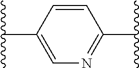 | 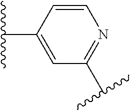 |
| 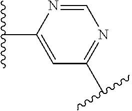 | 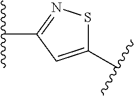 |
| 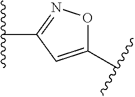 | 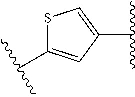 |
| 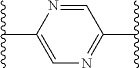 | 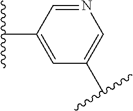 |
| 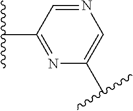 | 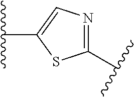 |
| 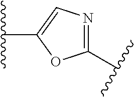 | 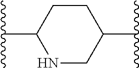 |
| 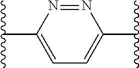 | 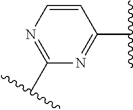 |
| 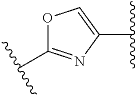 | 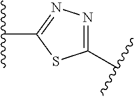 |
| 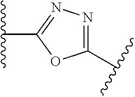 | 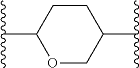 |
| 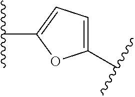 | 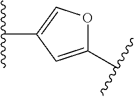 |
| 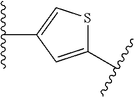 | 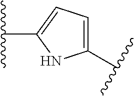 |
| 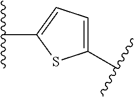 | 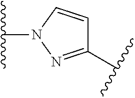 |
| 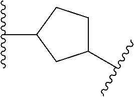 | 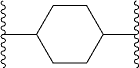 |
| 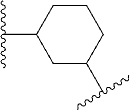 | 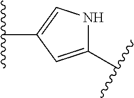 |
| 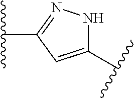 | 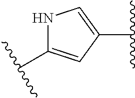 |
| 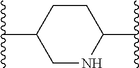 | 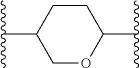 |
| 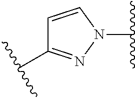 | 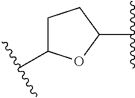 |
| 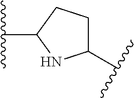 | 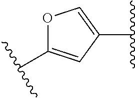 |
| 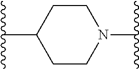 | 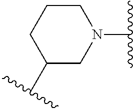 |
| 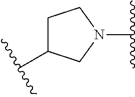 | 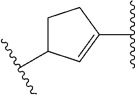 |
| 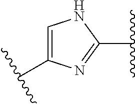 | 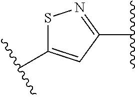 |
| 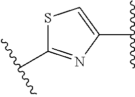 | 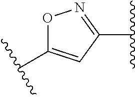 |
| 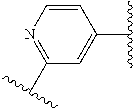 | 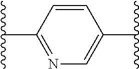 |
| 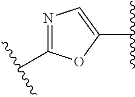 | 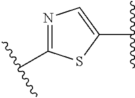 |
| 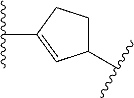 | 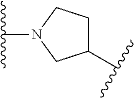 |
| 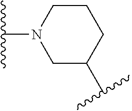 | 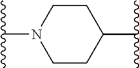 |
| 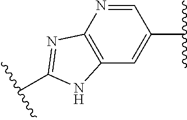 | 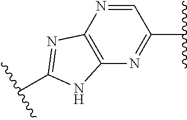 |
| 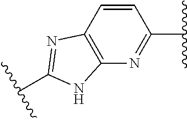 | 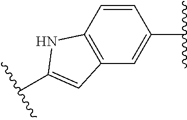 |
| 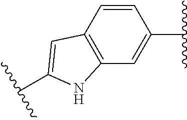 | 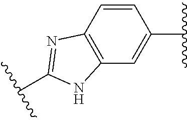 |
| 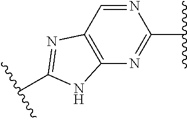 | 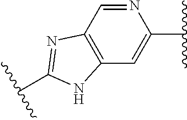 |
| 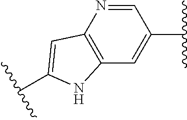 | 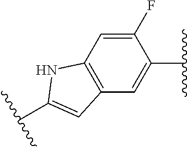 |
| 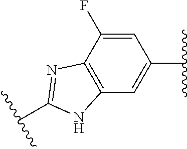 | 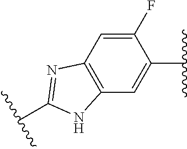 |

TABLE lb
B

| | |
|---|---|
| 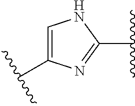 | 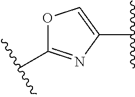 |
| 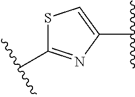 | 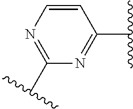 |
| 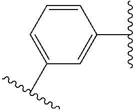 | 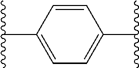 |
| 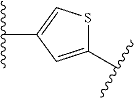 | 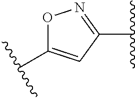 |
| 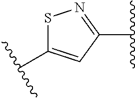 | 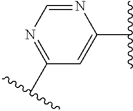 |
| 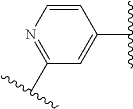 | 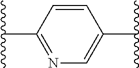 |
| 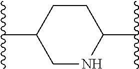 | 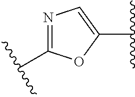 |
| 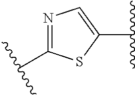 | 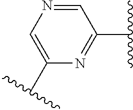 |
| 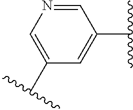 | 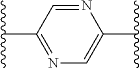 |
| 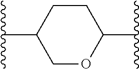 | 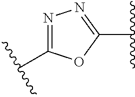 |
| 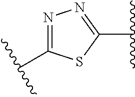 | 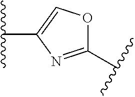 |
| 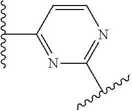 | 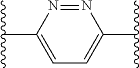 |
| 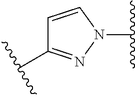 | 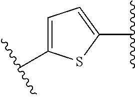 |
| 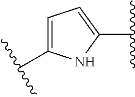 | 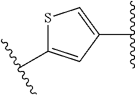 |
| 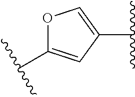 | 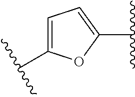 |
| 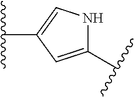 | 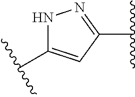 |
| 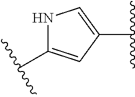 | 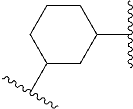 |
| 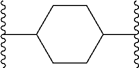 | 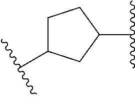 |
| 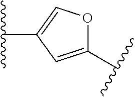 | 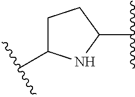 |
| 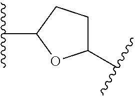 | 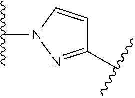 |
| 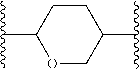 | 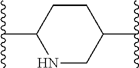 |
| 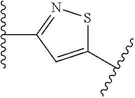 | 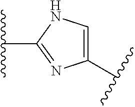 |
| 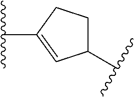 | 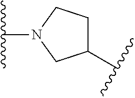 |
| 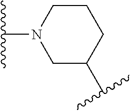 | 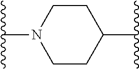 |
| 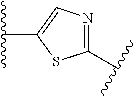 | 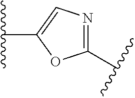 |
| 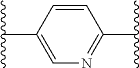 | 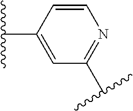 |
| 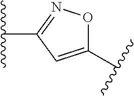 | 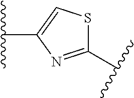 |
| 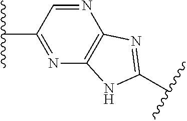 | 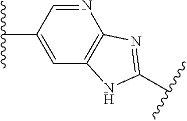 |
| 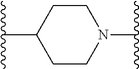 | 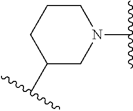 |
| 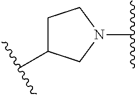 | 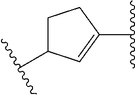 |
| 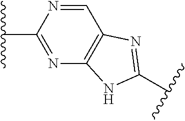 | 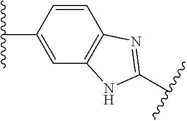 |
| 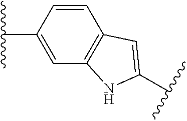 | 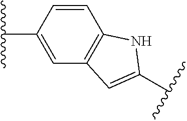 |
| 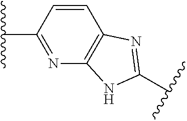 | 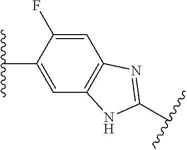 |
| 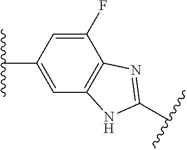 | 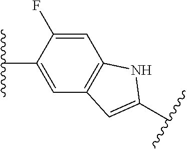 |
| 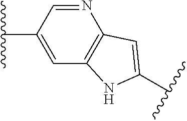 | 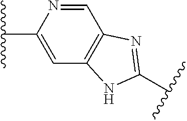 |

TABLE 2
D

| | | |
|---|---|---|
| 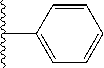 | 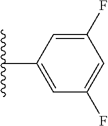 | 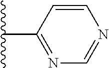 |
| 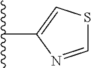 | 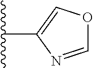 | 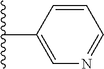 |
| 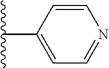 | 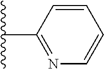 | 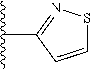 |
| 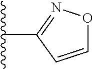 | 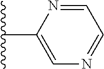 | 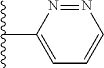 |
| 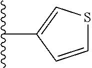 | 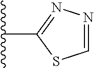 | 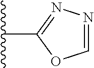 |
| 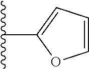 | 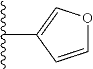 | 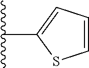 |
| 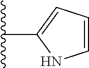 | 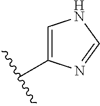 | 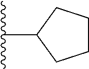 |
| 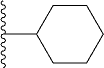 | 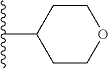 | 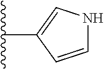 |
| 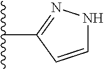 | 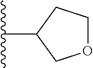 | 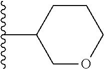 |
| 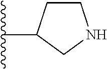 | 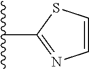 | 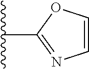 |
| 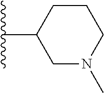 | 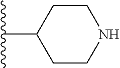 | 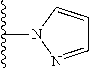 |
| 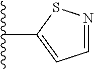 | 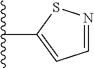 | 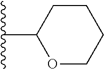 |
| 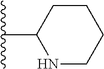 | 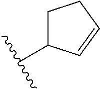 | 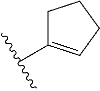 |
| 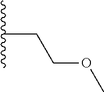 | 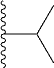 | 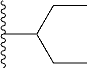 |
| 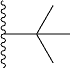 | 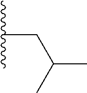 | 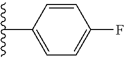 |
| 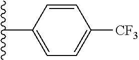 | 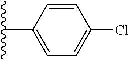 | 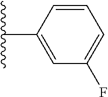 |

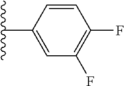

TABLE 3
Y and Z

| | |
|---|---|
| 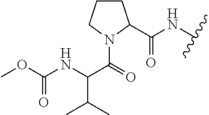 | 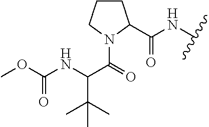 |
| 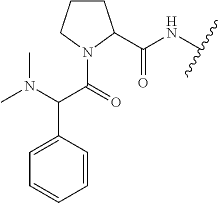 | 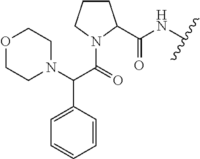 |
| 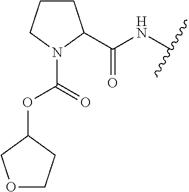 | 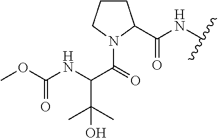 |
| 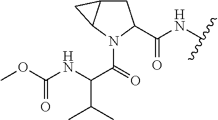 | 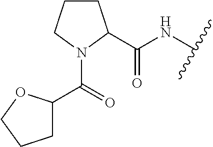 |
| 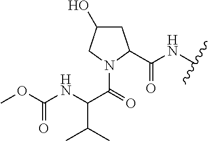 | 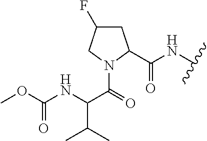 |
| 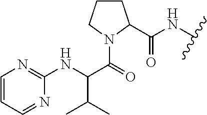 | 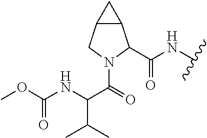 |
| 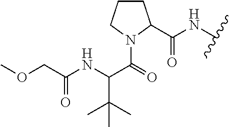 | 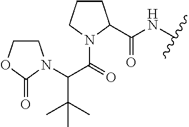 |
| 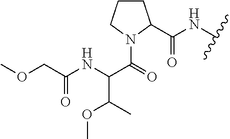 | 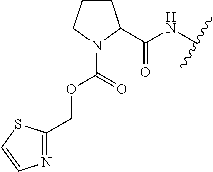 |
| 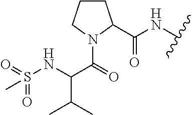 | 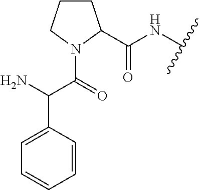 |
| 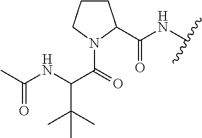 | 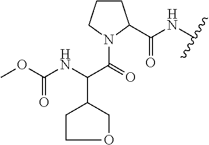 |
| 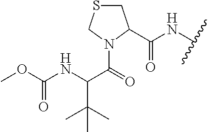 | 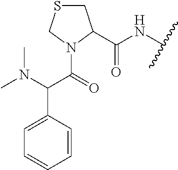 |
| 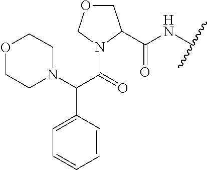 | 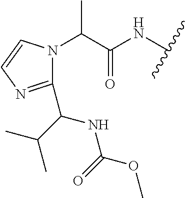 |
| 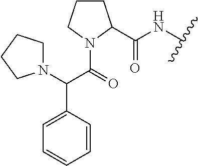 | 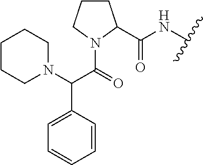 |
| 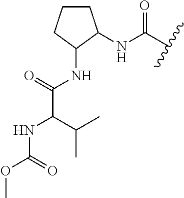 | 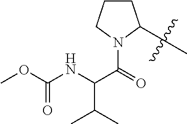 |
| 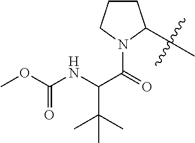 | 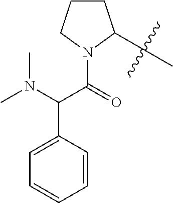 |
| 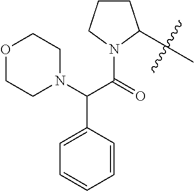 | 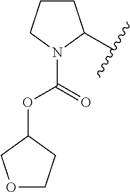 |
| 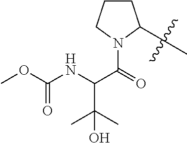 | 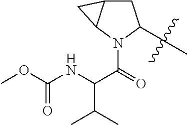 |
| 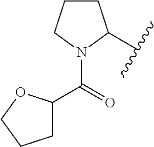 | 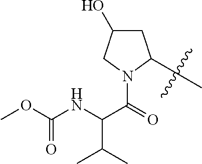 |
| 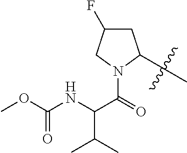 | 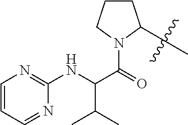 |
| 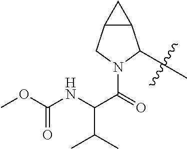 | 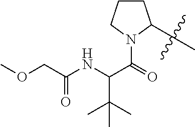 |
| 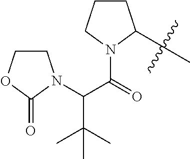 | 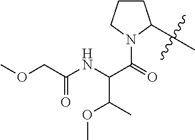 |
| 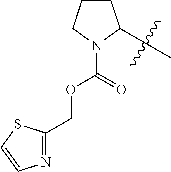 | 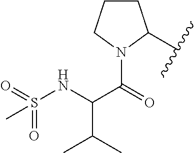 |
| 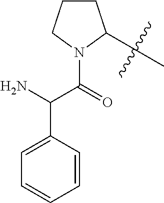 | 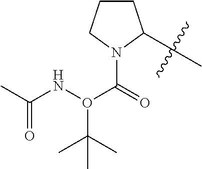 |
| 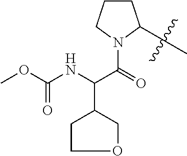 | 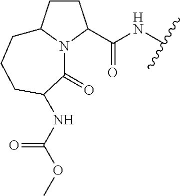 |
| 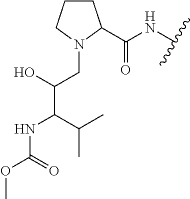 | 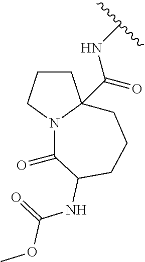 |
| 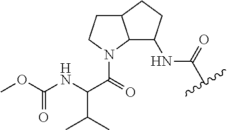 | 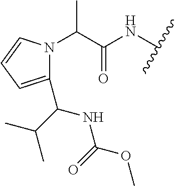 |
| 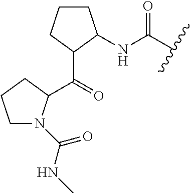 | 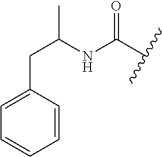 |
| 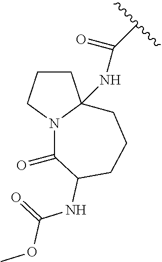 | 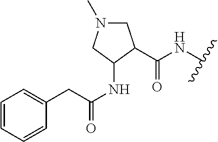 |
| 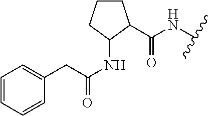 | 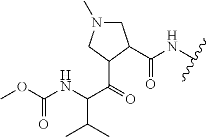 |
| 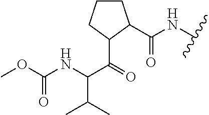 | 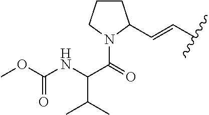 |
| 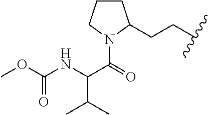 | 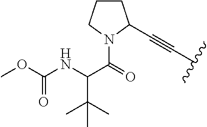 |
| 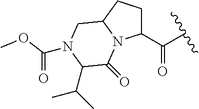 | 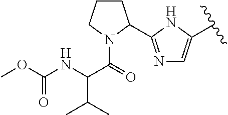 |
| 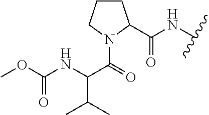 | 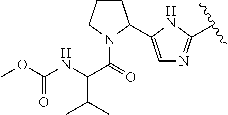 |

The inhibitory activities of the compounds of the present invention can be evaluated using a variety of assays known in the art. For instance, two stable subgenomic replicon cell lines can be used for compound characterization in cell culture: one derived from genotype 1a-H77 and the other derived from genotype 1b-Con1, obtained from University of Texas Medical Branch (Galveston, Tex.) and Apath, LLC (St. Louis, Mo.), respectively. The replicon constructs can be bicistronic subgenomic replicons. The genotype 1a replicon construct contains NS3-NS5B coding region derived from the H77 strain of HCV (1a-H77). The replicon also has a firefly luciferase reporter and a neomycin phosphotransferase (Neo) selectable marker. These two coding regions, separated by the FMDV 2a protease, comprise the first cistron of the bicistronic replicon construct, with the second cistron containing the NS3-NS5B coding region with addition of adaptive mutations E1202G, K1691R, K2040R and S2204I. The 1b-Con1 replicon construct is identical to the 1a-H77 replicon, except that HCV 5′ UTR, 3′ UTR, and the NS3-NS5B coding region are derived from the 1b-Con1 strain, and the adaptive mutations are K1609E, K1846T and Y3005C. In addition, the 1b-Con1 replicon construct contains a poliovirus IRES between the HCV IRES and the luciferase gene. Replicon cell lines can be maintained in Dulbecco's modified Eagles medium (DMEM) containing 10% (v/v) fetal bovine serum (FBS), 100 IU/ml penicillin, 100 mg/ml streptomycin (Invitrogen), and 200 mg/ml G418 (Invitrogen).

The inhibitory effects of the compounds of the invention on HCV replication can be determined by measuring activity of the luciferase reporter gene. For example, replicon-containing cells can be seeded into 96 well plates at a density of 5000 cells per well in 100 μl DMEM containing 5% FBS. The following day compounds can be diluted in dimethyl sulfoxide (DMSO) to generate a 200× stock in a series of eight half-log dilutions. The dilution series can then be further diluted 100-fold in the medium containing 5% FBS. Medium with the inhibitor is added to the overnight cell culture plates already containing 100 μl of DMEM with 5% FBS. In assays measuring inhibitory activity in the presence of human plasma, the medium from the overnight cell culture plates can be replaced with DMEM containing 40% human plasma and 5% FBS. The cells can be incubated for three days in the tissue culture incubators after which time 30 μl of Passive Lysis buffer (Promega) can be added to each well, and then the plates are incubated for 15 minutes with rocking to lyse the cells. Luciferin solution (100 μl, Promega) can be added to each well, and luciferase activity can be measured with a Victor II luminometer (Perkin-Elmer). The percent inhibition of HCV RNA replication can be calculated for each compound concentration and the EC₅₀ value can be calculated using nonlinear regression curve lifting to the 4-parameter logistic equation and GraphPad Prism 4 software. Using the above-described assays or similar cell-based replicon assays, representative compounds of the present invention showed significantly inhibitory activities against HCV replication.

The present invention also features pharmaceutical compositions comprising the compounds of the invention. A pharmaceutical composition of the present invention can comprise one or more compounds of the invention, each of which has Formula I (or I_{A}, I_{B}, I_{C}, I_{D}, I_{F}, I_{F}, I_{G}, I_{H} or I_{I}).

In addition, the present invention features pharmaceutical compositions comprising pharmaceutically acceptable salts, solvates, or prodrugs of the compounds of the invention. Without limitation, pharmaceutically acceptable salts can be zwitterions or derived from pharmaceutically acceptable inorganic or organic acids or bases. Preferably, a pharmaceutically acceptable salt retains the biological effectiveness of the free acid or base of the compound without undue toxicity, irritation, or allergic response, has a reasonable benefit/risk ratio, is effective for the intended use, and is not biologically or otherwise undesirable.

The present invention further features pharmaceutical compositions comprising a compound of the invention (or a salt, solvate or prodrug thereof) and another therapeutic agent. By way of illustration not limitation, these other therapeutic agents can be selected from antiviral agents (e.g., anti-HIV agents, anti-HBV agents, or other anti-HCV agents such as HCV protease inhibitors, HCV polymerase inhibitors, HCV helicase inhibitors, IRES inhibitors or NS5A inhibitors), anti-bacterial agents, anti-fungal agents, immunomodulators, anti-cancer or chemotherapeutic agents, anti-inflammation agents, antisense RNA, siRNA, antibodies, or agents for treating cirrhosis or inflammation of the liver. Specific examples of these other therapeutic agents include, but are not limited to, ribavirin, α-interferon, β-interferon, pegylated interferon-α, pegylated interferon-lambda, ribavirin, viramidine, R-5158, nitazoxanide, amantadine, Debio-025, NIM-811, R7128, R1626, R4048, T-1106, PSI-7851, PF-00868554, ANA-598, IDX184, IDX102, IDX375, GS-9190, VCH-759, VCH-916, MK-3281, BCX-4678, MK-3281, VBY708, ANA598, GL59728, GL60667, BMS-790052, BMS-791325, BMS-650032, GS-9132, ACH-1095, AP-H005, A-831, A-689, AZD2836, telaprevir, boceprevir, ITMN-191, BI-201335, VBY-376, VX-500 (Vertex), PHX-B, ACH-1625, IDX136, IDX316, VX-813 (Vertex), SCH 900518 (Schering-Plough), TMC-435 (Tibotec), ITMN-191 (Intermune, Roche), MK-7009 (Merck), IDX-PI (Novartis), BI-201335 (Boehringer Ingelheim), R7128 (Roche), PSI-7851 (Pharmasset), MK-3281 (Merck), PF-868554 (Pfizer), IDX-184 (Novartis), IDX-375 (Pharmasset), BILB-1941 (Boehringer Ingelheim), GS-9190 (Gilead), BMS-790052 (BMS), ABT-450, ABT-333, ABT-072, Albuferon (Novartis), ritonavir, another cytochrome P450 monooxygenase inhibitor, or any combination thereof.

In one embodiment, a pharmaceutical composition of the present invention comprises one or more compounds of the present invention (or salts, solvates or prodrugs thereof), and one or more other antiviral agents.

In another embodiment, a pharmaceutical composition of the present invention comprises one or more compounds of the present invention (or salts, solvates or prodrugs thereof), and one or more other anti-HCV agents. For example, a pharmaceutical composition of the present invention can comprise a compound(s) of the present invention having Formula I, I_{A}, I_{B}, I_{C}, I_{D}, I_{F}, I_{F}, I_{G}, I_{H} or I_{I} (or a salt, solvate or prodrug thereof), and an agent selected from HCV polymerase inhibitors (including nucleoside or non-nucleoside type of polymerase inhibitors), HCV protease inhibitors, HCV helicase inhibitors, CD81 inhibitors, cyclophilin inhibitors, IRES inhibitors, or NS5A inhibitors.

In yet another embodiment, a pharmaceutical composition of the present invention comprises one or more compounds of the present invention (or salts, solvates or prodrugs thereof), and one or more other antiviral agents, such as anti-HBV, anti-HIV agents, or anti-hepatitis A, anti-hepatitis D, anti-hepatitis E or anti-hepatitis G agents. Non-limiting examples of anti-HBV agents include adefovir, lamivudine, and tenofovir. Non-limiting examples of anti-HIV drugs include ritonavir, lopinavir, indinavir, nelfinavir, saquinavir, amprenavir, atazanavir, tipranavir, TMC-114, fosamprenavir, zidovudine, lamivudine, didanosine, stavudine, tenofovir, zalcitabine, abacavir, efavirenz, nevirapine, delavirdine, TMC-125, L-870812, S-1360, enfuvirtide, T-1249, or other HIV protease, reverse transcriptase, integrase or fusion inhibitors. Any other desirable antiviral agents can also be included in a pharmaceutical composition of the present invention, as appreciated by those skilled in the art.

A pharmaceutical composition of the present invention typically includes a pharmaceutically acceptable carrier or excipient. Non-limiting examples of suitable pharmaceutically acceptable carriers/excipients include sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, or phosphate buffer solutions. Lubricants, coloring agents, releasing agents, coating agents, sweetening, flavoring or perfuming agents, preservatives, or antioxidants can also be included in a pharmaceutical composition of the present invention.

The pharmaceutical compositions of the present invention can be formulated based on their routes of administration using methods well known in the art. For example, a sterile injectable preparation can be prepared as a sterile injectable aqueous or oleagenous suspension using suitable dispersing or wetting agents and suspending agents. Suppositories for rectal administration can be prepared by mixing drugs with a suitable nonirritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON'S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

Any compound described herein, or a pharmaceutically acceptable salt thereof, can be used to prepared pharmaceutical compositions of the present invention.

The present invention further features methods of using the compounds of the present invention (or salts, solvates or prodrugs thereof) to inhibit HCV replication. The methods comprise contacting cells infected with HCV virus with an effective amount of a compound of the present invention (or a salt, solvate or prodrug thereof), thereby inhibiting the replication of HCV virus in the cells. As used herein, “inhibiting” means significantly reducing, or abolishing, the activity being inhibited (e.g., viral replication). In many cases, representative compounds of the present invention can reduce the replication of HCV virus (e.g., in an HCV replicon assay as described above) by at least 10%, 20%, 30%, 40%, 50%, 60%, 70%, 80%, 90%, 95% or more.

The compounds of the present invention may inhibit one or more HCV subtypes. Examples of HCV subtypes that are amenable to the present invention include, but are not be limited to, HCV genotypes 1, 2, 3, 4, 5 and 6, including HCV genotypes 1a, 1b, 2a, 2b, 2c or 3a. In one embodiment, a compound or compounds of the present invention (or salts, solvates or prodrugs thereof) are used to inhibit the replication of HCV genotype 1a. In another embodiment, a compound or compounds of the present invention (or salts, solvates or prodrugs thereof) are used to inhibit the replication of HCV genotype 1b. In still another embodiment, a compound or compounds of the present invention (or salts, solvates or prodrugs thereof) are used to inhibit the replication of both HCV genotypes 1a and 1b.

The present invention also features methods of using the compounds of the present invention (or salts, solvates or prodrugs thereof) to treat HCV infection. The methods typically comprise administering a therapeutic effective amount of a compound of the present invention (or a salt, solvate or prodrug thereof), or a pharmaceutical composition comprising the same, to an HCV patient, thereby reducing the HCV viral level in the blood or liver of the patient. As used herein, the term “treating” refers to reversing, alleviating, inhibiting the progress of, or preventing the disorder or condition, or one or more symptoms of such disorder or condition to which such term applies. The term “treatment” refers to the act of treating. In one embodiment, the methods comprise administering a therapeutic effective amount of two or more compounds of the present invention (or salts, solvates or prodrugs thereof), or a pharmaceutical composition comprising the same, to an HCV patient, thereby reducing the HCV viral level in the blood or liver of the patient.

A compound of the present invention (or a salt, solvate or prodrug thereof) can be administered as the sole active pharmaceutical agent, or in combination with another desired drug, such as other anti-HCV agents, anti-HIV agents, anti-HBV agents, anti-hepatitis A agents, anti-hepatitis D agents, anti-hepatitis E agents, anti-hepatitis G agents, or other antiviral drugs. Any compound described herein, or a pharmaceutically acceptable salt thereof, can be employed in the methods of the present invention.

A compound of the present invention (or a salt, solvent or prodrug thereof) can be administered to a patient in a single dose or divided doses. A typical daily dosage can range, without limitation, from 0.1 to 200 mg/kg body weight, such as from 0.25 to 100 mg/kg body weight. Single dose compositions can contain these amounts or submultiples thereof to make up the daily dose. Preferably, each dosage contains a sufficient amount of a compound of the present invention that is effective in reducing the HCV viral load in the blood or liver of the patient. The amount of the active ingredient, or the active ingredients that are combined, to produce a single dosage form may vary depending upon the host treated and the particular mode of administration. It will be understood that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The present invention further features methods of using the pharmaceutical compositions of the present invention to treat HCV infection. The methods typically comprise administering a pharmaceutical composition of the present invention to an HCV patient, thereby reducing the HCV viral level in the blood or liver of the patient. Any pharmaceutical composition described herein can be used in the methods of the present invention.

In addition, the present invention features use of the compounds or salts of the present invention for the manufacture of medicaments for the treatment of HCV infection. Any compound described herein, or a pharmaceutically acceptable salt thereof, can be used to make medicaments of the present invention.

The compounds of the present invention can also be isotopically substituted. Preferred isotopic substitution include substitutions with stable or nonradioactive isotopes such as deuterium, ¹³C, ¹⁵N or ¹⁸O. Incorporation of a heavy atom, such as a substitution of deuterium for hydrogen, can give rise to an isotope effect that could alter the pharmacokinetics of the drug. In one example, at least 10 mol % of hydrogen in a compound of the present invention is substituted with deuterium. In another example, at least 25 mole % of hydrogen in a compound of the present invention is substituted with deuterium. In a further example, at least 50, 60, 70, 80 or 90 mole % of hydrogen in a compound of the present invention is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A compound of Formula I, or a pharmaceutically acceptable salt thereof, 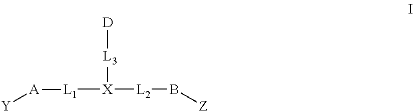
wherein:
A and B are each independently phenyl, and are each independently optionally substituted with one or more R_{A};
D is C₃-C₁₀carbocycle or 3- to 10-membered heterocycle, and is optionally substituted with one or more R_{A}; or D is selected from C₁-C₆alkyl, C₂-C₆alkenyl or C₂-C₆alkynyl, and is optionally substituted with one or more substituents selected from halogen, R_{T}, —O—R_{S}; —S—R_{S}, —N(R_{S}R_{S}′), —OC(O)R_{S}, —C(O)OR_{S}, nitro, phosphonoxy, phosphono, oxo, thioxo, formyl or cyano;
X is C(R_{C});
one of L₁ and L₂ is a bond and the other is —(CH₂)—, wherein the —(CH₂)— is optionally substituted with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
L₃ is bond;
Y is selected from —N(R_{B})C(O)C(R₁R₂)N(R₅)-T-R_{D} or —N(R_{B})C(O)C(R₃R₄)C(R₆R₇)-T-R_{D};
R₁ is R_{C}, and R₂ and R₅, taken together with the atoms to which they are attached, form a 3- to 8-membered heterocyclic ring which is optionally substituted with one or more R_{A};
R₃ and R₆ are each independently R_{C}, and R₄ and R₇, taken together with the atoms to which they are attached, form a 3- to 8-membered carbocyclic or heterocyclic ring which is optionally substituted with one or more R_{A};
Z is selected from —N(R_{B})C(O)C(R₈R₉)N(R₁₂)-T-R_{D} or —N(R_{B})C(O)C(R₁₀R₁₁)C(R₁₃R₁₄)-T-R_{D};
R₈ is R_{C}, and R₉ and R₁₂, taken together with the atoms to which they are attached, form a 3- to 8-membered heterocyclic ring which is optionally substituted with one or more R_{A};
R₁₀ and R₁₃ are each independently R_{C}, and R₁₁ and R₁₄, taken together with the atoms to which they are attached, form a 3- to 8-membered carbocyclic or heterocyclic ring which is optionally substituted with one or more R_{A};
T is each independently selected at each occurrence from a bond, -L_{S}-, -L_{S}-M-L_{S}′-, -L_{S}-M-L_{S}′-M′-L_{S}″-, wherein M and M′ are each independently selected at each occurrence from a bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₀carbocycle, or 3- to 10-membered heterocycle, and wherein said C₃-C₁₀carbocycle and 3- to 10-membered heterocycle are each independently optionally substituted at each occurrence with one or more R_{A};
R_{A} is independently selected at each occurrence from halogen, hydroxy, mercapto, amino, carboxy, nitro, oxo, phosphonoxy, phosphono, thioxo, formyl, cyano, -L_{A}, or -L_{S}-R_{E};
R_{B} and R_{B}′ are each independently selected at each occurrence from hydrogen or R_{F};
R_{C} is independently selected at each occurrence from hydrogen, halogen, hydroxy, mercapto, amino, carboxy, nitro, oxo, phosphonoxy, phosphono, thioxo, formyl, cyano, or R_{F};
R_{D} is each independently selected at each occurrence from hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, C₃-C₁₀carbocyclyl, or 3- to 10-membered heterocyclyl, wherein said C₃-C₁₀carbocyclyl and 3- to 10-membered heterocyclyl are each independently optionally substituted at each occurrence with one or more substituents selected from halogen, R_{T}, —O—R_{B}, —S—R_{B}, —N(R_{B}R_{B}′), —OC(O)R_{B}, —C(O)OR_{B}, nitro, phosphonoxy, phosphono, oxo, thioxo, formyl or cyano;
R_{F} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl or (3- or 6-membered heterocyclyl)C₁-C₆alkyl, each of which is independently optionally substituted at each occurrence with one or more substituents selected from halogen, hydroxy, mercapto, amino, carboxy, nitro, oxo, phosphonoxy, phosphono, thioxo, formyl or cyano;
L_{A} is independently selected at each occurrence from C₁-C₆alkyl, C₂-C₆alkenyl, or C₂-C₆alkynyl, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
L_{S}, L_{S}′ and L_{S}″ are each independently selected at each occurrence from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
R_{S}, R_{S}′ and R_{S}″ are each independently selected at each occurrence from hydrogen or R_{T};
R_{T} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl, or (3- or 6-membered heterocyclyl)C₁-C₆alkyl, each of which is independently optionally substituted at each occurrence with one or more substituents selected from halogen, R_{F}, —O—R_{B}, —S—R_{B}, —N(R_{B}R_{B}′), —OC(O)R_{B}, —C(O)OR_{B}, nitro, oxo, phosphonoxy, phosphono, thioxo, formyl or cyano.

2. The compound or salt of claim 1, wherein:
X is CH;
T is independently selected at each occurrence from —C(O)-L_{S}′-M′-L_{S}″- or —N(R_{B})C(O)-L_{S}′-M′-L_{S}″-; and
L_{S}′ is independently C₁-C₆alkylene, and is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano.

3. The compound or salt of claim 1, wherein:
Y is —N(R_{B})C(O)C(R₁R₂)N(R₅)-T-R_{D};
Z is —N(R_{B})C(O)C(R₈R₉)N(R₁₂)-T-R_{D};
T is independently selected at each occurrence from —C(O)-L_{S}′-M′-L_{S}″-; and
D is C₅-C₆carbocycle, 5- to 6-membered heterocycle, or 6- to 10-membered bicycles, and is optionally substituted with one or more R_{M}, where R_{M} is halogen, nitro, oxo, phosphonoxy, phosphono, thioxo, cyano, or -L_{S}-R_{E}.

4. The compound or salt of claim 3, wherein T is independently selected at each occurrence from —C(O)-L_{S}′-N(R_{B})C(O)-L_{S}″- or —C(O)-L_{S}′-N(R_{B})C(O)O-L_{S}″-.

5. The compound or salt of claim 3, wherein R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

6. A compound of Formula I, or a pharmaceutically acceptable salt thereof, 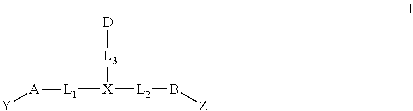
wherein: 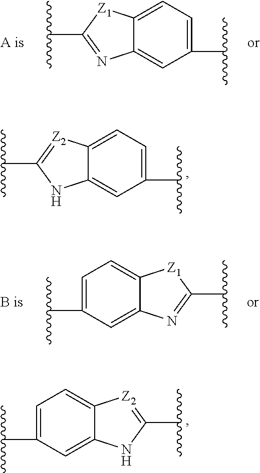
Z₁ is independently selected at each occurrence from O, S, NH or CH₂, Z₂ is independently selected at each occurrence from N or CH, wherein A and B are each independently optionally substituted with one or more R_{A};
D is C₃-C₁₀carbocycle or 3- to 10-membered heterocycle, and is optionally substituted with one or more R_{A}; or D is selected from C₁-C₆alkyl, C₂-C₆alkenyl or C₂-C₆alkynyl, and is optionally substituted with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
X is C(R_{C});
one of L₁ and L₂ is a bond and the other is —(CH₂)—, wherein the —(CH₂)— is optionally substituted with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
L₃ is bond;
Y is selected from —C(R₁R₂)N(R₅)-T-R_{D} or —C(R₃R₄)C(R₆R₇)-T-R_{D},
R₁ is R_{C}, and R₂ and R₅, taken together with the atoms to which they are attached, form a 3- to 8-membered heterocyclic ring which is optionally substituted with one or more R_{A};
R₃ and R₆ are each independently R_{C}, and R₄ and R₇, taken together with the atoms to which they are attached, form a 3- to 8-membered carbocyclic or heterocyclic ring which is optionally substituted with one or more R_{A};
Z is selected from —C(R₈R₉)N(R₁₂)-T-R_{D} or —C(R₁₀R₁₁)C(R₁₃R₁₄)-T-R_{D};
R₈ is R_{C}, and R₉ and R₁₂, taken together with the atoms to which they are attached, form a 3- to 8-membered heterocyclic ring which is optionally substituted with one or more R_{A};
R₁₀ and R₁₃ are each independently R_{C}, and R₁₁ and R₁₄, taken together with the atoms to which they are attached, form a 3- to 8-membered carbocyclic or heterocyclic ring which is optionally substituted with one or more R_{A};
T is each independently selected at each occurrence from a bond, -L_{S}-, -L_{S}-M-L_{S}′-, -L_{S}-M-L_{S}′-M′-L_{S}″-, wherein M and M′ are each independently selected at each occurrence from a bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₀carbocycle, or 3- to 10-membered heterocycle, and wherein said C₃-C₁₀carbocycle and 3- to 10-membered heterocycle are each independently optionally substituted at each occurrence with one or more R_{A};
R_{A} is independently selected at each occurrence from halogen, hydroxy, mercapto, amino, carboxy, nitro, oxo, phosphonoxy, phosphono, thioxo, formyl, cyano, -L_{A}, or -L_{S}-R_{E};
R_{B} and R_{B}′ are each independently selected at each occurrence from hydrogen or R_{F};
R_{C} is independently selected at each occurrence from hydrogen, halogen, hydroxy, mercapto, amino, carboxy, nitro, oxo, phosphonoxy, phosphono, thioxo, formyl, cyano, or R_{F};
R_{D} is each independently selected at each occurrence from hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, C₃-C₁₀carbocyclyl, or 3- to 10-membered heterocyclyl, wherein said C₃-C₁₀carbocyclyl and 3- to 10-membered heterocyclyl are each independently optionally substituted at each occurrence with one or more substituents selected from halogen, R_{T}, —O—R_{B}, —S—R_{B}, —N(R_{B}R_{B}′), —OC(O)R_{B}, —C(O)OR_{B}, nitro, phosphonoxy, phosphono, oxo, thioxo, formyl or cyano;
R_{F} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl or (3- or 6-membered heterocyclyl)C₁-C₆alkyl, each of which is independently optionally substituted at each occurrence with one or more substituents selected from halogen, hydroxy, mercapto, amino, carboxy, nitro, oxo, phosphonoxy, phosphono, thioxo, formyl or cyano;
L_{A} is independently selected at each occurrence from C₁-C₆alkyl, C₂-C₆alkenyl, or C₂-C₆alkynyl, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
L_{S}, L_{S}′ and L_{S}″ are each independently selected at each occurrence from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
R_{S}, R_{S}′ and R_{S}″ are each independently selected at each occurrence from hydrogen or R_{T};
R_{T} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl, or (3- or 6-membered heterocyclyl)C₁-C₆alkyl, each of which is independently optionally substituted at each occurrence with one or more substituents selected from halogen, R_{F}, —O—R_{B}, —S—R_{B}, —N(R_{B}R_{B}′), —OC(O)R_{B}, —C(O)OR_{B}, nitro, oxo, phosphonoxy, phosphono, thioxo, formyl or cyano.

7. The compound or salt of claim 6, wherein:
X is CH;
Z₁ is NH, and Z₂ is N;
T is independently selected at each occurrence from —C(O)-L_{S}′-M′-L_{S}″- or —N(R_{B})C(O)-L_{S}′-M′-L_{S}″-; and
L_{S}′ is independently C₁-C₆alkylene, and is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano.

8. The compound or salt of claim 6, wherein:
Z₁ is NH, and Z₂ is N;
Y is —C(R₁R₂)N(R₅)-T-R_{D};
Z is —C(R₈R₉)N(R₁₂)-T-R_{D};
T is independently selected at each occurrence from —C(O)-L_{S}′-M′-L_{S}″-; and
D is C₅-C₆carbocycle, 5- to 6-membered heterocycle, or 6- to 10-membered bicycles, and is optionally substituted with one or more R_{M}, where R_{M} is halogen, nitro, oxo, phosphonoxy, phosphono, thioxo, cyano, or -L_{S}-R_{E}.

9. The compound or salt of claim 8, wherein T is independently selected at each occurrence from —C(O)-L_{S}′-N(R_{B})C(O)-L_{S}″- or —C(O)-L_{S}′-N(R_{B})C(O)O-L_{S}″-.

10. The compound or salt of claim 8, wherein R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

11. A compound of Formula I, or a pharmaceutically acceptable salt thereof, 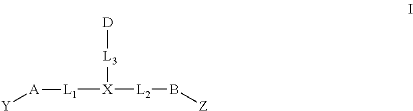
wherein:
A and B are each independently phenyl, and are each independently optionally substituted with one or more R_{A};
D is C₃-C₁₀carbocycle or 3- to 10-membered heterocycle, and is optionally substituted with one or more R_{A}; or D is selected from C₁-C₆alkyl, C₂-C₆alkenyl or C₂-C₆alkynyl, and is optionally substituted with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
X is C(R_{C});
one of L₁ and L₂ is a bond and the other is —(CH₂)—, wherein the —(CH₂)— is optionally substituted with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
L₃ is bond;
Y is selected from -G-C(R₁R₂)N(R₅)-T-R_{D};
R₁ is R_{C}, and R₂ and R₅, taken together with the atoms to which they are attached, form a 3- to 8-membered heterocyclic ring which is optionally substituted with one or more R_{A};
R₃ and R₆ are each independently R_{C}, and R₄ and R₇, taken together with the atoms to which they are attached, form a 3- to 8-membered carbocyclic or heterocyclic ring which is optionally substituted with one or more R_{A};
Z is selected from -G-C(R₈R₉)N(R₁₂)-T-R_{D};
R₈ is R_{C}, and R₉ and R₁₂, taken together with the atoms to which they are attached, form a 3- to 8-membered heterocyclic ring which is optionally substituted with one or more R_{A};
R₁₀ and R₁₃ are each independently R_{C}, and R₁₁ and R₁₄, taken together with the atoms to which they are attached, form a 3- to 8-membered carbocyclic or heterocyclic ring which is optionally substituted with one or more R_{A};
G is 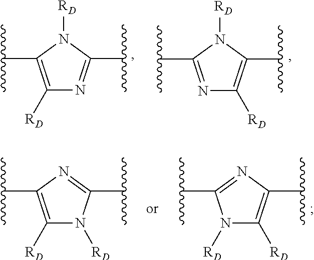
T is each independently selected at each occurrence from a bond, -L_{S}-, -L_{S}-M-L_{S}′-, -L_{S}-M-L_{S}′-M′-L_{S}″-, wherein M and M′ are each independently selected at each occurrence from a bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₀carbocycle, or 3- to 10-membered heterocycle, and wherein said C₃-C₁₀carbocycle and 3- to 10-membered heterocycle are each independently optionally substituted at each occurrence with one or more R_{A};
R_{A} is independently selected at each occurrence from halogen, hydroxy, mercapto, amino, carboxy, nitro, oxo, phosphonoxy, phosphono, thioxo, formyl, cyano, -L_{A}, or -L_{S}-R_{E};
R_{B} and R_{B}′ are each independently selected at each occurrence from hydrogen or R_{F};
R_{C} is independently selected at each occurrence from hydrogen, halogen, hydroxy, mercapto, amino, carboxy, nitro, oxo, phosphonoxy, phosphono, thioxo, formyl, cyano, or R_{F};
R_{D} is each independently selected at each occurrence from hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, C₃-C₁₀carbocyclyl, or 3- to 10-membered heterocyclyl, wherein said C₃-C₁₀carbocyclyl and 3- to 10-membered heterocyclyl are each independently optionally substituted at each occurrence with one or more substituents selected from halogen, R_{T}, —O—R_{B}, —S—R_{B}, —N(R_{B}R_{B}′), —OC(O)R_{B}, —C(O)OR_{B}, nitro, phosphonoxy, phosphono, oxo, thioxo, formyl or cyano;
R_{F} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl or (3- or 6-membered heterocyclyl)C₁-C₆alkyl, each of which is independently optionally substituted at each occurrence with one or more substituents selected from halogen, hydroxy, mercapto, amino, carboxy, nitro, oxo, phosphonoxy, phosphono, thioxo, formyl or cyano;
L_{A} is independently selected at each occurrence from C₁-C₆alkyl, C₂-C₆alkenyl, or C₂-C₆alkynyl, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
L_{S}, L_{S}′ and L_{S}″ are each independently selected at each occurrence from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
R_{S}, R_{S}′ and R_{S}″ are each independently selected at each occurrence from hydrogen or R_{T}; and
R_{T} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl, or (3- or 6-membered heterocyclyl)C₁-C₆alkyl, each of which is independently optionally substituted at each occurrence with one or more substituents selected from halogen, R_{F}, —O—R_{B}, —S—R_{B}, —N(R_{B}R_{B}′), —OC(O)R_{B}, —C(O)OR_{B}, nitro, oxo, phosphonoxy, phosphono, thioxo, formyl or cyano.

12. The compound or salt of claim 11, wherein:
X is CH;
G is 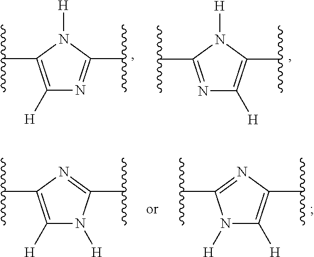
T is independently selected at each occurrence from —C(O)-L_{S}′-M′-L_{S}″- or —N(R_{B})C(O)-L_{S}′-M′-L_{S}″-; and
L_{S}′ is independently C₁-C₆alkylene, and is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano.

13. The compound or salt of claim 11, wherein:
X is CH; 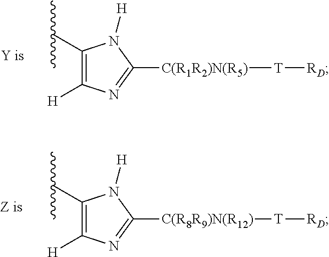
T is independently selected at each occurrence from —C(O)-L_{S}′-M′-L_{S}″-; and
D is C₅-C₆carbocycle, 5- to 6-membered heterocycle, or 6- to 10-membered bicycles, and is optionally substituted with one or more R_{M}, where R_{M} is halogen, nitro, oxo, phosphonoxy, phosphono, thioxo, cyano, or -L_{S}-R_{E}.

14. The compound or salt of claim 12, wherein T is independently selected at each occurrence from —C(O)-L_{S}′-N(R_{B})C(O)-L_{S}″- or —C(O)-L_{S}′-N(R_{B})C(O)O-L_{S}″-.

15. The compound or salt of claim 12, wherein R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

16. A compound of Formula I, or a pharmaceutically acceptable salt thereof, 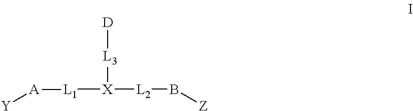
wherein:
A and B are each independently phenyl, and are each independently optionally substituted with one or more R_{A};
D is C₃-C₁₀carbocycle or 3- to 10-membered heterocycle, and is optionally substituted with one or more R_{A}; or D is selected from C₁-C₆alkyl, C₂-C₆alkenyl or C₂-C₆alkynyl, and is optionally substituted with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
X is C(R_{C});
one of L₁ and L₂ is a bond and the other is —(CH₂)—, wherein the —(CH₂)— is optionally substituted with one or more substituents selected from halogen, R_{T}, —O—R_{S}, —S—R_{S}, —N(R_{S}R_{S}′), —OC(O)R_{S}, —C(O)OR_{S}, nitro, oxo, phosphonoxy, phosphono, thioxo, formyl or cyano;
L₃ is bond;
Y is —N(R_{B})C(O)C(R₁R₂)N(R₅)-T-R_{D} and Z is -G-C(R₈R₉)N(R₁₂)-T-R_{D}; or
Y is -G-C(R₁R₂)N(R₅)-T-R_{D} and Z is —N(R_{B})C(O)C(R₈R₉)N(R₁₂)-T-R_{D};
R₁ is R_{C}, and R₂ and R₅, taken together with the atoms to which they are attached, form a 3- to 8-membered heterocyclic ring which is optionally substituted with one or more R_{A};
R₃ and R₆ are each independently R_{C}, and R₄ and R₇, taken together with the atoms to which they are attached, form a 3- to 8-membered carbocyclic or heterocyclic ring which is optionally substituted with one or more R_{A};
R₈ is R_{C}, and R₉ and R₁₂, taken together with the atoms to which they are attached, form a 3- to 8-membered heterocyclic ring which is optionally substituted with one or more R_{A};
R₁₀ and R₁₃ are each independently R_{C}, and R₁₁ and R₁₄, taken together with the atoms to which they are attached, form a 3- to 8-membered carbocyclic or heterocyclic ring which is optionally substituted with one or more R_{A};
T is each independently selected at each occurrence from a bond, -L_{S}-, -L_{S}-M-L_{S}′-, -L_{S}-M-L_{S}′-M′-L_{S}″-, wherein M and M′ are each independently selected at each occurrence from a bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, —N(R_{B})S(O)N(R_{B}′)—, C₃-C₁₀carbocycle, or 3- to 10-membered heterocycle, and wherein said C₃-C₁₀carbocycle and 3- to 10-membered heterocycle are each independently optionally substituted at each occurrence with one or more R_{A};
R_{A} is independently selected at each occurrence from halogen, hydroxy, mercapto, amino, carboxy, nitro, oxo, phosphonoxy, phosphono, thioxo, formyl, cyano, -L_{A}, or -L_{S}-R_{E};
R_{B} and R_{B}′ are each independently selected at each occurrence from hydrogen or R_{F};
R_{C} is independently selected at each occurrence from hydrogen, halogen, hydroxy, mercapto, amino, carboxy, nitro, oxo, phosphonoxy, phosphono, thioxo, formyl, cyano, or R_{F};
R_{D} is each independently selected at each occurrence from hydrogen or R_{A};
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —C(O)N(R_{S})C(O)—R_{S}′, C₃-C₁₀carbocyclyl, or 3- to 10-membered heterocyclyl, wherein said C₃-C₁₀carbocyclyl and 3- to 10-membered heterocyclyl are each independently optionally substituted at each occurrence with one or more substituents selected from halogen, R_{T}, —O—R_{B}, —S—R_{B}, —N(R_{B}R_{B}′), —OC(O)R_{B}, —C(O)OR_{B}, nitro, phosphonoxy, phosphono, oxo, thioxo, formyl or cyano;
R_{F} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl or (3- or 6-membered heterocyclyl)C₁-C₆alkyl, each of which is independently optionally substituted at each occurrence with one or more substituents selected from halogen, hydroxy, mercapto, amino, carboxy, nitro, oxo, phosphonoxy, phosphono, thioxo, formyl or cyano;
L_{A} is independently selected at each occurrence from C₁-C₆alkyl, C₂-C₆alkenyl, or C₂-C₆alkynyl, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
L_{S}, L_{S}′ and L_{S}″ are each independently selected at each occurrence from a bond; or C₁-C₆alkylene, C₂-C₆alkenylene, or C₂-C₆alkynylene, each of which is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano;
R_{S}, R_{S}′ and R_{S}″ are each independently selected at each occurrence from hydrogen or R_{T}; and
R_{T} is independently selected at each occurrence from C₁-C₆alkyl, C₂-C₆alkenyl, C₂-C₆alkynyl, C₃-C₆carbocyclyl, C₃-C₆carbocyclylC₁-C₆alkyl, 3- to 6-membered heterocyclyl, or (3- or 6-membered heterocyclyl)C₁-C₆alkyl, each of which is independently optionally substituted at each occurrence with one or more substituents selected from halogen, R_{F}, —O—R_{B}, —S—R_{B}, —N(R_{B}R_{B}′), —OC(O)R_{B}, —C(O)OR_{B}, nitro, oxo, phosphonoxy, phosphono, thioxo, formyl or cyano.

17. The compound or salt of claim 16, wherein:
X is CH;
G is 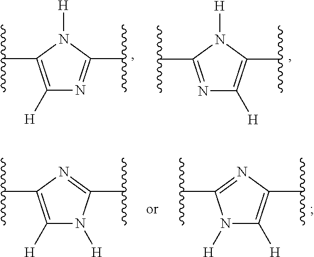
T is independently selected at each occurrence from —C(O)-L_{S}′-M′-L_{S}″- or —N(R_{B})C(O)-L_{S}′-M′-L_{S}″-; and
L_{S}′ is independently C₁-C₆alkylene, and is independently optionally substituted at each occurrence with one or more substituents selected from halogen, R_{T}, —O—R_{S}, —S—R_{S}, —N(R_{S}R_{S}′), —OC(O)R_{S}, —C(O)OR_{S}, nitro, phosphonoxy, phosphono, oxo, thioxo, formyl or cyano.

18. The compound or salt of claim 16, wherein:
X is CH;
Y is 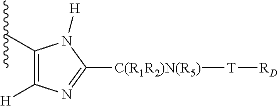
and Z is —N(R_{B})C(O)C(R₈R₉)N(R₁₂)-T-R_{D}; or
Y is —N(R_{B})C(O)C(R₁R₂)N(R₅)-T-R_{D} and Z is 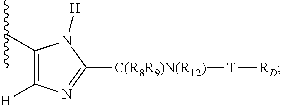
T is independently selected at each occurrence from —C(O)-L_{S}′-M′-L_{S}″-; and
D is C₅-C₆carbocycle, 5- to 6-membered heterocycle, or 6- to 10-membered bicycles, and is optionally substituted with one or more R_{M}, where R_{M} is halogen, nitro, oxo, phosphonoxy, phosphono, thioxo, cyano, or -L_{S}-R_{E}.

19. The compound or salt of claim 17, wherein T is independently selected at each occurrence from —C(O)-L_{S}′-N(R_{B})C(O)-L_{S}″- or —C(O)-L_{S}′-N(R_{B})C(O)O-L_{S}″-.

20. The compound or salt of claim 17, wherein R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or

21. A pharmaceutical composition comprising a compound or salt of claim 1.

22. The pharmaceutical composition of claim 21, further comprising a HCV protease inhibitor, a HCV plolymerase inhibitor, or another anti-HCV agent.

23. A method of treating HCV infection, comprising administering to an HCV patient a compound or salt of claim 1.

24. A process of making a compound of claim 1, comprising a step described in one of the schemes described hereinabove.
